(19) United States
(12) Patent Application Publication
(10) Pub. No.: US 2025/0059185 A1
(43) Pub. Date: Feb. 20, 2025

(54) BICYCLIC HETEROCYCLIC COMPOUNDS USEFUL AS MONOACYLGLYCEROL LIPASE INHIBITORS

(71) Applicant: Hoffmann-La Roche Inc., Little Falls, NJ (US)

(72) Inventors: Joerg BENZ, Rheinfelden (DE); Maude GIROUD, Basel (CH); Uwe GRETHER, Efringen-Kirchen (DE); Bernd KUHN, Reinach BL (CH); Fionn Susannah O'HARA, Basel (CH); Matthias Beat WITTWER, Riehen (CH)

(73) Assignee: Hoffmann-La Roche Inc., Little Falls, NJ (US)

(21) Appl. No.: 18/720,022

(22) PCT Filed: Dec. 14, 2022
(86) PCT No.: PCT/EP2022/085744

§ 371 Date: Jun. 14, 2024

(30) Foreign Application Priority Data
Dec. 16, 2021 (EP) 21215130.2

(51) Int. Cl. C07D 471/04; A61K 9/20; A61K 9/48; A61K 31/437; A61K 31/444; A61K 31/497; A61K 31/4985; A61K 31/506; A61K 31/675; C07D 487/04; C07D 519/00; C07F 9/6561

(52) U.S. Cl. CPC C07D 471/04; A61K 9/2054; A61K 9/2059; A61K 9/4858; A61K 9/4866; A61K 31/437; A61K 31/444; A61K 31/497; A61K 31/4985; A61K 31/506; A61K 31/675; C07D 487/04; C07D 519/00; C07F 9/6561

(57) ABSTRACT

The invention provides new MAGL inhibitors having the general formula (II)AB(II) wherein the variables are as described herein, compositions including the compounds, processes of manufacturing the compounds and methods of using the compounds.

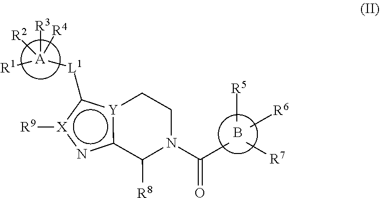

### FIELD OF THE INVENTION

The present invention relates to organic compounds useful for therapy or prophylaxis in a mammal, and in particular to monoacylglycerol lipase (MAGL) inhibitors that are useful for the treatment or prophylaxis of diseases or conditions that are associated with MAGL, e.g., neuroinflammation, neurodegenerative diseases, pain, cancer, mental disorders, multiple sclerosis, Alzheimer's disease, Parkinson's disease, amyotrophic lateral sclerosis, traumatic brain injury, neurotoxicity, stroke, epilepsy, anxiety, migraine, depression, inflammatory bowel disease, inflammatory bowel symptoms, gut motility, visceral pain, fibromyalgia, endometriosis, abdominal pain, abdominal pain associated with irritable bowel syndrome, asthma, COPD, and/or visceral pain.

### BACKGROUND OF THE INVENTION

Endocannabinoids (ECs) are signaling lipids that exert their biological actions by interacting with cannabinoid receptors (CBRs), CB1 and CB2. They modulate multiple physiological processes including neuroinflammation, neurodegeneration and tissue regeneration (Iannotti, F. A., et al., Progress in lipid research 2016, 62, 107-28.). In the brain, the main endocannabinoid, 2-arachidonoylglycerol (2-AG), is produced by diacyglycerol lipases (DAGL) and hydrolyzed by the monoacylglycerol lipase, MAGL. MAGL hydrolyses 85% of 2-AG; the remaining 15% being hydrolysed by ABHD6 and ABDH12 (Nomura, D. K., et al., Science 2011, 334, 809.). MAGL is expressed throughout the brain and in most brain cell types, including neurons, astrocytes, oligodendrocytes and microglia cells (Chanda, P. K., et al., Molecular pharmacology 2010, 78, 996; Viader, A., et al., Cell reports 2015, 12, 798.). 2-AG hydrolysis results in the formation of arachidonic acid (AA), the precursor of prostaglandins (PGs) and leukotrienes (LTs). Oxidative metabolism of AA is increased in inflamed tissues. There are two principal enzyme pathways of arachidonic acid oxygenation involved in inflammatory processes, the cyclooxygenase which produces PGs and the 5-lipoxygenase which produces LTs. Of the various cyclooxygenase products formed during inflammation, PGE2 is one of the most important. These products have been detected at sites of inflammation, e.g. in the cerebrospinal fluid of patients suffering from neurodegenerative disorders and are believed to contribute to inflammatory response and disease progression. Mice lacking MAGL (Mgll−/−) exhibit dramatically reduced 2-AG hydrolase activity and elevated 2-AG levels in the nervous system while other arachidonoyl-containing phospho- and neutral lipid species including anandamide (AEA), as well as other free fatty acids, are unaltered. Conversely, levels of AA and AA-derived prostaglandins and other eicosanoids, including prostaglandin E2 (PGE2), D2 (PGD2), F2 (PGF2), and thromboxane B2 (TXB2), are strongly decreased. Phospholipase A₂ (PLA₂) enzymes have been viewed as the principal source of AA, but cPLA₂-deficient mice have unaltered AA levels in their brain, reinforcing the key role of MAGL in the brain for AA production and regulation of the brain inflammatory process.

Neuroinflammation is a common pathological change characteristic of diseases of the brain including, but not restricted to, neurodegenerative diseases (e.g. multiple sclerosis, Alzheimer's disease, Parkinson disease, amyotrophic lateral sclerosis, traumatic brain injury, neurotoxicity, stroke, epilepsy and mental disorders such as anxiety and migraine). In the brain, production of eicosanoids and prostaglandins controls the neuroinflammation process. The pro-inflammatory agent lipopolysaccharide (LPS) produces a robust, time-dependent increase in brain eicosanoids that is markedly blunted in Mgll−/− mice. LPS treatment also induces a widespread elevation in pro-inflammatory cytokines including interleukin-1-a (IL-1-a), IL-1b, IL-6, and tumor necrosis factor-a (TNF-α) that is prevented in Mgll−/− mice.

Neuroinflammation is characterized by the activation of the innate immune cells of the central nervous system, the microglia and the astrocytes. It has been reported that anti-inflammatory drugs can suppress in preclinical models the activation of glia cells and the progression of disease including Alzheimer's disease and multiple sclerosis (Lleo A., Cell Mol Life Sci. 2007, 64, 1403.). Importantly, genetic and/or pharmacological disruption of MAGL activity also blocks LPS-induced activation of microglial cells in the brain (Nomura, D. K., et al., Science 2011, 334, 809.).

In addition, genetic and/or pharmacological disruption of MAGL activity was shown to be protective in several animal models of neurodegeneration including, but not restricted to, Alzheimer's disease, Parkinson's disease and multiple sclerosis. For example, an irreversible MAGL inhibitor has been widely used in preclinical models of neuroinflammation and neurodegeneration (Long, J. Z., et al., Nature chemical biology 2009, 5, 37.). Systemic injection of such inhibitor recapitulates the Mgll−/− mice phenotype in the brain, including an increase in 2-AG levels, a reduction in AA levels and related eicosanoids production, as well as the prevention of cytokines production and microglia activation following LPS-induced neuroinflammation (Nomura, D. K., et al., Science 2011, 334, 809.), altogether confirming that MAGL is a druggable target.

Consecutive to the genetic and/or pharmacological disruption of MAGL activity, the endogenous levels of the MAGL natural substrate in the brain, 2-AG, are increased. 2-AG has been reported to show beneficial effects on pain with, for example, anti-nociceptive effects in mice (Ignatowska-Jankowska B. et al., J. Pharmacol. Exp. Ther. 2015, 353, 424.) and on mental disorders, such as depression in chronic stress models (Zhong P. et al., Neuropsychopharmacology 2014, 39, 1763.).

Furthermore, oligodendrocytes (OLs), the myelinating cells of the central nervous system, and their precursors (OPCs) express the cannabinoid receptor 2 (CB2) on their membrane. 2-AG is the endogenous ligand of CB1 and CB2 receptors. It has been reported that both cannabinoids and pharmacological inhibition of MAGL attenuate OLs's and OPCs's vulnerability to excitotoxic insults and therefore may be neuroprotective (Bernal-Chico, A., et al., Glia 2015, 63, 163.). Additionally, pharmacological inhibition of MAGL increases the number of myelinating OLs in the brain of mice, suggesting that MAGL inhibition may promote differentiation of OPCs in myelinating OLs in vivo (Alpar, A., et al., Nature communications 2014, 5, 4421.). Inhibition of MAGL was also shown to promote remyelination and functional recovery in a mouse model of progressive multiple sclerosis (Feliu A. et al., Journal of Neuroscience 2017, 37 (35), 8385.).

In addition, in recent years, metabolism is talked highly important in cancer research, especially the lipid metabolism. Researchers believe that the de novo fatty acid synthesis plays an important role in tumor development. Many studies illustrated that endocannabinoids have anti-tumorigenic actions, including anti-proliferation, apoptosis induction and anti-metastatic effects. MAGL as an important decomposing enzyme for both lipid metabolism and the endocannabinoids system, additionally as a part of a gene expression signature, contributes to different aspects of tumourigenesis, including in glioblastoma (Qin, H., et al., Cell Biochem. Biophys. 2014, 70, 33; Nomura D K et al., Cell 2009, 140(1), 49-61; Nomura D K et al., Chem. Biol. 2011, 18(7), 846-856, Jinlong Yin et al, Nature Communications 2020, 11, 2978).

The endocannabinoid system is also involved in many gastrointestinal physiological and physiopathological actions (Marquez, Suarez et al. 2009). All these effects are driven mainly via cannabinoid receptors (CBRs), CB1 and CB2. CB1 receptors are present throughout the GI tract of animals and healthy humans, especially in the enteric nervous system (ENS) and the epithelial lining, as well as smooth muscle cells of blood vessels in the colonic wall (Wright, Rooney et al. 2005), (Duncan, Davison et al. 2005). Activation of CB1 produces anti-emetic, anti-motility, and anti-inflammatory effect, and help to modulate pain (Perisetti, Rimu et al. 2020). CB2 receptors are expressed in immune cells such as plasma cells and macrophages, in the lamina propria of the GI tract (Wright, Rooney et al. 2005), and primarily on the epithelium of human colonic tissue associated with inflammatory bowel disease (IBD). Activation of CB2 exerts anti-inflammatory effect by reducing pro-inflammatory cytokines. Expression of MAGL is increased in colonic tissue in UC patients (Marquez, Suarez et al. 2009) and 2-AG levels are increased in plasma of IBD patients (Grill, Hogenauer et al. 2019). Several animal studies have demonstrated the potential of MAGL inhibitors for symptomatic treatment of IBD. MAGL inhibition prevents TNBS-induced mouse colitis and decreases local and circulating inflammatory markers via a CB1/CB2 MoA (Marquez, Suarez et al. 2009). Furthermore, MAGL inhibition improves gut wall integrity and intestinal permeability via a CB1 driven MoA (Wang, Zhang et al. 2020).

In conclusion, suppressing the action and/or the activation of MAGL is a promising new therapeutic strategy for the treatment or prevention of a multitude of diseases and disorders.

WO2020065613 discloses certain MAGL inhibitors. However, it was found that, while those MAGL inhibitors have properties that may make them suitable for the treatment of CNS indications, such as depression and pain, some properties (e.g. high passive permeability, Pₐₚₚ) mean that they are less suitable for indications that require or at least would benefit from achieving higher exposures in target tissues than in the rest of the body, such as inflammatory bowel disease.

Accordingly, there continues to be a high unmet medical need for new MAGL inhibitors, especially for new MAGL inhibitors with distinct properties such that different exposures can be achieved in different tissues.

### SUMMARY OF THE INVENTION

Surprisingly, starting from the compounds disclosed in WO2020065613, a number of chemical modifications enabled a marked reduction in passive permeability (Pₐₚₚ), while maintaining high cellular potency and excellent overall drug-like properties.

In a first aspect, the present invention provides compounds of formula (II)

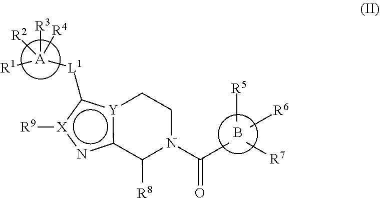

wherein the variables are as defined herein.

In further aspects, the present invention provides pharmaceutical compositions including the compounds, processes for manufacturing the compounds and methods of using the compounds in the treatment or prevention of diseases and disorders that are associated with MAGL.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The compound of formula (II) as described herein, wherein X is NR⁹ and Y is C, is represented by formula (I)

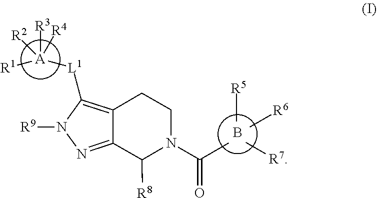

The compound of formula (II) as described herein, wherein X is CR⁹ and Y is N, is represented by formula (IIa)

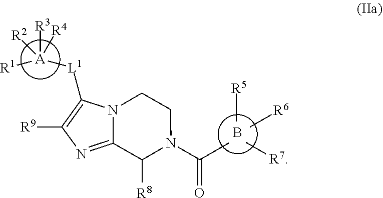

The compound of formula (II) as described herein, wherein X and Y are both N, is represented by formula (IIb)

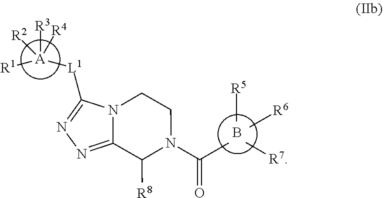

The term “alkyl” refers to a mono- or multivalent, e.g., a mono- or bivalent, linear or branched saturated hydrocarbon group of 1 to 12 carbon atoms. In some preferred embodiments, the alkyl group contains 1 to 6 carbon atoms (“C₁₋₆-alkyl”), e.g., 1, 2, 3, 4, 5, or 6 carbon atoms. In other embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. Particularly preferred, yet non-limiting examples of alkyl are methyl, tert-butyl, and 2,2-dimethylpropyl.

The term “alkoxy” refers to an alkyl group, as previously defined, attached to the parent molecular moiety via an oxygen atom. Unless otherwise specified, the alkoxy group contains 1 to 12 carbon atoms. In some preferred embodiments, the alkoxy group contains 1 to 6 carbon atoms (“C₁0.6-alkoxy”). In other embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “alkoxyalkoxy” refers to an alkoxy group, wherein at least one of the hydrogen atoms of the alkoxy group has been replaced by an alkoxy group. Preferably, “alkoxyalkoxy” refers to an alkoxy group wherein 1, 2 or 3 hydrogen atoms of the alkoxy group have been replaced by an alkoxy group. A particularly preferred, yet non-limiting example of alkoxyalkoxy is 2-methoxyethoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “hydroxy” refers to a group —OH.

The term “carbamoyl” refers to a group —C(O)NH₂.

The term “alkylsulfonimidoyl” refers to a group

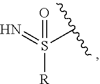

wherein R is alkyl.

The term “cycloalkyl” as used herein refers to a saturated or partly unsaturated monocyclic or bicyclic hydrocarbon group of 3 to 10 ring carbon atoms (“C₃₋₁₀-cycloalkyl”). In some preferred embodiments, the cycloalkyl group is a saturated monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom.

Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl, cycloheptyl, 1-bicyclo[1.1.1]pentanyl, norbornanyl, and 1-bicyclo[2.2.2]octanyl. A particularly preferred, yet non-limiting example of cycloalkyl is cyclopropyl.

The term “triazolyl” is understood to include all possible permutations of three nitrogen atoms in a 5-membered heteroaromatic cycle. In particular, “triazolyl” includes 2H-triazol-4-yl, 1H-1,2,4-triazolyl, 1H-triazolyl, and 4H-1,2,4-triazolyl.

The term “halocycloalkyl” refers to a cycloalkyl group, wherein at least one of the hydrogen atoms of the cycloalkyl group has been replaced by a halogen atom, preferably fluoro. Preferably, “halocycloalkyl” refers to a cycloalkyl group wherein 1, 2 or 3 hydrogen atoms of the cycloalkyl group have been replaced by a halogen atom, most preferably fluoro. Particularly preferred, yet non-limiting examples of halocycloalkyl are 2-fluorocyclopropyl and 2,2-difluorocyclopropyl.

The term “cyano” refers to a —CN (nitrile) group.

The term “oxo” refers to a group ═O.

The term “(halo)alkylsulfonimidoyl” refers to a group

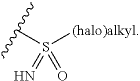

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Particularly preferred, yet non-limiting examples of haloalkyl are trifluoromethyl, difluoromethyl, 1,1-difluoroethyl, 2,2-difluoroethyl, and 2,2,2-trifluoroethyl.

The term “hydroxyalkyl” refers to an alkyl group, wherein at least one of the hydrogen atoms of the alkyl group has been replaced by a hydroxy group. Preferably, “hydroxyalkyl” refers to an alkyl group wherein 1, 2 or 3 hydrogen atoms of the alkyl group have been replaced by a hydroxy group. A particularly preferred, yet non-limiting example of hydroxyalkyl is 1-hydroxyethyl.

The term “carbamoylalkyl” refers to an alkyl group, wherein at least one of the hydrogen atoms of the alkyl group has been replaced by a carbamoyl group. Preferably, “carbamoylalkyl” refers to an alkyl group wherein 1, 2 or 3 hydrogen atoms of the alkyl group have been replaced by a carbamoyl group. A particularly preferred, yet non-limiting examples of carbamoylalkyl is 2-amino-2-oxo-ethyl.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The abbreviation “MAGL” refers to the enzyme monoacylglycerol lipase. The terms “MAGL” and “monoacylglycerol lipase” are used herein interchangeably.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “neuroinflammation” as used herein relates to acute and chronic inflammation of the nervous tissue, which is the main tissue component of the two parts of the nervous system; the brain and spinal cord of the central nervous system (CNS), and the branching peripheral nerves of the peripheral nervous system (PNS). Chronic neuroinflammation is associated with neurodegenerative diseases such as Alzheimer's disease, Parkinson's disease and multiple sclerosis. Acute neuroinflammation usually follows injury to the central nervous system immediately, e.g., as a result of traumatic brain injury (TBI).

The term “traumatic brain injury” (“TBI”, also known as “intracranial injury”), relates to damage to the brain resulting from external mechanical force, such as rapid acceleration or deceleration, impact, blast waves, or penetration by a projectile.

The term “neurodegenerative diseases” relates to diseases that are related to the progressive loss of structure or function of neurons, including death of neurons. Examples of neurodegenerative diseases include, but are not limited to, multiple sclerosis, Alzheimer's disease, Parkinson's disease and amyotrophic lateral sclerosis.

The term “mental disorders” (also called mental illnesses or psychiatric disorders) relates to behavioral or mental patterns that may cause suffering or a poor ability to function in life. Such features may be persistent, relapsing and remitting, or occur as a single episode. Examples of mental disorders include, but are not limited to, anxiety and depression.

The term “pain” relates to an unpleasant sensory and emotional experience associated with actual or potential tissue damage. Examples of pain include, but are not limited to, nociceptive pain, chronic pain (including idiopathic pain), neuropathic pain including chemotherapy induced neuropathy, phantom pain and phsychogenic pain. A particular example of pain is neuropathic pain, which is caused by damage or disease affecting any part of the nervous system involved in bodily feelings (i.e., the somatosensory system). In one embodiment, “pain” is neuropathic pain resulting from amputation or thoracotomy. In one embodiment, “pain” is chemotherapy induced neuropathy.

The term “neurotoxicity” relates to toxicity in the nervous system. It occurs when exposure to natural or artificial toxic substances (neurotoxins) alter the normal activity of the nervous system in such a way as to cause damage to nervous tissue. Examples of neurotoxicity include, but are not limited to, neurotoxicity resulting from exposure to substances used in chemotherapy, radiation treatment, drug therapies, drug abuse, and organ transplants, as well as exposure to heavy metals, certain foods and food additives, pesticides, industrial and/or cleaning solvents, cosmetics, and some naturally occurring substances.

The term “cancer” refers to a disease characterized by the presence of a neoplasm or tumor resulting from abnormal uncontrolled growth of cells (such cells being “cancer cells”). As used herein, the term cancer explicitly includes, but is not limited to, hepatocellular carcinoma, colon carcinogenesis and ovarian cancer.

The term “mammal” as used herein includes both humans and non-humans and includes but is not limited to humans, non-human primates, canines, felines, murines, bovines, equines, and porcines. In a particularly preferred embodiment, the term “mammal” refers to humans.

### Compounds of the Invention

In a first aspect, the present invention provides a compound of formula (II)

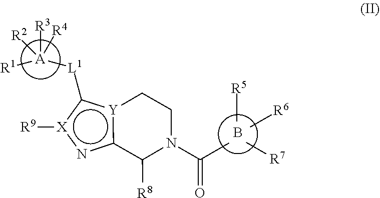

or a pharmaceutically acceptable salt thereof, wherein:
L₁ is selected from a covalent bond, NHCH₂, and CH₂NH;
R⁸ is selected from C₁-C₆-alkyl, halo-C₁-C₆-alkyl, hydroxy-C₁-C₆-alkyl, C₃-C₁₀-cycloalkyl, and halo-C₃-C₁₀-cycloalkyl;
R⁹ is selected from hydrogen, C₁-C₆-alkyl, halo-C₁-C₆-alkyl, hydroxy-C₁-C₆-alkyl, C₃-C₁₀-cycloalkyl, and halo-C₃-C₁₀-cycloalkyl;
(a) X is NR⁹ and Y is C; and
(i) A is selected from furanyl, thienyl, phenyl, and pyridyl;
   B is selected from phenyl, pyridyl, pyrimidinyl, pyrazinyl, triazolyl, and imidazolyl;
   L² is selected from a covalent bond CH₂O, OCH₂, CH₂NH, NHCH₂, CH₂, CH₂CH₂, CF₂CH₂, and CH₂CF₂;
   C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, and oxazolidine;
   R¹ is selected from hydrogen, halogen, C₁-C₆-alkyl, halo-C₁-C₆-alkyl, C₁-C₆-alkoxy, and halo-C₁-C₆-alkoxy;
   R², R³, and R⁴ are each independently either absent or selected from hydrogen, halogen, cyano, and C₁-C₆-alkyl;
   R⁵ is a group

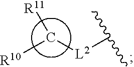

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, oxo, and C₁-C₆-alkyl; or
(ii) A is selected from furanyl, thienyl, phenyl, and pyridyl;
   B is selected from phenyl, pyridyl, pyrimidinyl, pyrazinyl, triazolyl, and imidazolyl;
   L² is selected from a covalent bond CH₂O, OCH₂, CH₂NH, NHCH₂, CH₂, CH₂CH₂, CF₂CH₂, and CH₂CF₂;
   C is selected from phenyl and pyridyl;
   R¹ is selected from hydrogen, halogen, C₁-C₆-alkyl, halo-C₁-C₆-alkyl, C₁-C₆-alkoxy, and halo-C₁-C₆-alkoxy;
   R², R³, and R⁴ are each independently either absent or selected from hydrogen, halogen, cyano, and C₁-C₆-alkyl;
   R⁵ is a group

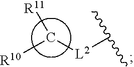

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from cyano and hydroxy; and
R¹¹ is selected from hydrogen, hydroxy, and C₁-C₆-alkyl; or
(iii) A is selected from oxazolyl, pyrrolyl, pyrazinyl, cyclopropyl, 1,3,4-oxadiazolyl, pyrimidinyl;
   B is selected from phenyl, pyridyl, pyrimidinyl, pyrazinyl, triazolyl, and imidazolyl;
   L² is selected from a covalent bond, CH₂O, OCH₂, CH₂NH, NHCH₂, CH₂, CH₂CH₂, CF₂CH₂, and CH₂CF₂;
   C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine, phenyl, and pyridyl;
   R¹ is selected from hydrogen, halogen, C₁-C₆-alkyl, halo-C₁-C₆-alkyl, C₁-C₆-alkoxy, and halo-C₁-C₆-alkoxy;
   R², R³, and R⁴ are each independently either absent or selected from hydrogen, halogen, cyano, and C₁-C₆-alkyl;
   R⁵ is a group

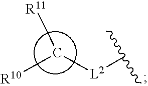

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, and C₁-C₆-alkyl; or
(iv) A is selected from oxazolyl, furanyl, thienyl, pyrrolyl, 1,3,4-oxadiazolyl, cyclopropyl, phenyl, pyrazinyl, pyridyl, and pyrimidinyl;
   B is selected from phenyl, pyridyl, pyrimidinyl, pyrazinyl, triazolyl, and imidazolyl;
   L² is selected from a covalent bond, CH₂O, OCH₂, CH₂NH, NHCH₂, CH₂, CH₂CH₂, CF₂CH₂, and CH₂CF₂;
   C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine, phenyl, and pyridyl;
   R¹ is selected from hydroxy, carbamoyl-C₁-C₆-alkyl, hydroxy-C₁-C₆-alkyl, C₁-C₆-alkoxy-C₁-C₆-alkoxy, C₁-C₆-alkoxy-C₁-C₆-alkyl, C₃-C₁₀-cycloalkyl, NH₂SO₂—, C₁-C₆-alkyl-SO₂—, C₁-C₆-alkyl-SO₂—C₁-C₆-alkyl-, (C₁-C₆-alkyl)₂-PO—C₁-C₆-alkyl-, C₁-C₆-alkyl-PO₂—C₁-C₆-alkyl-, C₁-C₆-alkyl-sulfonimidoyl-C₁-C₆-alkyl-, halo-C₁-C₆-alkyl-sulfonimidoyl-C₁-C₆-alkyl-, C₁-C₆-alkyl-NH—SO₂—C₁-C₆-alkyl-, and C₁-C₆-alkyl-SO₂—NH—C₁-C₆-alkyl-; wherein said C₃-C₁₀-cycloalkyl is optionally substituted with one substituent selected from carbamoyl, C₁-C₆-alkyl-SO₂—, and C₁-C₆-alkyl-SO₂—NH—;
   R², R³, and R⁴ are each independently either absent or selected from hydrogen, halogen, cyano, and C₁-C₆-alkyl;
   R⁵ is a group

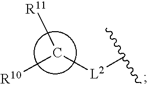

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, and C₁-C₆-alkyl; or
(b) X is CR⁹ or N and Y is N;
L² is selected from a covalent bond, CH₂O, OCH₂, CH₂NH, NHCH₂, CH₂, CH₂CH₂, CF₂CH₂, and CH₂CF₂;
A is selected from furanyl, thienyl, oxazolyl, 1,3,4-oxadiazolyl, cyclopropyl, phenyl, pyrazinyl, pyridyl, and pyrimidinyl;
B is selected from triazolyl, imidazolyl, phenyl, and pyridyl;
C is selected from azetidine, cyclopropyl, piperidine, piperazine, pyridyl, pyrazine, pyrimidine, 1,2-dihydropyridine, 2-thia-6-azaspiro[3.3]heptane, 1,6-diazaspiro[3.3]heptane, 2,6-diazaspiro[3.3]heptane, 4,5-dihydroisoxazole, imidazolidine, oxazolidine, phenyl, pyrrolyl, pyrrolidinyl, pyrazolyl, and triazolyl;
R¹ is selected from halogen, hydroxy, carbamoyl-C₁-C₆-alkyl, C₁-C₆-alkyl, halo-C₁-C₆-alkyl, hydroxy-C₁-C₆-alkyl, C₁-C₆-alkoxy, halo-C₁-C₆-alkoxy, C₁-C₆-alkoxy-C₁-C₆-alkoxy, C₃-C₁₀-cycloalkyl, NH₂SO₂—, C₁-C₆-alkyl-SO₂—, C₁-C₆-alkyl-SO₂—C₁-C₆-alkyl-, (C₁-C₆-alkyl)₂-PO—C₁-C₆-alkyl-, C₁-C₆-alkyl-PO₂—C₁-C₆-alkyl-, C₁-C₆-alkyl-sulfonimidoyl-C₁-C₆-alkyl-, halo-C₁-C₆-alkyl-sulfonimidoyl-C₁-C₆-alkyl-, C₁-C₆-alkyl-NH—SO₂—C₁-C₆-alkyl-, and C₁-C₆-alkyl-SO₂—NH—C₁-C₆-alkyl-; wherein said C₃-C₁₀-cycloalkyl is optionally substituted with one substituent selected from carbamoyl, C₁-C₆-alkyl-SO₂—, and C₁-C₆-alkyl-SO₂—NH—;
R², R³, and R⁴ are each independently absent or selected from hydrogen, halogen, and cyano;
R⁵ is selected from C₁-C₆-alkoxy, C₁-C₆-alkoxy-C₁-C₆-alkyl, and a group

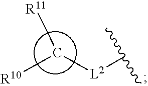

R⁶ is selected from hydrogen, halogen, and C₁-C₆-alkyl;
R⁷ is selected from hydrogen and halogen;
R⁸ is selected from C₁-C₆-alkyl, halo-C₁-C₆-alkyl, hydroxy-C₁-C₆-alkyl, C₃-C₁₀-cycloalkyl, and halo-C₃-C₁₀-cycloalkyl;
R⁹ is selected from hydrogen, C₁-C₆-alkyl, halo-C₁-C₆-alkyl, hydroxy-C₁-C₆-alkyl, C₃-C₁₀-cycloalkyl, and halo-C₃-C₁₀-cycloalkyl;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, oxo, and C₁-C₆-alkyl.

In a preferred embodiment, the present invention provides a compound of formula (II) as described herein, or a pharmaceutically acceptable salt thereof, wherein:
X is NR and Y is C; or
X is CR⁹ or N and Y is N;
L¹ is selected from a covalent bond, NHCH₂, and CH₂NH;
R⁸ is selected from C₁-C₆-alkyl, halo-C₁-C₆-alkyl, hydroxy-C₁-C₆-alkyl, C₃-C₁₀-cycloalkyl, and halo-C₃-C₁₀-cycloalkyl;
R⁹ is selected from hydrogen, C₁-C₆-alkyl, halo-C₁-C₆-alkyl, hydroxy-C₁-C₆-alkyl, C₃-C₁₀-cycloalkyl, and halo-C₃-C₁₀-cycloalkyl; and
(i) A is selected from furanyl, thienyl, phenyl, and pyridyl;
   B is selected from phenyl, pyridyl, pyrimidinyl, pyrazinyl, triazolyl, and imidazolyl;
   L² is selected from a covalent bond CH₂O, OCH₂, CH₂NH, NHCH₂, CH₂, CH₂CH₂, CF₂CH₂, and CH₂CF₂;
   C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, and oxazolidine;
   R¹ is selected from hydrogen, halogen, C₁-C₆-alkyl, halo-C₁-C₆-alkyl, C₁-C₆-alkoxy, and halo-C₁-C₆-alkoxy;
   R², R³, and R⁴ are each independently either absent or selected from hydrogen, halogen, cyano, and C₁-C₆-alkyl;
   R⁵ is a group

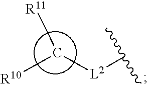

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, oxo, and C₁-C₆-alkyl; or
(ii) A is selected from furanyl, thienyl, phenyl, and pyridyl;
   B is selected from phenyl, pyridyl, pyrimidinyl, pyrazinyl, triazolyl, and imidazolyl;
   L² is selected from a covalent bond CH₂O, OCH₂, CH₂NH, NHCH₂, CH₂, CH₂CH₂, CF₂CH₂, and CH₂CF₂;
   C is selected from phenyl and pyridyl;
   R¹ is selected from hydrogen, halogen, C₁-C₆-alkyl, halo-C₁-C₆-alkyl, C₁-C₆-alkoxy, and halo-C₁-C₆-alkoxy;
   R², R³, and R⁴ are each independently either absent or selected from hydrogen, halogen, cyano, and C₁-C₆-alkyl;
   R⁵ is a group

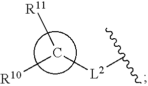

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from cyano and hydroxy; and
R¹¹ is selected from hydrogen, hydroxy, and C₁-C₆-alkyl; or
(iii) A is selected from oxazolyl, pyrrolyl, pyrazinyl, cyclopropyl, 1,3,4-oxadiazolyl, pyrimidinyl;
   B is selected from phenyl, pyridyl, pyrimidinyl, pyrazinyl, triazolyl, and imidazolyl;
   L² is selected from a covalent bond, CH₂O, OCH₂, CH₂NH, NHCH₂, CH₂, CH₂CH₂, CF₂CH₂, and CH₂CF₂;
   C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine, phenyl, and pyridyl;
   R¹ is selected from hydrogen, halogen, C₁-C₆-alkyl, halo-C₁-C₆-alkyl, C₁-C₆-alkoxy, and halo-C₁-C₆-alkoxy;
   R², R³, and R⁴ are each independently either absent or selected from hydrogen, halogen, cyano, and C₁-C₆-alkyl;

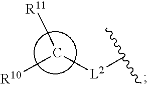

R⁵ is a group
R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, and C₁-C₆-alkyl; or
(iv) A is selected from oxazolyl, furanyl, thienyl, pyrrolyl, 1,3,4-oxadiazolyl, cyclopropyl, phenyl, pyrazinyl, pyridyl, and pyrimidinyl;
   B is selected from phenyl, pyridyl, pyrimidinyl, pyrazinyl, triazolyl, and imidazolyl;
   L² is selected from a covalent bond, CH₂O, OCH₂, CH₂NH, NHCH₂, CH₂, CH₂CH₂, CF₂CH₂, and CH₂CF₂;
   C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine, phenyl, and pyridyl;
   R¹ is selected from hydroxy, carbamoyl-C₁-C₆-alkyl, hydroxy-C₁-C₆-alkyl, C₁-C₆-alkoxy-C₁-C₆-alkoxy, C₁-C₆-alkoxy-C₁-C₆-alkyl, C₃-C₁₀-cycloalkyl, NH₂SO₂—, C₁-C₆-alkyl-SO₂—, C₁-C₆-alkyl-SO₂—C₁-C₆-alkyl-, (C₁-C₆-alkyl)₂-PO—C₁-C₆-alkyl-, C₁-C₆-alkyl-PO₂—C₁-C₆-alkyl-, C₁-C₆-alkyl-sulfonimidoyl-C₁-C₆-alkyl-, halo-C₁-C₆-alkyl-sulfonimidoyl-C₁-C₆-alkyl-, C₁-C₆-alkyl-NH—SO₂—C₁-C₆-alkyl-, and C₁-C₆-alkyl-SO₂—NH—C₁-C₆-alkyl-; wherein said C₃-C₁₀-cycloalkyl is optionally substituted with one substituent selected from carbamoyl, C₁-C₆-alkyl-SO₂—, and C₁-C₆-alkyl-SO₂—NH—;
   R², R³, and R⁴ are each independently either absent or selected from hydrogen, halogen, cyano, and C₁-C₆-alkyl;
   R⁵ is a group

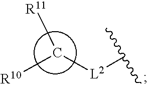

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, and C₁-C₆-alkyl.

In one embodiment, the present invention provides a compound of formula (II) as described herein, or a pharmaceutically acceptable salt thereof, wherein:
X is CR⁹ or N and Y is N;
L¹ is selected from a covalent bond, NHCH₂, and CH₂NH;
L² is selected from a covalent bond, CH₂O, OCH₂, CH₂NH, NHCH₂, CH₂, CH₂CH₂, CF₂CH₂, and CH₂CF₂;
A is selected from furanyl, thienyl, oxazolyl, 1,3,4-oxadiazolyl, cyclopropyl, phenyl, pyrazinyl, pyridyl, and pyrimidinyl;
B is selected from triazolyl, imidazolyl, phenyl, and pyridyl;
C is selected from azetidine, cyclopropyl, piperidine, piperazine, pyridyl, pyrazine, pyrimidine, 1,2-dihydropyridine, 2-thia-6-azaspiro[3.3]heptane, 1,6-diazaspiro[3.3]heptane, 2,6-diazaspiro[3.3]heptane, 4,5-dihydroisoxazole, imidazolidine, oxazolidine, phenyl, pyrrolyl, pyrrolidinyl, pyrazolyl, and triazolyl;
R¹ is selected from halogen, hydroxy, carbamoyl-C₁-C₆-alkyl, C₁-C₆-alkyl, halo-C₁-C₆-alkyl, hydroxy-C₁-C₆-alkyl, C₁-C₆-alkoxy, halo-C₁-C₆-alkoxy, C₁-C₆-alkoxy-C₁-C₆-alkoxy, C₃-C₁₀-cycloalkyl, NH₂SO₂—, C₁-C₆-alkyl-SO₂—, C₁-C₆-alkyl-SO₂—C₁-C₆-alkyl-, (C₁-C₆-alkyl)₂-PO—C₁-C₆-alkyl-, C₁-C₆-alkyl-PO₂—C₁-C₆-alkyl-, C₁-C₆-alkyl-sulfonimidoyl-C₁-C₆-alkyl-, halo-C₁-C₆-alkyl-sulfonimidoyl-C₁-C₆-alkyl-, C₁-C₆-alkyl-NH—SO₂—C₁-C₆-alkyl-, and C₁-C₆-alkyl-SO₂—NH—C₁-C₆-alkyl-; wherein said C₃-C₁₀-cycloalkyl is optionally substituted with one substituent selected from carbamoyl, C₁-C₆-alkyl-SO₂—, and C₁-C₆-alkyl-SO₂—NH—;
R², R³, and R⁴ are each independently absent or selected from hydrogen, halogen, and cyano;
R⁵ is selected from C₁-C₆-alkoxy, C₁-C₆-alkoxy-C₁-C₆-alkyl, and a group

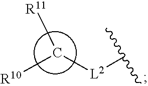

R⁶ is selected from hydrogen, halogen, and C₁-C₆-alkyl;
R⁷ is selected from hydrogen and halogen;
R⁸ is selected from C₁-C₆-alkyl, halo-C₁-C₆-alkyl, hydroxy-C₁-C₆-alkyl, C₃-C₁₀-cycloalkyl, and halo-C₃-C₁₀-cycloalkyl;
R⁹ is selected from hydrogen, C₁-C₆-alkyl, halo-C₁-C₆-alkyl, hydroxy-C₁-C₆-alkyl, C₃-C₁₀-cycloalkyl, and halo-C₃-C₁₀-cycloalkyl;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, oxo, and C₁-C₆-alkyl.

In a particularly preferred embodiment, the present invention provides a compound of formula (II) as described herein, or a pharmaceutically acceptable salt thereof, wherein X is NR⁹ and Y is C, represented by formula (I)

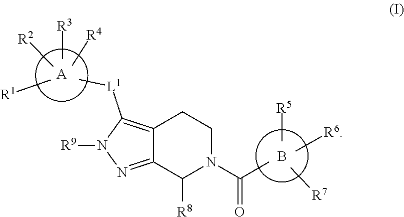

In one embodiment, the present invention provides a compound of formula (II) as described herein, or a pharmaceutically acceptable salt thereof, wherein X is CR⁹ and Y is N, represented by formula (IIa)

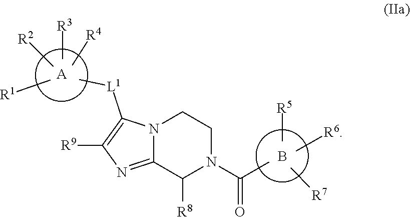

In one embodiment, the present invention provides a compound of formula (II) as described herein, or a pharmaceutically acceptable salt thereof, wherein X and Y are both N, represented by formula (IIb)

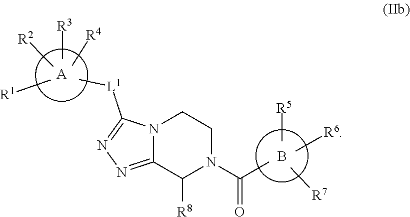

In one embodiment, the present invention provides a compound of formula (II) as described herein, or a pharmaceutically acceptable salt thereof, wherein:
(i) A is selected from furanyl, thienyl, phenyl, and pyridyl;
   B is selected from phenyl, pyridyl, pyrimidinyl, pyrazinyl, triazolyl, and imidazolyl;
   L² is selected from a covalent bond CH₂O, OCH₂, CH₂NH, NHCH₂, CH₂, CH₂CH₂, CF₂CH₂, and CH₂CF₂;
   C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, and oxazolidine;
   R¹ is selected from hydrogen, halogen, C₁-C₆-alkyl, halo-C₁-C₆-alkyl, C₁-C₆-alkoxy, and halo-C₁-C₆-alkoxy;
   R², R³, and R⁴ are each independently either absent or selected from hydrogen, halogen, cyano, and C₁-C₆-alkyl;
   R⁵ is a group

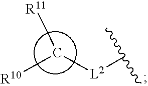

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, oxo, and C₁-C₆-alkyl; or
(ii) A is selected from furanyl, thienyl, phenyl, and pyridyl;
   B is selected from phenyl, pyridyl, pyrimidinyl, pyrazinyl, triazolyl, and imidazolyl;
   L² is selected from a covalent bond CH₂O, OCH₂, CH₂NH, NHCH₂, CH₂, CH₂CH₂, CF₂CH₂, and CH₂CF₂;
   C is selected from phenyl and pyridyl;
   R¹ is selected from hydrogen, halogen, C₁-C₆-alkyl, halo-C₁-C₆-alkyl, C₁-C₆-alkoxy, and halo-C₁-C₆-alkoxy;
   R², R³, and R⁴ are each independently either absent or selected from hydrogen, halogen, cyano, and C₁-C₆-alkyl;
   R⁵ is a group

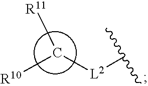

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from cyano and hydroxy; and
R¹¹ is selected from hydrogen, hydroxy, and C₁-C₆-alkyl.

In one embodiment, the present invention provides a compound of formula (II) as described herein, or a pharmaceutically acceptable salt thereof, wherein:
(i) A is selected from oxazolyl, pyrrolyl, pyrazinyl, cyclopropyl, 1,3,4-oxadiazolyl, pyrimidinyl;
   B is selected from phenyl, pyridyl, pyrimidinyl, pyrazinyl, triazolyl, and imidazolyl;
   L² is selected from a covalent bond, CH₂O, OCH₂, CH₂NH, NHCH₂, CH₂, CH₂CH₂, CF₂CH₂, and CH₂CF₂;
   C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine, phenyl, and pyridyl;
   R¹ is selected from hydrogen, halogen, C₁-C₆-alkyl, halo-C₁-C₆-alkyl, C₁-C₆-alkoxy, and halo-C₁-C₆-alkoxy;
   R², R³, and R⁴ are each independently either absent or selected from hydrogen, halogen, cyano, and C₁-C₆-alkyl;
   R⁵ is a group

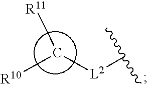

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, and C₁-C₆-alkyl; or
(ii) A is selected from oxazolyl, furanyl, thienyl, pyrrolyl, 1,3,4-oxadiazolyl, cyclopropyl, phenyl, pyrazinyl, pyridyl, and pyrimidinyl;
   B is selected from phenyl, pyridyl, pyrimidinyl, pyrazinyl, triazolyl, and imidazolyl;
   L² is selected from a covalent bond, CH₂O, OCH₂, CH₂NH, NHCH₂, CH₂, CH₂CH₂, CF₂CH₂, and CH₂CF₂;
   C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine, phenyl, and pyridyl;
   R¹ is selected from hydroxy, carbamoyl-C₁-C₆-alkyl, hydroxy-C₁-C₆-alkyl, C₁-C₆-alkoxy-C₁-C₆-alkoxy, C₁-C₆-alkoxy-C₁-C₆-alkyl, C₃-C₁₀-cycloalkyl, NH₂SO₂—, C₁-C₆-alkyl-SO₂—, C₁-C₆-alkyl-SO₂—C₁-C₆-alkyl-, (C₁-C₆-alkyl)₂-PO—C₁-C₆-alkyl-, C₁-C₆-alkyl-PO₂—C₁-C₆-alkyl-, C₁-C₆-alkyl-sulfonimidoyl-C₁-C₆-alkyl-, halo-C₁-C₆-alkyl-sulfonimidoyl-C₁-C₆-alkyl-, C₁-C₆-alkyl-NH—SO₂—C₁-C₆-alkyl-, and C₁-C₆-alkyl-SO₂—NH—C₁-C₆-alkyl-; wherein said C₃-C₁₀-cycloalkyl is optionally substituted with one substituent selected from carbamoyl, C₁-C₆-alkyl-SO₂—, and C₁-C₆-alkyl-SO₂—NH—;
   R², R³, and R⁴ are each independently either absent or selected from hydrogen, halogen, cyano, and C₁-C₆-alkyl;
   R⁵ is a group

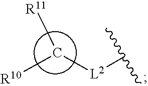

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, and C₁-C₆-alkyl.

In one embodiment, the present invention provides a compound of formula (II) as described herein, or a pharmaceutically acceptable salt thereof, wherein
A is selected from furanyl, thienyl, phenyl, and pyridyl;
B is selected from phenyl, pyridyl, pyrimidinyl, pyrazinyl, triazolyl, and imidazolyl;
   L² is selected from a covalent bond CH₂O, OCH₂, CH₂NH, NHCH₂, CH₂, CH₂CH₂, CF₂CH₂, and CH₂CF₂;
   C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, and oxazolidine;
   R¹ is selected from hydrogen, halogen, C₁-C₆-alkyl, halo-C₁-C₆-alkyl, C₁-C₆-alkoxy, and halo-C₁-C₆-alkoxy;
   R², R³, and R⁴ are each independently either absent or selected from hydrogen, halogen, cyano, and C₁-C₆-alkyl;
   R⁵ is a group

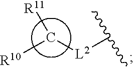

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, oxo, and C₁-C₆-alkyl.

In one embodiment, the present invention provides a compound of formula (II) as described herein, or a pharmaceutically acceptable salt thereof, wherein
A is selected from furanyl, thienyl, phenyl, and pyridyl;
B is selected from phenyl, pyridyl, pyrimidinyl, pyrazinyl, triazolyl, and imidazolyl;
L² is selected from a covalent bond CH₂O, OCH₂, CH₂NH, NHCH₂, CH₂, CH₂CH₂, CF₂CH₂, and CH₂CF₂;
C is selected from phenyl and pyridyl;
R¹ is selected from hydrogen, halogen, C₁-C₆-alkyl, halo-C₁-C₆-alkyl, C₁-C₆-alkoxy, and halo-C₁-C₆-alkoxy;
R², R³, and R⁴ are each independently either absent or selected from hydrogen, halogen, cyano, and C₁-C₆-alkyl;
R⁵ is a group

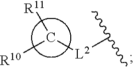

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from cyano and hydroxy; and
R¹¹ is selected from hydrogen, hydroxy, and C₁-C₆-alkyl.

In one embodiment, the present invention provides a compound of formula (II) as described herein, or a pharmaceutically acceptable salt thereof, wherein
A is selected from oxazolyl, pyrrolyl, pyrazinyl, cyclopropyl, 1,3,4-oxadiazolyl, pyrimidinyl;
B is selected from phenyl, pyridyl, pyrimidinyl, pyrazinyl, triazolyl, and imidazolyl;
L² is selected from a covalent bond, CH₂O, OCH₂, CH₂NH, NHCH₂, CH₂, CH₂CH₂, CF₂CH₂, and CH₂CF₂;
C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine, phenyl, and pyridyl;
R¹ is selected from hydrogen, halogen, C₁-C₆-alkyl, halo-C₁-C₆-alkyl, C₁-C₆-alkoxy, and halo-C₁-C₆-alkoxy;
R², R³, and R⁴ are each independently either absent or selected from hydrogen, halogen, cyano, and C₁-C₆-alkyl;
R⁵ is a group

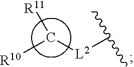

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, and C₁-C₆-alkyl.

In one embodiment, the present invention provides a compound of formula (II) as described herein, or a pharmaceutically acceptable salt thereof, wherein
A is selected from oxazolyl, furanyl, thienyl, pyrrolyl, 1,3,4-oxadiazolyl, cyclopropyl, phenyl, pyrazinyl, pyridyl, and pyrimidinyl;
B is selected from phenyl, pyridyl, pyrimidinyl, pyrazinyl, triazolyl, and imidazolyl;
L² is selected from a covalent bond, CH₂O, OCH₂, CH₂NH, NHCH₂, CH₂, CH₂CH₂, CF₂CH₂, and CH₂CF₂;
C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine, phenyl, and pyridyl;
R¹ is selected from hydroxy, carbamoyl-C₁-C₆-alkyl, hydroxy-C₁-C₆-alkyl, C₁-C₆-alkoxy-C₁-C₆-alkoxy, C₁-C₆-alkoxy-C₁-C₆-alkyl, C₃-C₁₀-cycloalkyl, NH₂SO₂—, C₁-C₆-alkyl-SO₂—, C₁-C₆-alkyl-SO₂—C₁-C₆-alkyl-, (C₁-C₆-alkyl)₂-PO—C₁-C₆-alkyl-, C₁-C₆-alkyl-PO₂—C₁-C₆-alkyl-, C₁-C₆-alkyl-sulfonimidoyl-C₁-C₆-alkyl-, halo-C₁-C₆-alkyl-sulfonimidoyl-C₁-C₆-alkyl-, C₁-C₆-alkyl-NH—SO₂—C₁-C₆-alkyl-, and C₁-C₆-alkyl-SO₂—NH—C₁-C₆-alkyl-; wherein said C₃-C₁₀-cycloalkyl is optionally substituted with one substituent selected from carbamoyl, C₁-C₆-alkyl-SO₂—, and C₁-C₆-alkyl-SO₂—NH—;
R², R³, and R⁴ are each independently either absent or selected from hydrogen, halogen, cyano, and C₁-C₆-alkyl;
R⁵ is a group

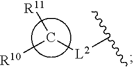

R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, and C₁-C₆-alkyl.

In one embodiment, the present invention provides a compound of formula (II) as described herein, or a pharmaceutically acceptable salt thereof, wherein
(i) L² is selected from a covalent bond, OCH₂, CH₂O, NHCH₂, CH₂CH₂,
   A is phenyl;
   B is selected from phenyl, pyridyl, triazolyl, imidazolyl;
   C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine;
   R¹ is halogen;
   R² is halogen;
   R³ is hydrogen or halogen;
   R⁴ is hydrogen or halogen;
   R⁵ is a group

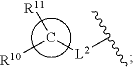

R⁶ is halogen or C₁-C₆-alkyl;
R⁷ is hydrogen or halogen;
R¹⁰ is selected from hydrogen, C₁-C₆-alkyl, halo-C₁-C₆-alkyl, C₁-C₆-alkyl-SO₂—, oxo, hydroxy, and cyano; and
R¹¹ is selected from hydrogen, C₁-C₆-alkyl, oxo; or
(ii) L² is a covalent bond;
   A is phenyl;
   B is triazolyl or phenyl;
   C is pyridyl or phenyl;
   R¹ is halogen;
   R² is halogen;
   R³ is hydrogen;
   R⁴ is hydrogen;
   R⁵ is a group

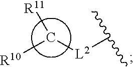

R⁶ is hydrogen or halogen;
R⁷ is hydrogen;
R¹⁰ is hydroxy or cyano; and
R¹¹ is hydrogen or halogen; or
(iii) L² is CH₂ or CH₂CH₂;
   A is phenyl;
   B is phenyl;
   C is triazolyl or pyrazolyl;
   R¹ is halogen;
   R² is halogen;
   R³ is hydrogen;
   R⁴ is hydrogen;
   R⁵ is a group

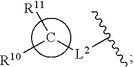

R⁶ is hydrogen or halogen;
R⁷ is hydrogen or halogen;
R¹⁰ is hydrogen; and
R¹¹ is hydrogen; or
(iv) A is selected from pyrazinyl, cyclopropyl, 1,3,4-oxadiazolyl, and pyrimidinyl;
   B is phenyl;
   R¹ is halo-C₁-C₆-alkyl;
   R² is hydrogen;
   R³ is hydrogen;
   R⁴ is hydrogen;
   R⁵ is C₁-C₆-alkoxy;
   R⁶ is halogen; and
   R⁷ is hydrogen; or
(v) L² is a covalent bond;
   A is pyridyl or phenyl;
   B is phenyl, triazolyl;
   C is triazolyl, pyrazolyl, pyridyl;
   R¹ is hydroxy, hydroxy-C₁-C₆-alkyl, C₁-C₆-alkoxy-C₁-C₆-alkoxy, C₁-C₆-alkyl-SO₂—, C₁-C₆-alkyl-SO₂—C₁-C₆-alkyl-, C₁-C₆-alkyl-SO₂—NH—C₁-C₆-alkyl-, C₁-C₆-alkyl-NH—SO₂—C₁-C₆-alkyl-, carbamoyl-C₁-C₆-alkyl, NH₂SO₂—, C₃-C₁₀-cycloalkyl; wherein said C₃-C₁₀-cycloalkyl is optionally substituted with one substituent selected from carbamoyl and C₁-C₆-alkyl-SO₂—NH—;
   R² is hydrogen, halogen;
   R³ is hydrogen;
   R⁴ is hydrogen;
   R⁵ is C₁-C₆-alkoxy or a group

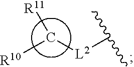

R⁶ is halogen;
R⁷ is hydrogen or halogen;
R¹⁰ is hydrogen, hydroxy; and
R¹¹ is hydrogen, halogen; or
(vi) L² is a covalent bond;
   A is pyrazinyl;
   B is phenyl or triazolyl;
   C is selected from azetidinyl, 1,2-dihydropyridine, pyrrolyl, and 2-thia-6-azaspiro[3.3]heptane;
   R¹ is halo-C₁-C₆-alkyl;
   R² is hydrogen;
   R³ is hydrogen;
   R⁴ is hydrogen;
   R⁵ is a group

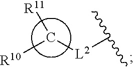

R⁶ is halogen;
R⁷ is halogen;
R¹⁰ is selected from NH₂SO₂—, cyano, and oxo; and
R¹¹ is hydrogen or oxo; or
(vii) L² is selected from a covalent bond, CH₂O, and CH₂CH₂;
   A is phenyl;
   B is phenyl or triazolyl;
   C is selected from 1,2-dihydropyridine, pyrrolidinyl, pyrrolyl, 2-thia-6-azaspiro[3.3]heptane, and triazolyl;
   R¹ is selected from NH₂SO₂—, C₁-C₆-alkyl-PO₂—C₁-C₆-alkyl-, C₃-C₁₀-cycloalkyl; wherein said C₃-C₁₀-cycloalkyl is optionally substituted with one substituent selected from carbamoyl, C₁-C₆-alkyl-SO₂—, and C₁-C₆-alkyl-SO₂—NH—;
   R² is halogen;
   R³ is hydrogen;
   R⁴ is hydrogen;
   R⁵ is a group

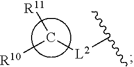

R⁶ is halogen;
R⁷ is halogen;
R¹⁰ is oxo or cyano; and
R¹¹ is hydrogen or oxo.

In one embodiment, the present invention provides a compound of formula (II) as described herein, or a pharmaceutically acceptable salt thereof, wherein:
L² is selected from a covalent bond, OCH₂, CH₂O, NHCH₂, CH₂CH₂, A is phenyl;
B is selected from phenyl, pyridyl, triazolyl, imidazolyl;
C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine;
R¹ is halogen;
R² is halogen;
R³ is hydrogen or halogen;
R⁴ is hydrogen or halogen;
R⁵ is a group

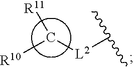

R⁶ is halogen or C₁-C₆-alkyl;
R⁷ is hydrogen or halogen;
R¹⁰ is selected from hydrogen, C₁-C₆-alkyl, halo-C₁-C₆-alkyl, C₁-C₆-alkyl-SO₂—, oxo, hydroxy, and cyano; and
R¹¹ is selected from hydrogen, C₁-C₆-alkyl, oxo.

In one embodiment, the present invention provides a compound of formula (II) as described herein, or a pharmaceutically acceptable salt thereof, wherein:
L² is a covalent bond;
A is phenyl;
B is triazolyl or phenyl;
C is pyridyl or phenyl;
R¹ is halogen;
R² is halogen;
R³ is hydrogen;
R⁴ is hydrogen;
   R⁵ is a group

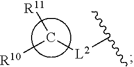

R⁶ is hydrogen or halogen;
R⁷ is hydrogen;
R¹⁰ is hydroxy or cyano; and
R¹¹ is hydrogen or halogen.

In one embodiment, the present invention provides a compound of formula (II) as described herein, or a pharmaceutically acceptable salt thereof, wherein:
L² is CH₂ or CH₂CH₂;
A is phenyl;
B is phenyl;
C is triazolyl or pyrazolyl;
R¹ is halogen;
R² is halogen;
R³ is hydrogen;
R⁴ is hydrogen;
R⁵ is a group

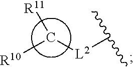

R⁶ is hydrogen or halogen;
R⁷ is hydrogen or halogen;
R¹⁰ is hydrogen; and
R¹¹ is hydrogen.

In one embodiment, the present invention provides a compound of formula (II) as described herein, or a pharmaceutically acceptable salt thereof, wherein:
A is selected from pyrazinyl, cyclopropyl, 1,3,4-oxadiazolyl, and pyrimidinyl;
B is phenyl;
R¹ is halo-C₁-C₆-alkyl;
R² is hydrogen;
R³ is hydrogen;
R⁴ is hydrogen;
R⁵ is C₁-C₆-alkoxy;
R⁶ is halogen; and
R⁷ is hydrogen.

In one embodiment, the present invention provides a compound of formula (II) as described herein, or a pharmaceutically acceptable salt thereof, wherein:
L² is a covalent bond;
A is pyridyl or phenyl;
B is phenyl, triazolyl;
C is triazolyl, pyrazolyl, pyridyl;
R¹ is hydroxy, hydroxy-C₁-C₆-alkyl, C₁-C₆-alkoxy-C₁-C₆-alkoxy, C₁-C₆-alkyl-SO₂—, C₁-C₆-alkyl-SO₂—C₁-C₆-alkyl-, C₁-C₆-alkyl-SO₂—NH—C₁-C₆-alkyl-, C₁-C₆-alkyl-NH—SO₂—C₁-C₆-alkyl-, carbamoyl-C₁-C₆-alkyl, NH₂SO₂—, C₃-C₁₀-cycloalkyl; wherein said C₃-C₁₀-cycloalkyl is optionally substituted with one substituent selected from carbamoyl and C₁-C₆-alkyl-SO₂—NH—;
R² is hydrogen, halogen;
R³ is hydrogen;
R⁴ is hydrogen;
R⁵ is C₁-C₆-alkoxy or a group

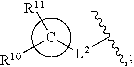

R⁶ is halogen;
R⁷ is hydrogen or halogen;
R¹⁰ is hydrogen, hydroxy; and
R¹¹ is hydrogen, halogen.

In one embodiment, the present invention provides a compound of formula (II) as described herein, or a pharmaceutically acceptable salt thereof, wherein:
L² is a covalent bond;
A is pyrazinyl;
B is phenyl or triazolyl;
C is selected from azetidinyl, 1,2-dihydropyridine, pyrrolyl, and 2-thia-6-azaspiro[3.3]heptane;
R¹ is halo-C₁-C₆-alkyl;
R² is hydrogen;
R³ is hydrogen;
R⁴ is hydrogen;
R⁵ is a group

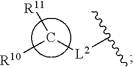

R⁶ is halogen;
R⁷ is halogen;
R¹⁰ is selected from NH₂SO₂—, cyano, and oxo; and
R¹¹ is hydrogen or oxo.

In one embodiment, the present invention provides a compound of formula (II) as described herein, or a pharmaceutically acceptable salt thereof, wherein:
L² is selected from a covalent bond, CH₂O, and CH₂CH₂;
A is phenyl;
B is phenyl or triazolyl;
C is selected from 1,2-dihydropyridine, pyrrolidinyl, pyrrolyl, 2-thia-6-azaspiro[3.3]heptane, and triazolyl;
   R¹ is selected from NH₂SO₂—, C₁-C₆-alkyl-PO₂—C₁-C₆-alkyl-, C₃-C₁₀-cycloalkyl; wherein said C₃-C₁₀-cycloalkyl is optionally substituted with one substituent selected from carbamoyl, C₁-C₆-alkyl-SO₂—, and C₁-C₆-alkyl-SO₂—NH—;
R² is halogen;
R³ is hydrogen;
R⁴ is hydrogen;
R⁵ is a group

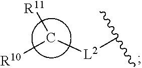

R⁶ is halogen;
R⁷ is halogen;
R¹⁰ is oxo or cyano; and
R¹¹ is hydrogen or oxo.

In a preferred embodiment, the present invention provides a compound of formula (II) as described herein, or a pharmaceutically acceptable salt thereof, wherein
(i) L² is selected from a covalent bond, CH₂O, and NHCH₂;
   A is phenyl;
   B is phenyl;
   C is selected from pyrrolidinyl, 1,2-dihydropyridine, imidazolidine, oxazolidine;
   R¹ is halogen;
   R² is halogen;
   R³ is hydrogen;
   R⁴ is hydrogen;
   R⁵ is a group

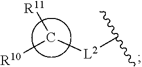

R⁶ is halogen;
R⁷ is halogen;
R¹⁰ is oxo; and
R¹¹ is hydrogen; or
(ii) L² is a covalent bond;
   A is phenyl;
   B is triazolyl;
   C is pyridyl;
   R¹ is halogen;
   R² is halogen;
   R³ is hydrogen;
   R⁴ is hydrogen;
   R⁵ is a group

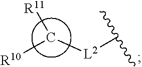

R⁶ is hydrogen;
R⁷ is absent;
R¹⁰ is hydroxy; and
R¹¹ is hydrogen or halogen; or
(iii) L² is CH₂CH₂;
   A is phenyl;
   B is phenyl;
   C is triazolyl or pyrazolyl;
   R¹ is halogen;
   R² is halogen;
   R³ is hydrogen;
   R⁴ is hydrogen;
   R⁵ is a group

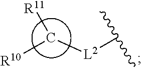

R⁶ is halogen;
R⁷ is halogen;
R¹⁰ is hydrogen; and
R¹¹ is hydrogen; or
(iv) L² is a covalent bond;
   A is phenyl;
   B is phenyl;
   C is pyrrolyl;
   R¹ is C₃-C₁₀-cycloalkyl; wherein said C₃-C₁₀-cycloalkyl is substituted with one C₁-C₆-alkyl-SO₂—NH— substituent;
   R² is halogen;
   R³ is hydrogen;
   R⁴ is hydrogen;
   R⁵ is a group

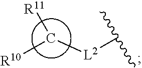

R⁶ is halogen;
R⁷ is halogen;
R¹⁰ is cyano; and
R¹¹ is hydrogen.

In a preferred embodiment, the present invention provides a compound of formula (II) as described herein, or a pharmaceutically acceptable salt thereof, wherein
L² is selected from a covalent bond, CH₂O, and NHCH₂;
A is phenyl;
B is phenyl;
C is selected from pyrrolidinyl, 1,2-dihydropyridine, imidazolidine, oxazolidine;
R¹ is halogen;
R² is halogen;
R³ is hydrogen;
R⁴ is hydrogen;
R⁵ is a group

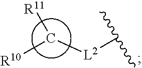

R⁶ is halogen;
R⁷ is halogen;
R¹⁰ is oxo; and
R¹¹ is hydrogen.

In a preferred embodiment, the present invention provides a compound of formula (II) as described herein, or a pharmaceutically acceptable salt thereof, wherein
L² is a covalent bond;
A is phenyl;
B is triazolyl;
C is pyridyl;
R¹ is halogen;
R² is halogen;
R³ is hydrogen;
R⁴ is hydrogen;
R⁵ is a group

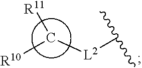

R⁶ is hydrogen;
R⁷ is absent;
R¹⁰ is hydroxy; and
R¹¹ is hydrogen or halogen.

In a preferred embodiment, the present invention provides a compound of formula (II) as described herein, or a pharmaceutically acceptable salt thereof, wherein
L² is CH₂CH₂;
A is phenyl;
B is phenyl;
C is triazolyl or pyrazolyl;
R¹ is halogen;
R² is halogen;
R³ is hydrogen;
R⁴ is hydrogen;
R⁵ is a group

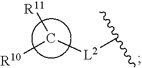

R⁶ is halogen;
R⁷ is halogen;
R¹⁰ is hydrogen; and
R¹¹ is hydrogen.

In a preferred embodiment, the present invention provides a compound of formula (II) as described herein, or a pharmaceutically acceptable salt thereof, wherein
L² is a covalent bond;
A is phenyl;
B is phenyl;
C is pyrrolyl;
R¹ is C₃-C₁₀-cycloalkyl; wherein said C₃-C₁₀-cycloalkyl is substituted with one C₁-C₆-alkyl-SO₂—NH— substituent;
R² is halogen;
R³ is hydrogen;
R⁴ is hydrogen;
R⁵ is a group

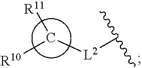

R⁶ is halogen;
R⁷ is halogen;
R¹⁰ is cyano; and
R¹¹ is hydrogen.

In a particularly preferred embodiment, the present invention provides a compound of formula (II) as described herein, or a pharmaceutically acceptable salt thereof, wherein
(i) L² is selected from a covalent bond, CH₂O, and NHCH₂;
   A is phenyl;
   B is phenyl;
   C is selected from pyrrolidinyl, 1,2-dihydropyridine, imidazolidine, oxazolidine;
   R¹ is fluoro;
   R² is fluoro;
   R³ is hydrogen;
   R⁴ is hydrogen;
   R⁵ is a group

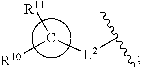

R⁶ is fluoro;
R⁷ is chloro;
R¹⁰ is oxo; and
R¹¹ is hydrogen; or
(ii) L² is a covalent bond;
   A is phenyl;
   B is triazolyl;
   C is pyridyl;
   R¹ is fluoro;
   R² is fluoro;
   R³ is hydrogen;
   R⁴ is hydrogen;
   R⁵ is a group

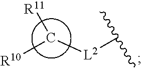

R⁶ is hydrogen;
R⁷ is absent;
R¹⁰ is hydroxy; and
R¹¹ is hydrogen or chloro; or
(iii) L² is CH₂CH₂;
   A is phenyl;
   B is phenyl;
   C is triazolyl or pyrazolyl;
   R¹ is fluoro;
   R² is fluoro;
   R³ is hydrogen;
   R⁴ is hydrogen;
   R⁵ is a group

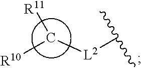

R⁶ is fluoro;
R⁷ is chloro;
R¹⁰ is hydrogen; and
R¹¹ is hydrogen; or
(iv) L² is a covalent bond;
   A is phenyl;
   B is phenyl;
   C is pyrrolyl;
   R¹ is cyclopropyl; wherein said cyclopropyl is substituted with one methyl-SO₂—NH— substituent;
   R² is chloro;
   R³ is hydrogen;
   R⁴ is hydrogen;
   R⁵ is a group

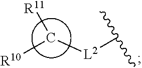

R⁶ is fluoro;
R⁷ is chloro;
R¹⁰ is cyano; and
R¹¹ is hydrogen.

In a particularly preferred embodiment, the present invention provides a compound of formula (II) as described herein, or a pharmaceutically acceptable salt thereof, wherein
L² is selected from a covalent bond, CH₂O, and NHCH₂;
A is phenyl;
B is phenyl;
C is selected from pyrrolidinyl, 1,2-dihydropyridine, imidazolidine, oxazolidine;
R¹ is fluoro;
R² is fluoro;
R³ is hydrogen;
R⁴ is hydrogen;
R⁵ is a group

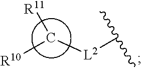

R⁶ is fluoro;
R⁷ is chloro;
R¹⁰ is oxo; and
R¹¹ is hydrogen.

In a particularly preferred embodiment, the present invention provides a compound of formula (II) as described herein, or a pharmaceutically acceptable salt thereof, wherein
L² is a covalent bond;
A is phenyl;
B is triazolyl;
C is pyridyl;
R¹ is fluoro;
R² is fluoro;
R³ is hydrogen;
R⁴ is hydrogen;
R⁵ is a group

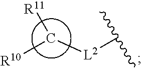

R⁶ is hydrogen;
R⁷ is absent;
R¹⁰ is hydroxy; and
R¹¹ is hydrogen or chloro.

In a particularly preferred embodiment, the present invention provides a compound of formula (II) as described herein, or a pharmaceutically acceptable salt thereof, wherein
L² is CH₂CH₂;
A is phenyl;
B is phenyl;
C is triazolyl or pyrazolyl;
R¹ is fluoro;
R² is fluoro;
R³ is hydrogen;
R⁴ is hydrogen;
R⁵ is a group

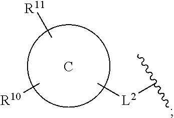

R⁶ is fluoro;
R⁷ is chloro;
R¹⁰ is hydrogen; and
R¹¹ is hydrogen.

In a particularly preferred embodiment, the present invention provides a compound of formula (II) as described herein, or a pharmaceutically acceptable salt thereof, wherein
L² is a covalent bond;
A is phenyl;
B is phenyl;
C is pyrrolyl;
R¹ is cyclopropyl; wherein said cyclopropyl is substituted with one methyl-SO₂—NH— substituent;
R² is chloro;
R³ is hydrogen;
R⁴ is hydrogen;
R⁵ is a group

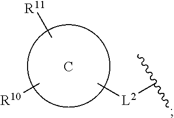

R⁶ is fluoro;
R⁷ is chloro;
R¹⁰ is cyano; and
R¹¹ is hydrogen.

In a preferred embodiment, the present invention provides a compound of formula (II) as described herein, or a pharmaceutically acceptable salt thereof, wherein L¹ is selected from a covalent bond and and NHCH₂.

In a preferred embodiment, the present invention provides a compound of formula (II) as described herein, or a pharmaceutically acceptable salt thereof, wherein L¹ is NHCH₂.

In a particularly preferred embodiment, the present invention provides a compound of formula (II) as described herein, or a pharmaceutically acceptable salt thereof, wherein L¹ is a covalent bond.

In a preferred embodiment, the present invention provides a compound of formula (II) as described herein, or a pharmaceutically acceptable salt thereof, wherein R⁸ is C₁-C₆-alkyl.

In a particularly preferred embodiment, the present invention provides a compound of formula (II) as described herein, or a pharmaceutically acceptable salt thereof, wherein R⁸ is methyl.

In a preferred embodiment, the present invention provides a compound of formula (II) as described herein, or a pharmaceutically acceptable salt thereof, wherein R⁹ is C₁-C₆-alkyl.

In a particularly preferred embodiment, the present invention provides a compound of formula (II) as described herein, or a pharmaceutically acceptable salt thereof, wherein R⁹ is methyl.

In one embodiment, the present invention provides a compound of formula (II) as described herein, or a pharmaceutically acceptable salt thereof, wherein:
L² is selected from a covalent bond, CH₂O, OCH₂, CH₂NH, NHCH₂, CH₂, CH₂CH₂, CF₂CH₂, and CH₂CF₂;
A is selected from furanyl, thienyl, oxazolyl, 1,3,4-oxadiazolyl, cyclopropyl, phenyl, pyrazinyl, pyridyl, and pyrimidinyl;
B is selected from triazolyl, imidazolyl, phenyl, and pyridyl;
C is selected from azetidine, cyclopropyl, piperidine, piperazine, pyridyl, pyrazine, pyrimidine, 1,2-dihydropyridine, 2-thia-6-azaspiro[3.3]heptane, 1,6-diazaspiro[3.3]heptane, 2,6-diazaspiro[3.3]heptane, 4,5-dihydroisoxazole, imidazolidine, oxazolidine, phenyl, pyrrolyl, pyrrolidinyl, pyrazolyl, and triazolyl;
R¹ is selected from halogen, hydroxy, carbamoyl-C₁-C₆-alkyl, C₁-C₆-alkyl, halo-C₁-C₆-alkyl, hydroxy-C₁-C₆-alkyl, C₁-C₆-alkoxy, halo-C₁-C₆-alkoxy, C₁-C₆-alkoxy-C₁-C₆-alkoxy, C₃-C₁₀-cycloalkyl, NH₂SO₂—, C₁-C₆-alkyl-SO₂—, C₁-C₆-alkyl-SO₂—C₁-C₆-alkyl-, (C₁-C₆-alkyl)₂-PO—C₁-C₆-alkyl-, C₁-C₆-alkyl-PO₂—C₁-C₆-alkyl-, C₁-C₆-alkyl-sulfonimidoyl-C₁-C₆-alkyl-, halo-C₁-C₆-alkyl-sulfonimidoyl-C₁-C₆-alkyl-, C₁-C₆-alkyl-NH—SO₂—C₁-C₆-alkyl-, and C₁-C₆-alkyl-SO₂—NH—C₁-C₆-alkyl-; wherein said C₃-C₁₀-cycloalkyl is optionally substituted with one substituent selected from carbamoyl, C₁-C₆-alkyl-SO₂—, and C₁-C₆-alkyl-SO₂—NH—;
   R², R³, and R⁴ are each independently absent or selected from hydrogen, halogen, and cyano;
   R⁵ is selected from C₁-C₆-alkoxy, C₁-C₆-alkoxy-C₁-C₆-alkyl, and a group

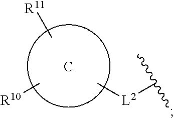

R⁶ is selected from hydrogen, halogen, and C₁-C₆-alkyl;
R⁷ is selected from hydrogen and halogen;
R⁸ is selected from C₁-C₆-alkyl, halo-C₁-C₆-alkyl, hydroxy-C₁-C₆-alkyl, C₃-C₁₀-cycloalkyl, and halo-C₃-C₁₀-cycloalkyl;
R⁹ is selected from hydrogen, C₁-C₆-alkyl, halo-C₁-C₆-alkyl, hydroxy-C₁-C₆-alkyl, C₃-C₁₀-cycloalkyl, and halo-C₃-C₁₀-cycloalkyl;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, oxo, and C₁-C₆-alkyl.

In one embodiment, the present invention provides a compound of formula (II) as described herein, or a pharmaceutically acceptable salt thereof, wherein A is:
(i)

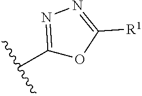

and R¹ is halo-C₁-C₆-alkyl; or
(ii) cyclopropyl; R¹ is halo-C₁-C₆-alkyl; and R², R³, and R⁴ are all hydrogen; or
(iii) phenyl; R¹ is selected from halogen, carbamoyl-C₁-C₆-alkyl, C₃-C₁₀-cycloalkyl, C₁-C₆-alkoxy, C₁-C₆-alkoxy-C₁-C₆-alkoxy, NH₂SO₂—, C₁-C₆-alkyl-SO₂—C₁-C₆-alkyl-, C₁-C₆-alkyl-NH—SO₂—C₁-C₆-alkyl-, C₁-C₆-alkyl-SO₂—NH—C₁-C₆-alkyl-, hydroxy-C₁-C₆-alkyl, and (C₁-C₆-alkyl)₂-PO—C₁-C₆-alkyl-; wherein said C₃-C₁₀-cycloalkyl is substituted with one substituent selected from carbamoyl, C₁-C₆-alkyl-SO₂—, and C₁-C₆-alkyl-SO₂—NH—; and R², R³, and R⁴ are each independently selected from hydrogen and halogen; or
(iv) pyrazinyl; R¹ is halo-C₁-C₆-alkyl; R² and R³ are both hydrogen; and R⁴ is absent; or
(v) pyridyl; R¹ is selected from hydroxy, carbamoyl-C₁-C₆-alkyl, C₁-C₆-alkyl-SO₂—, and C₁-C₆-alkyl-SO₂—NH—C₁-C₆-alkyl-; R² is selected from hydrogen and halogen; and R³ and R⁴ are both hydrogen; or
(vi) pyrimidinyl; R¹ is halo-C₁-C₆-alkyl; R² and R³ are both hydrogen; and R⁴ is absent.

In one embodiment, the present invention provides a compound of formula (II) as described herein, or a pharmaceutically acceptable salt thereof, wherein the group

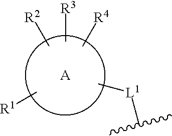

is selected from

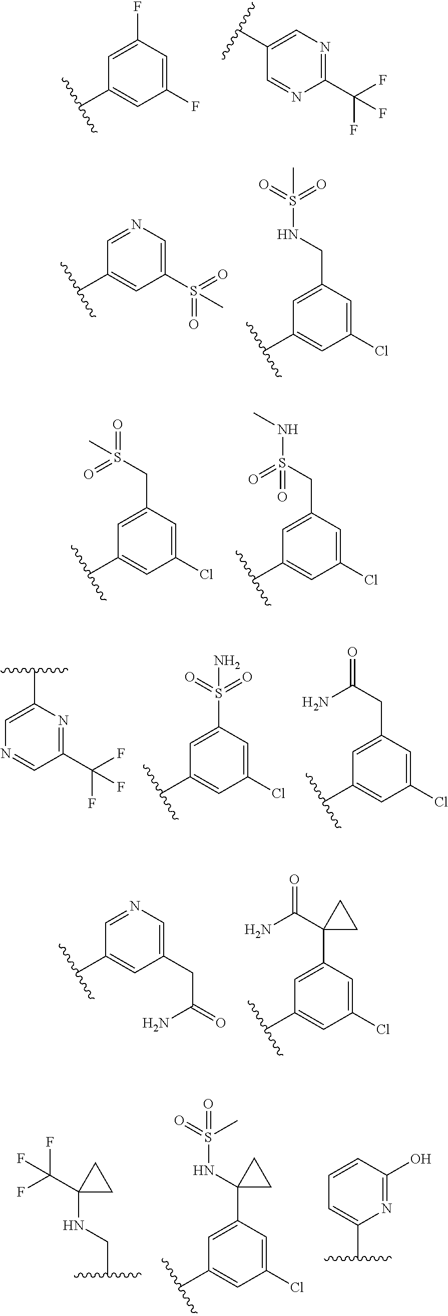

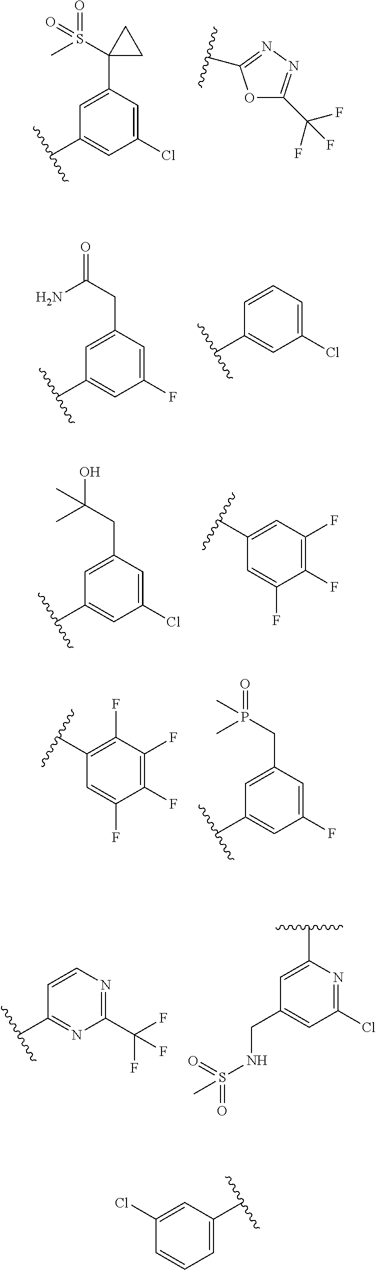

In a preferred embodiment, the present invention provides a compound of formula (II) as described herein, or a pharmaceutically acceptable salt thereof, wherein:
A is phenyl;
R¹ is halogen or C₃-C₁₀-cycloalkyl substituted with one C₁-C₆-alkyl-SO₂—NH— substituent;
R² is halogen; and
R³ and R⁴ are both hydrogen.

In a particularly preferred embodiment, the present invention provides a compound of formula (II) as described herein, or a pharmaceutically acceptable salt thereof, wherein:
A is phenyl;
R¹ is fluoro or cyclopropyl substituted with one methyl-SO₂—NH— substituent;
R² is fluoro or chloro; and
R³ and R⁴ are both hydrogen.

In one embodiment, the present invention provides a compound of formula (II) as described herein, or a pharmaceutically acceptable salt thereof, wherein:
B is selected from triazolyl, phenyl, and pyridyl;
C is selected from azetidine, cyclopropyl, pyridyl, 1,2-dihydropyridine, 2-thia-6-azaspiro[3.3]heptane, 1,6-diazaspiro[3.3]heptane, 4,5-dihydroisoxazole, imidazolidine, oxazolidine, phenyl, pyrrolyl, pyrrolidinyl, pyrazolyl, and triazolyl;
L² is selected from a covalent bond, CH₂O, CH₂, NHCH₂, and CH₂CH₂;
R⁵ is selected from C₁-C₆-alkoxy and a group

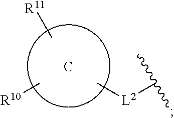

R⁶ is selected from hydrogen, halogen, and C₁-C₆-alkyl;
R⁷ is selected from hydrogen and halogen;
R¹⁰ is selected from hydrogen, cyano, hydroxy, halogen, oxo, C₁-C₆-alkyl-SO₂—, NH₂SO₂—, and halo-C₁-C₆-alkyl; and
R¹¹ is selected from hydrogen, hydroxy, and oxo.

In one embodiment, the present invention provides a compound of formula (II) as described herein, or a pharmaceutically acceptable salt thereof, wherein the group

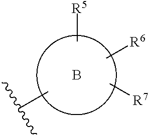

is selected from:

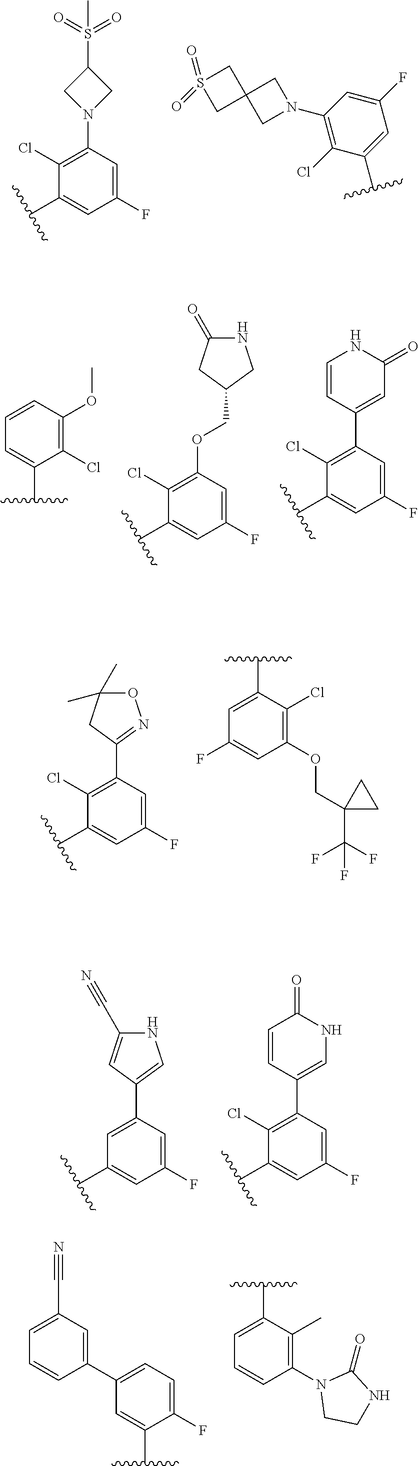

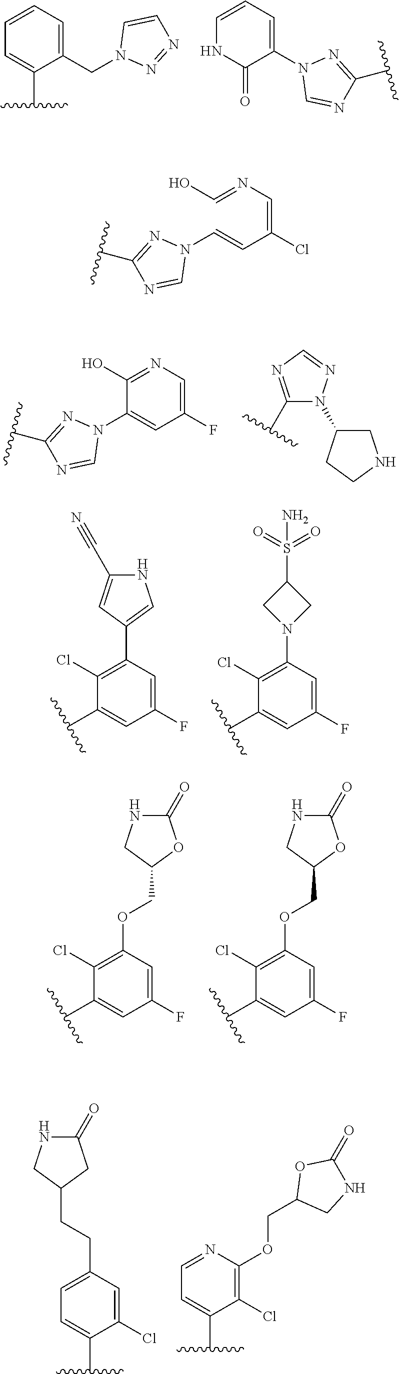

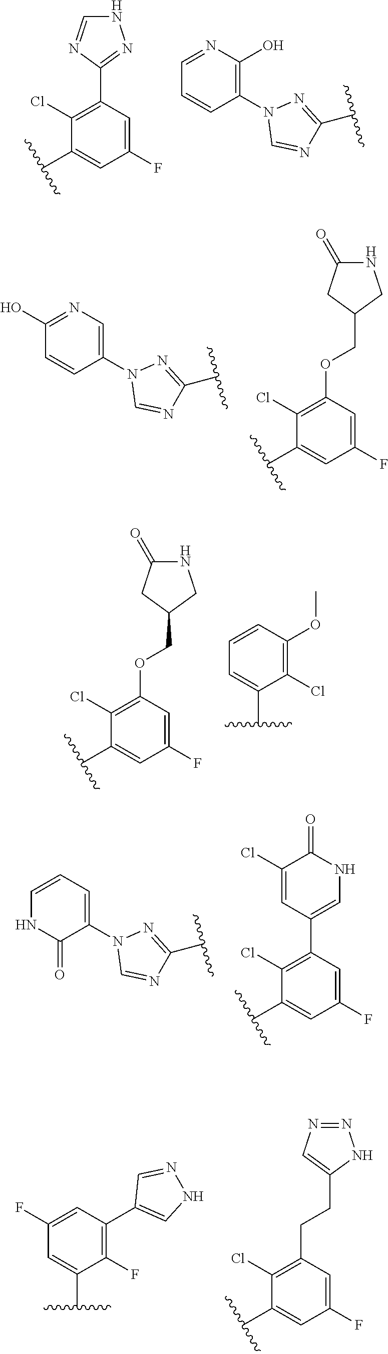

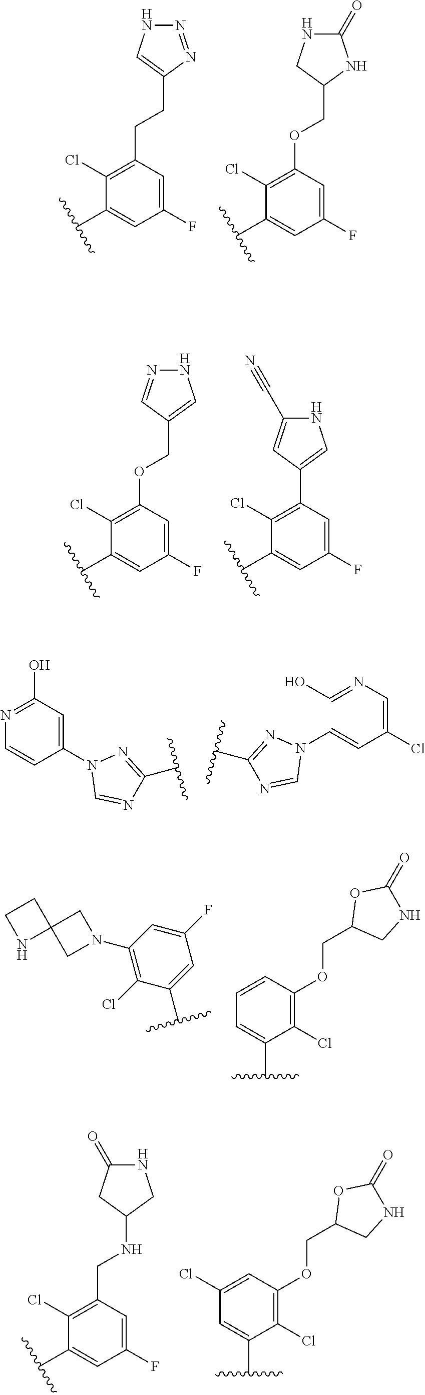

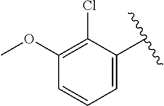 [SMILES: *c1cccc(OC)c1Cl]

In a preferred embodiment, the present invention provides a compound of formula (II) as described herein, or a pharmaceutically acceptable salt thereof, wherein:
B is selected from triazolyl and phenyl;
C is selected from imidazolidine, triazolyl, pyrrolyl, pyrrolidinyl, pyrazolyl, pyridyl, 1,2-dihydropyridine, and oxazolidine;
L² is selected from a covalent bond, CH₂O, NHCH₂, and CH₂CH₂;
R⁵ is selected from C₁-C₆-alkoxy and a group

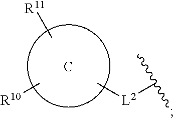

R⁶ is selected from hydrogen and halogen;
R⁷ is selected from hydrogen and halogen;
R¹⁰ is selected from hydrogen, halogen, cyano, and oxo; and
R¹¹ is selected from hydrogen and hydroxy.

In a particularly preferred embodiment, the present invention provides a compound of formula (II) as described herein, or a pharmaceutically acceptable salt thereof, wherein:
B is selected from triazolyl and phenyl;
C is selected from imidazolidine, triazolyl, pyrrolyl, pyrrolidinyl, pyrazolyl, pyridyl, 1,2-dihydropyridine, and oxazolidine;
L² is selected from a covalent bond, CH₂O, NHCH₂, and CH₂CH₂;
R⁵ is selected from methoxy and a group

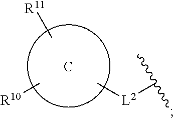

R⁶ is selected from hydrogen and fluoro;
R⁷ is selected from hydrogen and chloro;
R¹⁰ is selected from hydrogen, chloro, cyano, and oxo; and
R¹¹ is selected from hydrogen and hydroxy.

In one embodiment, the present invention provides a compound of formula (II) as described herein, or a pharmaceutically acceptable salt thereof, wherein:
X is NR⁹ and Y is C; or
X is CR⁹ or N and Y is N;
L¹ is selected from a covalent bond and NHCH₂;
L² is selected from a covalent bond, CH₂O, CH₂, NHCH₂, and CH₂CH₂;
A is:
   (i)

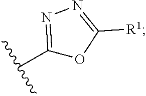

R¹ is halo-C₁-C₆-alkyl; and R², R³, and R⁴ are all absent; or
(ii) cyclopropyl; R¹ is halo-C₁-C₆-alkyl; and R², R³, and R⁴ are all hydrogen; or
(iii) phenyl; R¹ is selected from halogen, carbamoyl-C₁-C₆-alkyl, C₃-C₁₀-cycloalkyl, C₁-C₆-alkoxy-C₁-C₆-alkoxy, NH₂SO₂—, C₁-C₆-alkyl-SO₂—C₁-C₆-alkyl-, C₁-C₆-alkyl-NH—SO₂—C₁-C₆-alkyl-, C₁-C₆-alkyl-SO₂—NH—C₁-C₆-alkyl-, hydroxy-C₁-C₆-alkyl, and (C₁-C₆-alkyl)₂-PO—C₁-C₆-alkyl-; wherein said C₃-C₁₀-cycloalkyl is substituted with one substituent selected from carbamoyl, C₁-C₆-alkyl-SO₂—, and C₁-C₆-alkyl-SO₂—NH—; and R², R³, and R⁴ are each independently selected from hydrogen and halogen; or
(iv) pyrazinyl; R¹ is halo-C₁-C₆-alkyl; R² and R³ are both hydrogen; and R⁴ is absent; or
(v) pyridyl; R¹ is selected from hydroxy, carbamoyl-C₁-C₆-alkyl, C₁-C₆-alkyl-SO₂—, and C₁-C₆-alkyl-SO₂—NH—C₁-C₆-alkyl-; R² is selected from hydrogen and halogen; and R³ and R⁴ are both hydrogen; or
(vi) pyrimidinyl; R¹ is halo-C₁-C₆-alkyl; R² and R³ are both hydrogen; and R⁴ is absent;
B is selected from triazolyl, phenyl, and pyridyl;
C is selected from azetidine, cyclopropyl, pyridyl, 1,2-dihydropyridine, 2-thia-6-azaspiro[3.3]heptane, 1,6-diazaspiro[3.3]heptane, 4,5-dihydroisoxazole, imidazolidine, oxazolidine, phenyl, pyrrolyl, pyrrolidinyl, pyrazolyl, and triazolyl;
R⁵ is selected from C₁-C₆-alkoxy and a group

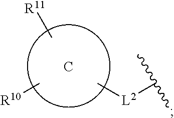

R⁶ is selected from hydrogen, halogen, and C₁-C₆-alkyl;
R⁷ is selected from hydrogen and halogen;
R⁸ and R⁹ are both C₁-C₆-alkyl;
R¹⁰ is selected from hydrogen, cyano, hydroxy, halogen, oxo, C₁-C₆-alkyl-SO₂—, NH₂SO₂—, and halo-C₁-C₆-alkyl; and
R¹¹ is selected from hydrogen, hydroxy, and oxo.

In a preferred embodiment, the present invention provides a compound of formula (II) as described herein, or a pharmaceutically acceptable salt thereof, wherein:
X is NR⁹ and Y is C;
L¹ is a covalent bond;
L² is selected from a covalent bond, CH₂O, NHCH₂, and CH₂CH₂;
A is phenyl;
B is selected from triazolyl and phenyl;
C is selected from imidazolidine, triazolyl, pyrrolyl, pyrrolidinyl, pyrazolyl, pyridyl, 1,2-dihydropyridine, and oxazolidine;
R¹ is halogen or C₃-C₁₀-cycloalkyl substituted with one C₁-C₆-alkyl-SO₂—NH— substituent;
R² is halogen;
R³ and R⁴ are both hydrogen;
R⁵ is selected from C₁-C₆-alkoxy and a group

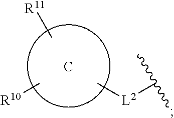

R⁶ is selected from hydrogen and halogen;
R⁷ is selected from hydrogen and halogen;
R⁸ and R⁹ are both C₁-C₆-alkyl;
R¹⁰ is selected from hydrogen, halogen, cyano, and oxo; and
R¹¹ is selected from hydrogen and hydroxy.

In a particularly preferred embodiment, the present invention provides a compound of formula (II) as described herein, or a pharmaceutically acceptable salt thereof, wherein:
X is NR⁹ and Y is C;
L¹ is a covalent bond;
L² is selected from a covalent bond, CH₂O, NHCH₂, and CH₂CH₂;
A is phenyl;
B is selected from triazolyl and phenyl;
C is selected from imidazolidine, triazolyl, pyrrolyl, pyrrolidinyl, pyrazolyl, pyridyl, 1,2-dihydropyridine, and oxazolidine;
   R¹ is fluoro or cyclopropyl substituted with one methyl-SO₂—NH— substituent;
R² is fluoro or chloro;
R³ and R⁴ are both hydrogen;
R⁵ is selected from methoxy and a group

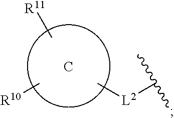

R⁶ is selected from hydrogen and fluoro;
R⁷ is selected from hydrogen and chloro;
R⁸ and R⁹ are both methyl;
R¹⁰ is selected from hydrogen, chloro, cyano, and oxo; and
R¹¹ is selected from hydrogen and hydroxy.

In one embodiment, the present invention provides a compound of formula (II) as described herein, or a pharmaceutically acceptable salt thereof, wherein L¹ is selected from a covalent bond and NHCH₂.

In one embodiment, the present invention provides a compound of formula (II) as described herein, or a pharmaceutically acceptable salt thereof, wherein L² is selected from a covalent bond, CH₂O, CH₂, NHCH₂, and CH₂CH₂.

In one embodiment, the present invention provides a compound of formula (II) as described herein, or a pharmaceutically acceptable salt thereof, wherein A is

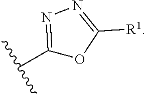

In one embodiment, the present invention provides a compound of formula (II) as described herein, or a pharmaceutically acceptable salt thereof, wherein A is cyclopropyl.

In a particularly preferred embodiment, the present invention provides a compound of formula (II) as described herein, or a pharmaceutically acceptable salt thereof, wherein A is phenyl.

In one embodiment, the present invention provides a compound of formula (II) as described herein, or a pharmaceutically acceptable salt thereof, wherein A is pyrazinyl. In one embodiment, the present invention provides a compound of formula (II) as described herein, or a pharmaceutically acceptable salt thereof, wherein A is pyridyl.

In one embodiment, the present invention provides a compound of formula (II) as described herein, or a pharmaceutically acceptable salt thereof, wherein A is pyrimidinyl.

In one embodiment, the present invention provides a compound of formula (II) as described herein, or a pharmaceutically acceptable salt thereof, wherein B is selected from triazolyl, phenyl, and pyridyl.

In one embodiment, the present invention provides a compound of formula (II) as described herein, or a pharmaceutically acceptable salt thereof, wherein C is selected from azetidine, cyclopropyl, pyridyl, 1,2-dihydropyridine, 2-thia-6-azaspiro[3.3]heptane, 1,6-diazaspiro[3.3]heptane, 4,5-dihydroisoxazole, imidazolidine, oxazolidine, phenyl, pyrrolyl, pyrrolidinyl, pyrazolyl, and triazolyl.

In one embodiment, the present invention provides a compound of formula (II) as described herein, or a pharmaceutically acceptable salt thereof, wherein R⁵ is selected from C₁-C₆-alkoxy and a group

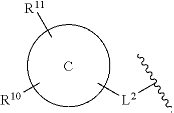

In one embodiment, the present invention provides a compound of formula (II) as described herein, or a pharmaceutically acceptable salt thereof, wherein R⁶ is selected from hydrogen, halogen, and C₁-C₆-alkyl.

In one embodiment, the present invention provides a compound of formula (II) as described herein, or a pharmaceutically acceptable salt thereof, wherein R⁷ is selected from hydrogen and halogen.

In one embodiment, the present invention provides a compound of formula (II) as described herein, or a pharmaceutically acceptable salt thereof, wherein R¹⁰ is selected from hydrogen, cyano, hydroxy, halogen, oxo, C₁-C₆-alkyl-SO₂—, NH₂SO₂—, and halo-C₁-C₆-alkyl.

In one embodiment, the present invention provides a compound of formula (II) as described herein, or a pharmaceutically acceptable salt thereof, wherein R¹¹ is selected from hydrogen, hydroxy, and oxo.

In a preferred embodiment, the present invention provides a compound of formula (II) as described herein, or a pharmaceutically acceptable salt thereof, wherein L² is selected from a covalent bond, CH₂O, NHCH₂, and CH₂CH₂.

In a preferred embodiment, the present invention provides a compound of formula (II) as described herein, or a pharmaceutically acceptable salt thereof, wherein B is selected from triazolyl and phenyl.

In a preferred embodiment, the present invention provides a compound of formula (II) as described herein, or a pharmaceutically acceptable salt thereof, wherein B is triazolyl.

In a particularly preferred embodiment, the present invention provides a compound of formula (II) as described herein, or a pharmaceutically acceptable salt thereof, wherein B is phenyl.

In a preferred embodiment, the present invention provides a compound of formula (II) as described herein, or a pharmaceutically acceptable salt thereof, wherein C is selected from imidazolidine, triazolyl, pyrrolyl, pyrrolidinyl, pyrazolyl, pyridyl, 1,2-dihydropyridine, and oxazolidine.

In a preferred embodiment, the present invention provides a compound of formula (II) as described herein, or a pharmaceutically acceptable salt thereof, wherein R¹ is halogen or C₃-C₁₀-cycloalkyl substituted with one C₁-C₆-alkyl-SO₂—NH— substituent.

In a preferred embodiment, the present invention provides a compound of formula (II) as described herein, or a pharmaceutically acceptable salt thereof, wherein R² is halogen.

In a preferred embodiment, the present invention provides a compound of formula (II) as described herein, or a pharmaceutically acceptable salt thereof, wherein R³ is hydrogen.

In a preferred embodiment, the present invention provides a compound of formula (II) as described herein, or a pharmaceutically acceptable salt thereof, wherein R⁴ is hydrogen.

In a preferred embodiment, the present invention provides a compound of formula (II) as described herein, or a pharmaceutically acceptable salt thereof, wherein R⁵ is selected from C₁-C₆-alkoxy and a group

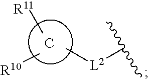

wherein C, L², R¹⁰, and R¹¹ are as described herein.

In a preferred embodiment, the present invention provides a compound of formula (II) as described herein, or a pharmaceutically acceptable salt thereof, wherein R⁵ is C₁-C₆-alkoxy.

In a particularly preferred embodiment, the present invention provides a compound of formula (II) as described herein, or a pharmaceutically acceptable salt thereof, wherein R⁵ is a group

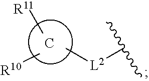

wherein C, L², R¹⁰, and R¹¹ are as described herein.

In a preferred embodiment, the present invention provides a compound of formula (II) as described herein, or a pharmaceutically acceptable salt thereof, wherein R⁶ is selected from hydrogen and halogen.

In a preferred embodiment, the present invention provides a compound of formula (II) as described herein, or a pharmaceutically acceptable salt thereof, wherein R⁷ is selected from hydrogen and halogen.

In a preferred embodiment, the present invention provides a compound of formula (II) as described herein, or a pharmaceutically acceptable salt thereof, wherein R¹⁰ is selected from hydrogen, halogen, cyano, and oxo.

In a preferred embodiment, the present invention provides a compound of formula (II) as described herein, or a pharmaceutically acceptable salt thereof, wherein R¹¹ is selected from hydrogen and hydroxy.

In a particularly preferred embodiment, the present invention provides a compound of formula (II) as described herein, or a pharmaceutically acceptable salt thereof, wherein R¹ is fluoro or cyclopropyl substituted with one methyl-SO₂—NH— substituent.

In a particularly preferred embodiment, the present invention provides a compound of formula (II) as described herein, or a pharmaceutically acceptable salt thereof, wherein R² is fluoro or chloro.

In a particularly preferred embodiment, the present invention provides a compound of formula (II) as described herein, or a pharmaceutically acceptable salt thereof, wherein R⁵ is selected from methoxy and a group

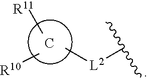

In a particularly preferred embodiment, the present invention provides a compound of formula (II) as described herein, or a pharmaceutically acceptable salt thereof, wherein R⁶ is selected from hydrogen and fluoro.

In a particularly preferred embodiment, the present invention provides a compound of formula (II) as described herein, or a pharmaceutically acceptable salt thereof, wherein R⁷ is selected from hydrogen and chloro.

In a particularly preferred embodiment, the present invention provides a compound of formula (II) as described herein, or a pharmaceutically acceptable salt thereof, wherein R¹⁰ is selected from hydrogen, chloro, cyano, and oxo.

In a particularly preferred embodiment, the present invention provides a compound of formula (II) as described herein, or a pharmaceutically acceptable salt thereof, wherein the group

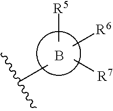

is

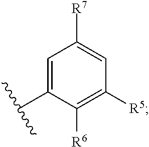

wherein R⁵, R⁶, and R⁷ are as defined herein.

In one embodiment, the compound of formula (II) according to the invention is a compound of formula (IIa) or (IIb), or a pharmaceutically acceptable salt thereof,
A is phenyl;
R¹ is halogen; and
R², R³, and R⁴ are each hydrogen.

In one embodiment, the compound of formula (II) according to the invention is a compound of formula (IIa) or (IIb), or a pharmaceutically acceptable salt thereof,
B is phenyl;
R⁵ is C₁-C₆-alkoxy;
R⁶ is halogen; and
R⁷ is hydrogen.

In one embodiment, the compound of formula (II) according to the invention is a compound of formula (IIa) or (IIb), or a pharmaceutically acceptable salt thereof,
A is phenyl;
R¹ is chloro; and
R², R³, and R⁴ are each hydrogen.

In one embodiment, the compound of formula (II) according to the invention is a compound of formula (IIa) or (IIb), or a pharmaceutically acceptable salt thereof,
B is phenyl;
R⁵ is methoxy;
R⁶ is chloro; and
R⁷ is hydrogen.

In one embodiment, the compound of formula (II) according to the invention is a compound of formula (IIa) or (IIb), or a pharmaceutically acceptable salt thereof,
A is phenyl;
B is phenyl;
R¹ is halogen;
R², R³, and R⁴ are each hydrogen;
R⁵ is C₁-C₆-alkoxy;
R⁶ is halogen; and
R⁷ is hydrogen.

In one embodiment, the compound of formula (II) according to the invention is a compound of formula (IIa) or (IIb), or a pharmaceutically acceptable salt thereof,
A is phenyl;
B is phenyl;
R¹ is chloro;
R², R³, and R⁴ are each hydrogen;
R⁵ is methoxy;
R⁶ is chloro; and
R⁷ is hydrogen.

In a further aspect, the present invention provides a compound of Formula (I)

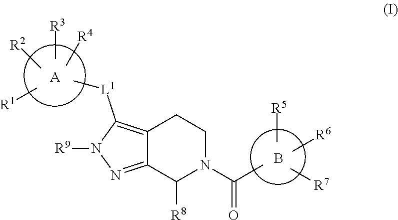

or a pharmaceutically acceptable salt thereof, wherein:
L¹ is selected from a covalent bond, NHCH₂, and CH₂NH;
L² is selected from a covalent bond, CH₂O, OCH₂, CH₂, CH₂CH₂, CF₂CH₂, and CH₂CF₂;
A is selected from oxazolyl, 1,3,4-oxadiazolyl, cyclopropyl, phenyl, pyrazinyl, pyridyl, and pyrimidinyl;
B is selected from 2H-triazol-4-yl, imidazolyl, 1H-1,2,4-triazol-3-yl, phenyl, and pyridyl;
C is selected from azetidine, cyclopropyl, piperidine, piperazine, pyridyl, pyrazine, pyrimidine, 1,2-dihydropyridine, 2-thia-6-azaspiro[3.3]heptane, 1,6-diazaspiro[3.3]heptane, 4,5-dihydroisoxazole, imidazolidine, oxazolidine, phenyl, pyrrolyl, pyrrolidinyl, pyrazolyl, triazol-1-yl, 1H-triazol-4-yl, and 1H-1,2,4-triazol-3-yl;
R¹ is selected from halogen, hydroxy, carbamoyl-C₁-C₆-alkyl, C₁-C₆-alkyl, halo-C₁-C₆-alkyl, C₁-C₆-alkoxy-C₁-C₆-alkoxy, C₃-C₁₀-cycloalkyl, NH₂SO₂—, C₁-C₆-alkyl-SO₂—, C₁-C₆-alkyl-SO₂—C₁-C₆-alkyl-, (C₁-C₆-alkyl)₂-PO—C₁-C₆-alkyl-, C₁-C₆-alkyl-sulfonimidoyl-C₁-C₆-alkyl-, halo-C₁-C₆-alkyl-sulfonimidoyl-C₁-C₆-alkyl-, C₁-C₆-alkyl-NH—SO₂—C₁-C₆-alkyl-, and C₁-C₆-alkyl-SO₂—NH—C₁-C₆-alkyl-; wherein said C₃-C₁₀-cycloalkyl is substituted with one substituent selected from carbamoyl, C₁-C₆-alkyl-SO₂—, and C₁-C₆-alkyl-SO₂—NH—;
R², R³, and R⁴ are each independently selected from hydrogen, halogen, and cyano;
R⁵ is selected from C₁-C₆-alkoxy and a group

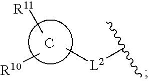

R⁶ is selected from hydrogen, halogen, and C₁-C₆-alkyl;
R⁷ is selected from hydrogen and halogen;
R⁸ is selected from C₁-C₆-alkyl, halo-C₁-C₆-alkyl, hydroxy-C₁-C₆-alkyl, C₃-C₁₀-cycloalkyl, and halo-C₃-C₁₀-cycloalkyl;
R⁹ is selected from C₁-C₆-alkyl, halo-C₁-C₆-alkyl, hydroxy-C₁-C₆-alkyl, C₃-C₁₀-cycloalkyl, and halo-C₃-C₁₀-cycloalkyl;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, oxo, and C₁-C₆-alkyl.

In a preferred embodiment, the present invention provides a compound of formula (I) as described herein, or a pharmaceutically acceptable salt thereof, wherein L¹ is a covalent bond.

In one embodiment, the present invention provides a compound of formula (I) as described herein, or a pharmaceutically acceptable salt thereof, wherein A is:
(i)

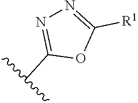

and R¹ is halo-C₁-C₆-alkyl; or
(ii) cyclopropyl; R¹ is halo-C₁-C₆-alkyl; and R², R³, and R⁴ are all hydrogen; or
(iii) phenyl; R¹ is selected from halogen, carbamoyl-C₁-C₆-alkyl, C₃-C₁₀-cycloalkyl, C₁-C₆-alkoxy-C₁-C₆-alkoxy, NH₂SO₂—, C₁-C₆-alkyl-SO₂—C₁-C₆-alkyl-, C₁-C₆-alkyl-NH—SO₂—C₁-C₆-alkyl-, and C₁-C₆-alkyl-SO₂—NH—C₁-C₆-alkyl-; wherein said C₃-C₁₀-cycloalkyl is substituted with one substituent selected from carbamoyl, C₁-C₆-alkyl-SO₂—, and C₁-C₆-alkyl-SO₂—NH—; R² is halogen; and R³ and R⁴ are both hydrogen; or
(iv) pyrazinyl; R¹ is halo-C₁-C₆-alkyl; R² and R³ are both hydrogen; and R⁴ is absent; or
(v) pyridyl; R¹ is selected from hydroxy, carbamoyl-C₁-C₆-alkyl, and C₁-C₆-alkyl-SO₂—; and R², R³, and R⁴ are all hydrogen; or
(vi) pyrimidinyl; R¹ is halo-C₁-C₆-alkyl; R² and R³ are both hydrogen; and R⁴ is absent.

In one embodiment, the present invention provides a compound of formula (I) as described herein, or a pharmaceutically acceptable salt thereof, wherein A is:
(i)

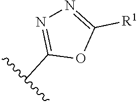

and R¹ is halo-C₁-C₆-alkyl; or
(ii) phenyl; R¹ is selected from carbamoyl-C₁-C₆-alkyl, C₃-C₁₀-cycloalkyl, C₁-C₆-alkoxy-C₁-C₆-alkoxy, NH₂SO₂—, C₁-C₆-alkyl-SO₂—C₁-C₆-alkyl-, C₁-C₆-alkyl-NH—SO₂—C₁-C₆-alkyl-, and C₁-C₆-alkyl-SO₂—NH—C₁-C₆-alkyl-; wherein said C₃-C₁₀-cycloalkyl is substituted with one substituent selected from carbamoyl, C₁-C₆-alkyl-SO₂—, and C₁-C₆-alkyl-SO₂—NH—; R² is halogen; and R³ and R⁴ are both hydrogen; or
(iii) pyrazinyl; R¹ is halo-C₁-C₆-alkyl; R² and R³ are both hydrogen; and R⁴ is absent; or
(iv) pyridyl; R¹ is selected from hydroxy, carbamoyl-C₁-C₆-alkyl, and C₁-C₆-alkyl-SO₂—; and R², R³, and R⁴ are all hydrogen; or
(v) pyrimidinyl; R¹ is halo-C₁-C₆-alkyl; R² and R³ are both hydrogen; and R⁴ is absent.

In one embodiment, the present invention provides a compound of formula (I) as described herein, or a pharmaceutically acceptable salt thereof, wherein the group

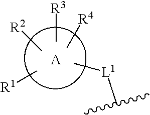

is selected from:

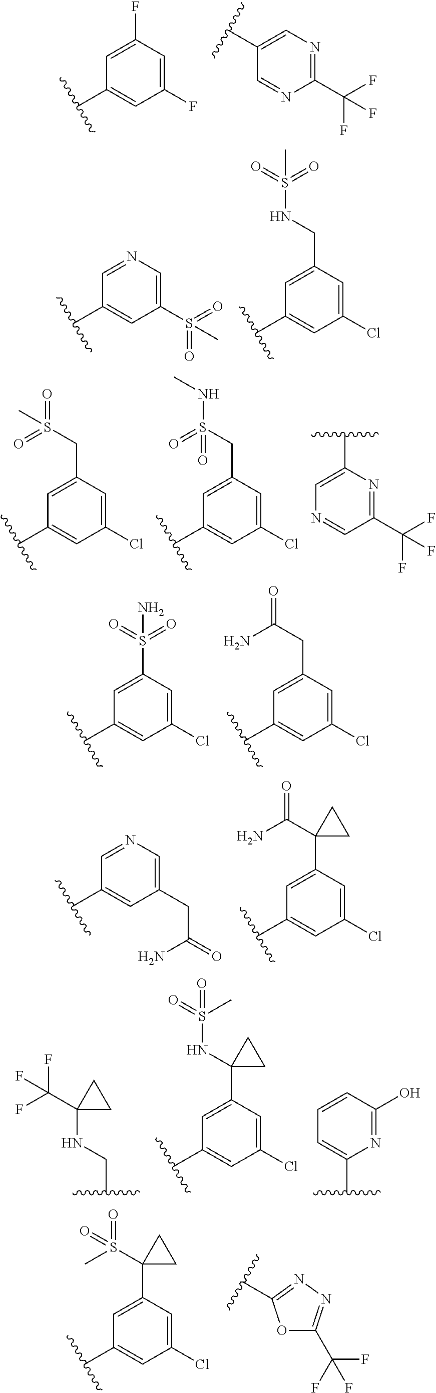

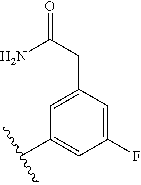 [SMILES: *c1cc(F)cc(CC(N)=O)c1]

In a preferred embodiment, the present invention provides a compound of formula (I) as described herein, or a pharmaceutically acceptable salt thereof, wherein:
A is phenyl;
R¹ and R² are both halogen; and
R³ and R⁴ are both hydrogen.

In a particularly preferred embodiment, the present invention provides a compound of formula (I) as described herein, or a pharmaceutically acceptable salt thereof, wherein:
A is phenyl;
R¹ and R² are both fluoro; and
R³ and R⁴ are both hydrogen.

In one embodiment, the present invention provides a compound of formula (I) as described herein, or a pharmaceutically acceptable salt thereof, wherein:
B is selected from 1H-1,2,4-triazol-3-yl, phenyl, and pyridyl;
C is selected from azetidine, cyclopropyl, pyridyl, 1,2-dihydropyridine, 2-thia-6-azaspiro[3.3]heptane, 4,5-dihydroisoxazole, imidazolidine, oxazolidine, phenyl, pyrrolyl, pyrrolidinyl, triazol-1-yl, and 1H-1,2,4-triazol-3-yl;
   L² is selected from a covalent bond, CH₂O, CH₂, and CH₂CH₂;
R⁵ is selected from C₁-C₆-alkoxy and a group

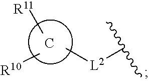

R⁶ is selected from hydrogen, halogen, and C₁-C₆-alkyl;
R⁷ is selected from hydrogen and halogen;
R¹⁰ is selected from hydrogen, cyano, hydroxy, halogen, oxo, C₁-C₆-alkyl-SO₂—, NH₂SO₂—, and halo-C₁-C₆-alkyl; and
R¹¹ is selected from hydrogen, hydroxy, and oxo.

In one embodiment, the present invention provides a compound of formula (I) as described herein, or a pharmaceutically acceptable salt thereof, wherein the group

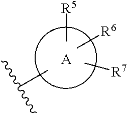

is selected from:

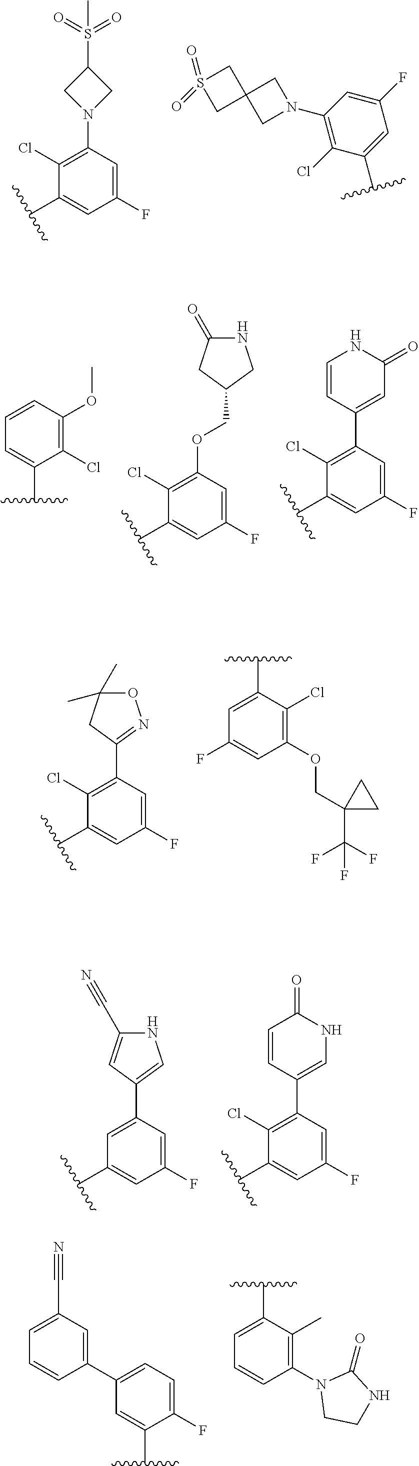

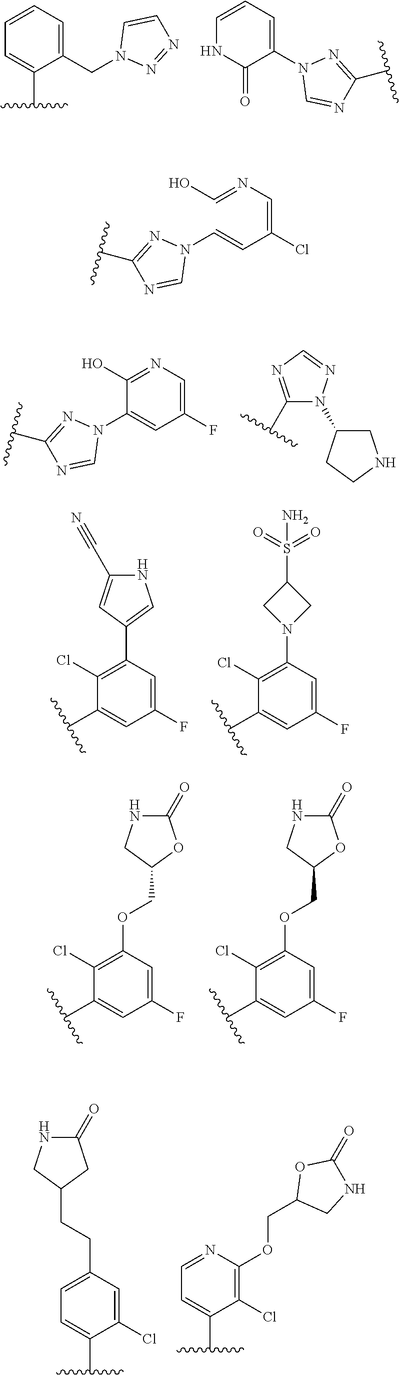

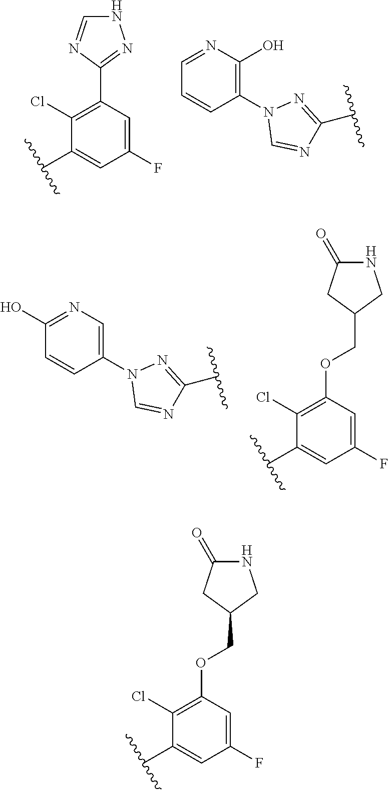 [SMILES: *c1cc(F)cc(-c2nc[nH]n2)c1Cl ; *c1ncn(-c2cccnc2O)n1 ; *c1ncn(-c2ccc(O)nc2)n1 ; *c1cc(F)cc(OCC2CNC(=O)C2)c1Cl ; *c1cc(F)cc(OC[C@@H]2CNC(=O)C2)c1Cl]

In a preferred embodiment, the present invention provides a compound of formula (I) as described herein, or a pharmaceutically acceptable salt thereof, wherein:
B is selected from 1H-1,2,4-triazol-3-yl and phenyl;
C is selected from pyridyl, 1,2-dihydropyridine, and oxazolidine;
L² is selected from a covalent bond and CH₂O;
R⁵ is selected from C₁-C₆-alkoxy and a group

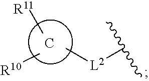

R⁶ is selected from hydrogen and halogen;
R⁷ is selected from hydrogen and halogen;
R¹⁰ is selected from hydrogen, halogen, and oxo; and
R¹¹ is selected from hydrogen and hydroxy.

In a particularly preferred embodiment, the present invention provides a compound of formula (I) as described herein, or a pharmaceutically acceptable salt thereof, wherein:
B is selected from 1H-1,2,4-triazol-3-yl and phenyl;
C is selected from pyridyl, 1,2-dihydropyridine, and oxazolidine;
L² is selected from a covalent bond and CH₂O;
R⁵ is selected from C₁-C₆-alkoxy and a group

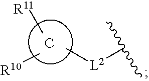

R⁶ is selected from hydrogen and fluoro;
R⁷ is selected from hydrogen and chloro;
R¹⁰ is selected from hydrogen, chloro, and oxo; and
R¹¹ is selected from hydrogen and hydroxy.

In a preferred embodiment, the present invention provides a compound of formula (I) as described herein, or a pharmaceutically acceptable salt thereof, wherein R⁸ is C₁-C₆-alkyl.

In a particularly preferred embodiment, the present invention provides a compound of formula (I) as described herein, or a pharmaceutically acceptable salt thereof, wherein R⁸ is methyl.

In a preferred embodiment, the present invention provides a compound of formula (I) as described herein, or a pharmaceutically acceptable salt thereof, wherein R⁹ is C₁-C₆-alkyl.

In a particularly preferred embodiment, the present invention provides a compound of formula (I) as described herein, or a pharmaceutically acceptable salt thereof, wherein R⁹ is methyl.

In one embodiment, the present invention provides a compound of formula (I) as described herein, or a pharmaceutically acceptable salt thereof, selected from:
4-[[2-Chloro-3-[3-(3,5-difluorophenyl)-2,7-dimethyl-5,7-dihydro-4H-pyrazolo[3,4-c]pyridine-6-carbonyl]-5-fluoro-phenoxy]methyl]pyrrolidin-2-one;
5-[2-Chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-1H-pyridin-2-one;
5-[[2-Chloro-3-[3-(3,5-difluorophenyl)-2,7-dimethyl-5,7-dihydro-4H-pyrazolo[3,4-c]pyridine-6-carbonyl]-5-fluoro-phenoxy]methyl]oxazolidin-2-one;
4-[3-[(7S)-3-(3,5-Difluorophenyl)-2,7-dimethyl-5,7-dihydro-4H-pyrazolo[3,4-c]pyridine-6-carbonyl]-5-fluoro-phenyl]-1H-pyrrole-2-carbonitrile;
4-[2-Chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-1H-pyridin-2-one;
N-[1-[3-Chloro-5-[6-[2-chloro-5-fluoro-3-(6-oxo-1H-pyridin-3-yl)benzoyl]-2,7-dimethyl-5,7-dihydro-4H-pyrazolo[3,4-c]pyridin-3-yl]phenyl]cyclopropyl]methanesulfonamide;
4-[2-Chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-1H-pyrrole-2-carbonitrile; 3-Chloro-5-[6-[2-chloro-5-fluoro-3-(6-oxo-1H-pyridin-3-yl)benzoyl]-2,7-dimethyl-5,7-dihydro-4H-pyrazolo[3,4-c]pyridin-3-yl]benzenesulfonamide;
4-[2-Chloro-3-[(7S)-2,7-dimethyl-3-[6-(trifluoromethyl)pyrazin-2-yl]-5,7-dihydro-4H-pyrazolo[3,4-c]pyridine-6-carbonyl]-5-fluoro-phenyl]-1H-pyrrole-2-carbonitrile;
N-[[3-Chloro-5-[(7S)-6-(2-chloro-3-methoxy-benzoyl)-2,7-dimethyl-5,7-dihydro-4H-pyrazolo[3,4-c]pyridin-3-yl]phenyl]methyl]methanesulfonamide;
N-[1-[3-Chloro-5-[(7S)-6-(2-chloro-3-methoxy-benzoyl)-2,7-dimethyl-5,7-dihydro-4H-pyrazolo[3,4-c]pyridin-3-yl]phenyl]cyclopropyl]methanesulfonamide;
5-[[3-Chloro-4-[(7S)-3-(3,5-difluorophenyl)-2,7-dimethyl-5,7-dihydro-4H-pyrazolo[3,4-c]pyridine-6-carbonyl]-2-pyridyl]oxymethyl]oxazolidin-2-one;
[2-Chloro-3-(2,2-dioxo-2λ⁶-thia-6-azaspiro[3.3]heptan-6-yl)-5-fluoro-phenyl]-[(7S)-3-(3,5-difluorophenyl)-2,7-dimethyl-5,7-dihydro-4H-pyrazolo[3,4-c]pyridin-6-yl]methanone;
[2-Chloro-5-fluoro-3-(3-methylsulfonylazetidin-1-yl)phenyl]-[(7S)-3-(3,5-difluorophenyl)-2,7-dimethyl-5,7-dihydro-4H-pyrazolo[3,4-c]pyridin-6-yl]methanone;
3-Chloro-5-[(7S)-6-(2-chloro-3-methoxy-benzoyl)-2,7-dimethyl-5,7-dihydro-4H-pyrazolo[3,4-c]pyridin-3-yl]benzenesulfonamide;
2-[3-Chloro-5-[(7S)-6-(2-chloro-3-methoxy-benzoyl)-2,7-dimethyl-5,7-dihydro-4H-pyrazolo[3,4-c]pyridin-3-yl]phenyl]acetamide;
[2-Chloro-3-(2,2-dioxo-2λ⁶-thia-6-azaspiro[3.3]heptan-6-yl)-5-fluoro-phenyl]-[3-(3,5-difluorophenyl)-2,7-dimethyl-5,7-dihydro-4H-pyrazolo[3,4-c]pyridin-6-yl]methanone;
5-[[3-Chloro-4-[3-(3,5-difluorophenyl)-2,7-dimethyl-5,7-dihydro-4H-pyrazolo[3,4-c]pyridine-6-carbonyl]-2-pyridyl]oxymethyl]oxazolidin-2-one;
(2-Chloro-3-methoxy-phenyl)-[(7S)-3-[3-chloro-5-(1-methylsulfonylcyclopropyl)phenyl]-2,7-dimethyl-5,7-dihydro-4H-pyrazolo[3,4-c]pyridin-6-yl]methanone;
1-[3-Chloro-5-[(7S)-6-(2-chloro-3-methoxy-benzoyl)-2,7-dimethyl-5,7-dihydro-4H-pyrazolo[3,4-c]pyridin-3-yl]phenyl]-N-methyl-methanesulfonamide;
[2-Chloro-5-fluoro-3-(3-methylsulfonylazetidin-1-yl)phenyl]-[3-(3,5-difluorophenyl)-2,7-dimethyl-5,7-dihydro-4H-pyrazolo[3,4-c]pyridin-6-yl]methanone;
3-Chloro-5-[6-[2-chloro-5-fluoro-3-(1H-1,2,4-triazol-3-yl)benzoyl]-2,7-dimethyl-5,7-dihydro-4H-pyrazolo[3,4-c]pyridin-3-yl]benzenesulfonamide;
(2-Chloro-3-methoxy-phenyl)-[(7S)-3-[3-chloro-5-(methylsulfonylmethyl)phenyl]-2,7-dimethyl-5,7-dihydro-4H-pyrazolo[3,4-c]pyridin-6-yl]methanone;
3-[3-[3-(3,5-Difluorophenyl)-2,7-dimethyl-5,7-dihydro-4H-pyrazolo[3,4-c]pyridine-6-carbonyl]-4-fluoro-phenyl]benzonitrile;
[2-Chloro-5-fluoro-3-[[1-(trifluoromethyl)cyclopropyl]methoxy]phenyl]-[3-(3,5-difluorophenyl)-2,7-dimethyl-5,7-dihydro-4H-pyrazolo[3,4-c]pyridin-6-yl]methanone;
4-[2-[3-Chloro-4-[(7S)-3-(3,5-difluorophenyl)-2,7-dimethyl-5,7-dihydro-4H-pyrazolo[3,4-c]pyridine-6-carbonyl]phenyl]ethyl]pyrrolidin-2-one;
1-[3-Chloro-5-[(7S)-6-(2-chloro-3-methoxy-benzoyl)-2,7-dimethyl-5,7-dihydro-4H-pyrazolo[3,4-c]pyridin-3-yl]phenyl]cyclopropanecarboxamide;
[1-(5-Chloro-2-hydroxy-3-pyridyl)-1,2,4-triazol-3-yl]-[(7S)-3-(3,5-difluorophenyl)-2,7-dimethyl-5,7-dihydro-4H-pyrazolo[3,4-c]pyridin-6-yl]methanone;
N-[1-[3-Chloro-5-[6-[2-chloro-5-fluoro-3-(2-oxo-1H-pyridin-4-yl)benzoyl]-2,7-dimethyl-5,7-dihydro-4H-pyrazolo[3,4-c]pyridin-3-yl]phenyl]cyclopropyl]methanesulfonamide;
5-[2-Chloro-3-[3-[3-chloro-5-(1-methylsulfonylcyclopropyl)phenyl]-2,7-dimethyl-5,7-dihydro-4H-pyrazolo[3,4-c]pyridine-6-carbonyl]-5-fluoro-phenyl]-1H-pyridin-2-one;
[(7S)-3-(3,5-Difluorophenyl)-2,7-dimethyl-5,7-dihydro-4H-pyrazolo[3,4-c]pyridin-6-yl]-[1-(5-fluoro-2-hydroxy-3-pyridyl)-1,2,4-triazol-3-yl]methanone;
2-[3-Chloro-5-[(7R)-6-(2-chloro-3-methoxy-benzoyl)-2,7-dimethyl-5,7-dihydro-4H-pyrazolo[3,4-c]pyridin-3-yl]phenyl]acetamide;
[(7S)-3-(3,5-Difluorophenyl)-2,7-dimethyl-5,7-dihydro-4H-pyrazolo[3,4-c]pyridin-6-yl]-[1-(6-hydroxy-3-pyridyl)-1,2,4-triazol-3-yl]methanone;
5-[2-Chloro-3-[(7R)-3-(3,5-difluorophenyl)-2,7-dimethyl-5,7-dihydro-4H-pyrazolo[3,4-c]pyridine-6-carbonyl]-5-fluoro-phenyl]-1H-pyridin-2-one;
(2-Chloro-3-methoxy-phenyl)-[(7S)-2,7-dimethyl-3-[6-(trifluoromethyl)pyrazin-2-yl]-5,7-dihydro-4H-pyrazolo[3,4-c]pyridin-6-yl]methanone;
2-[3-[(7S)-6-(2-Chloro-3-methoxy-benzoyl)-2,7-dimethyl-5,7-dihydro-4H-pyrazolo[3,4-c]pyridin-3-yl]-5-fluoro-phenyl]acetamide;
[(7S)-3-(3,5-Difluorophenyl)-2,7-dimethyl-5,7-dihydro-4H-pyrazolo[3,4-c]pyridin-6-yl]-[1-(2-hydroxy-3-pyridyl)-1,2,4-triazol-3-yl]methanone;
4-[2-[3-Chloro-4-[3-(3,5-difluorophenyl)-2,7-dimethyl-5,7-dihydro-4H-pyrazolo[3,4-c]pyridine-6-carbonyl]phenyl]ethyl]pyrrolidin-2-one;
1-[2-Chloro-3-[2,7-dimethyl-3-[6-(trifluoromethyl)pyrazin-2-yl]-5,7-dihydro-4H-pyrazolo[3,4-c]pyridine-6-carbonyl]-5-fluoro-phenyl]azetidine-3-sulfonamide;
(2-Chloro-3-methoxy-phenyl)-[(7S)-2,7-dimethyl-3-[2-(trifluoromethyl)pyrimidin-5-yl]-5,7-dihydro-4H-pyrazolo[3,4-c]pyridin-6-yl]methanone;
3-Chloro-5-[(7S)-2,7-dimethyl-6-[1-(2-oxo-1H-pyridin-3-yl)-1,2,4-triazole-3-carbonyl]-5,7-dihydro-4H-pyrazolo[3,4-c]pyridin-3-yl]benzenesulfonamide;
[2-Chloro-3-(5,5-dimethyl-4H-isoxazol-3-yl)-5-fluoro-phenyl]-[3-(3,5-difluorophenyl)-2,7-dimethyl-5,7-dihydro-4H-pyrazolo[3,4-c]pyridin-6-yl]methanone;
(2-Chloro-3-methoxy-phenyl)-[(7S)-2,7-dimethyl-3-[5-(trifluoromethyl)-1,3,4-oxadiazol-2-yl]-5,7-dihydro-4H-pyrazolo[3,4-c]pyridin-6-yl]methanone;
(2-Chloro-3-methoxy-phenyl)-[(7S)-2,7-dimethyl-3-[[[1-(trifluoromethyl)cyclopropyl]amino]methyl]-5,7-dihydro-4H-pyrazolo[3,4-c]pyridin-6-yl]methanone;
3-Chloro-5-[(7R)-2,7-dimethyl-6-[1-(2-oxo-1H-pyridin-3-yl)-1,2,4-triazole-3-carbonyl]-5,7-dihydro-4H-pyrazolo[3,4-c]pyridin-3-yl]benzenesulfonamide;
(2-Chloro-3-methoxy-phenyl)-[(7S)-3-(6-hydroxy-2-pyridyl)-2,7-dimethyl-5,7-dihydro-4H-pyrazolo[3,4-c]pyridin-6-yl]methanone;
[3-(3,5-Difluorophenyl)-2,7-dimethyl-5,7-dihydro-4H-pyrazolo[3,4-c]pyridin-6-yl]-(2-pyrrolidin-3-yl-1,2,4-triazol-3-yl)methanone;hydrochloride;
1-[3-[3-(3,5-Difluorophenyl)-2,7-dimethyl-5,7-dihydro-4H-pyrazolo[3,4-c]pyridine-6-carbonyl]-2-methyl-phenyl]imidazolidin-2-one;
3-Chloro-5-[2,7-dimethyl-6-[1-(2-oxo-1H-pyridin-3-yl)-1,2,4-triazole-3-carbonyl]-5,7-dihydro-4H-pyrazolo[3,4-c]pyridin-3-yl]benzenesulfonamide;
[3-(3,5-Difluorophenyl)-2,7-dimethyl-5,7-dihydro-4H-pyrazolo[3,4-c]pyridin-6-yl]-[2-(triazol-1-ylmethyl)phenyl]methanone;
5-[[3-Chloro-4-[(7R)-3-(3,5-difluorophenyl)-2,7-dimethyl-5,7-dihydro-4H-pyrazolo[3,4-c]pyridine-6-carbonyl]-2-pyridyl]oxymethyl]oxazolidin-2-one;
[2-Chloro-3-(2,2-dioxo-2λ⁶-thia-6-azaspiro[3.3]heptan-6-yl)-5-fluoro-phenyl]-[(7R)-3-(3,5-difluorophenyl)-2,7-dimethyl-5,7-dihydro-4H-pyrazolo[3,4-c]pyridin-6-yl]methanone;
N-[[3-Chloro-5-[(7R)-6-(2-chloro-3-methoxy-benzoyl)-2,7-dimethyl-5,7-dihydro-4H-pyrazolo[3,4-c]pyridin-3-yl]phenyl]methyl]methanesulfonamide;
4-[2-[3-Chloro-4-[(7R)-3-(3,5-difluorophenyl)-2,7-dimethyl-5,7-dihydro-4H-pyrazolo[3,4-c]pyridine-6-carbonyl]phenyl]ethyl]pyrrolidin-2-one;
[2-Chloro-3-(2,2-dioxo-2λ⁶-thia-6-azaspiro[3.3]heptan-6-yl)-5-fluoro-phenyl]-[(7S)-2,7-dimethyl-3-[6-(trifluoromethyl)pyrazin-2-yl]-5,7-dihydro-4H-pyrazolo[3,4-c]pyridin-6-yl]methanone;
2-[5-[(7S)-6-(2-Chloro-3-methoxy-benzoyl)-2,7-dimethyl-5,7-dihydro-4H-pyrazolo[3,4-c]pyridin-3-yl]-3-pyridyl]acetamide;
[2-Chloro-3-(2,2-dioxo-2λ⁶-thia-6-azaspiro[3.3]heptan-6-yl)-5-fluoro-phenyl]-[(7R)-2,7-dimethyl-3-[6-(trifluoromethyl)pyrazin-2-yl]-5,7-dihydro-4H-pyrazolo[3,4-c]pyridin-6-yl]methanone;
5-[2-Chloro-3-[(7S)-2,7-dimethyl-3-[6-(trifluoromethyl)pyrazin-2-yl]-5,7-dihydro-4H-pyrazolo[3,4-c]pyridine-6-carbonyl]-5-fluoro-phenyl]-1H-pyridin-2-one;
3-[3-[(7S)-2,7-Dimethyl-3-[6-(trifluoromethyl)pyrazin-2-yl]-5,7-dihydro-4H-pyrazolo[3,4-c]pyridine-6-carbonyl]-1,2,4-triazol-1-yl]-1H-pyridin-2-one;
(2-Chloro-3-methoxy-phenyl)-[(7S)-2,7-dimethyl-3-(5-methylsulfonyl-3-pyridyl)-5,7-dihydro-4H-pyrazolo[3,4-c]pyridin-6-yl]methanone;
[(7S)-3-[3-chloro-5-(2-methoxyethoxy)phenyl]-2,7-dimethyl-5,7-dihydro-4H-pyrazolo[3,4-c]pyridin-6-yl]-(2-chloro-3-methoxy-phenyl)methanone;
1-[3-chloro-5-[6-[2-chloro-5-fluoro-3-[(5-oxopyrrolidin-3-yl)methoxy]benzoyl]-2,7-dimethyl-5,7-dihydro-4H-pyrazolo[3,4-c]pyridin-3-yl]phenyl]cyclopropanecarboxamide;
1-[3-chloro-5-[(7S)-6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropanecarboxamide;
1-[3-chloro-5-[(7R)-6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropanecarboxamide;
5-[2-chloro-3-[(7S)-3-[3-chloro-5-(1-methylsulfonylcyclopropyl)phenyl]-2,7-dimethyl-5,7-dihydro-4H-pyrazolo[3,4-c]pyridine-6-carbonyl]-5-fluoro-phenyl]-1H-pyridin-2-one;
5-[2-chloro-3-[(7R)-3-[3-chloro-5-(1-methylsulfonylcyclopropyl)phenyl]-2,7-dimethyl-5,7-dihydro-4H-pyrazolo[3,4-c]pyridine-6-carbonyl]-5-fluoro-phenyl]-1H-pyridin-2-one;
1-[3-chloro-5-[(7S)-6-[2-chloro-3-(2,2-dioxo-2λ⁶-thia-6-azaspiro[3.3]heptan-6-yl)-5-fluoro-benzoyl]-2,7-dimethyl-5,7-dihydro-4H-pyrazolo[3,4-c]pyridin-3-yl]phenyl]cyclopropanecarboxamide;
1-[3-chloro-5-[(7R)-6-[2-chloro-3-(2,2-dioxo-2λ⁶-thia-6-azaspiro[3.3]heptan-6-yl)-5-fluoro-benzoyl]-2,7-dimethyl-5,7-dihydro-4H-pyrazolo[3,4-c]pyridin-3-yl]phenyl]cyclopropanecarboxamide;
3-[5-chloro-4-[(7S)-3-(3,5-difluorophenyl)-2,7-dimethyl-5,7-dihydro-4H-pyrazolo[3,4-c]pyridine-6-carbonyl]imidazol-1-yl]-1H-pyridin-2-one;
3-[3-[(7S)-3-[3-chloro-5-(1-methylsulfonylcyclopropyl)phenyl]-2,7-dimethyl-5,7-dihydro-4H-pyrazolo[3,4-c]pyridine-6-carbonyl]-1,2,4-triazol-1-yl]-1H-pyridin-2-one;
[(7R)-3-[3-chloro-5-(2-methoxyethoxy)phenyl]-2,7-dimethyl-5,7-dihydro-4H-pyrazolo[3,4-c]pyridin-6-yl]-(2-chloro-3-methoxy-phenyl)methanone;
N-[[2-chloro-6-[6-(2-chloro-3-methoxy-benzoyl)-2,7-dimethyl-5,7-dihydro-4H-pyrazolo[3,4-c]pyridin-3-yl]-4-pyridyl]methyl]methanesulfonamide;
(2-chloro-3-methoxy-phenyl)-[(7S)-2,7-dimethyl-3-[2-(trifluoromethyl)pyrimidin-4-yl]-5,7-dihydro-4H-pyrazolo[3,4-c]pyridin-6-yl]methanone;
4-[2-chloro-5-fluoro-3-[(7S)-3-[3-fluoro-5-[[methyl(dioxo)-λ⁶-phosphanyl]methyl]phenyl]-2,7-dimethyl-5,7-dihydro-4H-pyrazolo[3,4-c]pyridine-6-carbonyl]phenyl]-1H-pyrrole-2-carbonitrile;
4-[2-chloro-5-fluoro-3-[(7R)-3-[3-fluoro-5-[[methyl(dioxo)-λ⁶-phosphanyl]methyl]phenyl]-2,7-dimethyl-5,7-dihydro-4H-pyrazolo[3,4-c]pyridine-6-carbonyl]phenyl]-1H-pyrrole-2-carbonitrile;
N-[1-[3-chloro-5-[6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropyl]methanesulfonamide;
N-[1-[3-chloro-5-[(7S)-6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropyl]methanesulfonamide;
N-[1-[3-chloro-5-[(7R)-6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropyl]methanesulfonamide;
N-[1-[3-chloro-5-[6-[2-chloro-5-fluoro-3-[2-(1H-triazol-4-yl)ethyl]benzoyl]-2,7-dimethyl-5,7-dihydro-4H-pyrazolo[3,4-c]pyridin-3-yl]phenyl]cyclopropyl]methanesulfonamide;
N-[1-[3-chloro-5-[(7S)-6-[2-chloro-5-fluoro-3-[2-(1H-triazol-4-yl)ethyl]benzoyl]-2,7-dimethyl-5,7-dihydro-4H-pyrazolo[3,4-c]pyridin-3-yl]phenyl]cyclopropyl]methanesulfonamide;
N-[1-[3-chloro-5-[(7R)-6-[2-chloro-5-fluoro-3-[2-(1H-triazol-4-yl)ethyl]benzoyl]-2,7-dimethyl-5,7-dihydro-4H-pyrazolo[3,4-c]pyridin-3-yl]phenyl]cyclopropyl]methanesulfonamide;
4-[2-chloro-3-[(7S)-2,7-dimethyl-3-(2,3,4,5-tetrafluorophenyl)-5,7-dihydro-4H-pyrazolo[3,4-c]pyridine-6-carbonyl]-5-fluoro-phenyl]-1H-pyrrole-2-carbonitrile;
N-[1-[3-chloro-5-[(7S)-6-[1-(5-chloro-2-hydroxy-3-pyridyl)-1,2,4-triazole-3-carbonyl]-2,7-dimethyl-5,7-dihydro-4H-pyrazolo[3,4-c]pyridin-3-yl]phenyl]cyclopropyl]methanesulfonamide;
4-[2-chloro-3-[(7S)-2,7-dimethyl-3-(3,4,5-trifluorophenyl)-5,7-dihydro-4H-pyrazolo[3,4-c]pyridine-6-carbonyl]-5-fluoro-phenyl]-1H-pyrrole-2-carbonitrile;
4-[2-chloro-3-[(7R)-2,7-dimethyl-3-(3,4,5-trifluorophenyl)-5,7-dihydro-4H-pyrazolo[3,4-c]pyridine-6-carbonyl]-5-fluoro-phenyl]-1H-pyrrole-2-carbonitrile;
N-[1-[3-chloro-5-[(7S)-6-[2,5-difluoro-3-(1H-pyrazol-4-yl)benzoyl]-2,7-dimethyl-5,7-dihydro-4H-pyrazolo[3,4-c]pyridin-3-yl]phenyl]cyclopropyl]methanesulfonamide;
[(7S)-3-[3-chloro-5-(2-hydroxy-2-methyl-propyl)phenyl]-2,7-dimethyl-5,7-dihydro-4H-pyrazolo[3,4-c]pyridin-6-yl]-(2-chloro-3-methoxy-phenyl)methanone;
3-[3-[(7R)-2,7-dimethyl-3-[6-(trifluoromethyl)pyrazin-2-yl]-5,7-dihydro-4H-pyrazolo[3,4-c]pyridine-6-carbonyl]-1,2,4-triazol-1-yl]-1H-pyridin-2-one;
3-chloro-5-[2-chloro-3-[3-(3,5-difluorophenyl)-2,7-dimethyl-5,7-dihydro-4H-pyrazolo[3,4-c]pyridine-6-carbonyl]-5-fluoro-phenyl]-3H-pyridin-2-one;
3-chloro-5-[2-chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-3H-pyridin-2-one;
3-chloro-5-[2-chloro-3-[(7R)-3-(3,5-difluorophenyl)-2,7-dimethyl-5,7-dihydro-4H-pyrazolo[3,4-c]pyridine-6-carbonyl]-5-fluoro-phenyl]-3H-pyridin-2-one;
[1-(5-chloro-2-hydroxy-3-pyridyl)-1,2,4-triazol-3-yl]-[(7S)-3-(3,5-difluorophenyl)-2,7-dimethyl-5,7-dihydro-4H-pyrazolo[3,4-c]pyridin-6-yl]methanone;
[1-(5-chloro-2-hydroxy-3-pyridyl)-1,2,4-triazol-3-yl]-[(7R)-3-(3,5-difluorophenyl)-2,7-dimethyl-5,7-dihydro-4H-pyrazolo[3,4-c]pyridin-6-yl]methanone;
[2-chloro-5-fluoro-3-[2-(1H-pyrazol-4-yl)ethyl]phenyl]-[(7S)-3-(3,5-difluorophenyl)-2,7-dimethyl-5,7-dihydro-4H-pyrazolo[3,4-c]pyridin-6-yl]methanone;
[2-chloro-5-fluoro-3-[2-(1H-pyrazol-4-yl)ethyl]phenyl]-[(7R)-3-(3,5-difluorophenyl)-2,7-dimethyl-5,7-dihydro-4H-pyrazolo[3,4-c]pyridin-6-yl]methanone;
[2-chloro-5-fluoro-3-[2-(1H-triazol-4-yl)ethyl]phenyl]-[(7S)-3-(3,5-difluorophenyl)-2,7-dimethyl-5,7-dihydro-4H-pyrazolo[3,4-c]pyridin-6-yl]methanone;
[2-chloro-5-fluoro-3-[2-(1H-triazol-4-yl)ethyl]phenyl]-[(7R)-3-(3,5-difluorophenyl)-2,7-dimethyl-5,7-dihydro-4H-pyrazolo[3,4-c]pyridin-6-yl]methanone;
4-[[2-chloro-3-[3-(3,5-difluorophenyl)-2,7-dimethyl-5,7-dihydro-4H-pyrazolo[3,4-c]pyridine-6-carbonyl]-5-fluoro-phenyl]methylamino]pyrrolidin-2-one;hydrochloride;
5-[[2,5-dichloro-3-[(7S)-3-(3,5-difluorophenyl)-2,7-dimethyl-5,7-dihydro-4H-pyrazolo[3,4-c]pyridine-6-carbonyl]phenoxy]methyl]oxazolidin-2-one;
(5S)-5-[[2,5-dichloro-3-[(7S)-3-(3,5-difluorophenyl)-2,7-dimethyl-5,7-dihydro-4H-pyrazolo[3,4-c]pyridine-6-carbonyl]phenoxy]methyl]oxazolidin-2-one;
(5R)-5-[[2,5-dichloro-3-[(7S)-3-(3,5-difluorophenyl)-2,7-dimethyl-5,7-dihydro-4H-pyrazolo[3,4-c]pyridine-6-carbonyl]phenoxy]methyl]oxazolidin-2-one;
4-[[2-chloro-3-[(7S)-3-(3,5-difluorophenyl)-2,7-dimethyl-5,7-dihydro-4H-pyrazolo[3,4-c]pyridine-6-carbonyl]phenoxy]methyl]pyrrolidin-2-one;
[2-chloro-3-(1,6-diazaspiro[3.3]heptan-6-yl)-5-fluoro-phenyl]-[(7S)-3-(3,5-difluorophenyl)-2,7-dimethyl-5,7-dihydro-4H-pyrazolo[3,4-c]pyridin-6-yl]methanone;2,2,2-trifluoroacetic acid;
[(7S)-3-(3,5-difluorophenyl)-2,7-dimethyl-5,7-dihydro-4H-pyrazolo[3,4-c]pyridin-6-yl]-[1-(2-hydroxy-4-pyridyl)-1,2,4-triazol-3-yl]methanone;
(4S)-4-[[2-chloro-3-[(7S)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one;
(4R)-4-[[2-chloro-3-[(7S)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one;
(4S)-4-[[2-chloro-3-[(7R)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one;
(2-chloro-3-methoxy-phenyl)-[(8S)-3-(3-chlorophenyl)-8-methyl-6,8-dihydro-5H-[1,2,4]triazolo[4,3-a]pyrazin-7-yl]methanone; and
(2-chloro-3-methoxy-phenyl)-[(8S)-3-(3-chlorophenyl)-8-methyl-6,8-dihydro-5H-imidazo[1,2-a]pyrazin-7-yl]methanone.

In a preferred embodiment, the present invention provides a compound of formula (I) as described herein, or a pharmaceutically acceptable salt thereof, selected from:
4-[[2-Chloro-3-[3-(3,5-difluorophenyl)-2,7-dimethyl-5,7-dihydro-4H-pyrazolo[3,4-c]pyridine-6-carbonyl]-5-fluoro-phenoxy]methyl]pyrrolidin-2-one;
5-[2-Chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-1H-pyridin-2-one;
5-[[2-Chloro-3-[3-(3,5-difluorophenyl)-2,7-dimethyl-5,7-dihydro-4H-pyrazolo[3,4-c]pyridine-6-carbonyl]-5-fluoro-phenoxy]methyl]oxazolidin-2-one;
4-[2-Chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-1H-pyridin-2-one;
[1-(5-Chloro-2-hydroxy-3-pyridyl)-1,2,4-triazol-3-yl]-[(7S)-3-(3,5-difluorophenyl)-2,7-dimethyl-5,7-dihydro-4H-pyrazolo[3,4-c]pyridin-6-yl]methanone;
[(7S)-3-(3,5-Difluorophenyl)-2,7-dimethyl-5,7-dihydro-4H-pyrazolo[3,4-c]pyridin-6-yl]-[1-(2-hydroxy-3-pyridyl)-1,2,4-triazol-3-yl]methanone;
N-[1-[3-chloro-5-[(7S)-6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropyl]methanesulfonamide;
[2-chloro-5-fluoro-3-[2-(1H-pyrazol-4-yl)ethyl]phenyl]-[(7S)-3-(3,5-difluorophenyl)-2,7-dimethyl-5,7-dihydro-4H-pyrazolo[3,4-c]pyridin-6-yl]methanone;
[2-chloro-5-fluoro-3-[2-(1H-triazol-4-yl)ethyl]phenyl]-[(7R)-3-(3,5-difluorophenyl)-2,7-dimethyl-5,7-dihydro-4H-pyrazolo[3,4-c]pyridin-6-yl]methanone;
4-[[2-chloro-3-[3-(3,5-difluorophenyl)-2,7-dimethyl-5,7-dihydro-4H-pyrazolo[3,4-c]pyridine-6-carbonyl]-5-fluoro-phenyl]methylamino]pyrrolidin-2-one;hydrochloride;
(4S)-4-[[2-chloro-3-[(7S)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one; and
(4S)-4-[[2-chloro-3-[(7R)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one.

In a particularly preferred embodiment, the present invention provides a compound of formula (I) as described herein, or a pharmaceutically acceptable salt thereof, wherein said compound of formula (I) is 4-[[2-Chloro-3-[3-(3,5-difluorophenyl)-2,7-dimethyl-5,7-dihydro-4H-pyrazolo[3,4-c]pyridine-6-carbonyl]-5-fluoro-phenoxy]methyl]pyrrolidin-2-one.

In a particularly preferred embodiment, the present invention provides a compound of formula (I) as described herein, or a pharmaceutically acceptable salt thereof, wherein said compound of formula (I) is 5-[2-Chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-1H-pyridin-2-one.

In a particularly preferred embodiment, the present invention provides a compound of formula (I) as described herein, or a pharmaceutically acceptable salt thereof, wherein said compound of formula (I) is 5-[[2-Chloro-3-[3-(3,5-difluorophenyl)-2,7-dimethyl-5,7-dihydro-4H-pyrazolo[3,4-c]pyridine-6-carbonyl]-5-fluoro-phenoxy]methyl]oxazolidin-2-one.

In a particularly preferred embodiment, the present invention provides a compound of formula (I) as described herein, or a pharmaceutically acceptable salt thereof, wherein said compound of formula (I) is 4-[2-Chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-1H-pyridin-2-one.

In a particularly preferred embodiment, the present invention provides a compound of formula (I) as described herein, or a pharmaceutically acceptable salt thereof, wherein said compound of formula (I) is [1-(5-Chloro-2-hydroxy-3-pyridyl)-1,2,4-triazol-3-yl]-[(7S)-3-(3,5-difluorophenyl)-2,7-dimethyl-5,7-dihydro-4H-pyrazolo[3,4-c]pyridin-6-yl]methanone.

In a particularly preferred embodiment, the present invention provides a compound of formula (I) as described herein, or a pharmaceutically acceptable salt thereof, wherein said compound of formula (I) is [(7S)-3-(3,5-Difluorophenyl)-2,7-dimethyl-5,7-dihydro-4H-pyrazolo[3,4-c]pyridin-6-yl]-[1-(2-hydroxy-3-pyridyl)-1,2,4-triazol-3-yl]methanone.

In a particularly preferred embodiment, the present invention provides a compound of formula (I) as described herein, or a pharmaceutically acceptable salt thereof, wherein said compound of formula (I) is N-[1-[3-chloro-5-[(7S)-6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropyl]methanesulfonamide.

In a particularly preferred embodiment, the present invention provides a compound of formula (I) as described herein, or a pharmaceutically acceptable salt thereof, wherein said compound of formula (I) is [2-chloro-5-fluoro-3-[2-(1H-pyrazol-4-yl)ethyl]phenyl]-[(7S)-3-(3,5-difluorophenyl)-2,7-dimethyl-5,7-dihydro-4H-pyrazolo[3,4-c]pyridin-6-yl]methanone.

In a particularly preferred embodiment, the present invention provides a compound of formula (I) as described herein, or a pharmaceutically acceptable salt thereof, wherein said compound of formula (I) is [2-chloro-5-fluoro-3-[2-(1H-triazol-4-yl)ethyl]phenyl]-[(7R)-3-(3,5-difluorophenyl)-2,7-dimethyl-5,7-dihydro-4H-pyrazolo[3,4-c]pyridin-6-yl]methanone.

In a particularly preferred embodiment, the present invention provides a compound of formula (I) as described herein, or a pharmaceutically acceptable salt thereof, wherein said compound of formula (I) is 4-[[2-chloro-3-[3-(3,5-difluorophenyl)-2,7-dimethyl-5,7-dihydro-4H-pyrazolo[3,4-c]pyridine-6-carbonyl]-5-fluoro-phenyl]methylamino]pyrrolidin-2-one;hydrochloride.

In a particularly preferred embodiment, the present invention provides a compound of formula (I) as described herein, or a pharmaceutically acceptable salt thereof, wherein said compound of formula (I) is (4S)-4-[[2-chloro-3-[(7S)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one.

In a particularly preferred embodiment, the present invention provides a compound of formula (I) as described herein, or a pharmaceutically acceptable salt thereof, wherein said compound of formula (I) is (4S)-4-[[2-chloro-3-[(7R)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one.

In a particular embodiment, the present invention provides pharmaceutically acceptable salts of the compounds according to formula (I) as described herein. In a further particular embodiment, the present invention provides compounds according to formula (I) as described herein in their free form (i.e., as free bases or acids).

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

The preparation of compounds of formula (II) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (II) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (II) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (II) will typically lead to the respective diastereomerically/enantiomerically enriched compounds of formula (II).

A person skilled in the art will acknowledge that in the synthesis of compounds of formula (II)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (II) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

The present compounds of formula (II), wherein X is NR⁹ and Y is C (hereinafter “formula (I)”), can be prepared by reacting an intermediate of formula I with a carboxylic acid of formula 2 by stirring them in a polar solvent such as DMF or DMA in the presence of a base such as DIPEA or TEA, and a coupling agent such as HATU or HOBT (Scheme 1). In some cases, the carboxylic acid 2 can be treated with thionyl chloride or oxalyl chloride to generate the corresponding acyl chloride. When treated with a base such as TEA in the presence of amine 1, it will also generate the compounds of formula I. In somes cases, when R¹ is is protected with a protecting group (e.g. when R¹═NH₂SO₂—), a subsequent deprotection step was carried out, also giving a compound of formula I.

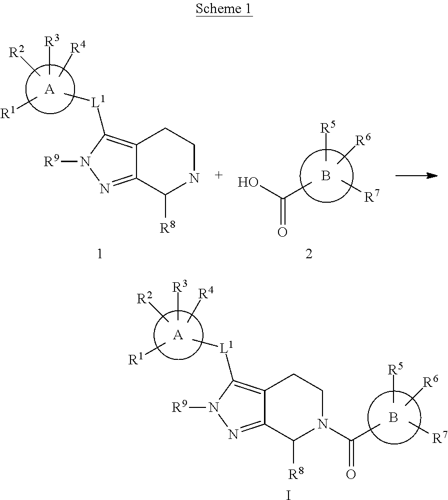

Alternatively, the compound of general formula I where L¹ is a covalent bond, A is a phenyl ring, R¹ is carbamoyl-C₁-C₆-alkyl (depicted here as compound 3), can be prepared from the ester 4 (Scheme 2). Ester 4 can be treated with ammonia and a coupling agent such as CDI. Alternatively, the ester 4 can be first saponified to the corresponding carboxylic acid, and then treated with ammonia and a suitable coupling agent to give compound 3.

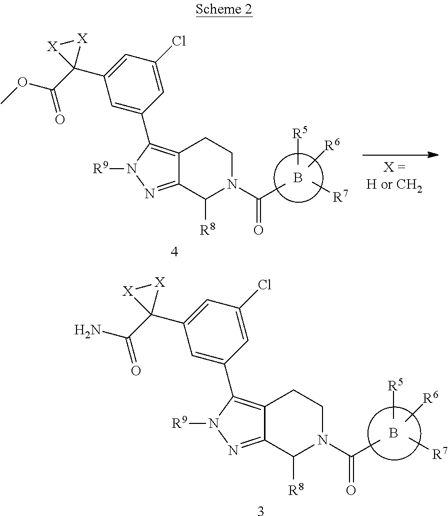

Alternatively, the compound of general formula I, where ring B=1H-1,2,4-triazol-3-yl, L²=a covalent bond, and R¹⁰=pyrrolidinyl (depicted here as compound 5), can be prepared as drawn in Scheme 3. N-alkylation of bromide 7 by triazole 6 in the presence of a base gives intermediate 8 (and the corresponding regioisomer(s), not shown here). Intermediate 9 can be generated via a saponification of ester 8, using a base such as NaOH or LiOH in a polar solvent such as THF, MeOH or water (or a mixture thereof). Intermediate 10 can be prepared from an amide coupling between acid 9 and amine 1, using suitable coupling reagents such as HATU or HOBT, a base (e.g. TEA or DIPEA), in polar protic solvent such as DMF or DMA. Intermediate 5 can be obtained after N-Boc deprotection of intermediate 10, under acidic conditions (e.g. HCl or TFA) in solvents such as 1,4-dioxane or DCM.

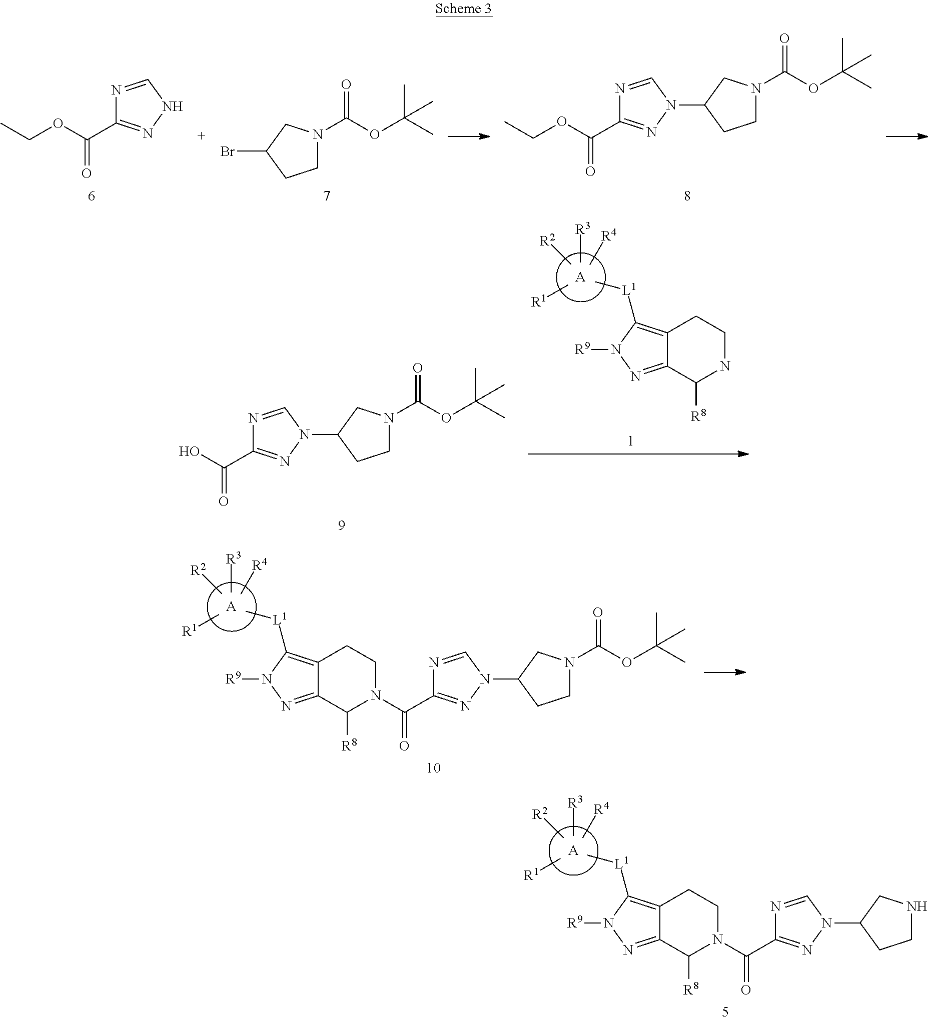

Alternatively, the compound of general formula I where L¹ is a covalent bond and ring A is 1,3,4-oxadiazolyl (depicted as compound 11), can be prepared via a palladium-catalyzed cyanation of intermediate 12 using standard reaction conditions such as 1,1′-bis(diphenylphosphino)ferrocene, zinc cyanide, tris(dibenzylideneacetone)dipalladium (0), and DMF (Scheme 4). N-Boc deprotection of the resulting intermediate 13 using an acid such as HCl, TFA or PTSA gives the corresponding amine 14 (or a salt thereof). Coupling of intermediate 14 with an acid of formula 2 using HATU or HOBT and TEA or DIPEA in DMF or DMA gives intermediate 15. A [2+3]cycloaddition of intermediate 15 with sodium azide results in intermediate 16. Compound 11 can be obtained from treatment of intermediate 16 with an anhydride for example.

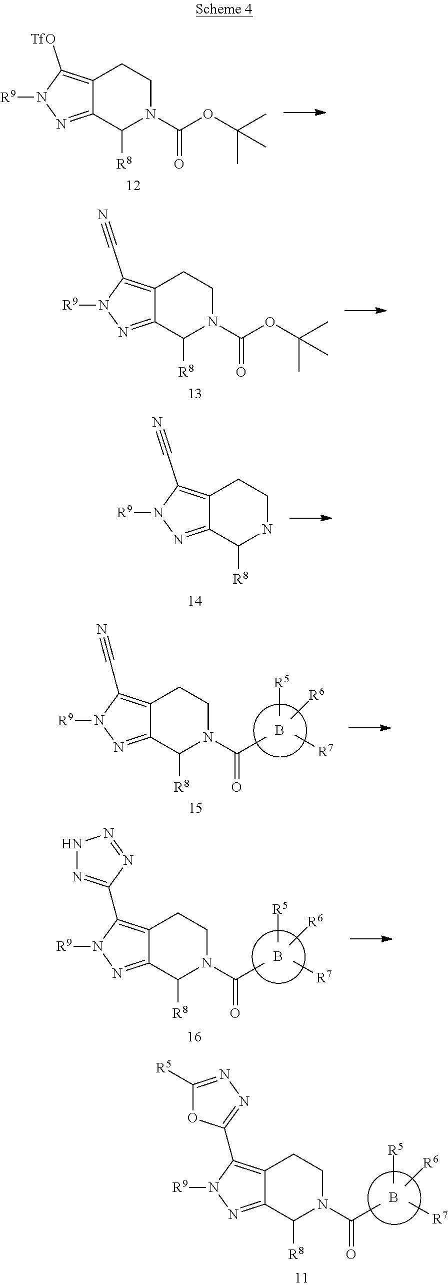

Building blocks of formula I can be prepared from intermediate 17 via N-Boc deprotection, using acids such as HCl or TFA in solvents such as 1,4-dioxane or DCM (Scheme 5). When ring A=pyridine and R¹=—OMe, intermediate 17 can be treated with 12 N HCl at 100° C., leading to both the deprotection of the N-Boc protecting group and removal of the methyl group on the methoxy, yielding intermediate 1 with a pyridone. In some cases, a protecting group might need to be introduced on R¹. This can be done before performing the N-Boc deprotection step, with deprotection occurring simultaneously/deprotection carried out subsequently using standard conditions. The chemistry described in Scheme 5 can also be applied to compounds of general formulas IIa and IIb.

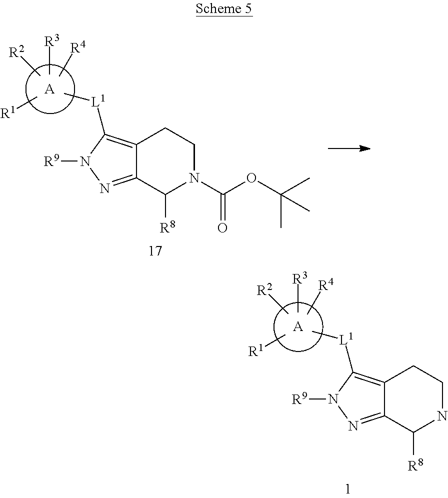

Intermediate 17 can be prepared via Pd-catalyzed Suzuki-Miyaura cross-coupling (e.g. XPhos G3, K₂CO₃, 1,4-dioxane/water or Pd(PPh₃)₄, Na₂CO₃, THF/water), between pinacol ester 18 (or the corresponding boronic acid), and triflate 12 (Scheme 6). The preparation of intermediate 12 was already described elsewhere (Patent nr. WO2020/065613 A1, 2020). The boronic acids are either commercially available or can be prepared from pinacol ester 18, following treatment with sodium metaperiodate and ammonium acetate. In some instances, the boronic ester could be installed on intermediate 12, which was then reacted with a (het)aryl bromide, also affording intermediate 17. In other instances, a bromide in place of the triflate on intermediate 12 was used for the preparation of intermediate 17.

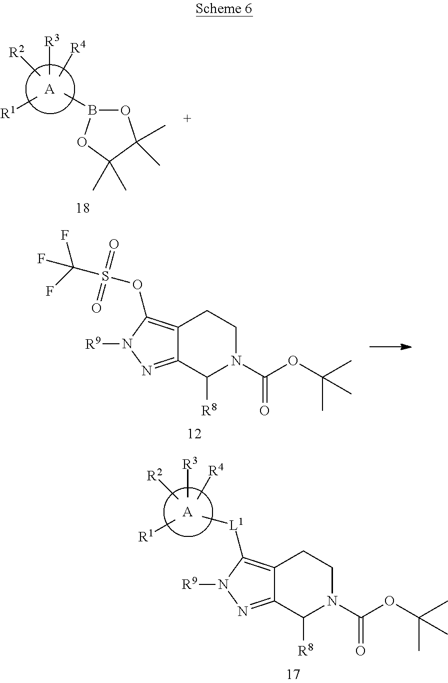

Alternatively, intermediate 19 where Y is —C₁-C₆-(halo)-alkyl or —C₃-C₁₀-(halo)-cycloalkyl, can be prepared from boronic acid 20 and triflate 12, using standard Suzuki-Miyaura cross-coupling conditions (e.g. Na₂CO₃, Pd(PPh₃)₄, DMF), to give alkene 21 (Scheme 7). Oxidative cleavage of alkene 21 using OsO₄ and NaOI₄ in 1,4-dioxane/water affords aldehyde 22. A reductive amination (e.g., using sodium cyanoborohydride in DCM) between intermediate 22 and a primary amine gives intermediate 19. In some instances, a bromide in place of the triflate on intermediate 12 was used for the preparation of intermediate 21.

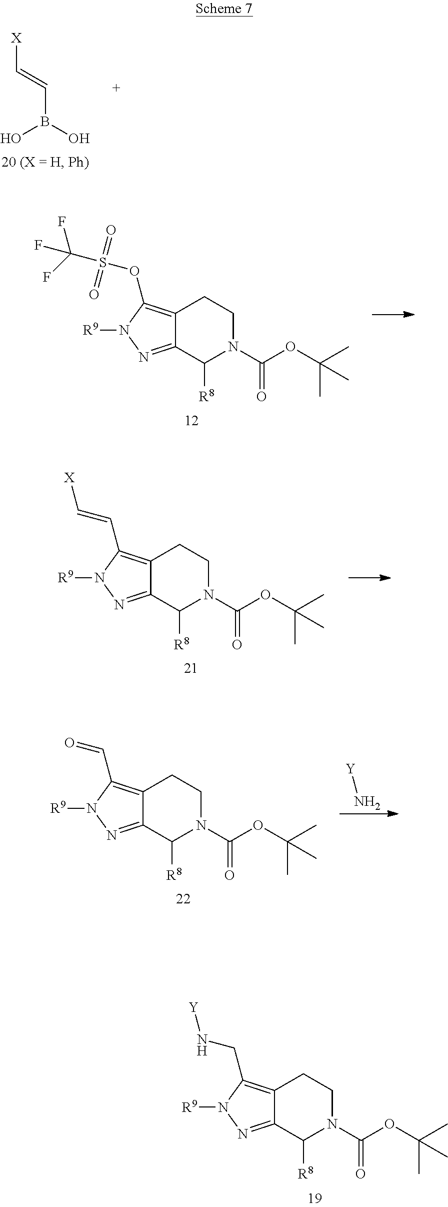

Building blocks of general formula 18 can be prepared from the corresponding bromides 23, via a Miyaura borylation (e.g. KOAc, bis(pinacolato)diboron, [1,1′-bis(diphenylphosphino)ferrocene]dichloropalladium(II), 1,4-dioxane) (Scheme 8).

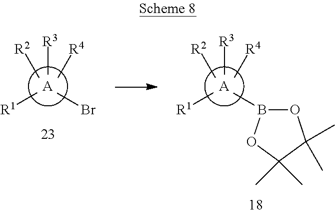

Intermediate 24 can be prepared from an (het)aryl bromide or chloride 25, using sodium methanesulfinate in DMF (Scheme 9).

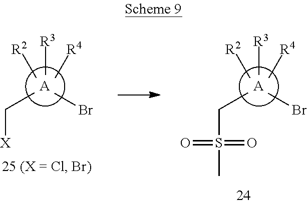

Intermediate 26 can be prepared from a benzylamine 27, upon treatment with TEA and a suitable sulfonyl chloride (Scheme 10).

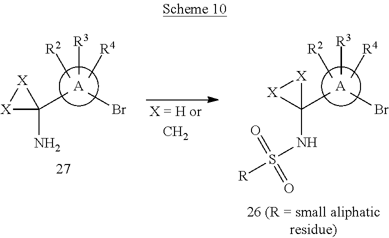

Intermediate 28 can be obtained from the corresponding sulfonyl chloride 29, upon treatment with a suitable amine (Scheme 11).

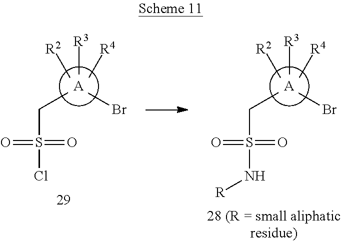

Alternatively, intermediate 30 can be prepared from carboxylic acid 31 using thionyl chloride in MeOH (X=OMe) or in ammonia (X=NH₂) (Scheme 12).

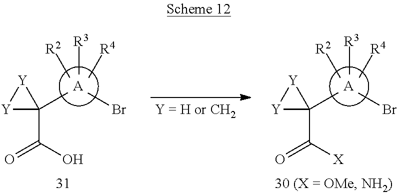

Intermediate 32 can be prepared from intermediate 33, using a sulfonium salt such as methyl(diphenyl)sulfonium;tetrafluoroborate and sodium;bis(trimethylsilyl)azanide (Scheme 13). Intermediate 33 can be prepared, among others, via a Suzuki-Miyaura cross-coupling between a halide and a boronic ester or a boronic acid, using standard reaction conditions.

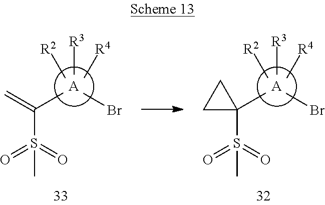

Intermediate 2 can be prepared via saponification of intermediate 34 in which R=Me or Et, using NaOH or LiOH in THF/MeOH/water or any combination of one or more of the three solvents (Scheme 14). Sometimes, when R⁵ contains a methoxypyridine, both the methoxypyridine and the methyl or ethyl ester can be deprotected at the same time, using concentrated HCl at elevated temperatures, to give the corresponding pyridine-containing carboxylic acid 2 in one step. In some instances, this sequence happens sequentially (deprotection followed by saponification, or vice versa). Similarly, for intermediates 34 bearing a protecting group (e.g. trityl), deprotection happens during saponification.

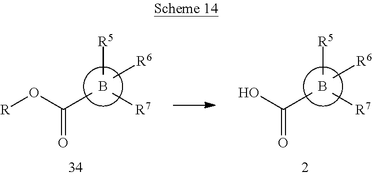

Intermediate 35 in which R=C₁-C₆-alkyl (e.g., Me, Et), ring B is a phenyl or pyridyl, and R⁵ is defined by L²=OCH₂, and ring C is a cyclic carbamate, a lactam, or a halocycloalky, can be prepared via alkylation of phenol 37 with intermediate 36, using a base such as cesium carbonate, potassium carbonate, or TEA, in DMF or DMSO (Scheme 15).

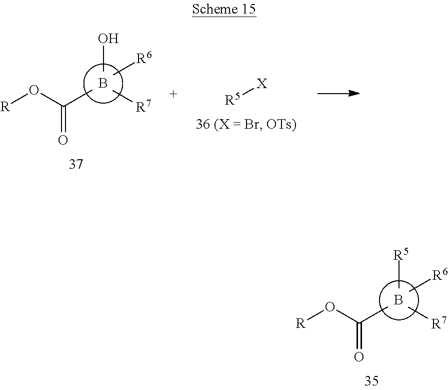

Intermediate 38 could be prepared starting from bromide 39 (Scheme 16). Bromide 39 can be treated with n-BuLi and DMF, to give aldehyde 40. Aldehyde 40 can be treated with hydroxylamine hydrochloride and sodium acetate, to give intermediate 41. Chlorination (using NCS) of intermediate 41 gives intermediate 42. Cycloaddition using 2-methylpropene and TEA gives intermediate 38.

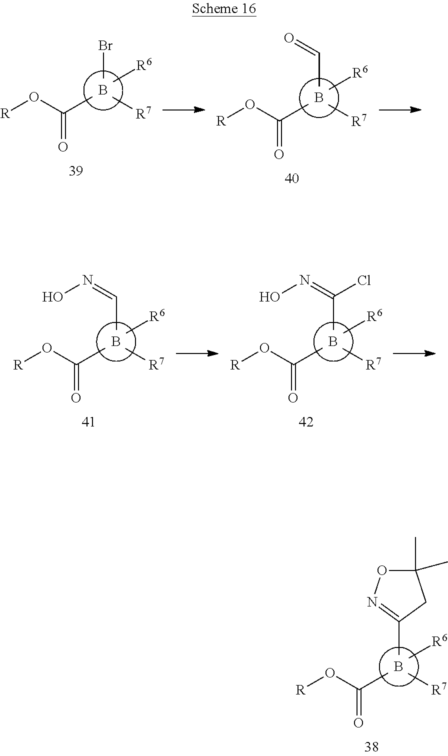

Intermediate 34 where R=Me, Et, ring B is a phenyl or pyridyl, R⁵ is defined with L² is a covalent bond, and C is a heterocyclic amine such as azetidine, piperidine, piperazine, 2-thia-6-azaspiro[3.3]heptane, or a 1,6-diazaspiro[3.3]heptane, can be prepared from the corresponding bromide 43 (Scheme 17). Introduction of these secondary amines can be made via a standard Buchwald-Hartwig cross-coupling, using the amine as free base or as a salt (e.g. HCl), Pd₂(dba)₃, a base such as cesium carbonate, a ligand such as xantphos, in a solvent such as 1,4-dioxane. In some cases, when an amine bearing an additional protected functionality is used for the Buchwald-Hartwig coupling, an additional deprotection step is needed after the cross-coupling and is not depicted here. Alternatively, when intermediate 34, where R=Me or Et, ring B is a (heteroyaryl) ring such as phenyl or pyridyl, R⁵ is defined with L²=covalent bond and ring C is a pyridyl, pyrazine, pyrimidine, 1,2-dihydropyridine, phenyl, pyrrolyl, pyrazolyl, triazol-1-yl, 1H-triazol-4-yl, and 1H-1,2,4-triazol-3-yl, reaction with intermediate 43 gives the corresponding bi(het)aryls 34 (Scheme 17). Intermediate 34 can be prepared via standard Suzuki-Miyaura cross-couplings using bromide 43 and the boronic esters or acids, with e.g. Pd(PPh₃)₄, sodium carbonate. In some cases, the bromide can also be transformed to the corresponding boronic esters or acids. In the case where ring C is a 1H-1,2,4-triazol-3-yl, a deprotection step is needed after the cross-coupling.

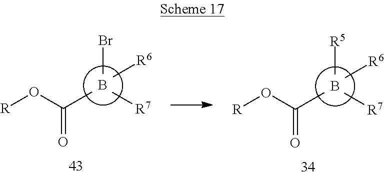

Intermediate 44, where R=Me, Et, ring B is 2H-triazol-4-yl, imidazolyl, 1H-1,2,4-triazol-3-yl, and R⁵ is defined with L²=a covalent bond and ring C is 1,2-dihydropyridine and R¹⁰ is oxo, can be prepared via a Chan-Lam coupling between intermediate 45 and a boronic acid 46, using standard conditions (e.g. Cu(OAc)₂, pyridine, in DCM) (Scheme 18).

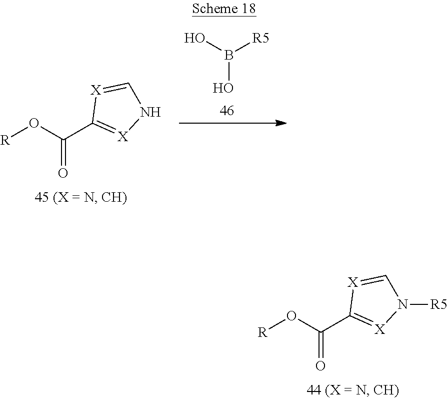

Intermediate 47, where R=Me or Et and X is an (hetero)cyclic or (hetero)aromatic compound, can be prepared via a Sonogashira cross-coupling between iodide 48 and alkyne 49, using standard reaction conditions, such as Pd(PPh₃)₄, CuI and TEA in ACN to give intermediate 50, followed by a hydrogenation (Scheme 19).

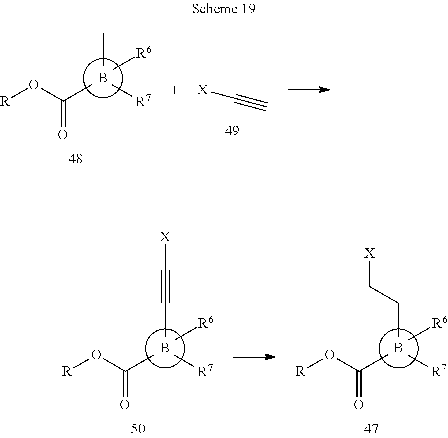

Intermediate 51, where Y=imidazolidine, oxazolidine, pyrrolidinyl, can be prepared from aldehyde 52 and amine 53, in the presence of a reducing agent such as sodium cyanoborohydride, in dichloromethane (Scheme 20).

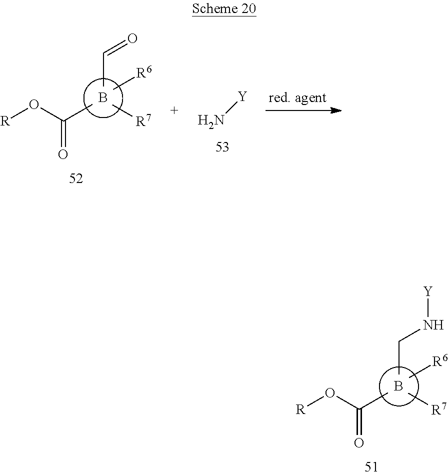

Intermediate 54 where L¹=covalent bond can be prepared starting from carboxylic acid 55 and hydrazine 56 in the presence of a base such as trimethylamine or DIPEA and a coupling agent (e.g. HATU, HOBT) in dichloromethane, giving intermediate 57 (Scheme 21). Treatment of intermediate 57 with an acid such as acetic acid and heat results in intermediate 58. Intermediate 54 can be obtained from building block 58 in a hydrogenation reaction using a heterogeneous catalyst such as Pt/C in a polar solvent such as MeOH, in a hydrogen atmosphere.

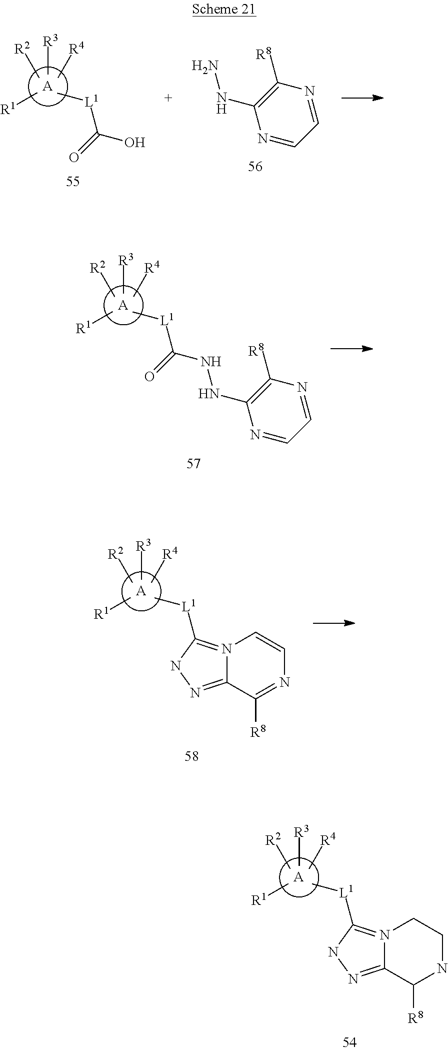

Compounds of general formula (II), wherein X is CR⁹ Y is N (hereinafter “formula (IIa)”) or X and Y are both N (hereinafter “formula (IIb)”), may be prepared in analogy to the procedures for manufacturing compounds of formula (I) outlined in Schemes 1 to 20 above. For example, the triflate as in intermediate 12 can be replaced by a bromide for the preparation of compounds of general formula IIb.

In some cases, compounds of formula I could be further functionalized to give other compounds of formula I. For example, a compound of formula I bearing a (hetero)aryl bromide or iodide can be further functionalized with other groups e.g. small amine, small alkyl using metal catalyzed cross-coupling conditions such as Buchwald or Suzuki reactions.

In some cases, compounds of formula I or building blocks bearing an ester could be further functionalized to the corresponding amides or carboxylic acids. Similarly, building blocks could be generated from commercially available fragments using standard functional group interconversion techniques (e.g. conversion of halides to other groups e.g. small amine, small alkyl using metal catalyzed cross-coupling conditions such as Buchwald or Suzuki reactions, conversion of boronic esters to boronic acids, conversion of bromides to boronic esters, alkylation of hydroxyl or amine groups via S_{N}2 reaction or reductive amination, acylation using an activated carbonyl derivative, or installation of —SO₂Me groups using literature techniques). Such techniques may also be used to elaborate fragments before, after, or within the synthetic sequences described above.

In some cases, the building blocks described as electrophile or nucleophile in cross-coupling reactions (e.g. Suzuki cross-coupling) could also be modified to act as the nucleophile or electrophile.

In one aspect, the present invention provides a process of manufacturing a compound of formula (I) described herein, or a pharmaceutically acceptable salt thereof, comprising:
(a) reacting an amine 1

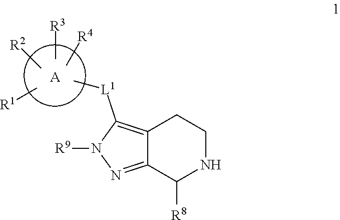

wherein the variables are as described herein;
with a carboxylic acid 2

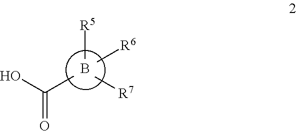

wherein the variables are as described herein;
in a solvent and in the presence of a base and a coupling agent to form said compound of formula (I); and optionally
(b) contacting said compound of formula (I) with an acid to form a pharmaceutically acceptable salt thereof

In one embodiment, the base used in said process is selected from DIPEA and TEA.

In one embodiment, the solvent used in said process is selected from DMF, DMA and CH₃CN.

In one embodiment, the coupling agent used in said process is selected from HATU and HOBT.

In one aspect, the present invention provides a compound of formula (II) as described herein, or a pharmaceutically acceptable salt thereof, when manufactured according to any one of the processes described herein.

### MAGL Inhibitory Activity

Compounds of the present invention are MAGL inhibitors. Thus, in one aspect, the present invention provides the use of compounds of formula (II) as described herein for inhibiting MAGL in a mammal.

In a further aspect, the present invention provides compounds of formula (II) as described herein for use in a method of inhibiting MAGL in a mammal.

In a further aspect, the present invention provides the use of compounds of formula (II) as described herein for the preparation of a medicament for inhibiting MAGL in a mammal.

In a further aspect, the present invention provides a method for inhibiting MAGL in a mammal, which method comprises administering an effective amount of a compound of formula (II) as described herein to the mammal.

Compounds of formula (II) were profiled for MAGL inhibitory activity by determining the enzymatic activity by following the hydrolysis of the natural substrate 2-arachidonoylglycerol (2-AG) resulting in arachidonic acid, which can be followed by mass spectrometry. This assay is hereinafter abbreviated “2-AG assay”.

Compounds of formula (II) were profiled for MAGL inhibitory activity by determining the enzymatic activity by following the hydrolysis of the natural substrate 2-arachidonoylglycerol (2-AG) resulting in arachidonic acid, which can be followed by mass spectrometry. This assay is hereinafter abbreviated “2-AG assay”. The 2-AG assay was carried out in 384 well polypropylene assay plates. Compound dilutions were made in 100% DMSO in a polypropylene plate in 3-fold dilution steps to give a final concentration range in the assay from 12.5 μM to 0.8 pM. Compound dilutions were added to MAGL protein in assay buffer (50 mM TRIS, 1 mM EDTA, 0.01% (v/v) Tween-20, 2.5% (v/v) DMSO). After shaking, the plate was incubated for 15 min at RT. To start the reaction, 2-arachidonoylglycerol in assay buffer was added. The final concentrations in the assay was 50 pM for MAGL protein and 8 pM 2-arachidonoylglyerol. After shaking and 30 min incubation at RT, the reaction was quenched by the addition of two assay volumes of acetonitrile containing 4 μM of d8-arachidonic acid. The amount of arachidonic acid formed was traced by an online SPE system (Agilent Rapidfire) coupled to a triple quadrupole mass spectrometer. A C18 SPE cartridge (Agilent G9205A) was used in an acetonitrile/water liquid setup. The mass spectrometer was operated in negative electrospray mode following the mass transitions 303.1→259.1 for arachidonic acid and 311.1→267.0 for d8-arachidonic acid. The activity of the compounds was calculated based on the ratio of intensities [arachidonic acid/d8-arachidonic acid].

TABLE 1

| | IC₅₀ |
|---|---|
| | MAGL |
| Example | [μM] |
| 1 | 0.014 |
| 2 | 1.650 |
| 3 | 0.063 |
| 4 | 0.027 |
| 5 | 0.059 |
| 6 | 0.084 |
| 7 | 0.017 |
| 8 | 0.183 |
| 9 | 0.311 |
| 10 | 4.871 |
| 11 | 0.464 |
| 12 | 0.390 |
| 13 | 0.622 |
| 14 | 0.041 |
| 15 | 0.152 |
| 16 | 0.101 |
| 17 | 0.188 |
| 18 | 3.085 |
| 19 | 0.041 |
| 20 | 0.027 |
| 21 | 1.427 |
| 22 | 0.076 |
| 23 | 0.027 |
| 24 | 1.234 |
| 25 | 0.892 |
| 26 | 0.084 |
| 27 | 0.088 |
| 28 | 0.385 |
| 29 | 0.001 |
| 30 | 0.388 |
| 31 | 0.003 |
| 32 | 0.232 |
| 33 | 0.163 |
| 34 | 0.149 |
| 35 | 0.104 |
| 36 | 0.017 |
| 37 | 0.005 |
| 38 | 0.0005 |
| 39 | 0.169 |
| 40 | 0.293 |
| 41 | 0.053 |
| 42 | 0.026 |
| 43 | 1.336 |
| 44 | 0.261 |
| 45 | 0.099 |
| 46 | 0.258 |
| 47 | 0.127 |
| 48 | 1.676 |
| 49 | 0.001 |
| 50 | 0.005 |
| 51 | 0.187 |
| 52 | 1.998 |
| 53 | 0.003 |
| 54 | 0.714 |
| 55 | 0.007 |
| 56 | 0.012 |
| 57 | 0.115 |
| 58 | 0.304 |
| 59 | 1.071 |
| 60 | 0.138 |
| 61 | 0.077 |
| 62 | 0.326 |
| 63 | 0.552 |
| 64 | 0.013 |
| 65 | 2.828 |
| 66 | 3.241 |
| 67 | 4.217 |
| 68 | 4.290 |
| 69 | 0.003 |
| 70 | 0.042 |
| 71 | 2.156 |
| 72 | 0.062 |
| 73 | 0.145 |
| 74 | 0.293 |
| 75 | 1.237 |
| 76 | 1.424 |
| 77 | 1.459 |
| 78 | 0.275 |
| 79 | 1.243 |
| 80 | 0.23989 |
| 81 | 0.02103 |
| 82 | 0.09864 |
| 83 | 4.45497 |
| 84 | 0.00618 |
| 85 | 0.01381 |
| 86 | 1.00500 |
| 87 | 0.00815 |
| 88 | 0.00177 |
| 89 | 0.02680 |
| 90 | 0.02461 |
| 91 | 1.72790 |
| 92 | 0.00435 |
| 93 | 0.73438 |
| 94 | 0.47185 |
| 95 | 0.01686 |
| 96 | 1.25560 |
| 97 | 0.00844 |
| 98 | 0.00597 |
| 99 | 0.76654 |
| 100 | 0.03190 |
| 101 | 3.01559 |
| 102 | 0.00153 |
| 103 | 0.43955 |
| 104 | 0.04242 |
| 105 | 0.00101 |
| 106 | 0.01238 |
| 107 | 0.00061 |
| 108 | 0.29428 |
| 109 | 0.37064 |
| 110 | 0.00209 |
| 111 | 0.05163 |
| 112 | 1.65766 |
| 113 | 0.00248 |
| 114 | 0.92907 |
| 115 | 0.00488 |
| 116 | 2.37954 |
| 117 | 0.12743 |

In one aspect, the present invention provides compounds of formula (II) and their pharmaceutically acceptable salts or esters as described herein, wherein said compounds of formula (II) and their pharmaceutically acceptable salts or esters have IC₅₀'s for MAGL inhibition below 25 μM, preferably below 10 μM, more preferably below 5 μM as measured in the MAGL assay described herein.

In one embodiment, compounds of formula (II) and their pharmaceutically acceptable salts or esters as described herein have IC₅₀ (MAGL inhibition) values between 0.000001 μM and 25 μM, particular compounds have IC₅₀ values between 0.000005 μM and 10 μM, further particular compounds have IC₅₀ values between 0.00005 μM and 5 μM, as measured in the MAGL assay described herein.

### Pₐₚₚ (Passive Permeability Measurements) Obtained as Part of Unidirectional P-gp Screen

### Experiment Description

The general assay uses transfected LLC-PK1 cells (porcine kidney epithelial cells) over-expressing human or mouse P-gp, cultured on 96 well semi-permeable filter membrane plates, where they form a polarized monolayer with tight junctions, and act as a barrier between the apical and basolateral compartment.

P-gp is expressed in the apical-facing membrane of the monolayer.

The tightness of the cell monolayer and functional activity of P-gp are confirmed by addition of a cell-impermeable marker, Lucifer yellow, and a reference P-gp substrate, edoxaban, respectively.

The assay is fully automated on a Tecan liquid handling robot.

### Data Analysis and Interpretation

For substrate testing the assay determines the unidirectional permeability (Pₐₚₚ A>B Equation 1) of a test compound by dosing to the apical (i.e. donor compartment) side of the cell monolayer, in the absence and presence of specific P-gp inhibitor, zosuquidar, and measuring the movement of the compound into the basolateral (i.e. receiver) compartment over a 3 hour incubation at 37° C. The effect of P-gp is measured by expressing the apical efflux ratio (AP-ER, Equation 2). The mean permeability (Pₐₚₚ) is determined in the absence of P-gp via the zosuquidar condition. The AP-ER and mean Pₐₚₚ are then used to categorize compound properties for degree of efflux and permeability (Table 2).

$${P}_{app} = \frac{1}{A * {C}_{0}} * \frac{dQ}{dt}$$

Equation 1. Pₐₚₚ, A, C0, and dQ/dt represent the apparent permeability, the filter surface area, the initial concentration, and the amount transported per time period, respectively. Pₐₚₚ values are calculated on the basis of a single time point.

$$AP - ER = \frac{Papp , inh ( A > B )}{Papp ( A > B )}$$

Equation 2. Calculation of the apical efflux ratio (AP-ER). Papp,inh (A>B) is the permeability value in the apical to basolateral direction in the presence of the inhibitor, and Papp (A>B)the permeability value in the apical-to-basolateral direction in the absence of the inhibitor.

### Using the Compounds of the Invention

In one aspect, the present invention provides compounds of formula (II), or pharmaceutically acceptable salts thereof, as described herein for use as therapeutically active substances.

In one embodiment, the compounds of the present invention are predominantly “peripherally” active, i.e., they are not penetrating the blood brain barrier, or only to a limited extent. Peripheral activity is characterized by low Pₐₚₚ values, as measured in the Pₐₚₚ assay described herein.

In a further aspect, the present invention provides a method for the treatment or prophylaxis of diseases or conditions associated with MAGL in a mammal, which method comprises administering an effective amount of a compound of formula (II), or a pharmaceutically acceptable salt thereof, as described herein to the mammal.

In one embodiment, said diseases or conditions associated with MAGL are selected from neuroinflammation, neurodegenerative diseases, pain, cancer, mental disorders and inflammatory bowel disease.

In one embodiment, said diseases or conditions associated with MAGL are selected from neuroinflammation and neurodegenerative diseases.

In one embodiment, said diseases or conditions associated with MAGL are neurodegenerative diseases.

In one embodiment, said disease or condition associated with MAGL is cancer.

In a particularly preferred embodiment, said disease or condition associated with MAGL is inflammatory bowel disease, such as ulcerative colitis or Crohn's disease.

In a particularly preferred embodiment, said disease or condition associated with MAGL is ulcerative colitis.

In a particularly preferred embodiment, said disease or condition associated with MAGL is Crohn's disease.

In one embodiment, said disease or condition associated with MAGL is irritable bowel syndrome.

The continued dysregulation of cellular systems associated with MAGL in IBD patients in clinical remission is thought to result in irritable bowel syndrome (IBS) like symptoms, such as abdominal pain and diarrhea. Therefore, in a particularly preferred embodiment, the present invention provides a method of treating or preventing IBS like symptoms, such as abdominal pain and diarrhea, in IBD patients in clinical remission.

In one embodiment, said disease or condition associated with MAGL is pain, in particular visceral pain.

In one embodiment, said diseases or conditions associated with MAGL are selected from multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and visceral pain.

In one embodiment, said diseases or conditions associated with MAGL are selected from multiple sclerosis, Alzheimer's disease and Parkinson's disease.

In one embodiment, said diseases or conditions associated with MAGL are selected from inflammatory bowel disease, inflammatory bowel disease symptoms, gut motility, visceral pain, fibromyalgia, endometriosis, COPD, and asthma.

In one aspect, the present invention provides a compound of formula (II), or a pharmaceutically acceptable salt thereof, for use in a method described above.

In one aspect, the present invention provides the use of a compound of formula (II), or of a pharmaceutically acceptable salt thereof, in a method described above.

In one aspect, the present invention provides the use of a compound of formula (II), or of a pharmaceutically acceptable salt thereof, in the manufacture of a medicament for the treatment or prophylaxis of a disease or condition associated with MAGL described herein.

Pharmaceutical Compositions and Administration In one aspect, the present invention provides a pharmaceutical composition comprising a compound of formula (II) as described herein and a therapeutically inert carrier.

In one embodiment, there is provided a pharmaceutical composition according to Example 118 or 119.

The compounds of formula (II) and their pharmaceutically acceptable salts and esters can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of formula (II) and their pharmaceutically acceptable salts and esters can be processed with pharmaceutically inert, inorganic or organic adjuvants for the production of tablets, coated tablets, dragées and hard gelatin capsules. Lactose, corn starch or derivatives thereof, talc, stearic acid or its salts etc. can be used, for example, as such adjuvants for tablets, dragées and hard gelatin capsules.

Suitable adjuvants for soft gelatin capsules are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization. The absolute stereochemistry of the examples was determined for Example 1, Example 3, Example 4, Example 16, and Example 64 by co-crystal structure of the ligand in complex with the protein. By analogy, the absolute stereochemistry was assigned to the remaining examples based on the biological activities, but was not verified experimentally. When a mixture of diastereomers was isolated, the absolute stereochemistry was arbitrarily assigned and the diastereomers were named A, B, C, or D.

All reaction examples and intermediates were prepared under an argon atmosphere if not specified otherwise.

### Abbreviations

ACN=acetonitrile, Ar=Argon, Boc=tert-butyloxycarbonyl, CAS RN=chemical abstracts registration number, CDI=N,N′-carbonyldiimidazole, CHCl₃=chloroform, Cu(OAc)₂=copper acetate, CuI=copper iodide, DCM=dihloromethane (CH₂Cl₂), DMA=N,N-Dimethylacetamide, DME=Dimethoxyethane, DMF=N,N-dimethylformamide, DMSO=dimethylsulfoxide, DIPEA=N,N-diisopropylethylamine, EI=electron ionization, ESI=electrospray ionization, EtOAc=ethyl acetate, EtOH=ethanol, FC=flash chromatography, g=gram(s), GC=gas chromatography, h=hour(s), HATU=1-[bis(dimethylamino)methylene]-1H-1,2,3-triazolo[4,5-b]pyridinium-3-oxide hexafluorophosphate, HCl=hydrogen chloride, HOBt=1-hydroxy-1H-benzotriazole; HPLC=high performance liquid chromatography, H₃PO₄=phosphoric acid, IPA=isopropyl alcohol, K₂CO₃=potassium carbonate, KOAc=potassium acetate, LiOH=lithium hydroxide, M=molar, MeOH=methanol, mg=milligram(s), MgSO₄=magnesium sulfate, min=minute(s), mL=milliliter(s), μL=microliter(s), mm Hg=millimeter of mercury, MS=mass spectrometry or molecular sieves, MTBE=methyl tertiary-butyl ether, N=normality, NaOH=sodium hydroxide, n-BuLi=n-butyllithium, NaHSO₄=sodium hydrogen sulfate, Na₂SO₄=sodium sulfate, NBS=N-bromosuccinimide, NCS=N-chlorosuccinimide, TEA=triethylamine, THF=tetrahydrofuran, NH₄Cl=ammonium chloride, PE=petroleum ether, Pd₂(dba)₃=tris(dibenzylideneacetone)dipalladium(0), PTSA=p-toluenesulfonic acid, R=any group, RP=reverse phase, SFC=supercritical fluid chromatography, SiO₂=silica, tBu=tert-butyl, TEA=trimethylamine, TFA=trifluoroacetic acid, THE=tetrahydrofuran, Xantphos=4,5-bis(diphenylphosphino)-9,9-dimethylxanthene, XPhos G3=(2-Dicyclohexylphosphino-2′,4′,6′-triisopropyl-1,1′-biphenyl)[2-(2′-amino-1,1′-biphenyl)]palladium(II) methanesulfonate.

### Example 1

### N-[[3-Chloro-5-[(7S)-6-(2-chloro-3-methoxy-benzoyl)-2,7-dimethyl-5,7-dihydro-4H-pyrazolo[3,4-c]pyridin-3-yl]phenyl]methyl]methanesulfonamide

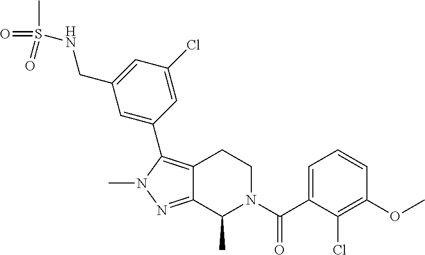 [SMILES: COc1cccc(C(=O)N2CCc3c(nn(C)c3-c3cc(Cl)cc(CNS(C)(=O)=O)c3)[C@@H]2C)c1Cl]

A solution of N-[[3-chloro-5-(2,7-dimethyl-4,5,6,7-tetrahydropyrazolo[3,4-c]pyridine-3-yl)phenyl]methyl]methanesulfonamide;hydrochloride (320.0 mg, 0.789 mmol) in dry DMF (5.0 mL) was treated with HATU (390.2 mg, 1.03 mmol), TEA (495 μL, 3.55 mmol), and 2-chloro-3-methoxybenzoic acid (CAS RN 33234-36-5; 147.3 mg, 0.789 mmol), at 25° C. under Ar. The mixture was stirred for 12 h at this temperature, before being diluted with water. The aqueous phase was extracted with Et₂O (3×), and the combined organic layers were dried over Na₂SO₄, filtered, and evaporated. Purification by RP-HPLC gave 65 mg of the racemate. Purification by chiral chromatography gave the title compound (20.9 mg, 4.9% yield) as a yellow oil. MS (ESI): m/z=537.0 [M+H]⁺.

### Step a): N-[[3-chloro-5-(4,4,5,5-tetramethyl-1,3,2-dioxaborolan-2-yl)phenyl]methyl]methanesulfonamide

A solution of N-[(3-bromo-5-chloro-phenyl)methyl]methanesulfonamide (A.1) (3.6 g, 12.06 mmol) in 1,4-dioxane (140 mL) was treated with KOAc (3.55 g, 36.17 mmol), bis(pinacolato)diboron (3.21 g, 12.66 mmol), and [1,1′-bis(diphenylphosphino)ferrocene]dichloropalladium(II) (0.88 g, 1.21 mmol), at 25° C. under Ar. The mixture was heated to 110° C. and stirred for 24 h at this temperature, before being filtered. The filtrate was evaporated, and diluted with water and EtOAc. The organic layer was washed with brine and water, dried over Na₂SO₄, filtered, and evaporated, to give the title compound (3.0 g, 61.2%) as a brown solid. MS (ESI): m/z=345.0 [M+H]⁺.

### Step b): tert-butyl 3-[3-chloro-5-(methanesulfonamidomethyl)phenyl]-2,7-dimethyl-5,7-dihydro-4H-pyrazolo[3,4-c]pyridine-6-carboxylate

A solution of tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (1.50 g, 3.76 mmol) in 5:1 1,4-dioxane/water (18 mL) was treated with N-[[3-chloro-5-(4,4,5,5-tetramethyl-1,3,2-dioxaborolan-2-yl)phenyl]methyl]methanesulfonamide (1.36 g, 3.94 mmol), K₂CO₃ (1.04 g, 7.51 mmol), and Xphos G3 (0.32 g, 0.376 mmol), at 25° C. under Ar. The mixture was heated to 110° C. and stirred for 24 h at this temperature, before being cooled down to 23° C., filtered, and evaporated. The residue was dissolved in water and EtOAc, and the aqueous phase was extracted with EtOAc (3×). The combined organic layers were dried over Na₂SO₄, filtered, and evaporated. Purification by FC gave the title compound (220 mg, 7.5%) as a yellow lyophilized solid. MS (ESI): m/z 469.0 [M+H]⁺.

### Step c) N-[[3-chloro-5-(2,7-dimethyl-4,5,6,7-tetrahydropyrazolo[3,4-c]pyridine-3-yl)phenyl]methyl]methanesulfonamide;hydrochloride

A solution of tert-butyl 3-[3-chloro-5-(methanesulfonamidomethyl)phenyl]-2,7-dimethyl-5,7-dihydro-4H-pyrazolo[3,4-c]pyridine-6-carboxylate (500.0 mg, 0.693 mmol) in MeOH (2.0 my) was treated with HCl (4 M in 1,4-dioxane; 693 μL, 2.77 mmol), at 25° C. The mixture was heated to 50° C., and stirred at this temperature for 24 h, before being evaporated, to give the title compound (320.0 mg, 30.4%) as a dark brown oil. MS (ESI): m/z=369.0 [M+H]⁺.

In analogy to Example 1, Examples in the following table were generated, using the respective building blocks A.1 or A.2. and 2-chloro-3-methoxybenzoic acid (CAS RN 33234-36-5).

| | | | | MS, |
|---|---|---|---|---|
| | | | Building | ESI: |
| Ex. | Structure | Systematic Name | Blocks | m/z |
| 2 | 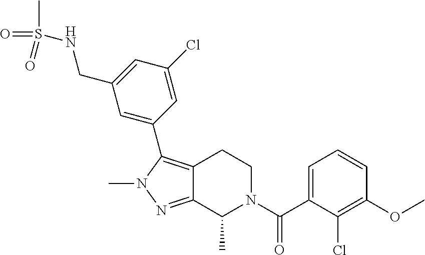 [SMILES: COc1cccc(C(=O)N2CCc3c(nn(C)c3-c3cc(Cl)cc(CNS(C)(=O)=O)c3)[C@H]2C)c1Cl] | N-[[3-Chloro-5-[(7R)-6-(2- chloro-3-methoxy-benzoyl)- 2,7-dimethyl-5,7-dihydro-4H- pyrazolo[3,4-c]pyridin-3- yl]phenyl]methyl] methanesulfonamide | A.1 and CAS RN 33234- 36-5 | 537.0 [M + H]⁺ |
| 3 | 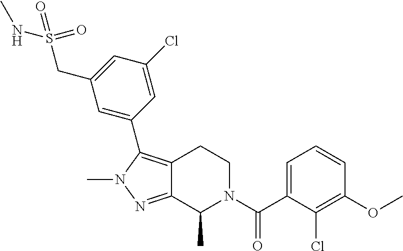 | 1-[3-Chloro-5-[(7S)-6-(2- chloro-3-methoxy-benzoyl)- 2,7-dimethyl-5,7-dihydro-4H- pyrazolo[3,4-c]pyridin-3- yl]phenyl]-N-methyl- methanesulfonamide | A.2 and CAS RN 33234- 36-5 | 537.0 [M + H]⁺ |

### Example 4

### 3-Chloro-5-[(7S)-6-(2-chloro-3-methoxy-benzoyl)-2,7-dimethyl-5,7-dihydro-4H-pyrazolo[3,4-c]pyridin-3-yl]benzenesulfonamide

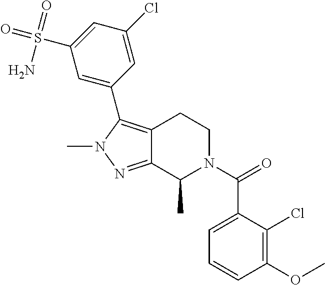 [SMILES: COc1cccc(C(=O)N2CCc3c(nn(C)c3-c3cc(Cl)cc(S(N)(=O)=O)c3)[C@@H]2C)c1Cl]

A solution of N′-[3-chloro-5-[6-(2-chloro-3-methoxy-benzoyl)-2,7-dimethyl-5,7-dihydro-4H-pyrazolo[3,4-c]pyridine-3-yl]phenyl]sulfonyl-N,N-dimethyl-formamidine (260.0 mg, 0.460 mmol) in MeOH (3 mL) was treated with 12 N HCl (0.38 mL, 4.61 mmol), at 23° C. The mixture was heated to 60° C., and stirred for 10 h at this temperature, before being concentrated. The residue was purified by RP-HPLC, to give the racemate (134.8 mg, 56.88% yield) as a white solid. Purification by chiral chromatography gave the title compound (55.2 mg, 40.95% yield) as a yellow solid. MS (ESI): m/z=511.0 [M+H]⁺.

### Step a): tert-Butyl 3-(3-chloro-5-sulfamoyl-phenyl)-2,7-dimethyl-5,7-dihydro-4H-pyrazolo[3,4-c]pyridine-6-carboxylate

A solution of tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (845.0 mg, 1.59 mmol) in dry DMF was treated with 3-chloro-5-(4,4,5,5-tetramethyl-1,3,2-dioxaborolan-2-yl)benzenesulfonamide (EN300-12593930; 655.16 mg, 2.06 mmol) and sodium carbonate (336.37 mg, 3.17 mmol), at 25° C. under Ar. The solution was sparged with Ar for 10 min, before being treated with tetrakis(triphenylphosphine)palladium(0) (91.68 mg, 0.080 mmol). The resulting mixture was stirred for 16 h at 100° C., cooled down to 25° C., and diluted with water (50 mL). The aqueous phase was extracted with EtOAc (2×), and the combined organic layers were washed with brine, dried over Na₂SO₄, filtered, and evaporated. Purification by FC (SiO₂; hexane/EtOAc) gave the title compound (350 mg, 50.02% yield) as a crude white solid. MS (ESI): m/z=385.0 [M−Bu+H]⁺.

### Step b): tert-butyl 3-[3-chloro-5-[(E)-dimethylaminomethyleneamino]sulfonyl-phenyl]-2,7-dimethyl-5,7-dihydro-4H-pyrazolo[3,4-c]pyridine-6-carboxylate

A solution of tert-butyl 3-(3-chloro-5-sulfamoyl-phenyl)-2,7-dimethyl-5,7-dihydro-4H-pyrazolo[3,4-c]pyridine-6-carboxylate (30.0 mg, 0.070 mmol) in 1,1-dimethoxy-N,N-dimethyl-methanamine (0.18 mL, 1.36 mmol) was stirred for 5 h at 20° C. before being evaporated, to give the title compound (30 mg, 88.9% yield) as a colorless foam. MS (ESI): m/z=440.0 [M-Bu+H]⁺.

### Step c): N′-[3-chloro-5-(2,7-dimethyl-4,5,6,7-tetrahydropyrazolo[3,4-c]pyridine-6-ium-3-yl)phenyl]sulfonyl-N,N-dimethyl-formamidine;2,2,2-trifluoroacetate

A solution of tert-butyl 3-[3-chloro-5-[(E)-dimethylaminomethyleneamino]sulfonyl-phenyl]-2,7-dimethyl-5,7-dihydro-4H-pyrazolo[3,4-c]pyridine-6-carboxylate (30.0 mg, 0.060 mmol) in DCM (1 mL) was treated with TFA (0.05 mL, 0.600 mmol), at 20° C. The mixture was stirred for 10 h at this temperature before being evaporated, to give the title compound (B.1; 28 mg, 81.89% yield) as a light yellow oil. MS (ESI): m/z=396.0/398.2 [M+H]⁺.

### Step d): N′-[3-chloro-5-[6-(2-chloro-3-methoxy-benzoyl)-2,7-dimethyl-5,7-dihydro-4H-pyrazolo[3,4-c]pyridine-3-yl]phenyl]sulfonyl-N,N-dimethyl-formamidine

A solution of 2-chloro-3-methoxybenzoic acid (CAS RN 33234-36-5; 93.31 mg, 0.500 mmol) in ACN (5 mL) was treated with HATU (209.16 mg, 0.550 mmol) and TEA (0.35 mL, 2.5 mmol), at 20° C. The mixture was stirred for 10 min at this temperature, before being treated with N′-[3-chloro-5-(2,7-dimethyl-4,5,6,7-tetrahydropyrazolo[3,4-c]pyridine-6-ium-3-yl)phenyl]sulfonyl-N,N-dimethyl-formamidine;2,2,2-trifluoroacetate (255.0 mg, 0.500 mmol) and stirred for 10 h at 20° C. The mixture was evaporated, and the residue was dissolved in EtOAc. The solution was washed with water, dried over Na₂SO₄, filtered, and evaporated, to give the title compound (260 mg, 79.4% yield) as a light yellow foam. MS (ESI): m/z=564.2/566.2/568.0 [M+H]⁺.

### Example 5

### (2-Chloro-3-methoxy-phenyl)-[(7S)-3-[3-chloro-5-(1-methylsulfonylcyclopropyl)phenyl]-2,7-dimethyl-5,7-dihydro-4H-pyrazolo[3,4-c]pyridin-6-yl]methanone

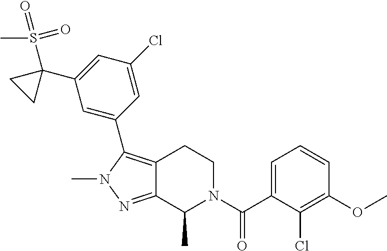

A solution of 3-[3-chloro-5-(1-methylsulfonylcyclopropyl)phenyl]-2,7-dimethyl-4,5,6,7-tetrahydropyrazolo[3,4-c]pyridine;hydrochloride (115.82 mg, 0.280 mmol) in DMF (5 mL) was treated with 2-chloro-3-methoxybenzoic acid (CAS RN 33234-36-5; 51.91 mg, 0.280 mmol), HATU (137.5 mg, 0.360 mmol), and DIPEA (0.12 mL, 0.830 mmol), at 25° C. under Ar. The mixture was stirred for 18 h at this temperature, before being poured onto water. The aqueous phase was extracted with EtOAc, and the combined organic layers were washed with brine, dried over Na₂SO₄, filtered, and evaporated. Purification by RP-HPLC and chiral chromatography gave the title compound (41.9 mg, 27.46% yield) as white solid. MS (ESI): m/z=563.2 [M+H]⁺.

### Step a): 2-[3-chloro-5-(1-methylsulfonylcyclopropyl)phenyl]-4,4,5,5-tetramethyl-1,3,2-dioxaborolane

A solution of 1-bromo-3-chloro-5-(1-methylsulfonylcyclopropyl)benzene (A.3; 2.0 g, 6.50 mmol) in 1,4-dioxane (40 mL) was treated with bis(pinacolato)diboron (1804.42 mg, 7.11 mmol), potassium acetate (1.21 mL, 19.38 mmol) and 1,1′-bis(diphenylphosphino)ferrocene-palladium(II)dichloride dichloromethane complex (527.11 mg, 0.650 mmol), at 25° C. under Ar. The mixture was heated to 80° C., and stirred at this temperature for 16 h before being cooled down, filtered, and evaporated. The residue was dissolved in EtOAc, and the organic layer was washed with water and brine, dried over Na₂SO₄, filtered, and evaporated. Purification by FC (SiO₂; hexane/MTBE) gave the title compound (1.2 g, 49.48% yield) as a crude light yellow solid, which was directly used in the next step.

### Step b): tert-Butyl 3-[3-chloro-5-(I-methylsulfonylcyclopropyl)phenyl]-2,7-dimethyl-5,7-dihydro-4H-pyrazolo[3,4-c]pyridine-6-carboxylate

A solution of 2-[3-chloro-5-(1-methylsulfonylcyclopropyl)phenyl]-4,4,5,5-tetramethyl-1,3,2-dioxaborolane (1270.0 mg, 3.56 mmol) in 4:1 THF/water (50 mL) was treated with tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (1422.11 mg, 3.56 mmol) and sodium carbonate (754.79 mg, 7.12 mmol), at 25° C. under Ar. The mixture was sparged with Ar for 5 min, before being treated with tetrakis(triphenylphosphine)palladium(0) (411.46 mg, 0.360 mmol). The mixture was heated to 70° C. and stirred for 16 h at this temperature, before being cooled down to 25° C. and filtered through a SiO₂ pad. The filtrate was evaporated and purified by FC (SiO₂; CHCl₃/ACN), to give the title compound (1.3 g, 66.93% yield) as a white solid. MS (ESI): m/z=424.0 [M-tBu+H]⁺.

### Step c): 3-[3-chloro-5-(I-methylsulfonylcyclopropyl)phenyl]-2,7-dimethyl-4,5,6,7-tetrahydro-pyrazolo[3,4-c]pyridine;hydrochloride

A solution of tert-butyl 3-[3-chloro-5-(1-methylsulfonylcyclopropyl)phenyl]-2,7-dimethyl-5,7-dihydro-4H-pyrazolo[3,4-c]pyridine-6-carboxylate (120.0 mg, 0.250 mmol) in 4 M HCl in 1,4-dioxane (1.0 mL, 4.0 mmol) was stirred for 16 h at 23° C., before being evaporated. Trituration with MTBE gave the title compound (B.2; 105 mg, 97.85% yield) as a light yellow solid. MS (ESI): m/z=380.0 [M+H]⁺.

### Example 6

### (2-Chloro-3-methoxy-phenyl)-[(7S)-3-[3-chloro-5-(methylsulfonylmethyl)phenyl]-2,7-dimethyl-5,7-dihydro-4H-pyrazolo[3,4-c]pyridin-6-yl]methanone

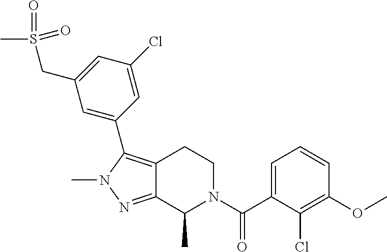

A solution of 3-[3-chloro-5-(methylsulfonylmethyl)phenyl]-2,7-dimethyl-4,5,6,7-tetrahydropyrazolo[3,4-c]pyridine;2,2,2-trifluoroacetic acid (550.0 mg, 1.18 mmol) in DMF (3 mL) was treated with 2-chloro-3-methoxybenzoic acid (CAS RN 33234-36-5; 219.33 mg, 1.18 mmol), DIPEA (0.82 mL, 4.7 mmol) and HATU (536.35 mg, 1.41 mmol), at 23° C. The mixture was stirred for 18 h at this temperature, before being purified by RP-HPLC and chiral chromatography, to give the title compound (37.4 mg, 6.09% yield) as a yellow gum. MS (ESI): m/z=522 [M+H]⁺.

### Step a): I-bromo-3-chloro-5-(methylsulfonylmethyl)benzene

A solution of 1-bromo-3-(bromomethyl)-5-chloro-benzene (CAS RN: 762292-63-7; 6.3 g, 22.15 mmol) in DMF (12 mL) was treated with sodium methanesulfinate (3.39 g, 33.23 mmol), at 23° C. The mixture was heated to 50° C. and stirred for 18 h at this temperature, before being cooled down. The mixture was diluted with water, and the resulting precipitate was filtered, rinsed with water, and dried, to give the title compound (5.7 g, 90.73% yield) as a white solid. MS (ESI): m/z=282.8 [M−H]⁻.

### Step b): 2-[3-chloro-5-(methylsulfonylmethyl)phenyl]-4,4,5,5-tetramethyl-1,3,2-dioxaborolane

A solution of 1-bromo-3-chloro-5-(methylsulfonylmethyl)benzene (5.6 g, 19.75 mmol) in 1,4-dioxane (200 mL) was treated with bis(pinacolato)diboron (5.27 g, 20.74 mmol), KOAc (3.7 mL, 59.24 mmol), and 1,1′-bis(diphenylphosphino)ferrocene-palladium(II)dichloride dichloromethane complex (0.81 g, 0.990 mmol), at 23° C. under Ar. The mixture was heated to 100° C. and stirred for 16 h at this temperature, before being cooled down and filtered over SiO₂. The filtrate was evaporated. Trituration in hexane gave the title compound (4.37 g, 63.58% yield) as a crude light brown solid, which was directly used in the next step.

### Step c): [3-chloro-5-(methylsulfonylmethyl)phenyl]boronic acid

A solution of 2-[3-chloro-5-(methylsulfonylmethyl)phenyl]-4,4,5,5-tetramethyl-1,3,2-dioxaborolane (4.37 g, 13.22 mmol) in 3:1 THF/water (100 mL) was treated with sodium metaperiodate (8.48 g, 39.65 mmol) and ammonium acetate buffer solution (2.04 g, 26.43 mmol), at 25° C. The mixture was stirred for 18 h at this temperature, before being filtered and evaporated. Trituration in hexane gave the title compound (3.4 g, 98.35% yield) as a crude grey solid, which was directly used in the next step.

### Step d): tert-butyl 3-[3-chloro-5-(methylsulfonylmethyl)phenyl]-2,7-dimethyl-5,7-dihydro-4H-pyrazolo[3,4-c]pyridine-6-carboxylate

A solution of [3-chloro-5-(methylsulfonylmethyl)phenyl]boronic acid (373.3 mg, 1.5 mmol) in dry DMF (10 mL) was treated with tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (500.0 mg, 1.25 mmol) and sodium carbonate (265.37 mg, 2.5 mmol), at 23° C. under Ar. The mixture was sparged with Ar for 10 min, before being treated with tetrakis(triphenylphosphine)palladium(0) (144.67 mg, 0.130 mmol), and heated to 100° C. The mixture was stirred for 16 h at this temperature, cooled down, filtered, and evaporated. Purification by RP-HPLC gave the title compound (236 mg, 41.52% yield) as a light yellow solid. MS (ESI): m/z=452.2 [M−H]⁻.

### Step e): 3-[3-chloro-5-(methylsulfonylmethyl)phenyl]-2,7-dimethyl-4,5,6,7-tetrahydropyrazolo[3,4-c]pyridine; 2,2,2-trifluoroacetic acid

A solution of tert-butyl 3-[3-chloro-5-(methylsulfonylmethyl)phenyl]-2,7-dimethyl-5,7-dihydro-4H-pyrazolo[3,4-c]pyridine-6-carboxylate (550.0 mg, 1.21 mmol) in DCM (5.5 mL) was treated with TFA (0.37 mL, 4.85 mmol), at 25° C. The mixture was stirred for 18 h at this temperature. Diethyl ether was added, and the resulting precipitate was filtered and dried, to give the title compound (500 mg, 80.09% yield) as a white solid. MS (ESI): m/z=354 [M+H]f.

### Example 7

### N-[1-[3-Chloro-5-[(7S)-6-(2-chloro-3-methoxy-benzoyl)-2,7-dimethyl-5,7-dihydro-4H-pyrazolo[3,4-c]pyridin-3-yl]phenyl]cyclopropyl]methanesulfonamide

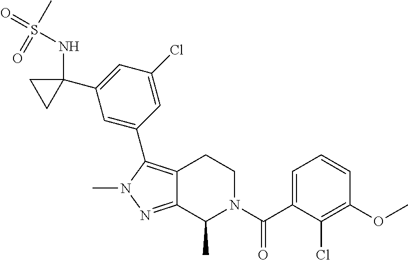 [SMILES: COc1cccc(C(=O)N2CCc3c(nn(C)c3-c3cc(Cl)cc(C4(NS(C)(=O)=O)CC4)c3)[C@@H]2C)c1Cl]

A solution of N-[1-[3-chloro-5-(2,7-dimethyl-4,5,6,7-tetrahydropyrazolo[3,4-c]pyridine-3-yl)phenyl]cyclopropyl]methanesulfonamide;hydrochloride (B.3; 150.0 mg, 0.350 mmol) in DMF (5 mL) was treated with 2-chloro-3-methoxybenzoic acid (CAS RN 33234-36-5; 64.88 mg, 0.350 mmol), HATU (171.88 mg, 0.450 mmol) and TEA (0.15 mL, 1.04 mmol), at 25° C. The mixture was stirred for 18 h at this temperature, before being poured onto water and extracted with EtOAc. The combined organic layers were washed with water, dried over Na₂SO₄, filtered, and evaporated. Purification by RP-HPLC gave 200 mg of racemate. Chiral chromatography gave the title compound (49.5 mg, 25.26% yield) as a light brown solid. MS (ESI): m/z=563.2 [M+H]⁺.

### Step a): N-[1-(3-bromo-5-chloro-phenyl)cyclopropyl]methanesulfonamide

A solution of 1-(3-bromo-5-chloro-phenyl)cyclopropanamine;hydrochloride (CAS RN: 2089255-58-1; 3.0 g, 10.6 mmol) in dry DCM (40 mL) was treated with TEA (3.69 mL, 26.5 mmol), at 23° C. The mixture was stirred for 10 min at this temperature and cooled down to 0° C. The mixture was treated dropwise with a solution of methanesulfonyl chloride (0.98 mL, 12.72 mmol) in DCM (4 mL), at 0° C. The mixture was stirred for 4 h at 23° C., before being treated with water and extracted with EtOAc. The combined organic layers were washed with brine, dried over Na₂SO₄, filtered and evaporated, to give the title compound (3 g, 82.82% yield) as a white solid. MS (ESI): m/z=324.0 [M+H]⁺.

### Step b): N-[1-[3-chloro-5-(4,4,5,5-tetramethyl-1,3,2-dioxaborolan-2-yl)phenyl]cyclopropyl]methane-sulfonamide

A solution of N-[1-(3-bromo-5-chloro-phenyl)cyclopropyl]methanesulfonamide (3.0 g, 9.24 mmol) in 1,4-dioxane (30 mL) was treated with bis(pinacolato)diboron (2581.49 mg, 10.17 mmol), 1,1′-bis(diphenylphosphino)ferrocene-palladium(II)dichloride dichloromethane complex (754.11 mg, 0.920 mmol) and potassium acetate (2.72 g, 27.72 mmol), at 23° C. under Ar. The mixture was heated to 80° C. and stirred for 16 h at this temperature, before being cooled down, filtered, and evaporated. The residue was partitioned between EtOAc and water. The organic layer was washed with brine, dried over Na₂SO₄, filtered, and evaporated. Purification by FC (SiO₂; hexane/MTBE) gave the title compound (2 g, 55.31% yield) as a light yellow solid. MS (ESI): m/z=289.0 [M+H]⁺.

### Step c): [3-chloro-5-[1-(methanesulfonamido)cyclopropyl]phenyl]boronic acid

A solution of N-[1-[3-chloro-5-(4,4,5,5-tetramethyl-1,3,2-dioxaborolan-2-yl)phenyl]cyclopropyl]methanesulfonamide (1.6 g, 4.3 mmol) in acetone (20 mL) was treated with sodium metaperiodate (2762.2 mg, 12.91 mmol) and ammonium acetate buffer solution (663.63 mg, 8.61 mmol) in water (8 mL), at 23° C. The mixture was stirred for 3 h at this temperature, and water (4 mL) was added. The mixture was stirred for 24 h at 40° C. After addition of 4 N aqueous HCl (3 mL), the organic phase was concentrated in vacuo and the remaining aqueous solution was extracted with EtOAc. The combined organic layers were washed with brine, dried over Na₂SO₄, filtered, and evaporated, to give the title compound (1.1 g, 79.43% yield) as a dark brown oil. MS (ESI): m/z=287.8 [M−H]⁻.

### Step d): tert-butyl 3-[3-chloro-5-[1-(methanesulfonamido)cyclopropyl]phenyl]-2,7-dimethyl-5,7-dihydro-4H-pyrazolo[3,4-c]pyridine-6-carboxylate

A solution of [3-chloro-5-[1-(methanesulfonamido)cyclopropyl]phenyl]boronic acid (942.44 mg, 3.25 mmol) in 4:1 THF/water (5 mL) was treated with tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (1.0 g, 2.5 mmol) and sodium carbonate (530.0 mg, 5 mmol), at 23° C. under Ar. The mixture was sparged with Ar for 10 min, and tetrakis(triphenylphosphine)palladium(0) (290.0 mg, 0.250 mmol) was added.

The mixture was heated to 70° C. and stirred for 16 h at this temperature, before being cooled down, filtered over SiO₂, and evaporated. Purification by FC (SiO₂; CHCl₃/ACN) gave the title compound (900 mg, 72.61% yield) as a white solid. MS (ESI): m/z=495.0 [M+H]⁺.

### Step e): N-[1-[3-chloro-5-(2,7-dimethyl-4,5,6,7-tetrahydropyrazolo[3,4-c]pyridine-3-yl)phenyl]cyclopropyl]methanesulfonamide;hydrochloride

A solution of tert-butyl 3-[3-chloro-5-[1-(methanesulfonamido)cyclopropyl]phenyl]-2,7-dimethyl-5,7-dihydro-4H-pyrazolo[3,4-c]pyridine-6-carboxylate (780.0 mg, 1.58 mmol) in MeOH (5 mL) was treated with 4 M HCl in 1,4-dioxane (2.34 mL, 9.36 mmol). The mixture was stirred for 16 h at 23° C., before being evaporated. Trituration with MTBE gave the title compound (B.3; 387.8 mg, 54.2% yield) as a light yellow solid. MS (ESI): m/z=395.0 [M+H]⁺.

### Example 8

### (2-Chloro-3-methoxy-phenyl)-[(7S)-2,7-dimethyl-3-[6-(trifluoromethyl)pyrazin-2-yl]-5,7-dihydro-4H-pyrazolo[3,4-c]pyridin-6-yl]methanone

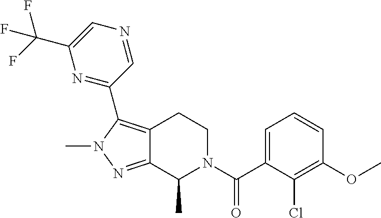 [SMILES: COc1cccc(C(=O)N2CCc3c(nn(C)c3-c3cncc(C(F)(F)F)n3)[C@@H]2C)c1Cl]

A solution of 2,7-dimethyl-3-[6-(trifluoromethyl)pyridine-2-yl]-4,5,6,7-tetrahydropyrazolo[3,4-c]pyridine (400.0 mg, 1.35 mmol) in DMF (3.0 mL) was treated with 2-chloro-3-methoxybenzoic acid (CAS RN 33234-36-5; 251.06 mg, 1.35 mmol), HATU (613.93 mg, 1.61 mmol) and DIPEA (0.94 mL, 5.38 mmol), at 25° C. under Ar. The mixture was stirred for another 18 h, before being purified by RP-HPLC and chiral chromatography, to give the title compound (117.4 mg, 18.73% yield) as a white solid. MS (ESI): m/z=466.0 [M+H]f.

### Step a): tert-butyl 2,7-dimethyl-3-[6-(trifluoromethyl)pyridine-2-yl]-5,7-dihydro-4H-pyrazolo[3,4-c]pyridine-6-carboxylate

A solution of 2-(4,4,5,5-tetramethyl-1,3,2-dioxaborolan-2-yl)-6-(trifluoromethyl)pyrazine (CAS RN 2223053-21-0; 3.92 g, 10.01 mmol) in dry DMF (95.99 mL) was treated with tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (4.0 g, 10.01 mmol) and sodium carbonate (2.12 g, 20.03 mmol), at 25° C. under Ar. The mixture was sparged with Ar for 10 min, before being treated with tetrakis(triphenylphosphine)palladium(0) (1.16 g, 1 mmol). The resulting mixture was stirred heated to 100° C. and stirred for 16 h at this temperature, before being cooled down, filtered, and evaporated. Purification by FC (SiO₂; hexane/MtBE) gave the title compound (1.3 g, 31% yield) as a light yellow solid. MS (ESI): m/z=342.0 [(M-tBu+H]⁺.

### Step b): 2,7-dimethyl-3-[6-(trifluoromethyl)pyridine-2-yl]-4,5,6,7-tetrahydropyrazolo[3,4-c]pyridine; hydrochloride

tert-Butyl 2,7-dimethyl-3-[6-(trifluoromethyl)pyridine-2-yl]-5,7-dihydro-4H-pyrazolo[3,4-c]pyridine-6-carboxylate (1298.4 mg, 3.27 mmol) was treated with 4 M HCl in 1,4-Dioxane (16.23 mL), at 25° C. The mixture was stirred for 18 h at this temperature. The resulting suspension was filtered off and washed with diethyl ether, to give the title compound (B.3; 950 mg, 87.12% yield) as a white powder. MS (ESI): m/z=298 [M+H]⁺.

In analogy to Example 8, Examples in the following table were generated, using commercially available building blocks.

| | | | | MS, |
|---|---|---|---|---|
| | | | Building | ESI: |
| Ex. | Structure | Systematic Name | Blocks | m/z |
| 9 | 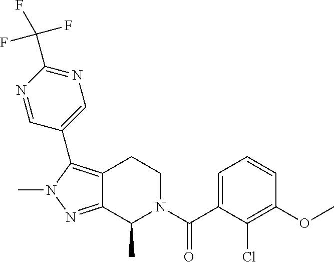 [SMILES: COc1cccc(C(=O)N2CCc3c(nn(C)c3-c3cnc(C(F)(F)F)nc3)[C@@H]2C)c1Cl] | (2-Chloro-3-methoxy-phenyl)- [(7S)-2,7-dimethyl-3-[2- (trifluoromethyl)pyrimidin-5-yl]- 5,7-dihydro-4H-pyrazolo[3,4- c]pyridin-6-yl]methanone | CAS RN 1701434- 52-7 and CAS RN 33234- 36-5 | 466.0 [M + H]⁺ |
| 10 | 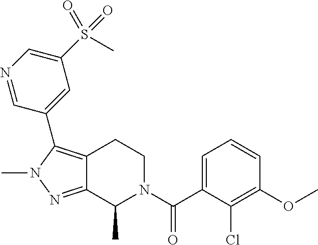 | (2-Chloro-3-methoxy-phenyl)- [(7S)-2,7-dimethyl-3-(5- methylsulfonyl-3-pyridyl)-5,7- dihydro-4H-pyrazolo[3,4- c]pyridin-6-yl]methanone | CAS 1206641- 26-0 and CAS RN 33234- 36-5 | 475.0 [M + H]⁺ |

### Example 11

### (2-Chloro-3-methoxy-phenyl)-[(7S)-2,7-dimethyl-3-[[[1-(trifluoromethyl)cyclopropyl]amino]methyl]-5,7-dihydro-4H-pyrazolo[3,4-c]pyridin-6-yl]methanone

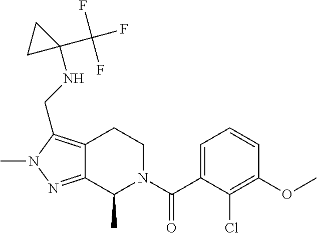

A solution of 2-chloro-3-methoxy-benzoic acid (CAS RN 33234-36-5; 320.9 mg, 1.72 mmol) in DCM (5 mL) was treated with a drop of DMF and oxalyl chloride (0.15 mL, 1.72 mmol), at 20° C. under Ar. The mixture was stirred for 1 h at this temperature, before being evaporated. A solution of (2,7-dimethyl-4,5,6,7-tetrahydropyrazolo[3,4-c]pyridine-6-ium-3-yl)methyl-[1-(trifluoromethyl)cyclopropyl]ammonium; di-chloride (310.0 mg, 0.860 mmol) in DCM (10 mL) was treated with DIPEA (0.6 mL, 3.43 mmol), at 20° C. The mixture was then cooled down to 0° C. and treated dropwise with above-prepared the acyl chloride. The mixture was allowed to warm up to 20° C. and stirred for 5 h at this temperature before being evaporated. Purification by RP-HPLC and chiral chromatography gave the title compound (134.6 mg, 52.3% yield) as a brown solid. MS (ESI): m/z=457.0/459.0 [M+H]⁺.

### Step a): tert-butyl 2,7-dimethyl-3-[(E)-styryl]-5,7-dihydro-4H-pyrazolo[3,4-c]pyridine-6-carboxylate

A solution of (E)-phenylethenylboronic acid (1867.27 mg, 12.62 mmol) in DMF (30 mL) was treated with tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (4200.0 mg, 10.52 mmol), sodium carbonate (2229.19 mg, 21.03 mmol) in DMF (30 mL) and tetrakis(triphenylphosphine)palladium(0) (607.6 mg, 0.530 mmol), at 20° C. under Ar. The resulting mixture was heated to 100° C. and stirred for 15 h at this temperature, before being cooled down and diluted with water. The aqueous layer was extracted with EtOAc, and the combined organic layers were washed with brine, dried over Na₂SO₄, filtered, and evaporated. Purification by FC (SiO₂; hexane/EtOAc 3:1) gave the title compound (3200 mg, 83.42% yield) as a yellow solid. ESI (MS): m/z=354.2 [M+H]⁺.

### Step b): tert-butyl 3-formyl-2,7-dimethyl-5,7-dihydro-4H-pyrazolo[3,4-c]pyridine-6-carboxylate

A solution of tert-butyl 2,7-dimethyl-3-[(E)-styryl]-5,7-dihydro-4H-pyrazolo[3,4-c]pyridine-6-carboxylate (3200.0 mg, 9.05 mmol) in 5:1 1,4-Dioxane/water (60 mL) was treated with osmium tetroxide (115.08 mg, 0.450 mmol) and sodium metaperiodate (5809.27 mg, 27.16 mmol), at 0° C. The mixture was allowed to warm up to 25° C. and stirred for 5 h at this temperature. A precipitate formed. The precipitate was filtered off, and the filtrate was diluted with brine and extracted with EtOAc. The combined organic layers were washed with brine, dried over Na₂SO₄, and evaporated. Purification by FC (SiO₂; hexane/EtOAc 3:1) gave the title compound (C.1) (1022 mg, 36.76% yield) as a yellow oil. MS (ESI): m/z=224.2 [M-Bu+H]⁺.

### Step c): tert-butyl 2,7-dimethyl-3-[[[1-(trifluoromethyl)cyclopropyl]amino]methyl]-5,7-dihydro-4H-pyrazolo[3,4-c]pyridine-6-carboxylate

A solution of tert-butyl 3-formyl-2,7-dimethyl-5,7-dihydro-4H-pyrazolo[3,4-c]pyridine-6-carboxylate (300.0 mg, 1.07 mmol) in 1,2-dichloroethane (8 mL) was treated with [1-(trifluoromethyl)cyclopropyl]ammonium;chloride (190.85 mg, 1.18 mmol) and acetic acid (144 μL, 0.25 mmol), at 25° C. The mixture was stirred for 30 min, before being treated with sodium triacetoxyborohydride (796.68 mg, 3.76 mmol). The resulting mixture was stirred for another 15 h at 20° C., before being diluted with DCM. The organic layer was washed with saturated aqueous sodium bicarbonate and water, dried over Na₂SO₄, filtered, and evaporated, to give the title compound (390 mg, 93.49% yield) as a light yellow oil. ESI (MS): m/z=389.3 [M+H]⁺.

### Step d): (2,7-dimethyl-4,5,6,7-tetrahydropyrazolo[3,4-c]112yridine-6-ium-3-yl)methyl-[1-(trifluoromethyl)cyclopropyl]ammonium;dichloride

A solution of tert-butyl 2,7-dimethyl-3-[[[1-(trifluoromethyl)cyclopropyl]amino]methyl]-5,7-dihydro-4H-pyrazolo[3,4-c]pyridine-6-carboxylate (340.0 mg, 0.880 mmol) in DCM (10 mL) was treated with hydrochloric acid (4 M in 1,4-dioxane) (2.19 mL, 8.75 mmol), at 20° C. The mixture was stirred for 10 h at this temperature before being evaporated, to give the title compound (310 mg, 98.04% yield) as a crude light yellow powder. MS (ESI): m/z=289.2 [M+H]⁺.

### Example 12

### (2-Chloro-3-methoxy-phenyl)-[(7S)-2,7-dimethyl-3-[5-(trifluoromethyl)-1,3,4-oxadiazol-2-yl]-5,7-dihydro-4H-pyrazolo[3,4-c]pyridin-6-yl]methanone

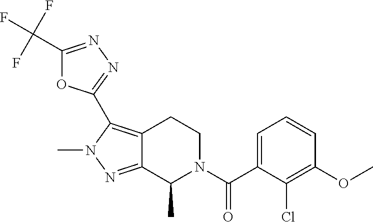 [SMILES: COc1cccc(C(=O)N2CCc3c(nn(C)c3-c3nnc(C(F)(F)F)o3)[C@@H]2C)c1Cl]

A solution of (2-chloro-3-methoxy-phenyl)-[2,7-dimethyl-3-(2H-tetrazol-5-yl)-5,7-dihydro-4H-pyrazolo[3,4-c]pyridine-6-yl]methanone (230.0 mg, 0.590 mmol) in toluene (10 mL) was treated with trifluoroacetic anhydride (0.1 mL, 0.710 mmol), at 20° C. The mixture was stirred for 1 h at this temperature, before being heated to 100° C. and stirred for another 10 h. The mixture was then cooled down and evaporated. Purification by RP-HPLC and chiral chromatography gave the title compound (51.8 mg, 29.3% yield) as a light brown foam. MS (ESI): m/z=456.2/458.2 [M+H]⁺.

### Step a): tert-butyl 3-cyano-2,7-dimethyl-5,7-dihydro-4H-pyrazolo[3,4-c]pyridine-6-carboxylate

A solution of tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (3000.0 mg, 7.51 mmol) in DMF (25 mL) was treated with zinc cyanide (0.52 mL, 8.26 mmol), 1,1′-bis(diphenylphosphino)ferrocene (416.42 mg, 0.750 mmol), and tris(dibenzylideneacetone)dipalladium (0) (343.92 mg, 0.380 mmol), at 20° C. under Ar. The resulting mixture was heated to 100° C. and stirred at this temperature for 10 h, before being cooled down and poured into water. The aqueous layer was extracted with EtOAc, and the combined organic layers were washed with brine, dried over Na₂SO₄, filtered and evaporated. Purification by FC (SiO₂; hexane/EtOAc 5:1) gave the title compound (1100 mg, 49.97% yield) as a light yellow solid. MS (ESI): m/z=221.0 [M-Bu+H]⁺.

### Step b): 2,7-dimethyl-4,5,6,7-tetrahydropyrazolo[3,4-c]pyridine-6-ium-3-carbonitrile;chloride

A solution of tert-butyl 3-cyano-2,7-dimethyl-5,7-dihydro-4H-pyrazolo[3,4-c]pyridine-6-carboxylate (300.0 mg, 1.09 mmol) in DCM (5 mL) was treated with HCl (4 M in 1,4-dioxane) (2.71 mL, 10.86 mmol), at 20° C. The mixture was stirred for 10 h at this temperature before being evaporated, to give the title compound (230 mg, 91.44% yield) as a white solid. MS (ESI): m/z=177.2 [M+H]⁺.

### Step c): 6-(2-chloro-3-methoxy-benzoyl)-2,7-dimethyl-5,7-dihydro-4H-pyrazolo[3,4-c]pyridine-3-carbonitrile

A solution of 2-chloro-3-methoxybenzoic acid (CAS RN 33234-36-5; 201.79 mg, 1.08 mmol) in DCM (5 mL) was treated dropwise with oxalyl chloride (0.14 mL, 1.62 mmol) at 20° C., stirred for 1 h at this temperature, and evaporated. The residue was dissolved in DCM (10 mL) and cooled down to 0° C., before being treated with 2,7-dimethyl-4,5,6,7-tetrahydropyrazolo[3,4-c]pyridine-6-ium-3-carbonitrile;chloride (230.0 mg, 1.08 mmol) and DIPEA (0.75 mL, 4.33 mmol). The mixture was allowed to warm up to 20° C., and stirred for 5 h at this temperature. The reaction mixture was diluted with water, and the layers were separated. The organic layer was washed with brine, dried over Na₂SO₄, filtered, and evaporated, to give the title compound (340 mg, 78.96% yield) as a yellow solid. MS (ESI): m/z=345.2/347.2 [M+H]⁺.

### Step d): (2-chloro-3-methoxy-phenyl)-[2,7-dimethyl-3-(2H-tetrazol-5-yl)-5,7-dihydro-4H-pyrazolo[3,4-c]pyridine-6-yl]methanone

A solution of 6-(2-chloro-3-methoxy-benzoyl)-2,7-dimethyl-5,7-dihydro-4H-pyrazolo[3,4-c]pyridine-3-carbonitrile (240.0 mg, 0.700 mmol) in DMF (5 mL) was treated with ammonium chloride (55.85 mg, 1.04 mmol) and sodium azide (0.04 mL, 1.04 mmol), at 20° C. The mixture was heated to 100° C. and stirred for 10 h at this temperature, before being cooled down. The mixture was diluted with brine and extracted with EtOAc (3×10 mL). The combined organic layers were washed with brine, dried over Na₂SO₄, filtered, and evaporated, to give the title compound (230 mg, 69.61% yield) as a pink solid. MS (ESI): m/z=386.0/388.0 [M−H]⁻.

### Example 13

### (2-Chloro-3-methoxy-phenyl)-[(7S)-3-(6-hydroxy-2-pyridyl)-2,7-dimethyl-5,7-dihydro-4H-pyrazolo[3,4-c]pyridin-6-yl]methanone

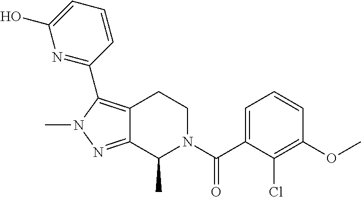 [SMILES: COc1cccc(C(=O)N2CCc3c(nn(C)c3-c3cccc(O)n3)[C@@H]2C)c1Cl]

A solution of 2-chloro-3-methoxybenzoic acid (CAS RN 33234-36-5; 152.76 mg, 0.820 mmol) in DMF (3 mL) was treated with 6-(2,7-dimethyl-4,5,6,7-tetrahydropyrazolo[3,4-c]pyridine-3-yl)pyridine-2-ol (200.0 mg, 0.820 mmol), TEA (0.57 mL, 4.09 mmol), and HATU (373.55 mg, 0.980 mmol), at 23° C. The mixture was stirred for 16 h at this temperature, before being purified by RP-HPLC and chiral chromatography, to give the title compound (105.8 mg, 38.09% yield) as a brown solid. ESI (MS): m/z=413.0/415.0 [M+H]⁺.

### Step a): tert-butyl 3-(6-methoxy-2-pyridyl)-2,7-dimethyl-5,7-dihydro-4H-pyrazolo[3,4-c]pyridine-6-carboxylate

A solution of tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (1.0 g, 2.25 mmol) in dry DMF (15 mL) was treated with 6-methoxypyridine-2-boronic acid pinacol ester (CAS RN: 1034297-69-2; 688.69 mg, 2.93 mmol) and sodium carbonate (477.67 mg, 4.51 mmol), at 23° C. under Ar. The mixture was sparged with Ar for 10 min, before being treated with tetrakis(triphenylphosphine)palladium(0) (260.4 mg, 0.230 mmol) and heated to 100° C. The mixture was stirred for 16 h at this temperature, before being cooled down and filtered off Purification by RP-HPLC gave the title compound (559 mg, 69.21% yield) as a light brown oil. ESI (MS): m/z=359.4 [M+H]⁺.

### Step b): 6-(2,7-dimethyl-4,5,6,7-tetrahydropyrazolo[3,4-c]pyridine-3-yl)pyridine-2-ol

A solution of tert-butyl 3-(6-methoxy-2-pyridyl)-2,7-dimethyl-5,7-dihydro-4H-pyrazolo[3,4-c]pyridine-6-carboxylate (559.0 mg, 1.56 mmol) in 12 N HCl (7.8 mL, 77.98 mmol) was heated to 100° C., and stirred for 16 h at this temperature. The mixture was then cooled down, evaporated, and purified by RP-HPLC, to give the title compound (159.5 mg, 41.86% yield) as a light brown solid. ESI (MS): m/z=245.2 [M+H]⁺.

### Example 14

### 2-[3-Chloro-5-[(7S)-6-(2-chloro-3-methoxy-benzoyl)-2,7-dimethyl-5,7-dihydro-4H-pyrazolo[3,4-c]pyridin-3-yl]phenyl]acetamide

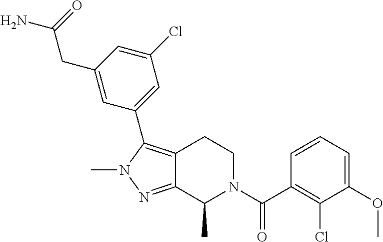 [SMILES: COc1cccc(C(=O)N2CCc3c(nn(C)c3-c3cc(Cl)cc(CC(N)=O)c3)[C@@H]2C)c1Cl]

A solution of methyl 2-[3-chloro-5-[(7S)-6-(2-chloro-3-methoxy-benzoyl)-2,7-dimethyl-5,7-dihydro-4H-pyrazolo[3,4-c]pyridine-3-yl]phenyl]acetate (80.0 mg, 0.160 mmol) in THE (10 mL) was treated with ammonia (1.0 mL, 14.68 mmol), before being heated to 50° C. The mixture was stirred for 30 min at this temperature, before being treated with CDI (51.64 mg, 0.320 mmol) in one portion. The mixture was stirred for another 18 h at this temperature, before being evaporated. Purification by RP-HPLC gave the title compound (57.2 mg, 72.68% yield) as a white solid. MS (ESI): m/z=488 [M+H]⁺.

### Step a): methyl 2-[3-chloro-5-(4,4,5,5-tetramethyl-1,3,2-dioxaborolan-2-yl)phenyl]acetate

A solution of methyl 2-(3-bromo-5-chloro-phenyl)acetate (CAS RN: 960305-70-8; 500.0 mg, 1.9 mmol) in 1,4-dioxane (20 mL) was treated with bis(pinacolato)diboron (505.91 mg, 1.99 mmol) and potassium acetate (0.36 mL, 5.69 mmol), at 23° C. under Ar. The mixture was sparged with Ar, before being treated with 1,1′-bis(diphenylphosphino)ferrocene-palladium(II)dichloride dichloromethane complex (154.83 mg, 0.190 mmol) and heated to 110° C. The mixture was stirred for 16 h at this temperature, cooled down, filtered, and evaporated. The residue was partitioned between EtOAc and water. The organic layer was washed with brine, dried over Na₂SO₄, filtered, and evaporated, to give the title compound (500 mg, 74.67% yield). MS (ESI): m/z=310.0 [M+H]⁺.

### Step b): [3-chloro-5-(2-methoxy-2-oxo-ethyl)phenyl]boronic acid

A solution of methyl 2-[3-chloro-5-(4,4,5,5-tetramethyl-1,3,2-dioxaborolan-2-yl)phenyl]acetate (2.5 g, 8.05 mmol) in 2.5:1 THF/water (56 mL) was treated with sodium metaperiodate (5.17 g, 24.15 mmol) and ammonium acetate buffer solution (1.24 g, 16.1 mmol), at 25° C. The mixture was stirred for 18 h at this temperature, before being filtered and evaporated. Purification by RP-HPLC gave the title compound (400 mg, 21.1% yield) as a light yellow solid. MS (ESI): m/z=227.8 [M−H]⁻.

### Step c): tert-butyl 3-[3-chloro-5-(2-methoxy-2-oxo-ethyl)phenyl]-2,7-dimethyl-5,7-dihydro-4H-pyrazolo[3,4-c]pyridine-6-carboxylate

A solution of [3-chloro-5-(2-methoxy-2-oxo-ethyl)phenyl]boronic acid (400.38 mg, 1.75 mmol) in dry DMF (10 mL) was treated with tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (700.0 mg, 1.75 mmol) and sodium carbonate (371.52 mg, 3.51 mmol), at 23° C. under Ar. The mixture was sparged with Ar, before being treated with tetrakis(triphenylphosphine)palladium(0) (202.53 mg, 0.180 mmol) and heated to 100° C. The mixture was stirred for 16 h at this temperature, cooled down, filtered, and evaporated. FC (SiO₂; hexane/EtOAc) gave the title compound (170 mg, 22.35% yield) as a light yellow solid. MS (ESI): m/z=434.0 [M+H]⁺.

### Step d): methyl 2-[3-chloro-5-(2,7-dimethyl-4,5,6,7-tetrahydropyrazolo[3,4-c]pyridine-3-yl)phenyl]acetate;hydrochloride

A solution of tert-butyl 3-[3-chloro-5-(2-methoxy-2-oxo-ethyl)phenyl]-2,7-dimethyl-5,7-dihydro-4H-pyrazolo[3,4-c]pyridine-6-carboxylate (170.0 mg, 0.390 mmol) in 4 M HCl in 1,4-dioxane (2 mL) was stirred for 18 h at 25° C. Et₂O was added, and the resulting precipitate was filtered and dried, to give the title compound (100 mg, 62.04% yield) as a white solid. MS (ESI): m/z=334.0/336.0 [M+H]⁺.

### Step e): methyl 2-[3-chloro-5-[6-(2-chloro-3-methoxy-benzoyl)-2,7-dimethyl-5,7-dihydro-4H-pyrazolo[3,4-c]pyridine-3-yl]phenyl]acetate

A solution of methyl 2-[3-chloro-5-(2,7-dimethyl-4,5,6,7-tetrahydropyrazolo[3,4-c]pyridine-3-yl)phenyl]acetate (1081.08 mg, 2.4 mmol) in DMF (8 mL) was treated with 2-chloro-3-methoxy-benzoic acid;hydrochloride (534.56 mg, 2.4 mmol), HATU (1093.5 mg, 2.88 mmol), and DIPEA (1.67 mL, 9.59 mmol), at 23° C. The mixture was stirred for 18 h at this temperature before being purified by RP-HPLC, to give the racemate (373 mg, 30.98% yield) as a light brown solid. Purification by chiral chromatography gave the title compound (153 mg, 12.35% yield) as a white solid. MS (ESI): m/z=503.0 [M+H]⁺.

### Example 15

### 2-[3-Chloro-5-[(7R)-6-(2-chloro-3-methoxy-benzoyl)-2,7-dimethyl-5,7-dihydro-4H-pyrazolo[3,4-c]pyridin-3-yl]phenyl]acetamide

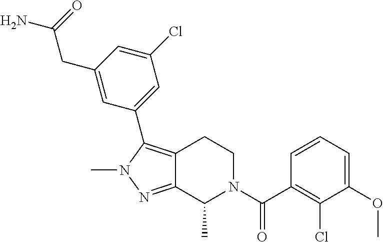 [SMILES: COc1cccc(C(=O)N2CCc3c(nn(C)c3-c3cc(Cl)cc(CC(N)=O)c3)[C@H]2C)c1Cl]

The title compound was prepared in analogy to Example 14: 149 mg, 12.2% yield, white solid. MS (ESI): m/z=503.0 [M+H]⁺.

### Example 16

### 1-[3-Chloro-5-[(7S)-6-(2-chloro-3-methoxy-benzoyl)-2,7-dimethyl-5,7-dihydro-4H-pyrazolo[3,4-c]pyridin-3-yl]phenyl]cyclopropanecarboxamide

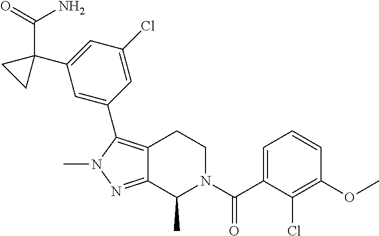 [SMILES: COc1cccc(C(=O)N2CCc3c(nn(C)c3-c3cc(Cl)cc(C4(C(N)=O)CC4)c3)[C@@H]2C)c1Cl]

A solution of 1-[3-chloro-5-[6-(2-chloro-3-methoxy-benzoyl)-2,7-dimethyl-5,7-dihydro-4H-pyrazolo[3,4-c]pyridine-3-yl]phenyl]cyclopropanecarboxylic acid (99.81 mg, 0.190 mmol) in dry THF (10 mL) was treated with CDI (47.28 mg, 0.290 mmol), at 25° C. The mixture was stirred for 15 h at this temperature, before being treated with ammonia hydrate (296.25 mg, 1.94 mmol) and stirred for 15 h. The mixture was evaporated, and the residue was dissolved in EtOAc. The organic layer was washed with 0.1 N HCl, saturated aqueous NaHCO₃, and brine, dried over Na₂SO₄, filtered, and evaporated. Purification by chiral chromatography gave the title compound (27.6 mg, 27.65% yield) as a light brown solid. MS (ESI): m/z=513.0 [M+H]f.

### Step a): methyl 1-(3-bromo-5-chloro-phenyl)cyclopropanecarboxylate

A solution of 1-(3-bromo-5-chloro-phenyl)cyclopropanecarboxylic acid (CAS RN: 1505800-30-5; 500.0 mg, 1.81 mmol) in MeOH (1 mL) was treated dropwise with thionyl chloride (259.07 mg, 2.18 mmol), at 0° C. The mixture was then heated to 50° C., and stirred for 16 h at this temperature before being evaporated. The residue was dissolved in DCM, washed with water, dried over Na₂SO₄, filtered, and evaporated, to give the title compound (520 mg, 94.02% yield) as a white solid. MS (ESI): m/z=289.0/290.8 [M+H]⁺.

### Step b): methyl 1-[3-chloro-5-(4,4,5,5-tetramethyl-1,3,2-dioxaborolan-2-yl)phenyl]cyclopropane-carboxylate

A solution of bis(pinacolato)diboron (456.05 mg, 1.8 mmol) in 1,4-dioxane (30 mL) was treated with methyl 1-(3-bromo-5-chloro-phenyl)cyclopropanecarboxylate (520.0 mg, 1.8 mmol), 1,1′-bis(diphenylphosphino)ferrocene-palladium(II)dichloride dichloromethane complex (1465.45 mg, 1.8 mmol), and potassium acetate (0.11 mL, 1.8 mmol), at 23° C. under Ar. The mixture was heated to 110° C. and stirred for 16 h at this temperature, before being filtered and evaporated. The residue was partitioned between EtOAc and water. The organic layer was washed with brine, dried over Na₂SO₄, filtered, and evaporated.

Purification by FC (SiO₂; hexane/MTBE) gave the title compound (400 mg, 62.86% yield) as a crude white solid, which was directly used in the next step.

### Step c): tert-butyl 3-[3-chloro-5-(1-methoxycarbonylcyclopropyl)phenyl]-2,7-dimethyl-5,7-dihydro-4H-pyrazolo[3,4-c]pyridine-6-carboxylate

A solution of tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (310.0 mg, 0.780 mmol) in dry DMF (30 mL) was treated with tetrakis(triphenylphosphine)palladium(0) (89.69 mg, 0.080 mmol), sodium carbonate (164.53 mg, 1.55 mmol) and methyl 1-[3-chloro-5-(4,4,5,5-tetramethyl-1,3,2-dioxaborolan-2-yl)phenyl]cyclopropanecarboxylate (313.53 mg, 0.930 mmol), at 23° C. under Ar. The mixture was heated to 100° C. and stirred for 16 h at this temperature, before being cooled down, filtered, and evaporated. Purification by FC (SiO₂; hexane/EtOAc) gave the title compound (300 mg, 84% yield) as a light brown solid. MS (ESI): m/z=460.2 [M+H]⁺.

### Step d): methyl 1-[3-chloro-5-(2,7-dimethyl-4,5,6,7-tetrahydropyrazolo[3,4-c]pyridine-3-yl)phenyl]cyclo-propanecarboxylate;hydrochloride

A solution of tert-butyl 3-[3-chloro-5-(1-methoxycarbonylcyclopropyl)phenyl]-2,7-dimethyl-5,7-dihydro-4H-pyrazolo[3,4-c]pyridine-6-carboxylate (300.0 mg, 0.650 mmol) in 4 M HCl in 1,4-dioxane (2.0 mL, 8 mmol), at 23° C. The mixture was stirred for 16 h, before being evaporated. Trituration with MTBE gave the title compound (200 mg, 73.51% yield) as a white powder. MS (ESI): m/z=360.0 [M+H]⁺.

### Step e): methyl 1-[3-chloro-5-[6-(2-chloro-3-methoxy-benzoyl)-2,7-dimethyl-5,7-dihydro-4H-pyrazolo[3,4-c]pyridine-3-yl]phenyl]cyclopropanecarboxylate

A solution of methyl 1-[3-chloro-5-(2,7-dimethyl-4,5,6,7-tetrahydropyrazolo[3,4-c]pyridine-3-yl)phenyl]cyclopropanecarboxylate;hydrochloride (200.0 mg, 0.500 mmol) in DMF (5 mL) was treated with 2-chloro-3-methoxybenzoic acid (CAS RN 33234-36-5; 94.16 mg, 0.500 mmol), HATU (287.8, g. 0.757 mmol) and TEA (0.25 mL, 1.77 mmol), at 23° C. The mixture was stirred for 16 h at this temperature, before being poured onto water. The aqueous layer was extracted with EtOAc, and the combined organic layers were washed with water, dried over Na₂SO₄, filtered, and evaporated. Purification by RP-HPLC gave the title compound (129 mg, 48.37% yield) as a light brown oil. MS (ESI): m/z=529.0 [M+H]⁺.

### Step f): 1-[3-chloro-5-[6-(2-chloro-3-methoxy-benzoyl)-2,7-dimethyl-5,7-dihydro-4H-pyrazolo[3,4-c]pyridine-3-yl]phenyl]cyclopropanecarboxylic acid

A solution of methyl 1-[3-chloro-5-[6-(2-chloro-3-methoxy-benzoyl)-2,7-dimethyl-5,7-dihydro-4H-pyrazolo[3,4-c]pyridine-3-yl]phenyl]cyclopropanecarboxylate (129.0 mg, 0.240 mmol) in 2.5:2.5:1 THF/MeOH/water (6 mL) was treated with LiOH monohydrate (25.61 mg, 0.610 mmol), at 25° C. The mixture was heated to 50° C., and stirred for 16 h at this temperature before being evaporated. The residue was diluted in water, and the pH was adjusted to 3. The mixture was extracted with MTBE, and the combined organic layers were dried over Na₂SO₄, filtered, and evaporated, to give the title compound (100 mg, 79.63% yield) as a white solid. MS (ESI): m/z=514.2 [M+H]⁺.

### Example 17

### 2-[3-[(7S)-6-(2-Chloro-3-methoxy-benzoyl)-2,7-dimethyl-5,7-dihydro-4H-pyrazolo[3,4-c]pyridin-3-yl]-5-fluoro-phenyl]acetamide

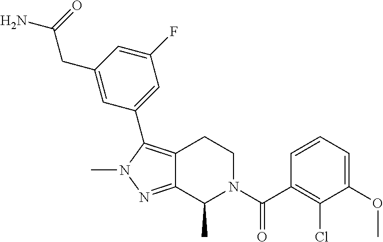 [SMILES: COc1cccc(C(=O)N2CCc3c(nn(C)c3-c3cc(F)cc(CC(N)=O)c3)[C@@H]2C)c1Cl]

A solution of 2-[3-(2,7-dimethyl-4,5,6,7-tetrahydropyrazolo[3,4-c]pyridine-6-ium-3-yl)-5-fluoro-phenyl]acetamide;chloride (86.0 mg, 0.250 mmol) in DMF (0.500 mL) was treated with 2-chloro-3-methoxybenzoic acid (CAS RN 33234-36-5; 47.36 mg, 0.250 mmol), HATU (115.82 mg, 0.300 mmol), and TEA (0.18 mL, 1.27 mmol), at 20° C. The mixture was stirred for 10 h, before being filtered and evaporated. Purification by RP-HPLC gave the title compound (70 mg, 58.56% yield) as a light brown solid. Chiral chromatography gave the desired enantiomer (23.1 mg, 33.0% yield) as a yellow solid. MS (ESI): m/z=471.0/473.0 [M+H]⁺.

### Step a): 2-(3-bromo-5-fluoro-phenyl)acetamide

A solution of 2-(3-bromo-5-fluoro-phenyl)acetic acid (CAS RN: 202001-01-2; 1.2 g, 5.15 mmol) in DCE (5 mL) was treated with one drop of DMF and thionyl chloride (0.56 mL, 7.72 mmol), at 23° C. The mixture was heated to 80° C. and stirred for 2 h at this temperature, before being evaporated. The residue was evaporated with toluene, and dissolved in THE (5 mL). The mixture was added dropwise to an aqueous solution of ammonia (25%; 30.0 mL, 5.15 mmol), at −5° C. The mixture was stirred for 2 h at 20° C.

The precipitate was filtered and dried, to give the title compound (900 mg, 73.66% yield) as a light yellow powder. MS (ESI): m/z=232.0/234.0 [M+H]⁺.

### Step b): 2-[3-fluoro-5-(4,4,5,5-tetramethyl-1,3,2-dioxaborolan-2-yl)phenyl]acetamide

A solution of 2-(3-bromo-5-fluoro-phenyl)acetamide (200.0 mg, 0.860 mmol) in 1,4-dioxane (5 mL) was treated with bis(pinacolato)diboron (262.64 mg, 1.03 mmol) and potassium acetate (0.13 mL, 2.15 mmol), at 23° C. under Ar. The mixture was sparged with Ar, before being treated with 1,1′-bis(diphenylphosphino)ferrocene-palladium(II)dichloride dichloromethane complex (70.38 mg, 0.090 mmol) and heated to 100° C. The mixture was stirred for 5 h at this temperature, cooled down, and filtered over celite. The filtrate was evaporated, to give the title compound (250 mg, 88.23% yield) as a dark brown solid. MS (ESI): m/z=279.0 [M+H]⁺.

### Step c): tert-butyl 3-[3-(2-amino-2-oxo-ethyl)-5-fluoro-phenyl]-2,7-dimethyl-5,7-dihydro-4H-pyrazolo[3,4-c]pyridine-6-carboxylate

A solution of tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (180.0 mg, 0.340 mmol) in dry DMF (2 mL) was treated with 2-[3-fluoro-5-(4,4,5,5-tetramethyl-1,3,2-dioxaborolan-2-yl)phenyl]acetamide (146.01 mg, 0.440 mmol) and sodium carbonate (38.26 mg, 0.360 mmol), at 23° C. under Ar. The mixture was sparged with Ar for 10 min, treated with tetrakis(triphenylphosphine)palladium(0) (10.43 mg, 0.010 mmol), and heated to 100° C. The mixture was stirred for 16 h at this temperature, before being cooled down, filtered, and evaporated. Purification by RP-HPLC gave the title compound (81.1 mg, 57.29% yield) as a light brown foam. MS (ESI): m/z=347.2 [M-Bu+H]⁺.

### Step d): 2-[3-(2,7-dimethyl-4,5,6,7-tetrahydropyrazolo[3,4-c]pyridine-6-ium-3-yl)-5-fluoro-phenyl]acetamide;chloride

A solution of tert-butyl 3-[3-(2-amino-2-oxo-ethyl)-5-fluoro-phenyl]-2,7-dimethyl-5,7-dihydro-4H-pyrazolo[3,4-c]pyridine-6-carboxylate (115.5 mg, 0.290 mmol) in DCM (1 mL) was treated with 4 M HCl in 1,4-dioxane (0.72 mL, 2.87 mmol), at 20° C. The mixture was stirred for 10 h at this temperature before being evaporated, to give the title compound (96 mg, 96.16% yield) as a light brown solid. MS (ESI): m/z=303.0 [M+H]⁺.

### Example 18

### 2-[5-[(7S)-6-(2-Chloro-3-methoxy-benzoyl)-2,7-dimethyl-5,7-dihydro-4H-pyrazolo[3,4-c]pyridin-3-yl]-3-pyridyl]acetamide

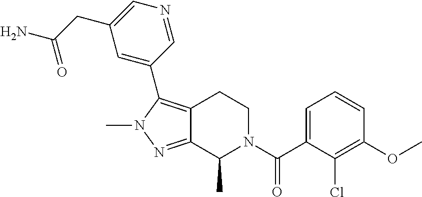 [SMILES: COc1cccc(C(=O)N2CCc3c(nn(C)c3-c3cncc(CC(N)=O)c3)[C@@H]2C)c1Cl]

A solution of 2-chloro-3-methoxybenzoic acid (CAS RN 33234-36-5; 102.6 mg, 0.550 mmol) in DCM (1 mL) was treated with one drop of DMF and oxalyl chloride (0.09 mL, 1.1 mmol), at 20° C. The mixture was stirred for 1 h at this temperature, before being concentrated. The residue was then dissolved in DCM (2 mL), and added dropwise to a solution of 2-[5-(2,7-dimethyl-4,5,6,7-tetrahydropyrazolo[3,4-c]pyridine-6-ium-3-yl)pyridine-1-ium-3-yl]acetamide;dichloride (197.0 mg, 0.550 mmol) and DIPEA (0.38 mL, 2.2 mmol) in DCM (5 mL), at 0° C. The mixture was warmed to 20° C. and stirred for 10 h at this temperature, before being evaporated. Purification by RP-HPLC followed by chiral chromatography gave the title compound (74.3 mg, 40.2% yield) as a yellow viscous oil. MS (ESI): m/z=454.0/456.2 [M+H]⁺.

### Step a): methyl 2-[5-(4,4,5,5-tetramethyl-1,3,2-dioxaborolan-2-yl)-3-pyridyl]acetate

A solution of methyl 2-(5-bromo-3-pyridyl)acetate (CAS RN: 118650-08-1; 750.0 mg, 3.26 mmol) in 1,4-dioxane (15 mL) was treated with bis(pinacolato)diboron (952.03 mg, 3.75 mmol) and potassium acetate (0.61 mL, 9.78 mmol), at 20° C. under Ar. The mixture was sparged with Ar, and treated with 1,1′-bis(diphenylphosphino)ferrocene-palladium(II)dichloride dichloromethane complex (266.23 mg, 0.330 mmol). The mixture was heated to 100° C. and stirred for 15 h at this temperature, before being evaporated. The residue was dissolved in EtOAc, filtered through celite, and evaporated. Trituration with hexane gave the title compound (770 mg, 80.54% yield) as a brown solid. MS (ESI): m/z=277.0 [M]⁺.

### Step b): tert-butyl 3-[5-(2-methoxy-2-oxo-ethyl)-3-pyridyl]-2,7-dimethyl-5,7-dihydro-4H-pyrazolo[3,4-c]pyridine-6-carboxylate

A solution of methyl 2-[5-(4,4,5,5-tetramethyl-1,3,2-dioxaborolan-2-yl)-3-pyridyl]acetate (767.87 mg, 2.49 mmol) in DMF (7 mL) was treated with tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (830.0 mg, 2.08 mmol) and sodium carbonate (440.53 mg, 4.16 mmol), at 20° C. under Ar. The mixture was sparged with Ar, before being treated with tetrakis(triphenylphosphine)palladium(0) (120.07 mg, 0.100 mmol). The resulting mixture was heated to 100° C. and stirred for 15 h at this temperature, before being cooled down and diluted with water (10 mL). The mixture was extracted with EtOAc (3×10 mL). The combined organic layers were washed with brine, dried over Na₂SO₄, and evaporated. Purification by RP-HPLC gave the title compound (225 mg, 27.04% yield) as a brown foam. MS (ESI): m/z=401.2 [M+H]⁺.

### Step c): tert-butyl 3-[5-(2-amino-2-oxo-ethyl)-3-pyridyl]-2,7-dimethyl-5,7-dihydro-4H-pyrazolo[3,4-c]pyridine-6-carboxylate

A solution of tert-butyl 3-[5-(2-methoxy-2-oxo-ethyl)-3-pyridyl]-2,7-dimethyl-5,7-dihydro-4H-pyrazolo[3,4-c]pyridine-6-carboxylate (225.0 mg, 0.560 mmol) in ammonia (25% in MeOH) (4.78 mL, 56.18 mmol) was stirred for 10 h at 20° C. before being evaporated, to give the title compound (214 mg, 98.81% yield) as a light brown foam. MS (ESI): m/z=386.2 [M+H]⁺.

### Step d): 2-[5-(2,7-dimethyl-4,5,6,7-tetrahydropyrazolo[3,4-c]pyridine-6-ium-3-yl)pyridine-1-ium-3-yl]acetamide;dichloride

A solution of tert-butyl 3-[5-(2-amino-2-oxo-ethyl)-3-pyridyl]-2,7-dimethyl-5,7-dihydro-4H-pyrazolo[3,4-c]pyridine-6-carboxylate (214.0 mg, 0.560 mmol) in DCM (2 mL) was treated with 4 M HCl in 1,4-dioxane (1.39 mL, 5.55 mmol), at 20° C. The mixture was stirred for another 15 h at this temperature before being evaporated, to give the title compound (197 mg, 99.04% yield) as a light yellow solid. MS (ESI): m/z=286.2 [M+H]⁺.

### Example 19

### [2-Chloro-3-(2,2-dioxo-2λ⁶-thia-6-azaspiro[3.3]heptan-6-yl)-5-fluoro-phenyl]-[3-(3,5-difluorophenyl)-2,7-dimethyl-5,7-dihydro-4H-pyrazolo[3,4-c]pyridine-6-yl]methanone

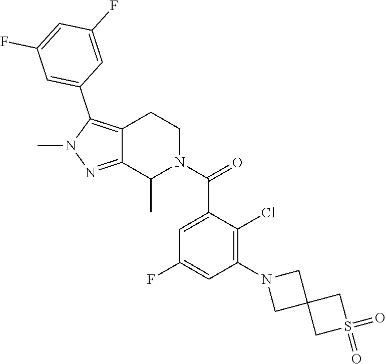 [SMILES: CC1c2nn(C)c(-c3cc(F)cc(F)c3)c2CCN1C(=O)c1cc(F)cc(N2CC3(C2)CS(=O)(=O)C3)c1Cl]

A solution of 2-chloro-3-(2,2-diketo-2λ6-thia-6-azaspiro[3.3]heptan-6-yl)-5-fluoro-benzoic acid (C.1) (54.67 mg, 0.171 mmol) in dry DMF (1.5 mL) was treated with HATU (78.02 mg, 0.205 mmol) and DIPEA (291.93 μL, 1.71 mmol), at 23° C. under Ar. The mixture was stirred for 10 min at this temperature, before being treated with 1:5 3-(3,5-difluorophenyl)-2,7-dimethyl-4,5,6,7-tetrahydropyrazolo[3,4-c]pyridine/2,2,2-trifluoroacetic acid (Patent nr. WO2020/065613 A1, 2020) (B.5; 150 mg, 0.171 mmol), and stirred for another 20 h. Purification by FC (SiO₂; DCM/MeOH) gave the title compound (28 mg, 43.47% yield) as a white solid. MS (ESI): m/z=565.2 [M+H]⁺.

### Examples 20 and 21

### [2-Chloro-3-(2,2-dioxo-2λ⁶-thia-6-azaspiro[3.3]heptan-6-yl)-5-fluoro-phenyl]-[(7S)-3-(3,5-difluorophenyl)-2,7-dimethyl-5,7-dihydro-4H-pyrazolo[3,4-c]pyridine-6-yl]methanone (Example 20) and [2-Chloro-3-(2,2-dioxo-2λ⁶-thia-6-azaspiro[3.3]heptan-6-yl)-5-fluoro-phenyl]-[(7R)-3-(3,5-difluorophenyl)-2,7-dimethyl-5,7-dihydro-4H-pyrazolo[3,4-c]pyridine-6-yl]methanone (Example 21)

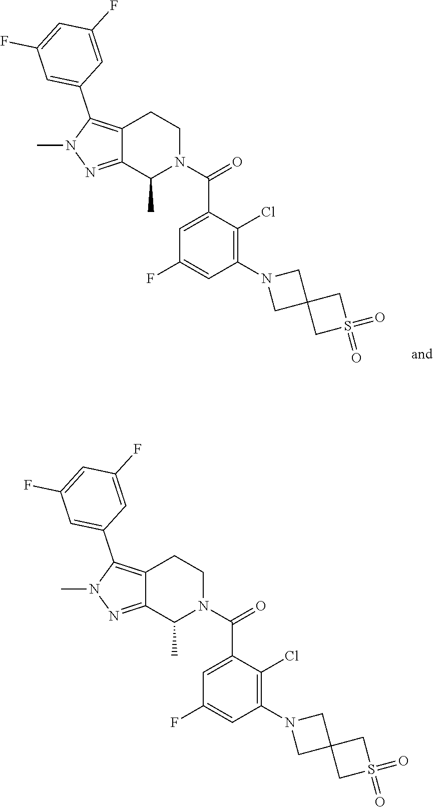 [SMILES: C[C@H]1c2nn(C)c(-c3cc(F)cc(F)c3)c2CCN1C(=O)c1cc(F)cc(N2CC3(C2)CS(=O)(=O)C3)c1Cl ; C[C@@H]1c2nn(C)c(-c3cc(F)cc(F)c3)c2CCN1C(=O)c1cc(F)cc(N2CC3(C2)CS(=O)(=O)C3)c1Cl]

Example 19 was purified by chiral chromatography, to give the title compound (Example 20) (26 mg, 26.91%) as a white solid and the title compound (Example 21) (27 mg, 27.95%) as a white solid. MS (ESI): m/z=565.2 [M+H]⁺.

In analogy to Examples 19-21, Examples in the following table were generated using amine B.5 and acids C.X or commercially available acids.

| | | | | MS, |
|---|---|---|---|---|
| | | | Building | ESI: |
| Ex. | Structure | Systematic Name | Blocks | m/z |
| 22 | 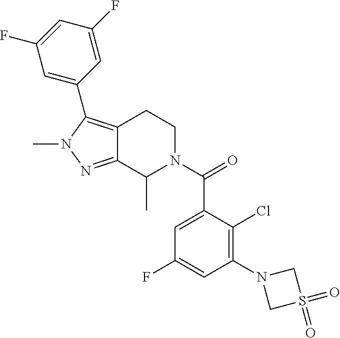 | [2-Chloro-5-fluoro-3-(3- methylsulfonylazetidin-1- yl)phenyl]-[3-(3,5- difluorophenyl)-2,7- dimethyl-5,7-dihydro-4H- pyrazolo[3,4-c]pyridin-6- yl]methanone | C.2 | 553.2 [M + H]⁺ |
| 23 | 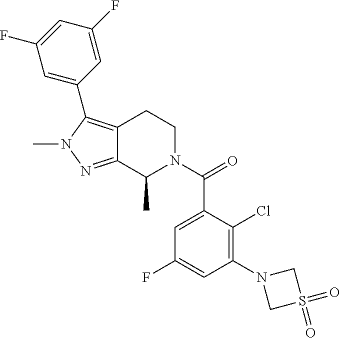 | [2-Chloro-5-fluoro-3-(3- methylsulfonylazetidin-1- yl)phenyl]-[(7S)-3-(3,5- difluorophenyl)-2,7- dimethyl-5,7-dihydro-4H- pyrazolo[3,4-c]pyridin-6- yl]methanone | C.2 | 553.2 [M + H]⁺ |
| 24 | 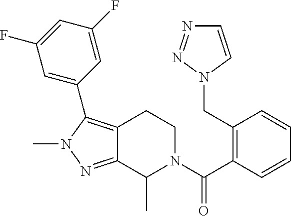 [SMILES: CC1c2nn(C)c(-c3cc(F)cc(F)c3)c2CCN1C(=O)c1ccccc1Cn1ccnn1] | [3-(3,5-Difluorophenyl)-2,7- dimethyl-5,7-dihydro-4H- pyrazolo[3,4-c]pyridin-6-yl]- [2-(triazol-1- ylmethyl)phenyl]methanone | 2-(triazol- 1- ylmethyl) benzoic acid (CAS RN 1369510- 36-0) | 449.3 [M + H]⁺ |
| 25 | 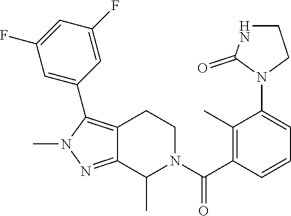 [SMILES: Cc1c(C(=O)N2CCc3c(nn(C)c3-c3cc(F)cc(F)c3)C2C)cccc1N1CCNC1=O] | 1-[3-[3-(3,5- Difluorophenyl)-2,7- dimethyl-5,7-dihydro-4H- pyrazolo[3,4-c]pyridine-6- carbonyl]-2-methyl- phenyl]imidazolidin-2-one | 2-methyl- 3-(2- oxoimidazolidin- 1- yl)benzoic acid (CAS RN 1343134- 58-6) | 466.3 [M + H]⁺ |
| 26 | 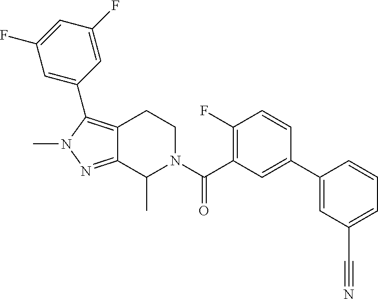 [SMILES: CC1c2nn(C)c(-c3cc(F)cc(F)c3)c2CCN1C(=O)c1cc(-c2cccc(C#N)c2)ccc1F] | 3-[3-[3-(3,5- Difluorophenyl)-2,7- dimethyl-5,7-dihydro-4H- pyrazolo[3,4-c]pyridine-6- carbonyl]-4-fluoro- phenyl]benzonitrile | 5-(3- cyanophenyl)- 2- fluoro- benzoic acid (CAS RN 1183926- 58-0) | 487.3 [M + H]⁺ |
| 27 | 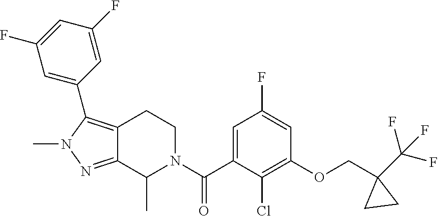 [SMILES: CC1c2nn(C)c(-c3cc(F)cc(F)c3)c2CCN1C(=O)c1cc(F)cc(OCC2(C(F)(F)F)CC2)c1Cl] | [2-Chloro-5-fluoro-3-[[1- (trifluoromethyl)cyclopropyl] methoxy]phenyl]-[3-(3,5- difluorophenyl)-2,7- dimethyl-5,7-dihydro-4H- pyrazolo[3,4-c]pyridin-6- yl]methanone | C.3 | 558.2 [M + H]⁺ |
| 28 | 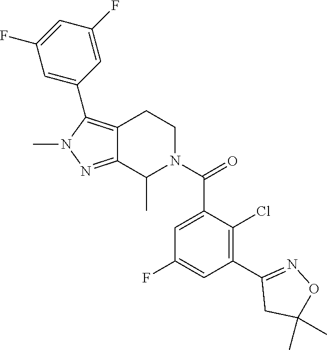 [SMILES: CC1c2nn(C)c(-c3cc(F)cc(F)c3)c2CCN1C(=O)c1cc(F)cc(C2=NOC(C)(C)C2)c1Cl] | [2-Chloro-3-(5,5-dimethyl- 4H-isoxazol-3-yl)-5-fluoro- phenyl]-[3-(3,5- difluorophenyl)-2,7- dimethyl-5,7-dihydro-4H- pyrazolo[3,4-c]pyridin-6- yl]methanone | C.4 | 517.3 [M + H]⁺ |
| 29 | 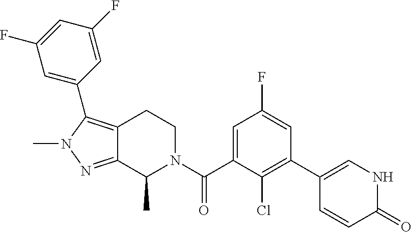 [SMILES: C[C@H]1c2nn(C)c(-c3cc(F)cc(F)c3)c2CCN1C(=O)c1cc(F)cc(-c2ccc(=O)[nH]c2)c1Cl] | 5-[2-Chloro-3-[(7S)-3-(3,5- difluorophenyl)-2,7- dimethyl-5,7-dihydro-4H- pyrazolo[3,4-c]pyridine-6- carbonyl]-5-fluoro-phenyl]- 1H-pyridin-2-one | C.5 | 513.0 [M + H]⁺ |
| 30 | 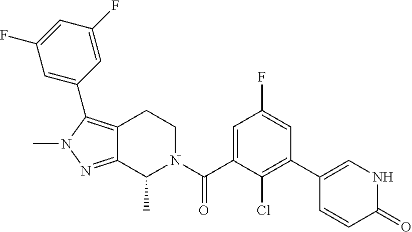 [SMILES: C[C@@H]1c2nn(C)c(-c3cc(F)cc(F)c3)c2CCN1C(=O)c1cc(F)cc(-c2ccc(=O)[nH]c2)c1Cl] | 5-[2-Chloro-3-[(7R)-3-(3,5- difluorophenyl)-2,7- dimethyl-5,7-dihydro-4H- pyrazolo[3,4-c]pyridine-6- carbonyl]-5-fluoro-phenyl]- 1H-pyridin-2-one | C.5 | 513.0 [M + H]⁺ |
| 31 | 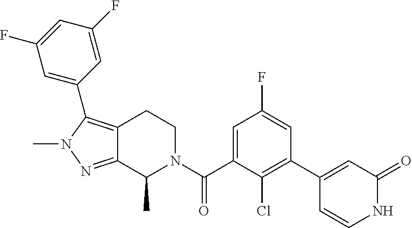 [SMILES: C[C@H]1c2nn(C)c(-c3cc(F)cc(F)c3)c2CCN1C(=O)c1cc(F)cc(-c2cc[nH]c(=O)c2)c1Cl] | 4-[2-Chloro-3-[(7S)-3-(3,5- difluorophenyl)-2,7- dimethyl-5,7-dihydro-4H- pyrazolo[3,4-c]pyridine-6- carbonyl]-5-fluoro-phenyl]- 1H-pyridin-2-one | C.6 | 513.0 [M + H]⁺ |
| 32 | 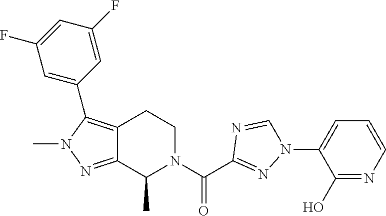 [SMILES: C[C@H]1c2nn(C)c(-c3cc(F)cc(F)c3)c2CCN1C(=O)c1ncn(-c2cccnc2O)n1] | [(7S)-3-(3,5- Difluorophenyl)-2,7- dimethyl-5,7-dihydro-4H- pyrazolo[3,4-c]pyridin-6-yl]- [1-(2-hydroxy-3-pyridyl)- 1,2,4-triazol-3-yl]methanone | C.7 | 452.0 [M + H]⁺ |
| 33 | 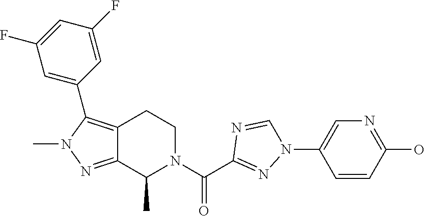 [SMILES: C[C@H]1c2nn(C)c(-c3cc(F)cc(F)c3)c2CCN1C(=O)c1ncn(-c2ccc(O)nc2)n1] | [(7S)-3-(3,5- Difluorophenyl)-2,7- dimethyl-5,7-dihydro-4H- pyrazolo[3,4-c]pyridin-6-yl]- [1-(6-hydroxy-3-pyridyl)- 1,2,4-triazol-3-yl]methanone | C.8 | 452.2 [M + H]⁺ |
| 34 | 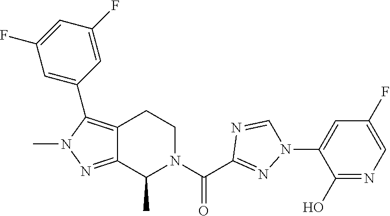 [SMILES: C[C@H]1c2nn(C)c(-c3cc(F)cc(F)c3)c2CCN1C(=O)c1ncn(-c2cc(F)cnc2O)n1] | [(7S)-3-(3,5- Difluorophenyl)-2,7- dimethyl-5,7-dihydro-4H- pyrazolo[3,4-c]pyridin-6-yl]- [1-(5-fluoro-2-hydroxy-3- pyridyl)-1,2,4-triazol-3- yl]methanone | C.9 | 470.0 [M + H]⁺ |
| 35 | 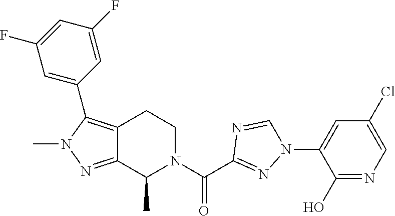 [SMILES: C[C@H]1c2nn(C)c(-c3cc(F)cc(F)c3)c2CCN1C(=O)c1ncn(-c2cc(Cl)cnc2O)n1] | [1-(5-Chloro-2-hydroxy-3- pyridyl)-1,2,4-triazol-3-yl]- [(7S)-3-(3,5-difluorophenyl)- 2,7-dimethyl-5,7-dihydro- 4H-pyrazolo[3,4-c]pyridin- 6-yl]methanone | C.10 | 486.0 [M + H]⁺ |
| 36 | 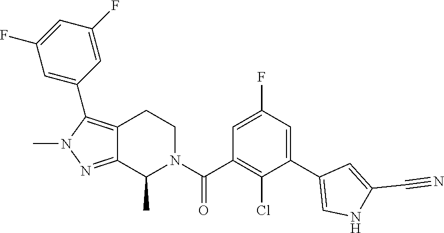 [SMILES: C[C@H]1c2nn(C)c(-c3cc(F)cc(F)c3)c2CCN1C(=O)c1cc(F)cc(-c2c[nH]c(C#N)c2)c1Cl] | 4-[2-Chloro-3-[(7S)-3-(3,5- difluorophenyl)-2,7- dimethyl-5,7-dihydro-4H- pyrazolo[3,4-c]pyridine-6- carbonyl]-5-fluoro-phenyl]- 1H-pyrrole-2-carbonitrile | C.11 | 510 [M + H]⁺ |
| 37 | 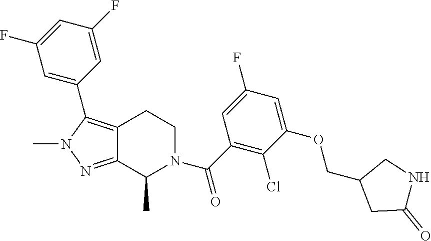 [SMILES: C[C@H]1c2nn(C)c(-c3cc(F)cc(F)c3)c2CCN1C(=O)c1cc(F)cc(OCC2CNC(=O)C2)c1Cl] | 4-[[2-Chloro-3-[3-(3,5- difluorophenyl)-2,7- dimethyl-5,7-dihydro-4H- pyrazolo[3,4-c]pyridine-6- carbonyl]-5-fluoro- phenoxy]methyl]pyrrolidin- 2-one | C.12 | 533.2 [M + H]⁺ |
| 38 | 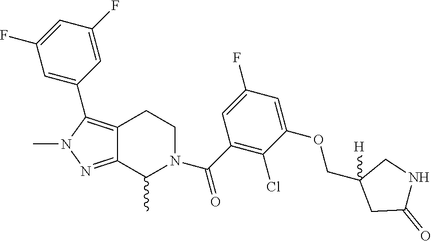 [SMILES: CC1c2nn(C)c(-c3cc(F)cc(F)c3)c2CCN1C(=O)c1cc(F)cc(OCC2CNC(=O)C2)c1Cl] | 4-[[2-Chloro-3-[3-(3,5- difluorophenyl)-2,7- dimethyl-5,7-dihydro-4H- pyrazolo[3,4-c]pyridine-6- carbonyl]-5-fluoro- phenoxy]methyl]pyrrolidin- 2-one (diastereomer A) | C.12 | 533.0 [M + H]⁺ |
| 39 | 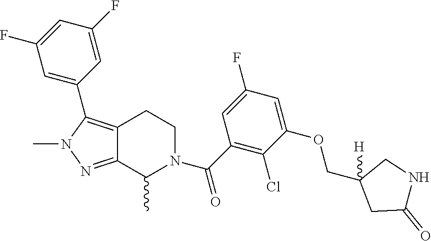 [SMILES: CC1c2nn(C)c(-c3cc(F)cc(F)c3)c2CCN1C(=O)c1cc(F)cc(OCC2CNC(=O)C2)c1Cl] | 4-[[2-Chloro-3-[3-(3,5- difluorophenyl)-2,7- dimethyl-5,7-dihydro-4H- pyrazolo[3,4-c]pyridine-6- carbonyl]-5-fluoro- phenoxy]methyl]pyrrolidin- 2-one (diastereomer B) | C.12 | 533.0 [M + H]⁺ |
| 40 | 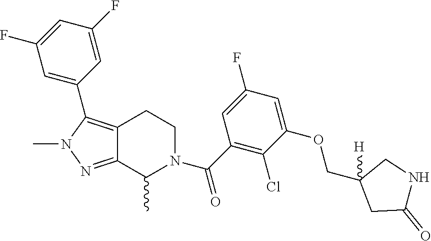 [SMILES: CC1c2nn(C)c(-c3cc(F)cc(F)c3)c2CCN1C(=O)c1cc(F)cc(OCC2CNC(=O)C2)c1Cl] | 4-[[2-Chloro-3-[3-(3,5- difluorophenyl)-2,7- dimethyl-5,7-dihydro-4H- pyrazolo[3,4-c]pyridine-6- carbonyl]-5-fluoro- phenoxy]methyl]pyrrolidin- 2-one (mixture of two diastereomers C, D) | C.12 | 533.0 [M + H]⁺ |
| 41 | 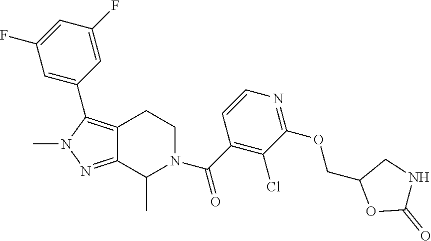 [SMILES: CC1c2nn(C)c(-c3cc(F)cc(F)c3)c2CCN1C(=O)c1ccnc(OCC2CNC(=O)O2)c1Cl] | 5-[[3-Chloro-4-[3-(3,5- difluorophenyl)-2,7- dimethyl-5,7-dihydro-4H- pyrazolo[3,4-c]pyridine-6- carbonyl]-2- pyridyl]oxymethyl] oxazolidin-2-one | C.13 | 518.3 [M + H]⁺ |
| 42 | 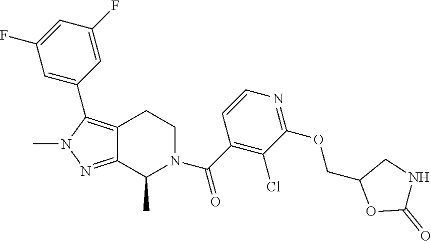 [SMILES: C[C@H]1c2nn(C)c(-c3cc(F)cc(F)c3)c2CCN1C(=O)c1ccnc(OCC2CNC(=O)O2)c1Cl] | 5-[[3-Chloro-4-[(7S)-3-(3,5- difluorophenyl)-2,7- dimethyl-5,7-dihydro-4H- pyrazolo[3,4-c]pyridine-6- carbonyl]-2- pyridyl]oxymethyl] oxazolidin-2-one | C.13 | 518.3 [M + H]⁺ |
| 43 | 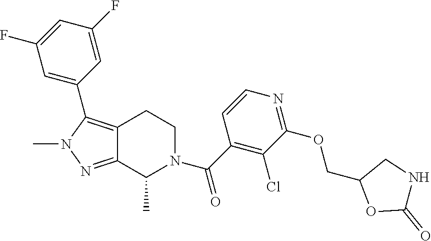 [SMILES: C[C@@H]1c2nn(C)c(-c3cc(F)cc(F)c3)c2CCN1C(=O)c1ccnc(OCC2CNC(=O)O2)c1Cl] | 5-[[3-Chloro-4-[(7R)-3-(3,5- difluorophenyl)-2,7- dimethyl-5,7-dihydro-4H- pyrazolo[3,4-c]pyridine-6- carbonyl]-2- pyridyl]oxymethyl] oxazolidin-2-one | C.13 | 518.3 [M + H]⁺ |
| 44 | 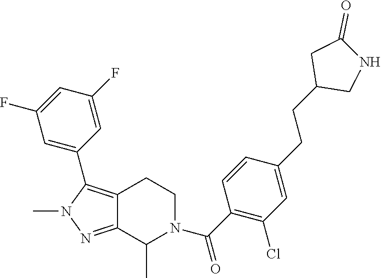 [SMILES: CC1c2nn(C)c(-c3cc(F)cc(F)c3)c2CCN1C(=O)c1ccc(CCC2CNC(=O)C2)cc1Cl] | 4-[2-[3-Chloro-4-[3-(3,5- difluorophenyl)-2,7- dimethyl-5,7-dihydro-4H- pyrazolo[3,4-c]pyridine-6- carbonyl]phenyl]ethyl] pyrrolidin-2-one | C.14 | 513.3 [M + H]⁺ |
| 45 | 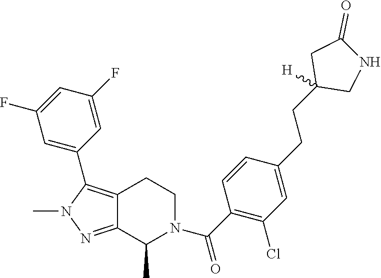 [SMILES: C[C@H]1c2nn(C)c(-c3cc(F)cc(F)c3)c2CCN1C(=O)c1ccc(CCC2CNC(=O)C2)cc1Cl] | 4-[2-[3-Chloro-4-[(7S)-3- (3,5-difluorophenyl)-2,7- dimethyl-5,7-dihydro-4H- pyrazolo[3,4-c]pyridine-6- carbonyl]phenyl]ethyl] pyrrolidin-2-one | C.14 | 513.3 [M + H]⁺ |
| 46 | 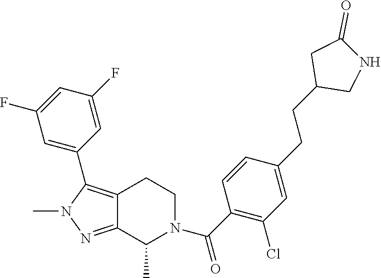 [SMILES: C[C@@H]1c2nn(C)c(-c3cc(F)cc(F)c3)c2CCN1C(=O)c1ccc(CCC2CNC(=O)C2)cc1Cl] | 4-[2-[3-Chloro-4-[3-(3,5- difluorophenyl)-2,7- dimethyl-5,7-dihydro-4H- pyrazolo[3,4-c]pyridine-6- carbonyl]phenyl]ethyl] pyrrolidin-2-one (mixture of two diastereomers A, B) | C.14 | 513.3 [M + H]⁺ |
| 47 | 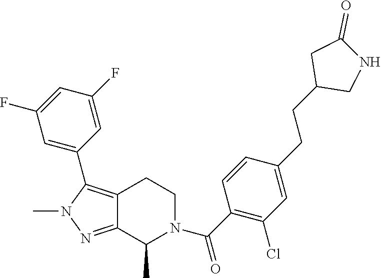 [SMILES: C[C@H]1c2nn(C)c(-c3cc(F)cc(F)c3)c2CCN1C(=O)c1ccc(CCC2CNC(=O)C2)cc1Cl] | 4-[2-[3-Chloro-4-[3-(3,5- difluorophenyl)-2,7- dimethyl-5,7-dihydro-4H- pyrazolo[3,4-c]pyridine-6- carbonyl]phenyl]ethyl] pyrrolidin-2-one (mixture of two diastereomers C + D) | C.14 | 513.3 [M + H]⁺ |
| 48 | 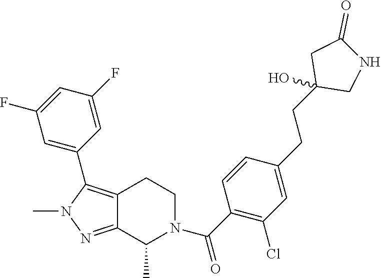 | 4-[2-[3-Chloro-4-[(7R)-3- (3,5-difluorophenyl)-2,7- dimethyl-5,7-dihydro-4H- pyrazolo[3,4-c]pyridine-6- carbonyl]phenyl]ethyl] pyrrolidin-2-one (one diastereomer, A or B) | C.14 | 513.3 [M + H]⁺ |
| 49 | 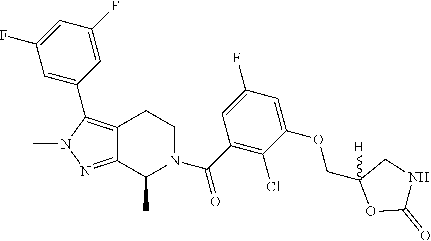 [SMILES: C[C@H]1c2nn(C)c(-c3cc(F)cc(F)c3)c2CCN1C(=O)c1cc(F)cc(OCC2CNC(=O)O2)c1Cl] | 5-[[2-Chloro-3-[3-(3,5- difluorophenyl)-2,7- dimethyl-5,7-dihydro-4H- pyrazolo[3,4-c]pyridine-6- carbonyl]-5-fluoro- phenoxy]methyl]oxazolidin- 2-one (diastereomer A) | C.15 | 535.2 [M + H]⁺ |
| 50 | 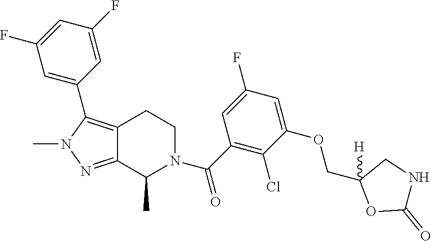 [SMILES: C[C@H]1c2nn(C)c(-c3cc(F)cc(F)c3)c2CCN1C(=O)c1cc(F)cc(OCC2CNC(=O)O2)c1Cl] | 5-[[2-Chloro-3-[3-(3,5- difluorophenyl)-2,7- dimethyl-5,7-dihydro-4H- pyrazolo[3,4-c]pyridine-6- carbonyl]-5-fluoro- phenoxy]methyl]oxazolidin- 2-one (diastereomer B) | C.15 | 535.2 [M + H]⁺ |
| 51 | 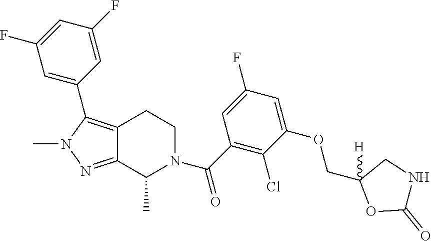 [SMILES: C[C@@H]1c2nn(C)c(-c3cc(F)cc(F)c3)c2CCN1C(=O)c1cc(F)cc(OCC2CNC(=O)O2)c1Cl] | 5-[[2-Chloro-3-[3-(3,5- difluorophenyl)-2,7- dimethyl-5,7-dihydro-4H- pyrazolo[3,4-c]pyridine-6- carbonyl]-5-fluoro- phenoxy]methyl]oxazolidin- 2-one (diastereomer C) | C.15 | 535.2 [M + H]⁺ |
| 52 | 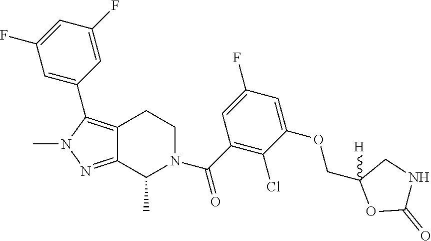 [SMILES: C[C@@H]1c2nn(C)c(-c3cc(F)cc(F)c3)c2CCN1C(=O)c1cc(F)cc(OCC2CNC(=O)O2)c1Cl] | 5-[[2-Chloro-3-[3-(3,5- difluorophenyl)-2,7- dimethyl-5,7-dihydro-4H- pyrazolo[3,4-c]pyridine-6- carbonyl]-5-fluoro- phenoxy]methyl]oxazolidin- 2-one (diastereomer D) | C.15 | 535.2 [M + H]⁺ |

### Example 53

### 4-[3-[(7S)-3-(3,5-Difluorophenyl)-2,7-dimethyl-5,7-dihydro-4H-pyrazolo[3,4-c]pyridine-6-carbonyl]-5-fluoro phenyl]-1H-pyrrole-2-carbonitrile

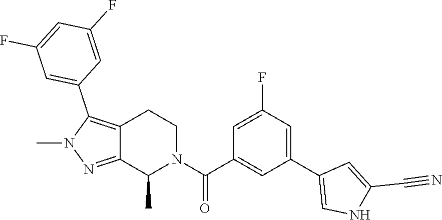

Example 53 was generated as a side product during the synthesis of Example 36, and isolated during the purification (34.7 mg, 10.0% yield).

### Example 54

### [3-(3,5-Difluorophenyl)-2,7-dimethyl-5,7-dihydro-4H-pyrazolo[3,4-c]pyridin-6-yl]-(2-pyrrolidin-3-yl-1,2,4-triazol-3-yl)methanone; hydrochloride (One Diastereomer, Unknown Stereochemistry)

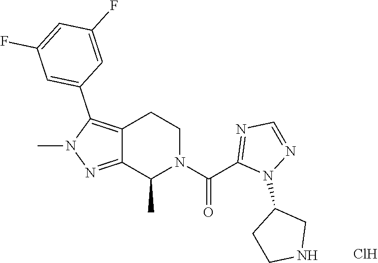 [SMILES: C[C@H]1c2nn(C)c(-c3cc(F)cc(F)c3)c2CCN1C(=O)c1ncnn1[C@H]1CCNC1 ; Cl]

A solution of tert-butyl (3S)-3-[5-[(7S)-3-(3,5-difluorophenyl)-2,7-dimethyl-5,7-dihydro-4H-pyrazolo[3,4-c]pyridine-6-carbonyl]-1,2,4-triazol-1-yl]pyrrolidine-1-carboxylate (104.0 mg, 0.200 mmol) in MeOH (0.500 mL) was treated with 10 M aqueous HCl (0.2 mL, 1.97 mmol), at 23° C. The mixture was stirred for 16 h at this temperature, before being evaporated. RP-HPLC gave the title compound (39.7 mg, 43.41% yield) as a light brown solid. MS (ESI): m/z=428.2 [M+H]⁺.

### Step a): ethyl 2-(1-tert-butoxycarbonylpyrrolidin-3-yl)-1,2,4-triazole-3-carboxylate and ethyl 1-(1-tert-butoxycarbonylpyrrolidin-3-yl)-1,2,4-triazole-3-carboxylate

A solution of ethyl 1H-1,2,4-triazole-3-carboxylate (CAS RN: 4928-88-5; 699.64 mg, 4.96 mmol) in DMF (10 mL) was treated with tert-butyl 3-bromopyrrolidine-1-carboxylate (CAS RN: 939793-16-5; 1.24 g, 4.96 mmol) and potassium carbonate (1370.33 mg, 9.91 mmol), at 25° C. The mixture was stirred for 16 h at this temperature, before being purified by FC (SiO₂; hexane/EtOAc), to give first eluting ethyl 2-(1-tert-butoxycarbonylpyrrolidin-3-yl)-1,2,4-triazole-3-carboxylate (390 mg, 25.35% yield) as a colorless oil and second eluting ethyl 1-(1-tert-butoxycarbonylpyrrolidin-3-yl)-1,2,4-triazole-3-carboxylate (210 mg, 13.65% yield) as a colorless oil. MS (ESI) m/z=311.2 [M+H]⁺ for both compounds.

### Step b): methyl 2-(1-tert-butoxycarbonylpyrrolidin-3-yl)-1,2,4-triazole-3-carboxylate

A solution of methyl 2-pyrrolidin-3-yl-1,2,4-triazole-3-carboxylate;dihydrochloride (1.0 g, 3.72 mmol) in DCM (15 mL) was treated with methyl 2-pyrrolidin-3-yl-1,2,4-triazole-3-carboxylate;dihydrochloride (1.0 g, 3.72 mmol) and Boc₂O (1.02 mL, 4.46 mmol), at 23° C. The mixture was stirred for another 16 h at this temperature, before being evaporated. Purification by FC (SiO₂; hexane/EtOAc 1:1) gave the title compound (950 mg, 86.28% yield) as a coloress oil. MS (ESI): m/z=297.2 [M+H]⁺.

### Step c): tert-butyl 3-[(]S)-5-[(7S)-3-(3,5-difluorophenyl)-2,7-dimethyl-5,7-dihydro-4H-pyrazolo[3,4-c]pyridine-6-carbonyl]-1,2,4-triazol-1-yl]pyrrolidine-1-carboxylate

A solution of 3-(3,5-difluorophenyl)-2,7-dimethyl-4,5,6,7-tetrahydropyrazolo[3,4-c]pyridine (399.84 mg, 1.52 mmol) (made from hydrochloride salt and NaOH solution, extracted with DCM) in 1,2-dichloroethane (5 mL) was treated with trimethylaluminum (2.0 M in heptane) (1.01 mL, 2.02 mmol) and methyl 2-(1-tert-butoxycarbonylpyrrolidin-3-yl)-1,2,4-triazole-3-carboxylate (300.0 mg, 1.01 mmol), at 0° C. under Ar. The mixture was heated to 60° C. and stirred for 16 h at this temperature, before being cooled down to 0° C. and treated with water (1 mL). The mixture was stirred for 10 min, filtered through celite, and evaporated. Purification by RP-HPLC and chiral chromatography gave the title compound (104 mg, 25.0% yield) as a brown oil. MS (ESI): m/z=529.4 [M+H]⁺.

### Example 55

### N-[1-[3-Chloro-5-[6-[2-chloro-5-fluoro-3-(6-oxo-1H-pyridin-3-yl)benzoyl]-2,7-dimethyl-5,7-dihydro-4H-pyrazolo[3,4-c]pyridin-3-yl]phenyl]cyclopropyl]methanesulfonamide

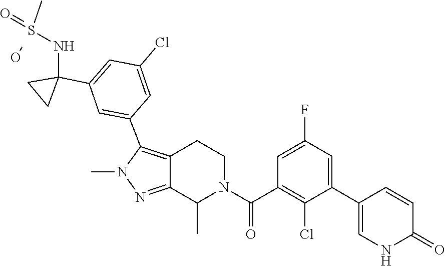

A solution of HATU (42.31 mg, 0.111 mmol) in DMF (0.813 mL) was treated with DIPEA (119.84 mg, 158.31 μL, 0.927 mmol) and 2-chloro-5-fluoro-3-(6-keto-1H-pyridin-3-yl)benzoic acid (C.5; 24.82 mg, 0.093 mmol), at 23° C. under Ar. The mixture was stirred for 10 min at this temperature, before being treated with N-[1-[3-chloro-5-(2,7-dimethyl-4,5,6,7-tetrahydropyrazolo[3,4-c]pyridin-3-yl)phenyl]cyclopropyl]methanesulfonamide;hydrochloride (B.2; 40 mg, 0.093 mmol), and stirred for another 18 h. Purification by RP-HPLC gave the title compound (29 mg, 48.52%) as an off-white lyophilized powder. MS (ESI): m/z=644.4 [M+H]⁺.

In analogy to Example 55, Examples in the following table were generated using amines B.X and carboxylic acids C.Y.

| | | | | MS, |
|---|---|---|---|---|
| | | | Building | ESI: |
| Ex. | Structure | Systematic Name | Blocks | m/z |
| 56 | 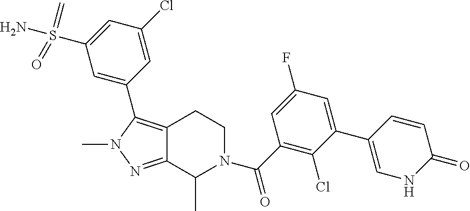 [SMILES: C=S(N)(=O)c1cc(Cl)cc(-c2c3c(nn2C)C(C)N(C(=O)c2cc(F)cc(-c4ccc(=O)[nH]c4)c2Cl)CC3)c1] | 3-Chloro-5-[6-[2-chloro-5- fluoro-3-(6-oxo-1H-pyridin- 3-yl)benzoyl]-2,7-dimethyl- 5,7-dihydro-4H- pyrazolo[3,4-c]pyridin-3- yl]benzenesulfonamide | B.1 and C.5 | 588.3 [M + H]⁺ |
| 57 | 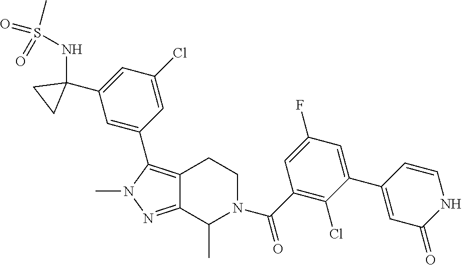 [SMILES: CC1c2nn(C)c(-c3cc(Cl)cc(C4(NS(C)(=O)=O)CC4)c3)c2CCN1C(=O)c1cc(F)cc(-c2cc[nH]c(=O)c2)c1Cl] | N-[1-[3-Chloro-5-[6-[2- chloro-5-fluoro-3-(2-oxo- 1H-pyridin-4-yl)benzoyl]- 2,7-dimethyl-5,7-dihydro- 4H-pyrazolo[3,4-c]pyridin- 3- yl]phenyl]cyclopropyl] methanesulfonamide | B.2 and C.6 | 644.5 [M + H]⁺ |
| 58 | 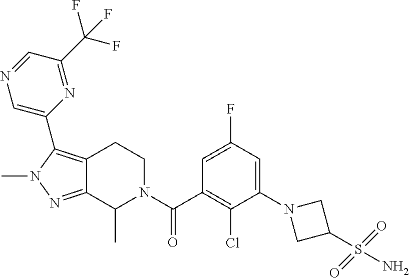 [SMILES: CC1c2nn(C)c(-c3cncc(C(F)(F)F)n3)c2CCN1C(=O)c1cc(F)cc(N2CC(S(N)(=O)=O)C2)c1Cl] | 1-[2-Chloro-3-[2,7-dimethyl- 3-[6- (trifluoromethyl)pyrazin-2- yl]-5,7-dihydro-4H- pyrazolo[3,4-c]pyridine-6- carbonyl]-5-fluoro- phenyl]azetidine-3- sulfonamide | B.3 and C.16 | 588.3 [M + H]⁺ |
| 59 | 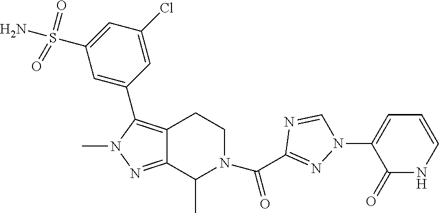 [SMILES: CC1c2nn(C)c(-c3cc(Cl)cc(S(N)(=O)=O)c3)c2CCN1C(=O)c1ncn(-c2ccc[nH]c2=O)n1] | 3-Chloro-5-[2,7-dimethyl-6- [1-(2-oxo-1H-pyridin-3-yl)- 1,2,4-triazole-3-carbonyl]- 5,7-dihydro-4H- pyrazolo[3,4-c]pyridin-3- yl]benzenesulfonamide | B.1 and C.7 | 527.3 [M + H]⁺ |
| 60 | 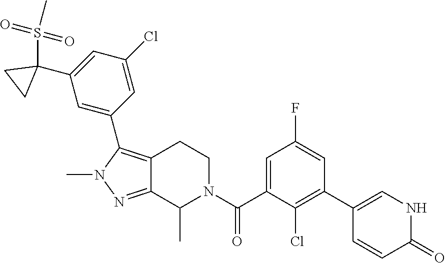 [SMILES: CC1c2nn(C)c(-c3cc(Cl)cc(C4(S(C)(=O)=O)CC4)c3)c2CCN1C(=O)c1cc(F)cc(-c2ccc(=O)[nH]c2)c1Cl] | 5-[2-Chloro-3-[3-[3-chloro- 5-(1- methylsulfonylcyclopropyl) phenyl]-2,7-dimethyl-5,7- dihydro-4H-pyrazolo[3,4- c]pyridine-6-carbonyl]-5- fluoro-phenyl]-1H-pyridin-2- one | B.4 and C.5 | 627.4 [M + H]⁺ |
| 61 | 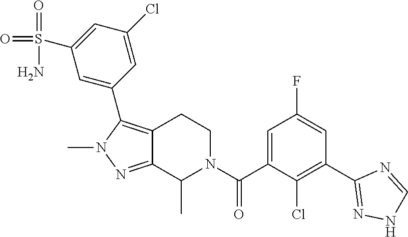 [SMILES: CC1c2nn(C)c(-c3cc(Cl)cc(S(N)(=O)=O)c3)c2CCN1C(=O)c1cc(F)cc(-c2nc[nH]n2)c1Cl] | 3-Chloro-5-[6-[2-chloro-5- fluoro-3-(1H-1,2,4-triazol-3- yl)benzoyl]-2,7-dimethyl-5,7- dihydro-4H-pyrazolo[3,4- c]pyridin-3- yl]benzenesulfonamide | B.1 and C.17 | 562.3 [M + H]⁺ |

### Example 62

### 3-Chloro-5-[(7S)-2,7-dimethyl-6-[1-(2-oxo-1H-pyridin-3 yl)-1,2,4-triazole-3-carbonyl]-5,7-dihydro-4H-pyrazolo[3,4-c]pyridin-3-yl]benzenesulfonamide

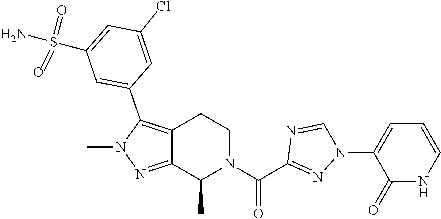

Example 59 (30 mg, 0.06 mmol) was separated by chiral chromatography, to give the title compound (6.7 mg, 22.3% yield) as an off-white amorphous solid. MS (ESI): m/z=527.3 [M+H]⁺.

### Example 63

### 3-Chloro-5-[(7R)-2,7-dimethyl-6-[1-(2-oxo-1H-pyridin-3-yl)-1,2,4-triazole-3-carbonyl]-5,7-dihydro-4H-pyrazolo[3,4-c]pyridin-3-yl]benzenesulfonamide

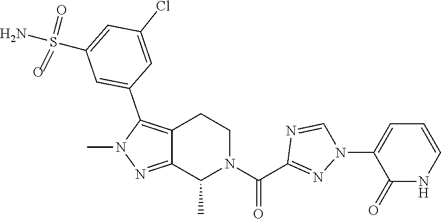

Example 59 (30 mg, 0.06 mmol) was separated by chiral chromatography, to give the title compound (5.7 mg, 19.0% yield) as an off-white amorphous solid. MS (ESI): m/z=527.3 [M+H]⁺.

### Example 64

### 4-[2-chloro-3-[(7S)-2,7-dimethyl-3-[6-(trifluoromethyl)pyrazin-2-yl]-5,7-dihydro-4H-pyrazolo[3,4-c]pyridine-6-carbonyl]-5-fluoro-phenyl]-1H-pyrrole-2-carbonitrile

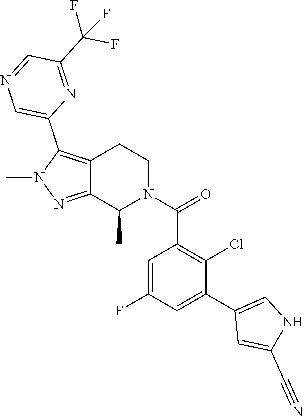 [SMILES: C[C@H]1c2nn(C)c(-c3cncc(C(F)(F)F)n3)c2CCN1C(=O)c1cc(F)cc(-c2c[nH]c(C#N)c2)c1Cl]

A solution of HATU (61.52 mg, 0.162 mmol) in DMF (1.2 mL) was treated with DIPEA (230.21 μL, 1.35 mmol) and 2-chloro-3-(5-cyano-1H-pyrrol-3-yl)-5-fluoro-benzoic acid (C.11; 35.68 mg, 0.135 mmol), at 23° C. under Ar. The mixture was stirred for 10 min at this temperature, before being treated with 2,7-dimethyl-3-[6-(trifluoromethyl)pyrazin-2-yl]-4,5,6,7-tetrahydropyrazolo[3,4-c]pyridine;hydrochloride (B.3; 45 mg, 0.135 mmol), and stirred for another 18 h. Purification by RP-HPLC gave the racemate as a white lyophilisate (45 mg). Purification by chiral chromatography gave the title compound (16.2 mg, 22.09%) as a white solid. MS (ESI): m/z=544.3 [M+H]⁺.

### Example 65

### [2-chloro-3-(2,2-dioxo-2λ⁶-thia-6-azaspiro[3.3]heptan-6-yl)-5-fluoro-phenyl]-[(7S)-2,7-dimethyl-3-[6-(trifluoromethyl)pyrazin-2-yl]-5,7-dihydro-4H-pyrazolo[3,4-c]pyridin-6-yl]methanone

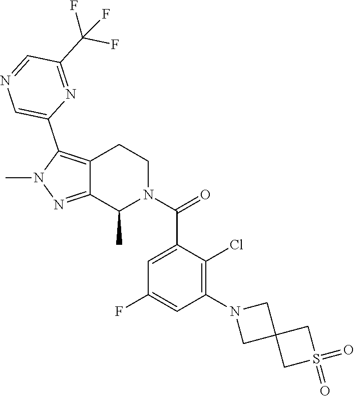 [SMILES: C[C@H]1c2nn(C)c(-c3cncc(C(F)(F)F)n3)c2CCN1C(=O)c1cc(F)cc(N2CC3(C2)CS(=O)(=O)C3)c1Cl]

A solution of HATU (61.52 mg, 0.162 mmol) in DMF (1.2 mL) was treated with DIPEA (230.21 μL, 1.35 mmol) and 2-chloro-3-(2,2-diketo-2λ⁶-thia-6-azaspiro[3.3]heptan-6-yl)-5-fluoro-benzoic acid (C.1; 43.11 mg, 0.135 mmol), at 23° C. under Ar. The mixture was stirred for 10 min at this temperature, before being treated with 2,7-dimethyl-3-[6-(trifluoromethyl)pyrazin-2-yl]-4,5,6,7-tetrahydropyrazolo[3,4-c]pyridine;hydrochloride (B.3; 45 mg, 0.135 mmol), and stirred for another 18 h. Purification by RP-HPLC gave racemic product as a white lyophilisate (62 mg). Purification by chiral chromatography gave the title compound (17 mg, 21.05%) as a white solid. MS (ESI): m/z=599.4 [M+H]⁺.

### Example 66

### [2-chloro-3-(2,2-dioxo-2λ⁶-thia-6-azaspiro[3.3]heptan-6-yl)-5-fluoro-phenyl]-[(7R)-2,7-dimethyl-3-[6-(trifluoromethyl)pyrazin-2-yl]-5,7-dihydro-4H-pyrazolo[3,4-c]pyridin-6-yl]methanone

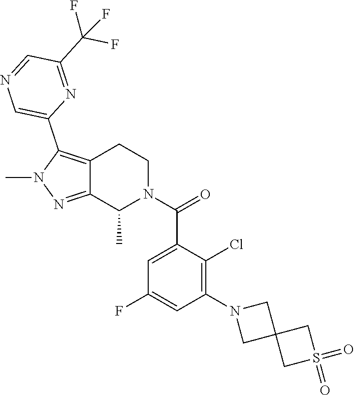 [SMILES: C[C@@H]1c2nn(C)c(-c3cncc(C(F)(F)F)n3)c2CCN1C(=O)c1cc(F)cc(N2CC3(C2)CS(=O)(=O)C3)c1Cl]

A solution of HATU (61.52 mg, 0.162 mmol) in DMF (1.2 mL) was treated with DIPEA (230.21 μL, 1.35 mmol) and 2-chloro-3-(2,2-diketo-2λ6-thia-6-azaspiro[3.3]heptan-6-yl)-5-fluoro-benzoic acid (C.1; 43.11 mg, 0.135 mmol), at 23° C. under Ar. The mixture was stirred for 10 min at this temperature, before being treated with 2,7-dimethyl-3-[6-(trifluoromethyl)pyrazin-2-yl]-4,5,6,7-tetrahydropyrazolo[3,4-c]pyridine;hydrochloride (B.3; 45 mg, 0.135 mmol), and stirred for another 18 h. Purification by RP-HPLC gave racemic product as a white lyophilisate (62 mg). Purification by chiral chromatography gave the title compound (23.4 mg, 29.0%) as a white solid. MS (ESI): m/z=599.4 [M+H]⁺.

### Example 67

### 3-[3-[(7S)-2,7-dimethyl-3-[6-(trifluoromethyl)pyrazin-2-yl]-5,7-dihydro-4H-pyrazolo[3,4-c]pyridine-6-carbonyl]-1,2,4-triazol-1-yl]-1H-pyridin-2-one

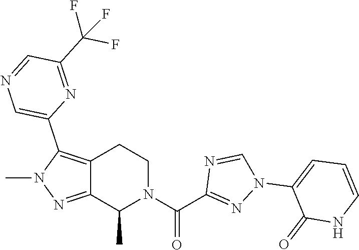 [SMILES: C[C@H]1c2nn(C)c(-c3cncc(C(F)(F)F)n3)c2CCN1C(=O)c1ncn(-c2ccc[nH]c2=O)n1]

A solution of HATU (61.52 mg, 0.162 mmol) in DMF (1.2 mL) was treated with DIPEA (230.21 μL, 1.35 mmol) and 1-(2-keto-1H-pyridin-3-yl)-1,2,4-triazole-3-carboxylic acid (C.7; 27.8 mg, 0.135 mmol), at 23° C. under Ar. The mixture was stirred for 10 min at this temperature, before being treated with 2,7-dimethyl-3-[6-(trifluoromethyl)pyrazin-2-yl]-4,5,6,7-tetrahydropyrazolo[3,4-c]pyridine;hydrochloride (B.3; 45 mg, 0.135 mmol), and stirred for another 18 h. Purification by RP-HPLC gave the racemate as a white lyophilisate (35 mg). Purification by chiral chromatography gave the title compound (10 mg, 15.28%) as an off-white solid. MS (ESI): m/z=486.4 [M+H]⁺.

### Example 68

### 5-[2-chloro-3-[(7S)-2,7-dimethyl-3-[6-(trifluoromethyl)pyrazin-2-yl]-5,7-dihydro-4H-pyrazolo[3,4-c]pyridine-6-carbonyl]-5-fluoro-phenyl]-1H-pyridin-2-one

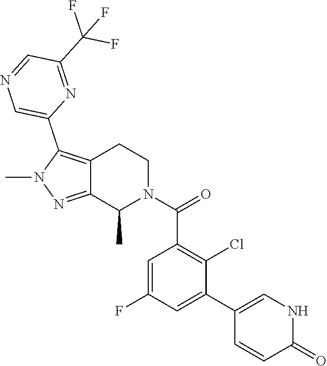 [SMILES: C[C@H]1c2nn(C)c(-c3cncc(C(F)(F)F)n3)c2CCN1C(=O)c1cc(F)cc(-c2ccc(=O)[nH]c2)c1Cl]

A solution of HATU (61.52 mg, 0.162 mmol) in DMF (1.2 mL) was treated with DIPEA (230.21 69, 1.35 mmol) and 2-chloro-5-fluoro-3-(6-keto-1H-pyridin-3-yl)benzoic acid (C.5; 36.09 mg, 0.135 mmol), at 23° C. under Ar. The mixture was stirred for 10 min at this temperature, before being treated with 2,7-dimethyl-3-[6-(trifluoromethyl)pyrazin-2-yl]-4,5,6,7-tetrahydropyrazolo[3,4-c]pyridine;hydrochloride (B.3; 45 mg, 0.135 mmol), and stirred for another 18 h. Purification by RP-HPLC and chiral chromatography gave the title compound (11 mg, 14.920%) as a light brown powder. MS (ESI): m/z=547.4 [M+H]⁺.

The following compounds were made in analogy to the compounds described above:

| | | | MS, |
|---|---|---|---|
| | | | ESI: |
| Ex. | Structure | IUPAC Name | m/z |
| 69 | 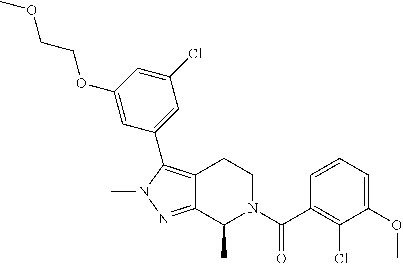 | [(7S)-3-[3-chloro-5-(2- methoxyethoxy)phenyl]-2,7- dimethyl-5,7-dihydro-4H- pyrazolo[3,4-c]pyridin-6-yl]-(2- chloro-3-methoxy- phenyl)methanone | 504.4 [M + H]⁺ |
| 70 | 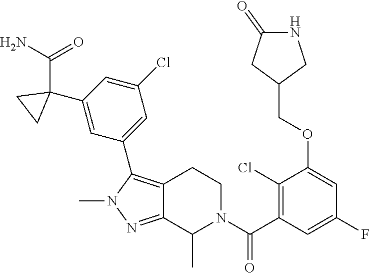 [SMILES: CC1c2nn(C)c(-c3cc(Cl)cc(C4(C(N)=O)CC4)c3)c2CCN1C(=O)c1cc(F)cc(OCC2CNC(=O)C2)c1Cl] | 1-[3-chloro-5-[6-[2-chloro-5-fluoro- 3-[(5-oxopyrrolidin-3- yl)methoxy]benzoyl]-2,7-dimethyl- 5,7-dihydro-4H-pyrazolo[3,4- c]pyridin-3- yl]phenyl]cyclopropanecarboxamide | 614.4 [M + H]⁺ |
| 71 | 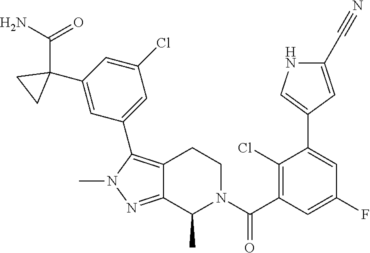 [SMILES: C[C@H]1c2nn(C)c(-c3cc(Cl)cc(C4(C(N)=O)CC4)c3)c2CCN1C(=O)c1cc(F)cc(-c2c[nH]c(C#N)c2)c1Cl] | 1-[3-chloro-5-[(7S)-6-[2-chloro-3- (5-cyano-1H-pyrrol-3-yl)-5-fluoro- benzoyl]-2,7-dimethyl-5,7-dihydro- 4H-pyrazolo[3,4-c]pyridin-3- yl]phenyl]cyclopropanecarboxamide | 591.3 [M + H]⁺ |
| 72 | 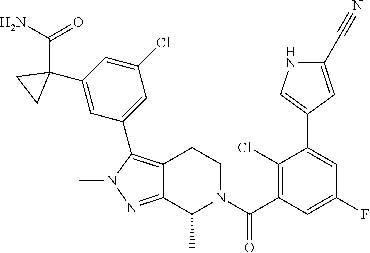 [SMILES: C[C@@H]1c2nn(C)c(-c3cc(Cl)cc(C4(C(N)=O)CC4)c3)c2CCN1C(=O)c1cc(F)cc(-c2c[nH]c(C#N)c2)c1Cl] | 1-[3-chloro-5-[(7R)-6-[2-chloro-3- (5-cyano-1H-pyrrol-3-yl)-5-fluoro- benzoyl]-2,7-dimethyl-5,7-dihydro- 4H-pyrazolo[3,4-c]pyridin-3- yl]phenyl]cyclopropanecarboxamide | 591.3 [M + H]⁺ |
| 73 | 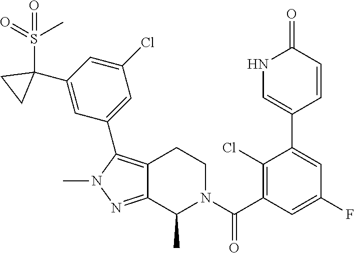 [SMILES: C[C@H]1c2nn(C)c(-c3cc(Cl)cc(C4(S(C)(=O)=O)CC4)c3)c2CCN1C(=O)c1cc(F)cc(-c2ccc(=O)[nH]c2)c1Cl] | 5-[2-chloro-3-[(7S)-3-[3-chloro-5- (1- methylsulfonylcyclopropyl)phenyl]- 2,7-dimethyl-5,7-dihydro-4H- pyrazolo[3,4-c]pyridine-6- carbonyl]-5-fluoro-phenyl]-1H- pyridin-2-one | 627.4 [M + H]⁺ |
| 74 | 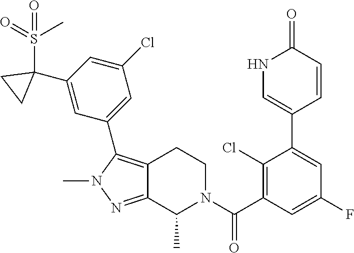 [SMILES: C[C@@H]1c2nn(C)c(-c3cc(Cl)cc(C4(S(C)(=O)=O)CC4)c3)c2CCN1C(=O)c1cc(F)cc(-c2ccc(=O)[nH]c2)c1Cl] | 5-[2-chloro-3-[(7R)-3-[3-chloro-5- (1- methylsulfonylcyclopropyl)phenyl]- 2,7-dimethyl-5,7-dihydro-4H- pyrazolo[3,4-c]pyridine-6- carbonyl]-5-fluoro-phenyl]-1H- pyridin-2-one | 627.4 [M + H]⁺ |
| 75 | 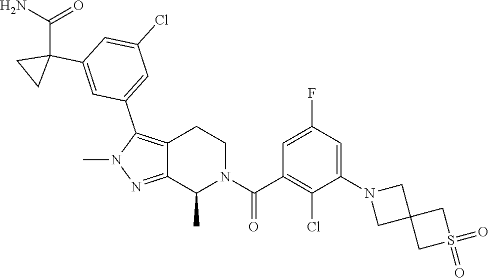 [SMILES: C[C@H]1c2nn(C)c(-c3cc(Cl)cc(C4(C(N)=O)CC4)c3)c2CCN1C(=O)c1cc(F)cc(N2CC3(C2)CS(=O)(=O)C3)c1Cl] | 1-[3-chloro-5-[(7S)-6-[2-chloro-3- (2,2-dioxo-2λ⁶-thia-6- azaspiro[3.3]heptan-6-yl)-5-fluoro- benzoyl]-2,7-dimethyl-5,7-dihydro- 4H-pyrazolo[3,4-c]pyridin-3- yl]phenyl]cyclopropanecarboxamide | 646.4 [M + H]⁺ |
| 76 | 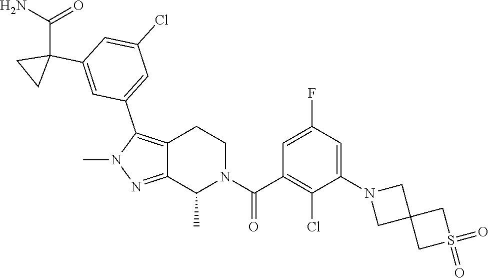 [SMILES: C[C@@H]1c2nn(C)c(-c3cc(Cl)cc(C4(C(N)=O)CC4)c3)c2CCN1C(=O)c1cc(F)cc(N2CC3(C2)CS(=O)(=O)C3)c1Cl] | 1-[3-chloro-5-[(7R)-6-[2-chloro-3- (2,2-dioxo-2λ⁶-thia-6- azaspiro[3.3]heptan-6-yl)-5-fluoro- benzoyl]-2,7-dimethyl-5,7-dihydro- 4H-pyrazolo[3,4-c]pyridin-3- yl]phenyl]cyclopropanecarboxamide | 646.4 [M + H]⁺ |
| 77 | 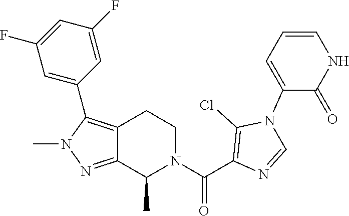 [SMILES: C[C@H]1c2nn(C)c(-c3cc(F)cc(F)c3)c2CCN1C(=O)c1ncn(-c2ccc[nH]c2=O)c1Cl] | 3-[5-chloro-4-[(7S)-3-(3,5- difluorophenyl)-2,7-dimethyl-5,7- dihydro-4H-pyrazolo[3,4- c]pyridine-6-carbonyl]imidazol-1- yl]-1H-pyridin-2-one | 485.2 [M + H]⁺ |
| 78 | 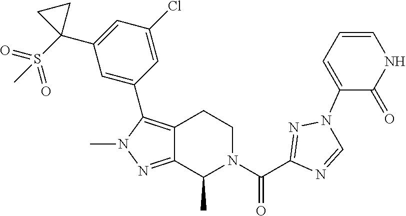 [SMILES: C[C@H]1c2nn(C)c(-c3cc(Cl)cc(C4(S(C)(=O)=O)CC4)c3)c2CCN1C(=O)c1ncn(-c2ccc[nH]c2=O)n1] | 3-[3-[(7S)-3-[3-chloro-5-(1- methylsulfonylcyclopropyl)phenyl]- 2,7-dimethyl-5,7-dihydro-4H- pyrazolo[3,4-c]pyridine-6- carbonyl]-1,2,4-triazol-1-yl]-1H- pyridin-2-one | 566.4 [M + H]⁺ |
| 79 | 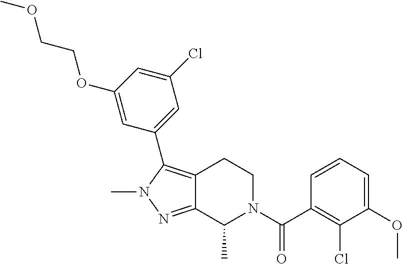 | [(7R)-3-[3-chloro-5-(2- methoxyethoxy)phenyl]-2,7- dimethyl-5,7-dihydro-4H- pyrazolo[3,4-c]pyridin-6-yl]-(2- chloro-3-methoxy- phenyl)methanone | 504.4 [M + H]⁺ |

### Example 80

### N-[[2-chloro-6-[6-(2-chloro-3-methoxy-benzoyl)-2,7-dimethyl-5,7-dihydro-4H-pyrazolo[3,4-c]pyridin-3-yl]-4-pyridyl]methyl]methanesulfonamide

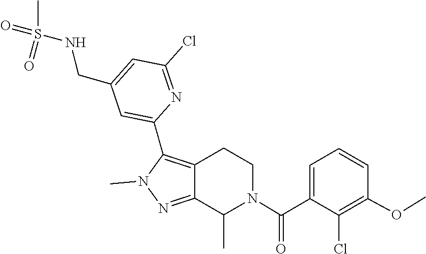 [SMILES: COc1cccc(C(=O)N2CCc3c(nn(C)c3-c3cc(CNS(C)(=O)=O)cc(Cl)n3)C2C)c1Cl]

A solution of 2-chloro-3-methoxybenzoic acid (21.07 mg, 0.11 mmol) in DCM (1 mL) was treated with oxalyl chloride (0.01 mL, 0.14 mmol) at 23° C. The mixture was stirred for 1 h at this temperature, before being evaporated. The residue was dissolved in DCM (1 mL). N-[[2-Chloro-6-(2,7-dimethyl-4,5,6,7-tetrahydropyrazolo[3,4-c]pyridin-6-ium-3-yl)pyridin-1l-ium-4-yl]methyl]methanesulfonamide;dichloride (50.0 mg, 0.11 mmol) and triethylamine (0.08 mL, 0.56 mmol) were added to the solution and the resulting mixture was stirred at 20° C. for 10 h, before being evaporated. The residue was purified by RP-HPLC to give the title compound (46.0 mg, 75.66%) as a light brown solid. MS (ESI): m/z=538.0 [M+H]⁺

### Step a): tert-butyl 2,7-dimethyl-5,7-dihydro-4H-pyrazolo[3,4-c]pyridine-6-carboxylate

A solution of tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (8.0 g, 20.03 mmol) in EtOAc (1.33 L) was treated with molecular sieves (17.64 g, 40.05 mmol), triethylamine (4.19 mL, 30.04 mmol) and Pd/C 10% (8.0 g), under Ar. The mixture was then hydrogenated at 1500 mm Hg (with balloon) for 18 h at 25° C. The mixture was filtered and evaporated, to give the title compound (5.0 g, 98.35% yield) as a light yellow viscous oil. MS (ESI): m/z=196 [M-tBu+H]⁺

### Step b): tert-butyl 3-bromo-2,7-dimethyl-5,7-dihydro-4H-pyrazolo[3,4-c]pyridine-6-carboxylate

A solution of tert-butyl 2,7-dimethyl-5,7-dihydro-4H-pyrazolo[3,4-c]pyridine-6-carboxylate (3.0 g, 11.94 mmol) in ACN (30 mL) was treated with NBS (2.1 g, 11.94 mmol), at 23° C. The mixture was stirred for 15 h at this temperature, before being evaporated and dissolved in EtOAc. The organic layer was washed with water and brine, before being dried over Na₂SO₄, filtered, and evaporated, to give the title compound (3.3 g, 78.44% yield) as a light yellow solid. MS (ESI): m/z=274.0/276.0 [M-Bu+H]⁺

### Step c): (6-tert-butoxycarbonyl-2,7-dimethyl-5,7-dihydro-4H-pyrazolo[3,4-c]pyridin-3-yl)boronic acid

A solution of tert-butyl 3-bromo-2,7-dimethyl-5,7-dihydro-4H-pyrazolo[3,4-c]pyridine-6-carboxylate (4.1 g, 12.42 mmol) in THF (50 mL) was treated dropwise with 2.5 M n-butyllithium solution in THF (6.21 mL, 15.52 mmol), at −78° C. under Ar. After stirring for 1 h at this temperature, tri-methyl borate (3.87 g, 37.25 mmol) was added, and the resulting solution was stirred for 8 h at −78° C., and for 10 h at 20° C. The mixture was then carefully treated with 1 N aqueous HCl, and extracted with EtOAc. The combined organic layers were dried over Na₂SO₄, filtered, and evaporated, to give the title compound (2.78 g, 66.31% yield) as a yellow foam. MS (ESI): m/z=296.2 [M+H]⁺

### Step d): N-[(2,6-dichloro-4-pyridyl)methyl]methanesulfonamide

A suspension of (2,6-dichloropyridin-1-ium-4-yl)methylammonium;dichloride (1.69 g, 6.76 mmol) in DCM (30 mL) was cooled to 0° C. and triethylamine (3.77 mL, 27.05 mmol) was added. The mixture was stirred for 30 min and methanesulfonyl chloride (0.58 mL, 7.44 mmol) was added dropwise, while keeping the temperature below 5° C. The mixture was then warmed up to 20° C. and stirred for 5 h at that temperature. The mixture was diluted with DCM, washed with water. The organic layer was dried over sodium sulfate and evaporated, to give the title compound (1.49 g, 85.52% yield) as a brown solid. MS (ESI): m/z=255.0/257.0 [M+H]⁺

### Step e): tert-butyl 3-[6-chloro-4-(methanesulfonamidomethyl)-2-pyridyl]-2,7-dimethyl-5,7-dihydro-4H-pyrazolo[3,4-c]pyridine-6-carboxylate

A solution of (6-tert-butoxycarbonyl-2,7-dimethyl-5,7-dihydro-4H-pyrazolo[3,4-c]pyridin-3-yl)boronic acid (450.0 mg, 1.52 mmol) in 1,4-Dioxane (15 mL) was treated with N-[(2,6-dichloro-4-pyridyl)methyl]methanesulfonamide (330.63 mg, 1.3 mmol) and potassium carbonate (526.81 mg, 3.81 mmol, 2.5 eq), at 23° C. The mixture was sparged with Ar before being treated with 1,1′-bis(diphenylphosphino)ferrocene-palladium(II)dichloride dichloromethane complex (124.42 mg, 0.15 mmol). The resulting mixture was stirred at 100° C. for 15 h in a sealed vessel, before being cooled down and evaporated. The residue was dissolved in EtOAc and the resulting suspension was filtered through celite. The filtrate was concentrated, and the residue was purified by RP-HPLC to give the title compound (130.3 mg, 17.67%) as a light brown foam. MS (ESI): m/z=414.0/416.0 [M-Bu+H]⁺

### Step j): N-[[2-chloro-6-(2,7-dimethyl-4,5,6,7-tetrahydropyrazolo[3,4-c]pyridin-6-ium-3-yl)pyridin-1-ium-4-yl]methyl]methanesulfonamide;dichloride

A solution of tert-butyl 3-[6-chloro-4-(methanesulfonamidomethyl)-2-pyridyl]-2,7-dimethyl-5,7-dihydro-4H-pyrazolo[3,4-c]pyridine-6-carboxylate (130.3 mg, 0.28 mmol) in DCM (2 mL) was treated with hydrochloric acid (4 M in 1,4-dioxane) (0.69 mL, 2.77 mmol), at 20° C. The mixture was stirred for 20 h at this temperature, before being evaporated to give the title compound (120.0 mg, 94.23%) as a light brown solid. MS (ESI): m/z=370.0/372.0 [M+H]⁺

### Example 81

### (2-chloro-3-methoxy-phenyl)-[(7S)-2,7-dimethyl-3-[2-(trifluoromethyl)pyrimidin-4-yl]-5,7-dihydro-4H-pyrazolo[3,4-c]pyridin-6-yl]methanone

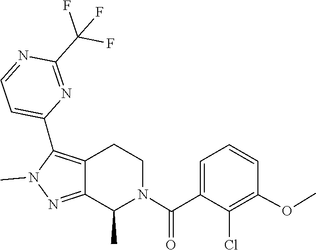

A solution of 2,7-dimethyl-3-[2-(trifluoromethyl)pyrimidin-4-yl]-4,5,6,7-tetrahydropyrazolo[3,4-c]pyridine;hydrochloride (B.3; 200.0 mg, 0.6 mmol) in DMF (3 mL) was treated with 2-chloro-3-methoxybenzoic acid (123.0 mg, 0.66 mmol), HATU (341.79 mg, 0.9 mmol) and N,N-diisopropylethylamine (0.52 mL, 3.0 mmol), at 23° C. The mixture was stirred for another 18 h at this temperature, before being purified by RP-HPLC. Chiral chromatography gave the title compound (44.2 mg, 15.83% yield) as a white solid. MS (ESI): m/z=466 [M+H]⁺

### Example 82 and Example 83

### 4-[2-chloro-5-fluoro-3-[(7S)-3-[3-fluoro-5-[[methyl(dioxo)-λ⁶-phosphanyl]methyl]phenyl]-2,7-dimethyl-5,7-dihydro-4H-pyrazolo[3,4-c]pyridine-6-carbonyl]phenyl]-1H-pyrrole-2-carbonitrile (Example 82) and 4-[2-chloro-5-fluoro-3-[(7R)-3-[3-fluoro-5-[[methyl(dioxo)-λ⁶-phosphanyl]methyl]phenyl]-2,7-dimethyl-5,7-dihydro-4H-pyrazolo[3,4-c]pyridine-6-carbonyl]phenyl]-1H-pyrrole-2-carbonitrile (Example 83)

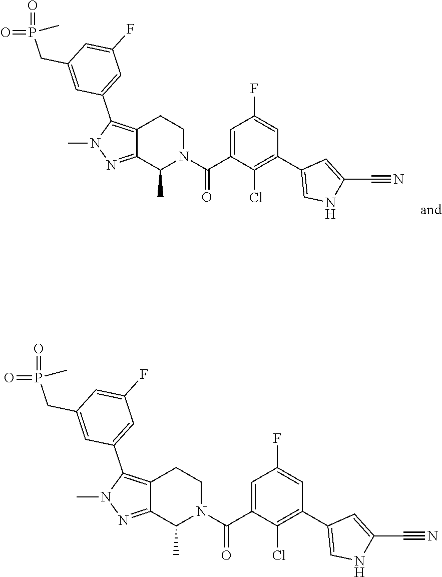

A solution of 2-chloro-3-(5-cyano-1H-pyrrol-3-yl)-5-fluoro-benzoic acid (C.11; 149.47 mg, 0.51 mmol), 3-[3-(dimethylphosphorylmethyl)-5-fluoro-phenyl]-2,7-dimethyl-4,5,6,7-tetrahydropyrazolo[3,4-c]pyridine;hydrochloride (189.0 mg, 0.51 mmol) and triethylamine (0.35 mL, 2.54 mmol) in DMF (3 mL) was treated with HATU (212.6 mg, 0.56 mmol), and stirred for 16 h at 20° C. Purification by RP-HPLC followed by chiral chromatography gave Example 82 (51.0 mg, 30.37% yield) and Example 83 (52.4 mg, 32.17% yield) as light brown solids. MS (ESI): m/z=582.2 [M+H]⁺

### Step a): 1-bromo-3-(dimethylphosphorylmethyl)-5-fluoro-benzene

A solution of 1-bromo-3-(bromomethyl)-5-fluoro-benzene (10.0 g, 37.32 mmol) in THF (150 mL) was treated with a solution of NaHMDS in THF (27.99 mL, 55.99 mmol). The mixture was stirred for 30 min, before being treated portionwise with dimethylphosphine oxide (4.37 g, 55.99 mmol) and stirred for 16 h. Water (1 mL) was then added, and the mixture was extracted with EtOAc. The combined organic layers were dried over Na₂SO₄, filtered, and evaporated. Trituration with hexane gave the title compound (1.15 g, 11.62%) as a white solid. MS (ESI): m/z=265/267 [M+H]⁺

### Step b): 2-[3-(dimethylphosphorylmethyl)-5-fluoro-phenyl]-4,4,5,5-tetramethyl-1,3,2-dioxaborolane

A solution of bis(pinacolato)diboron (1.44 g, 5.66 mmol), potassium acetate (0.71 mL, 11.32 mmol), 1-bromo-3-(dimethylphosphorylmethyl)-5-fluoro-benzene (1.0 g, 3.77 mmol) and 1,1′-bis(diphenylphosphino)ferrocene-palladium(II)dichloride dichloromethane complex (153.93 mg, 0.19 mmol) in 1,4-Dioxane (15 mL) was sparged with Ar, sealed, and heated at 100° C. for 16 h. The mixture was cooled down, filtered and evaporated, to give the crude title compound (1.07 g, 87.0% yield) as a brown solid. MS (ESI): m/z=313.2 [M+H]⁺

### Step c): tert-butyl 3-[3-(dimethylphosphorylmethyl)-5-fluoro-phenyl]-2,7-dimethyl-5,7-dihydro-4H-pyrazolo[3,4-c]pyridine-6-carboxylate

A degassed suspension of 2-[3-(dimethylphosphorylmethyl)-5-fluoro-phenyl]-4,4,5,5-tetramethyl-1,3,2-dioxaborolane (570.1 mg, 1.64 mmol), tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (505.0 mg, 1.26 mmol) and sodium carbonate (268.0 mg, 2.53 mmol) in Toluene (15 mL)/Water (1.5 mL) was treated with tetrakis(triphenylphosphine)palladium(0) (73.1 mg, 0.06 mmol). The resulting mixture was stirred at 80° C. for 15 h, before being cooled down and evaporated. Purification by RP-HPLC gave the title compound (287.0 mg, 52.12% yield) as a brown oil. MS (ESI): m/z=380.2 [M-Bu+H]⁺

### Step d): 3-[3-(dimethylphosphorylmethyl)-5-fluoro-phenyl]-2,7-dimethyl-4,5,6,7-tetrahydropyrazolo[3,4-c]pyridine;hydrochloride

A solution of tert-butyl 3-[3-(dimethylphosphorylmethyl)-5-fluoro-phenyl]-2,7-dimethyl-5,7-dihydro-4H-pyrazolo[3,4-c]pyridine-6-carboxylate (630.0 mg, 1.45 mmol) in DCM (3 mL) was treated with a HCl solution in dioxane (3.62 mL, 14.47 mmol). The mixture was stirred at 20° C. for 10 h, before being evaporated. Purification by RP-HPLC gave the title compound (398.5 mg, 70.38% yield) as a light brown solid. MS (ESI): m/z=336.2 [M+H]⁺

### Example 84

### N-[1-[3-chloro-5-[6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropyl]methanesulfonamide

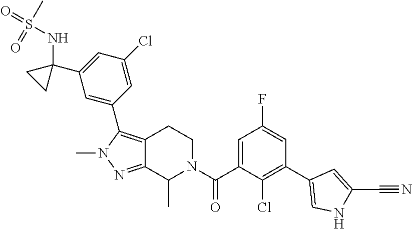 [SMILES: CC1c2nn(C)c(-c3cc(Cl)cc(C4(NS(C)(=O)=O)CC4)c3)c2CCN1C(=O)c1cc(F)cc(-c2c[nH]c(C#N)c2)c1Cl]

A solution of 2-chloro-3-(5-cyano-1H-pyrrol-3-yl)-5-fluoro-benzoic acid (C.11; 149.47 mg, 0.51 mmol), N-[1-[3-chloro-5-(2,7-dimethyl-4,5,6,7-tetrahydropyrazolo[3,4-c]pyridin-3-yl)phenyl]cyclopropyl]-methanesulfonamide;hydrochloride (B.2; 189.0 mg, 0.51 mmol) and triethylamine (0.35 mL, 2.54 mmol) in DMF (3 mL) was treated with HATU (212.6 mg, 0.56 mmol), at 20° C. The mixture was stirred for another 16 h at this temperature, before being purified by RP-HPLC, to give the title compound (136.0 mg, 44.13% yield) as a pink solid. MS (ESI): m/z=641.2 [M+H]⁺

### Example 85 and Example 86

### N-[1-[3-chloro-5-[(7S)-6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropyl]methanesulfonamide (Example 85) and N-[1-[3-chloro-5-[(7R)-6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropyl]methanesulfonamide Example 86

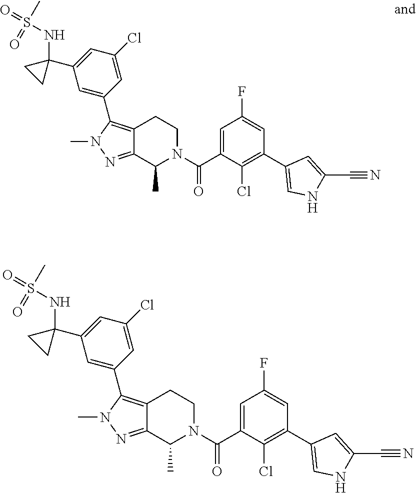 [SMILES: C[C@H]1c2nn(C)c(-c3cc(Cl)cc(C4(NS(C)(=O)=O)CC4)c3)c2CCN1C(=O)c1cc(F)cc(-c2c[nH]c(C#N)c2)c1Cl ; C[C@@H]1c2nn(C)c(-c3cc(Cl)cc(C4(NS(C)(=O)=O)CC4)c3)c2CCN1C(=O)c1cc(F)cc(-c2c[nH]c(C#N)c2)c1Cl]

N-[1-[3-Chloro-5-[6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropyl]methanesulfonamide (Example 84; 163 mg, 0.3 mmol) was purified by chiral HPLC, to give Example 85 (51.0 mg, 30.37% yield) and Example 86 (52.4 mg, 32.17% yield) as light brown solids. MS (ESI): m/z=641.2 [M+H]⁺

### Example 87

### N-[1-[3-chloro-5-[6-[2-chloro-5-fluoro-3-[2-(H-triazol-4-yl)ethyl]benzoyl]-2,7-dimethyl-5,7-dihydro-4H-pyrazolo[3,4-c]pyridin-3-yl]phenyl]cyclopropyl]methanesulfonamide

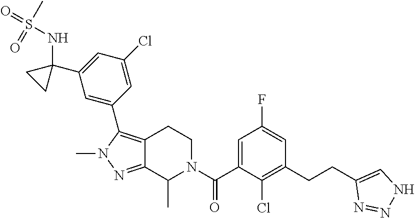 [SMILES: CC1c2nn(C)c(-c3cc(Cl)cc(C4(NS(C)(=O)=O)CC4)c3)c2CCN1C(=O)c1cc(F)cc(CCc2c[nH]nn2)c1Cl]

A solution of N-[1-[3-chloro-5-(2,7-dimethyl-4,5,6,7-tetrahydropyrazolo[3,4-c]pyridin-3-yl)phenyl]cyclopropyl]methanesulfonamide;hydrochloride (B.2; 60 mg, 0.139 mmol) in N,N-dimethylformamide (1.18 mL) and n-ethyldiisopropylamine (237.47 μL, 1.39 mmol) was treated with 2-chloro-5-fluoro-3-[2-(1H-triazol-4-yl)ethyl]benzoic acid;2,2,2-trifluoroacetic acid (C.18; 53.53 mg, 0.140 mmol) and HATU (106.52 mg, 0.280 mmol), at 23° C. The mixture was stirred for 18 h, before being purified by RP-HPLC, to give the title compound (19.4 mg, 21.6%) as a colorless foam. MS (ESI): m/z=644.3 [M+H]⁺

### Step a): 1-[(4-methoxyphenyl)methyl]-4-[2-(4,4,5,5-tetramethyl-1,3,2-dioxaborolan-2-yl)vinyl]triazole

A solution of 2,2,6,6-tetramethylpiperidine (9.32 mL, 55.24 mmol) in THF (550 mL) was cooled to −30° C. under Ar atmosphere, before being treated dropwise with a 2.5 M n-butyllithium solution in THF (22.1 mL, 55.24 mmol). The mixture was stirred for 30 min at this temperature, before being cooled down to −78° C. and treated dropwise with a solution of 1-[(4-methoxyphenyl)methyl]triazole-4-carbaldehyde (CAS RN: 853807-54-2; 10.0 g, 46.04 mmol) in THF (100 mL). The mixture was stirred for 30 min at this temperature, before being treated dropwise with a solution of 4,4,5,5-tetramethyl-2-[(4,4,5,5-tetramethyl-1,3,2-dioxaborolan-2-yl)methyl]-1,3,2-dioxaborolane (13.57 g, 50.64 mmol) in THF (50 mL). The mixture was allowed to slowly warm up to 23° C. and stirred for another 16 h at this temperature, before being cooled down to 0° C. and treated dropwise with saturated aqueous NH₄Cl solution (150 mL). The mixture was stirred for 1 h, before being filtered off Water (200 mL) was added to the filtrate, and the mixture was extracted with EtOAc (2×200 mL). The combined organic layers were washed with brine (100 mL), dried over Na₂SO₄, filtered and evaporated. Purification by FC (SiO₂; PE/MTBE) gave the title compound (4.4 g, 26.61% yield) as a white solid. MS (ESI): m/z=342.2 [M+H]⁺

### Step b): methyl 2-chloro-5-fluoro-3-[2-[1-[(4-methoxyphenyl)methyl]triazol-4-yl]vinyl]benzoate

A suspension of 1-[(4-methoxyphenyl)methyl]-4-[2-(4,4,5,5-tetramethyl-1,3,2-dioxaborolan-2-yl)vinyl]triazole (3.0 g, 8.79 mmol), methyl 3-bromo-2-chloro-5-fluoro-benzoate (2.59 g, 9.67 mmol) and potassium carbonate (2.43 g, 17.58 mmol) in 1,4-Dioxane (40 mL) and water (5 mL) was sparged with Ar for 5 minutes, before being treated with Pd(dppf)Cl₂ CH₂Cl₂ (1.44 g, 1.76 mmol). The mixture was stirred at 80° C. for 18 h under Ar, before being cooled down, filtered through SiO₂ (25 g), and washed with dioxane (100 mL). The filtrate was evaporated. Purification by FC (SiO₂; CHCl₃/MTBE) gave the title compound (1.65 g, 44.37% yield) as a light yellow solid. MS (ESI): m/z=402.0 [M+H]⁺

### Step c): methyl 2-chloro-5-fluoro-3-[2-[1-[(4-methoxyphenyl)methyl]triazol-4-yl]ethyl]benzoate

A solution of methyl 2-chloro-5-fluoro-3-[2-[1-[(4-methoxyphenyl)methyl]triazol-4-yl]vinyl]benzoate (1650.0 mg, 4.11 mmol) and Rh/C (10%) (500 mg) in EtOAc (150 mL) was hydrogenated at 7600 mmHg (10 bar) for 18 h at 25° C. The reaction mixture was filtered and evaporated, to give the title compound (1.60 g, 89.73% yield) as a dark green oil. MS (ESI): m/z=404.0 [M+H]⁺

### Step d): 2-chloro-5-fluoro-3-[2-[1-[(4-methoxyphenyl)methyl]triazol-4-yl]ethyl]benzoic acid

A mixture of methyl 2-chloro-5-fluoro-3-[2-[1-[(4-methoxyphenyl)methyl]triazol-4-yl]ethyl]benzoate (1.5 g, 3.71 mmol) in 4:1 Methanol/Water (25 mL) was treated with a solution of lithium hydroxide monohydrate (264.96 mg, 6.31 mmol) in THF (20 mL), at 23° C. The mixture was stirred for 18 h at this temperature, before being evaporated. The residue was treated with water, and the resulting solution was acidified with citric acid (aqueous solution) and extracted with TBME (2×50 mL). The combined organic layers were dried over Na₂SO₄, filtered and evaporated to give the title compound (1.35 g, 88.58% yield) as a light grey solid. MS (ESI): m/z=390.2 [M+H]⁺

### Step e): 2-chloro-5-fluoro-3-[2-(1H-triazol-4-yl)ethyl]benzoic acid;2,2,2-trifluoroacetic acid (C.18)

A solution of 2-chloro-5-fluoro-3-[2-[1-[(4-methoxyphenyl)methyl]triazol-4-yl]ethyl]benzoic acid (1350.0 mg, 3.46 mmol) and anisole (1.88 mL, 17.32 mmol) in trifluoroacetic acid (5.34 mL, 69.26 mmol) was stirred at 70° C. for 120 h (sealed tube). The mixture was then cooled down and purified by RP-HPLC, to give the title compound (C.18; 116.9 mg, 8.36% yield) as a white solid. MS (ESI): m/z=270.0 [M+H]⁺

### Example 88 and Example 89

### N-[1-[3-chloro-5-[(7S)-6-[2-chloro-5-fluoro-3-[2-(1H-triazol-4-yl)ethyl]benzoyl]-2,7-dimethyl-5,7-dihydro-4H-pyrazolo[3,4-c]pyridin-3-yl]phenyl]cyclopropyl]methanesulfonamide (Example 88) and N-[1-[3-chloro-5-[(7R)-6-[2-chloro-5-fluoro-3-[2-(1H-triazol-4-yl)ethyl]benzoyl]-2,7-dimethyl-5,7-dihydro-4H-pyrazolo[3,4-c]pyridin-3-yl]phenyl]cyclopropyl]methanesulfonamide (Example 89)

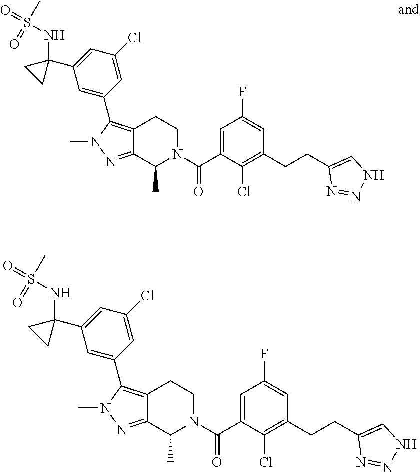 [SMILES: C[C@H]1c2nn(C)c(-c3cc(Cl)cc(C4(NS(C)(=O)=O)CC4)c3)c2CCN1C(=O)c1cc(F)cc(CCc2c[nH]nn2)c1Cl ; C[C@@H]1c2nn(C)c(-c3cc(Cl)cc(C4(NS(C)(=O)=O)CC4)c3)c2CCN1C(=O)c1cc(F)cc(CCc2c[nH]nn2)c1Cl]

N-[1-[3-Chloro-5-[6-[2-chloro-5-fluoro-3-[2-(1H-triazol-4-yl)ethyl]benzoyl]-2,7-dimethyl-5,7-dihydro-4H-pyrazolo[3,4-c]pyridin-3-yl]phenyl]cyclopropyl]methanesulfonamide (Example 87; 18 mg, 0.027 mmol) was separated by chiral SFC, to give Example 88 (4.4 mg, 24%) and Example 89 (6.9 mg, 38%) as white solids. MS (ESI): m/z=644.3 [M+H]⁺

### Example 90

### 4-[2-chloro-3-[(7S)-2,7-dimethyl-3-(2,3,4,5-tetrafluorophenyl)-5,7-dihydro-4H-pyrazolo[3,4-c]pyridine-6-carbonyl]-5-fluoro-phenyl]-1H-pyrrole-2-carbonitrile

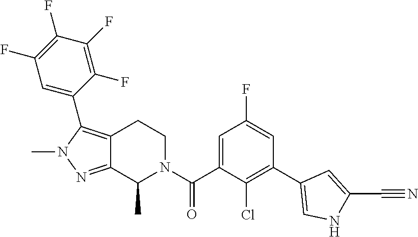 [SMILES: C[C@H]1c2nn(C)c(-c3cc(F)c(F)c(F)c3F)c2CCN1C(=O)c1cc(F)cc(-c2c[nH]c(C#N)c2)c1Cl]

A mixture of 2-chloro-3-(5-cyano-1H-pyrrol-3-yl)-5-fluoro-benzoic acid (C.11; 162.03 mg, 0.55 mmol), 2,7-dimethyl-3-(2,3,4,5-tetrafluorophenyl)-4,5,6,7-tetrahydropyrazolo[3,4-c]pyridine;hydrochloride (B.6; 185.0 mg, 0.55 mmol) and triethylamine (0.38 mL, 2.76 mmol) in DMF (3 mL) was treated with HATU (230.47 mg, 0.61 mmol). The mixture was stirred at 20° C. for 16 h, before being purified by RP-HPLC and chiral HPLC, to give the title compound (54.9 mg, 33%) as a white solid. MS (ESI): m/z=546.0 [M+H]⁺

### Step a): tert-butyl 2,7-dimethyl-3-(2,3,4,5-tetrafluorophenyl)-5,7-dihydro-4H-pyrazolo[3,4-c]pyridine-6-carboxylate

A degassed suspension of 2,3,4,5-tetrafluorophenylboronic acid (631.1 mg, 3.25 mmol), tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (1.0 g, 2.5 mmol) and sodium carbonate (530.75 mg, 5.01 mmol) in DMF (10 mL) was treated with tetrakis(triphenylphosphine)palladium(0) (144.67 mg, 0.13 mmol), at 23° C. The resulting mixture was stirred at 100° C. for 15 h, before being cooled down and purified by RP-HPLC, to give the title compound (223.0 mg, 22.3% yield) as a brown oil. MS (ESI): m/z=344.2 [M-Bu+H]⁺

### Step b): 2,7-dimethyl-3-(2,3,4,5-tetrafluorophenyl)-4,5,6,7-tetrahydropyrazolo[3,4-c]pyridine;hydrochloride (B.6)

A solution of tert-butyl 2,7-dimethyl-3-(2,3,4,5-tetrafluorophenyl)-5,7-dihydro-4H-pyrazolo[3,4-c]pyridine-6-carboxylate (223.0 mg, 0.56 mmol) in DCM (0.5 mL) was treated with 1,4-dioxane HCl solution (0.7 mL, 2.79 mmol), at 23° C. The resulting mixture was stirred for 16 h at this temperature, before being evaporated to give the title compound (B.6; 185 mg, 99% yield) as a brown solid. MS (ESI): m/z=300.0 [M−Bu+H]⁺

### Example 91

### N-[1-[3-chloro-5-[(7S)-6-[1-(5-chloro-2-hydroxy-3-pyridyl)-1,2,4-triazole-3-carbonyl]-2,7-dimethyl-5,7-dihydro-4H-pyrazolo[3,4-c]pyridin-3-yl]phenyl]cyclopropyl]methanesulfonamide

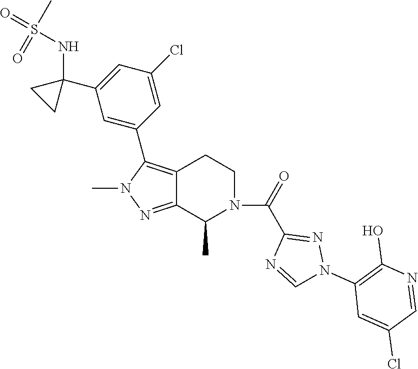 [SMILES: C[C@H]1c2nn(C)c(-c3cc(Cl)cc(C4(NS(C)(=O)=O)CC4)c3)c2CCN1C(=O)c1ncn(-c2cc(Cl)cnc2O)n1]

A solution of HATU (42.31 mg, 0.111 mmol) in N,N-dimethylformamide, extra dry (0.813 mL) was treated with n-ethyldiisopropylamine (158.31 μL, 0.927 mmol) and 1-(5-chloro-2-hydroxy-3-pyridyl)-1,2,4-triazole-3-carboxylic acid (C.10; 22.31 mg, 0.093 mmol), at 23° C. under Ar. The mixture was stirred for 10 min at this temperature, before being treated with N-[1-[3-chloro-5-(2,7-dimethyl-4,5,6,7-tetrahydropyrazolo[3,4-c]pyridin-3-yl)phenyl]cyclopropyl]methanesulfonamide;hydrochloride (B.2; 40 mg, 0.093 mmol), and stirred for another 18 h. Purification by RP-HPLC and chiral HPLC gave the title compound (15.6 mg, 25.88%) as a white solid. MS (ESI): m/z=615.2 [M−H]⁻

### Example 92 and Example 93

### 4-[2-chloro-3-[(7S)-2,7-dimethyl-3-(3,4,5-trifluorophenyl)-5,7-dihydro-4H-pyrazolo[3,4-c]pyridine-6-carbonyl]-5-fluoro-phenyl]-1H-pyrrole-2-carbonitrile (Example 92) and 4-[2-chloro-3-[(7R)-2,7-dimethyl-3-(3,4,5-trifluorophenyl)-5,7-dihydro-4H-pyrazolo[3,4-c]pyridine-6-carbonyl]-5-fluoro-phenyl]-1H-pyrrole-2-carbonitrile (Example 93)

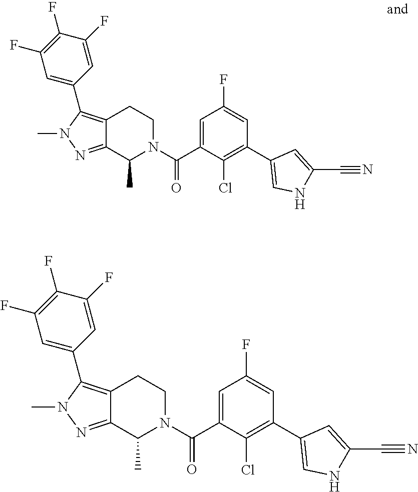 [SMILES: C[C@H]1c2nn(C)c(-c3cc(F)c(F)c(F)c3)c2CCN1C(=O)c1cc(F)cc(-c2c[nH]c(C#N)c2)c1Cl ; C[C@@H]1c2nn(C)c(-c3cc(F)c(F)c(F)c3)c2CCN1C(=O)c1cc(F)cc(-c2c[nH]c(C#N)c2)c1Cl]

A solution of 2-chloro-3-(5-cyano-1H-pyrrol-3-yl)-5-fluoro-benzoic acid (C.11; 150.0 mg, 0.57 mmol) in DMF (2 mL) was treated with 2,7-dimethyl-3-(3,4,5-trifluorophenyl)-4,5,6,7-tetrahydropyrazolo[3,4-c]pyridine;hydrochloride (B.7; 180.1 mg, 0.57 mmol), triethylamine (0.4 mL, 2.83 mmol), and HATU (237.07 mg, 0.62 mmol), at 20° C. The mixture was stirred for 16 h at this temperature. Purification by RP-HPLC and chiral chromatography gave Example 92 (67 mg, 42% yield) and Example 93 (64 mg, 40% yield) as white solids. MS (ESI): m/z=528.0 [M+H]⁺

### Step a): tert-butyl 2,7-dimethyl-3-(3,4,5-trifluorophenyl)-5,7-dihydro-4H-pyrazolo[3,4-c]pyridine-6-carboxylate

A degassed suspension of 3,4,5-trifluorophenylboronic acid (0.63 g, 3.61 mmol), tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (1.2 g, 3.0 mmol) and sodium carbonate (636.9 mg, 6.01 mmol) in DMF (15 mL) was treated with tetrakis(triphenylphosphine)palladium(0) (173.6 mg, 0.15 mmol). The resulting mixture was stirred at 100° C. for 15 h, before being cooled down and diluted with water (30 mL). The mixture was extracted with EtOAc (3×30 mL), and the combined organic layers were washed with brine, dried over sodium sulfate and evaporated. Purification by FC (SiO₂; hexane/EtOAc) gave the title compound (980.0 mg, 79.28% yield) as a white solid. MS (ESI): m/z=326.0 [M+H-tBu]⁺

### Step b): 2,7-dimethyl-3-(3,4,5-trifluorophenyl)-4,5,6,7-tetrahydropyrazolo[3,4-c]pyridine;hydrochloride (B.7)

A solution of tert-butyl 2,7-dimethyl-3-(3,4,5-trifluorophenyl)-5,7-dihydro-4H-pyrazolo[3,4-c]pyridine-6-carboxylate (980.0 mg, 2.57 mmol) in DCM (10 mL) was treated with hydrochloric acid (4 M in dioxane) (6.42 mL, 25.7 mmol). The mixture was stirred at 20° C. for 16 h, before being evaporated to give the title compound (B.7; 680.0 mg, 83.29% yield) as a light yellow solid. MS (ESI): m/z=282.0 [M+H]⁺

### Example 94

### N-[1-[3-chloro-5-[(7S)-6-[2,5-difluoro-3-(1H-pyrazol-4-yl)benzoyl]-2,7-dimethyl-5,7-dihydro-4H-pyrazolo[3,4-c]pyridin-3-yl]phenyl]cyclopropyl]methanesulfonamide

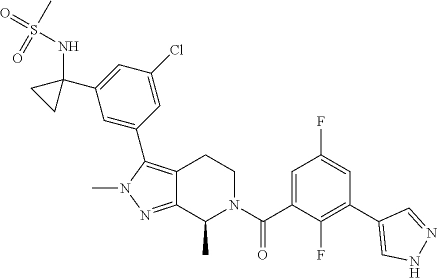 [SMILES: C[C@H]1c2nn(C)c(-c3cc(Cl)cc(C4(NS(C)(=O)=O)CC4)c3)c2CCN1C(=O)c1cc(F)cc(-c2cn[nH]c2)c1F]

A solution of 2,5-difluoro-3-(1H-pyrazol-4-yl)benzoic acid (C.19; 31.18 mg, 0.139 mmol) in tetrahydrofuran, extra dry (2 mL) was treated with HATU (63.46 mg, 0.167 mmol) and DIPEA (97.17 μL, 0.556 mmol) at 20° C. under Ar. The mixture was stirred for 10 min at this temperature before being treated with N-[1-[3-chloro-5-(2,7-dimethyl-4,5,6,7-tetrahydropyrazolo[3,4-c]pyridin-3-yl)phenyl]-cyclopropyl]methanesulfonamide;hydrochloride (B.2; 60 mg, 0.139 mmol). The mixture was stirred for 18 h at this temperature, before being diluted with EtOAc. The organic layer was washed with water (1×) and brine (3×), dried over Na₂SO₄, filtered, and evaporated. Purification by RP-HPLC and chiral SFC gave the title compound (4 mg, 4.55%) as an off-white solid. MS (ESI): m/z=599.4 [M−H]⁻

### Step a): methyl 2,5-difluoro-3-(1H-pyrazol-4-yl)benzoate

A solution of methyl 3-bromo-2,5-difluoro-benzoate (CAS RN: 1524902-93-9; 2.0 g, 7.97 mmol) and 4-pyrazoleboronic acid pinacol ester (CAS RN: 269410-08-4; 1.5 g, 7.97 mmol), and K₂CO₃ (2.2 g, 15.93 mmol) in 1,4-Dioxane (20 mL) and water (4 mL) was treated with 1,1′-bis(diphenylphosphino)ferrocene-palladium(II)dichloride dichloromethane complex (650.15 mg, 0.8 mmol), at 23° C. under N₂. The mixture was stirred for 12 h at 100° C. under N₂ atmosphere for 12 h, before being filtered and evaporated. Purification by RP-HPLC gave the title compound (110.0 mg, 5.8% yield) as a light yellow solid. MS (ESI): m/z=239.3 [M+H]⁺

### Step b): 2,5-difluoro-3-(1H-pyrazol-4-yl)benzoic acid (C.19)

A solution of methyl 2,5-difluoro-3-(1H-pyrazol-4-yl)benzoate (50.0 mg, 0.21 mmol) in methanol (0.5 mL), tetrahydrofuran (0.5 mL) and water (0.5 mL) was treated with LiOH monohydrate (44.04 mg, 1.05 mmol), at 23° C. The mixture was stirred for 6 h at that temperature, before being extracted with EtOAc. The aqueous phase was acidified to pH 4 with 1 N HCl aqueous solution, and the aqueous phase was extracted with EtOAc. The organic layer was dried over Na₂SO₄, filtered, and evaporated, to give the title compound (C.19; 35.0 mg, 74%) as a white solid. MS (ESI): m/z=225.0 [M+H]⁺

### Example 95

### [(7S)-3-[3-chloro-5-(2-hydroxy-2-methyl-propyl)phenyl]-2,7-dimethyl-5,7-dihydro-4H-pyrazolo[3,4-c]pyridin-6-yl]-(2-chloro-3-methoxy-phenyl)methanone

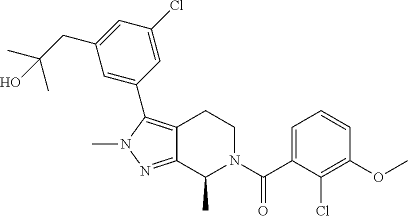 [SMILES: COc1cccc(C(=O)N2CCc3c(nn(C)c3-c3cc(Cl)cc(CC(C)(C)O)c3)[C@@H]2C)c1Cl]

A solution of 2-chloro-3-methoxybenzoic acid (150.0 mg, 0.8 mmol) in DMF (2 mL) was treated with 1-[3-chloro-5-(2,7-dimethyl-4,5,6,7-tetrahydropyrazolo[3,4-c]pyridin-3-yl)phenyl]-2-methyl-propan-2-ol;hydrochloride (B.8; 372.13 mg, 0.8 mmol), triethylamine (0.56 mL, 4.02 mmol), and HATU (336.23 mg, 0.88 mmol). The mixture was stirred at 20° C. for 16 h, and purified by RP-HPLC and chiral SFC, to give the title compound (110 mg, 38%) as a light yellow gum. MS (ESI): m/z=502.2 [M+H]⁺

### Step a): 1-[3-chloro-5-(4,4,5,5-tetramethyl-1,3,2-dioxaborolan-2-yl)phenyl]-2-methyl-propan-2-ol

A mixture of bis(pinacolato)diboron (1.88 g, 7.4 mmol), potassium acetate (0.92 mL, 14.8 mmol), 1-(3-bromo-5-chloro-phenyl)-2-methyl-propan-2-ol (CAS RN: 2229628-26-4; 1.3 g, 4.93 mmol) and 1,1′-bis(diphenylphosphino)ferrocene-palladium(II)dichloride dichloromethane complex (201.24 mg, 0.25 mmol) in 1,4-Dioxane (25 mL) was degassed with Ar and stirred at 100° C. for 16 h under Ar. The mixture was evaporated. Purification by FC (SiO₂; hexane/EtOAc) gave the title compound (1.01 g, 62.63% yield) as a white solid which was directly used in the next step. GC-MS (EI): m/z=294.1 [M-OH]⁺

### Step b): tert-butyl 3-[3-chloro-5-(2-hydroxy-2-methyl-propyl)phenyl]-2,7-dimethyl-5,7-dihydro-4H-pyrazolo[3,4-c]pyridine-6-carboxylate

A suspension of tert-butyl 2,7-dimethyl-3-(trifluoromethylsulfonyloxy)-5,7-dihydro-4H-pyrazolo[3,4-c]pyridine-6-carboxylate (Brevet no WO2020/065613 A1, 2020) (1.01 g, 2.53 mmol), 1-[3-chloro-5-(4,4,5,5-tetramethyl-1,3,2-dioxaborolan-2-yl)phenyl]-2-methyl-propan-2-ol (1.02 g, 3.29 mmol) and sodium carbonate (536.06 mg, 5.06 mmol) in dry DMF (6 mL) was sparged with Ar, before being treated with tetrakis(triphenylphosphine)palladium(0) (146.11 mg, 0.13 mmol). The mixture was stirred at 100° C. for 16 h, before being cooled down, diluted with water (50 mL), and extracted with EtOAc (2×20 mL). The combined organic layers were washed with brine, dried over sodium sulfate, filtered and evaporated. FC (SiO₂; hexane/EtOAc) gave the title compound (330.0 mg, 27.36% yield) as a light yellow oil. MS (ESI): m/z=434.2 [M+H]⁺

### Step c): 1-[3-chloro-5-(2,7-dimethyl-4,5,6,7-tetrahydropyrazolo[3,4-c]pyridin-3-yl)phenyl]-2-methyl-propan-2-ol;hydrochloride (B.8)

A solution of tert-butyl 3-[3-chloro-5-(2-hydroxy-2-methyl-propyl)phenyl]-2,7-dimethyl-5,7-dihydro-4H-pyrazolo[3,4-c]pyridine-6-carboxylate (375.0 mg, 0.79 mmol) in DCM (2 mL) was treated with hydrochloric acid (4 M in dioxane) (1.97 mL, 7.86 mmol). The mixture was stirred at 20° C. for 16 h and evaporated, to give the title compound (B.4; 311.0 mg, 85.44% yield) as a light yellow solid. MS (ESI): m/z=334.2 [M+H]⁺

### Example 96

### 3-[3-[(7R)-2,7-dimethyl-3-[6-(trifluoromethyl)pyrazin-2-yl]-5,7-dihydro-4H-pyrazolo[3,4-c]pyridine-6-carbonyl]-1,2,4-triazol-1-yl]-1H-pyridin-2-one

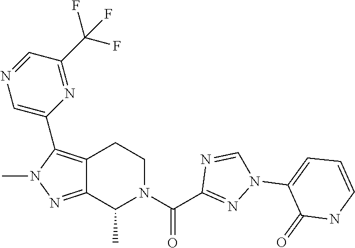 [SMILES: C[C@@H]1c2nn(C)c(-c3cncc(C(F)(F)F)n3)c2CCN1C(=O)c1ncn(-c2ccc[nH]c2=O)n1]

A solution of HATU (61.52 mg, 0.162 mmol) in DMF (1.2 mL) was treated with DIPEA (230.21 μL, 1.35 mmol) and 1-(2-keto-1H-pyridin-3-yl)-1,2,4-triazole-3-carboxylic acid (C.7; 27.8 mg, 0.135 mmol), at 23° C. under Ar. The mixture was stirred for 10 min at this temperature, before being treated with 2,7-dimethyl-3-[6-(trifluoromethyl)pyrazin-2-yl]-4,5,6,7-tetrahydropyrazolo[3,4-c]pyridine;hydrochloride (B.3; 45 mg, 0.135 mmol), and stirred for another 18 h. Purification by RP-HPLC gave the racemate as a white lyophilisate (35 mg). Purification by chiral chromatography gave the title compound (10 mg, 15.28%) as an off-white solid. MS (ESI): m/z=486.4 [M+H]⁺

### Example 97

### 3-chloro-5-[2-chloro-3-[3-(3,5-difluorophenyl)-2,7-dimethyl-5,7-dihydro-4H-pyrazolo[3,4-c]pyridine-6-carbonyl]-5-fluoro-phenyl]-3H-pyridin-2-one

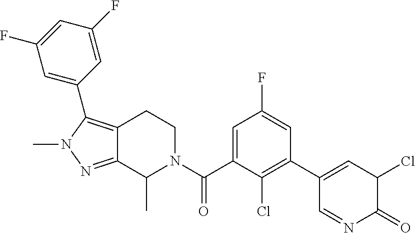 [SMILES: CC1c2nn(C)c(-c3cc(F)cc(F)c3)c2CCN1C(=O)c1cc(F)cc(C2=CC(Cl)C(=O)N=C2)c1Cl]

A suspension of 2-chloro-3-(5-chloro-6-oxo-1H-pyridin-3-yl)-5-fluoro-benzoic acid (C.20; 323.0 mg, 1.07 mmol) in DMF (5 mL) was treated with HATU (487.86 mg, 1.28 mmol), triethylamine (0.6 mL, 4.28 mmol) and 3-(3,5-difluorophenyl)-2,7-dimethyl-4,5,6,7-tetrahydropyrazolo[3,4-c]pyridin-6-ium;chloride (B.5; 320.5 mg, 1.07 mmol). The mixture was stirred at 20° C. for 10 h, before being filtered. The filtrate was evaporated and purified by RP-HPLC, to give the title compound (244.4 mg, 41.76% yield) as a light brown solid. MS (ESI): m/z=547.2 [M+H]⁺

### Step a): methyl 2-chloro-3-(5-chloro-6-methoxy-3-pyridyl)-5-fluoro-benzoate

A solution of methyl 3-bromo-2-chloro-5-fluoro-benzoate (CAS RN: 1805582-40-4; 800.0 mg, 2.99 mmol) and 3-chloro-2-methoxy-5-(4,4,5,5-tetramethyl-1,3,2-dioxaborolan-2-yl)pyridine (CAS RN: 1083168-91-5; 1.05 g, 3.89 mmol) in 1,4-Dioxane (25 mL) was treated with a solution of potassium carbonate (1.24 g, 8.97 mmol) in water (5 mL). The resulting mixture was sparged with Ar and treated with 1,1′-bis(diphenylphosphino)ferrocene-palladium(II)dichloride dichloromethane complex (244.06 mg, 0.3 mmol). The mixture was stirred at 110° C. for 15 h, cooled down, and evaporated. The residue was dissolved in EtOAc and filtered through celite. The filtrate was evaporated, and purification by RP-HPLC gave the title compound (405.0 mg, 41.02% yield) as a white solid. MS (ESI): m/z=330.0 [M+H]⁺

### Step b): 2-chloro-3-(5-chloro-6-oxo-1H-pyridin-3-yl)-5-fluoro-benzoic acid

A mixture of methyl 2-chloro-3-(5-chloro-6-methoxy-3-pyridyl)-5-fluoro-benzoate (405.0 mg, 1.23 mmol) and 12 N aqueous HCl (10.22 mL, 122.68 mmol) was heated at 90° C. for 15 h, before being cooled down, diluted with water, and filtered. The precipitate was washed with ACN and dried, to give the title compound (C.20; 323.0 mg, 81.75% yield) as a light brown solid. MS (ESI): m/z=302.0 [M+H]⁺

### Example 98 and Example 99

### 3-chloro-5-[2-chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-3H-pyridin-2-one (Example 98) and 3-chloro-5-[2-chloro-3-[(7R)-3-(3,5-difluorophenyl)-2,7-dimethyl-5,7-dihydro-4H-pyrazolo[3,4-c]pyridine-6-carbonyl]-5-fluoro-phenyl]-3H-pyridin-2-one (Example 99)

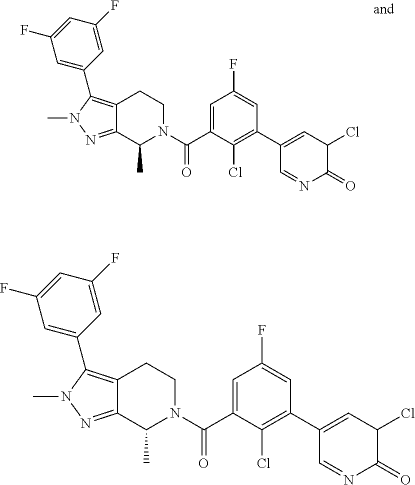 [SMILES: C[C@H]1c2nn(C)c(-c3cc(F)cc(F)c3)c2CCN1C(=O)c1cc(F)cc(C2=CC(Cl)C(=O)N=C2)c1Cl ; C[C@@H]1c2nn(C)c(-c3cc(F)cc(F)c3)c2CCN1C(=O)c1cc(F)cc(C2=CC(Cl)C(=O)N=C2)c1Cl]

Example 97 (87.7 mg, 0.16 mmol) was purified by chiral SFC, to give Example 98 (35.1 mg, 40.34% yield) and Example 99 (38.1 mg, 41.6% yield). MS (ESI): m/z=547.2 [M+H]⁺

### Example 100 and Example 101

### [1-(5-chloro-2-hydroxy-3-pyridyl)-1,2,4-triazol-3-yl]-[(7S)-3-(3,5-difluorophenyl)-2,7-dimethyl-5,7-dihydro-4H-pyrazolo[3,4-c]pyridin-6-yl]methanone (Example 100) and [1-(5-chloro-2-hydroxy-3-pyridyl)-1,2,4-triazol-3-yl]-[(7R)-3-(3,5-difluorophenyl)-2,7-dimethyl-5,7-dihydro-4H-pyrazolo[3,4-c]pyridin-6-yl]methanone (Example 101)

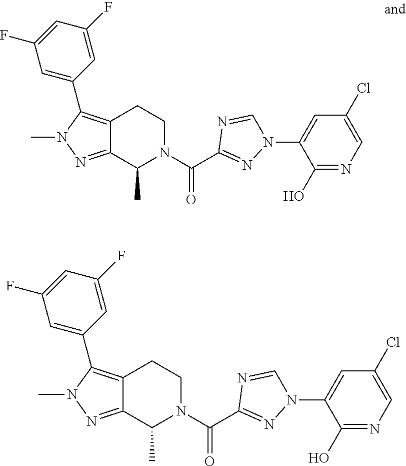 [SMILES: C[C@H]1c2nn(C)c(-c3cc(F)cc(F)c3)c2CCN1C(=O)c1ncn(-c2cc(Cl)cnc2O)n1 ; C[C@@H]1c2nn(C)c(-c3cc(F)cc(F)c3)c2CCN1C(=O)c1ncn(-c2cc(Cl)cnc2O)n1]

A solution of 3-(3,5-difluorophenyl)-2,7-dimethyl-4,5,6,7-tetrahydropyrazolo[3,4-c]pyridine;hydrochloride (B.5; 168.19 mg, 0.56 mmol), HATU (256.02 mg, 0.67 mmol) and triethylamine (0.39 mL, 2.81 mmol) in DMF (2 mL) was treated with 1-(5-chloro-2-hydroxy-3-pyridyl)-1,2,4-triazole-3-carboxylic acid (C.10; 135.0 mg, 0.56 mmol). The resulting mixture was stirred for 16 h at 20° C., before being purified by RP-HPLC to give the racemic mixture (150.0 mg, 55.02% yield) as an orange solid. Chiral separation gave Example 100 (86.5 mg, 25.28% yield) and Example 101 (121.4 mg, 35.49% yield) as light brown solids. MS (ESI): m/z=486.2 [M+H]⁺

### Example 102 and Example 103

### [2-chloro-5-fluoro-3-[2-(JH-pyrazol-4-yl)ethyl]phenyl]-[(7S)-3-(3,5-difluorophenyl)-2,7-dimethyl-5,7-dihydro-4H-pyrazolo[3,4-c]pyridin-6-yl]methanone (Example 102) and [2-chloro-5-fluoro-3-[2-(1H-pyrazol-4-yl)ethyl]phenyl]-[(7R)-3-(3,5-difluorophenyl)-2,7-dimethyl-5,7-dihydro-4H-pyrazolo[3,4-c]pyridin-6-yl]methanone (Example 103)

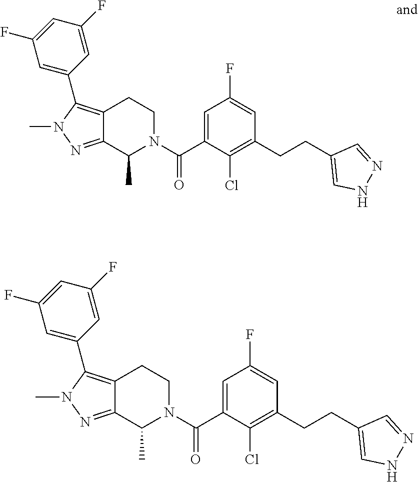 [SMILES: C[C@H]1c2nn(C)c(-c3cc(F)cc(F)c3)c2CCN1C(=O)c1cc(F)cc(CCc2cn[nH]c2)c1Cl ; C[C@@H]1c2nn(C)c(-c3cc(F)cc(F)c3)c2CCN1C(=O)c1cc(F)cc(CCc2cn[nH]c2)c1Cl]

A solution of 3-(3,5-difluorophenyl)-2,7-dimethyl-4,5,6,7-tetrahydropyrazolo[3,4-c]pyridine;hydrochloride (B.5; 200.0 mg, 0.67 mmol), 2-chloro-5-fluoro-3-[2-(1H-pyrazol-4-yl)ethyl]benzoic acid;hydrochloride (C.21; 223.95 mg, 0.73 mmol) and HATU (380.55 mg, 1.0 mmol) in DMF (3 mL) was treated with N,N-diisopropylethylamine (0.58 mL, 3.34 mmol), at 23° C. The mixture was stirred for 18 h at this temperature. Purification by RP-HPLC and chiral chromatography gave Example 102 (36.0 mg, 10.5% yield) and Example 103 (30.3 mg, 8.84% yield) as light brown solids. MS (ESI): m/z=514.0 [M+H]⁺

### Step a): methyl 2-chloro-5-fluoro-3-(2-trimethylsilylethynyl)benzoate

A solution of trimethylsilylacetylene (5.59 mL, 39.26 mmol) in DMF (14 mL) and triethylamine (182.38 mL, 1308.51 mmol) was treated with 1,1′-bis(diphenylphosphino)ferrocene-palladium(II)dichloride dichloromethane complex (2.13 g, 2.62 mmol), copper iodide (249.21 mg, 1.31 mmol) and triethylamine (182.38 mL, 1308.51 mmol), at 23° C. The mixture was sparged with Ar and stirred for 18 h at 88° C., before being cooled down, filtered, and evaporated. The residue was dissolved in water and extracted with MTBE, and the combined organic layers were dried over Na₂SO₄, filtered and evaporated, to give the title compound (6.7 g, 86.3% yield) as a brown oil. GC-MS (EI): m/z=284.0 [M]⁺

### Step b): methyl 2-chloro-3-ethynyl-5-fluoro-benzoate

A solution of methyl 2-chloro-5-fluoro-3-(2-trimethylsilylethynyl)benzoate (3.81 g, 13.38 mmol) in THF (127 mL) was treated dropwise with tetrabutylammonium fluoride (20.07 mL, 20.07 mmol) and stirred for 3 h at 23° C., before being evaporated. The residue was dissolved in EtOAc (500 mL) and the organic layer was washed with water (2×1 L) and brine (1×500 mL). The organic layer was dried over Na₂SO₄, filtered and evaporated, to give the title compound (2.8 g, 98.45% yield) as a dark brown liquid. GC-MS (EI): m/z=212.0 [M]⁺

### Step c): methyl 2-chloro-5-fluoro-3-[2-(1-tetrahydropyran-2-ylpyrazol-4-yl)ethynyl]benzoate

A solution of 4-iodo-1-tetrahydropyran-2-yl-pyrazole (1.96 g, 7.06 mmol) in DMF (5.6998 mL) and triethylamine (32.78 mL, 235.18 mmol) was treated with 1,1′-bis(diphenylphosphino)ferrocene-palladium(II)dichloride dichloromethane complex (384 mg, 0.47 mmol), copper iodide (89.58 mg, 0.47 mmol) and methyl 2-chloro-3-ethynyl-5-fluoro-benzoate (1.0 g, 4.7 mmol), and sparged with Ar. The mixture was stirred for 18 h at 88° C., before being cooled down and filtered. The filtrate was evaporated and the residue was dissolved in water. The aqueous layer was extracted with MTBE, and the organic layer was dried over Na₂SO₄, filtered and evaporated. Purification by FC (SiO₂; hexane/EtOAc) gave the title compound (900.0 mg, 2.48 mmol, 52.74% yield) as a light yellow viscous oil. MS (ESI): m/z=362.1 [M+H]⁺

### Step d): methyl 2-chloro-5-fluoro-3-[2-(1-tetrahydropyran-2-ylpyrazol-4-yl)ethyl]benzoate

A solution of methyl 2-chloro-5-fluoro-3-[2-(1-tetrahydropyran-2-ylpyrazol-4-yl)ethynyl]benzoate (900.0 mg, 2.48 mmol) and Rh/C (10%) (150.0 mg) in EtOAc (100 mL) was hydrogenated at 3800 mmHg (in an autoclave) for 18 h at 25° C. The mixture was filtered and evaporated, to give the title compound (900.0 mg, 90.99% yield) as a light yellow viscous oil. MS (ESI): m/z=367.0 [M+H]⁺

### Step e): 2-chloro-5-fluoro-3-[2-(1-tetrahydropyran-2-ylpyrazol-4-yl)ethyl]benzoicacid

A solution of methyl 2-chloro-5-fluoro-3-[2-(1-tetrahydropyran-2-ylpyrazol-4-yl)ethyl]benzoate (900.0 mg, 2.45 mmol) in THF (48 mL), Methanol (48 mL) and water (12 mL) was treated with lithium hydroxide monohydrate (412 mg, 9.81 mmol) at 23° C. The mixture was stirred for 18 h at this temperature, before being diluted with EtOAc and treated with a saturated citric acid solution. The layers were separated, and the organic layer was washed with water. The combined aqueous layers were extracted with EtOAc. The combined organic layers were dried over Na₂SO₄, filtered and evaporated, to give the title compound (900.0 mg, 96.7% yield) as a light yellow solid. MS (ESI): m/z=353.0 [M+H]⁺

### Step f): 2-chloro-5-fluoro-3-[2-(1H-pyrazol-4-yl)ethyl]benzoic acid:hydrochloride

2-Chloro-5-fluoro-3-[2-(1-tetrahydropyran-2-ylpyrazol-4-yl)ethyl]benzoic acid (600.0 mg, 1.7 mmol) was treated with 4 N HCl in 1,4-Dioxane (10 mL), and the mixture was stirred for 48 h at 23° C. before being filtered. The precipitate was washed with ether and dried, to give the title compound (C.21; 407.7 mg, 78.6% yield) as a white solid. MS (ESI): m/z=270.0 [M+H]⁺

### Example 104 and Example 105

### [2-chloro-5-fluoro-3-[2-(JH-triazol-4-yl)ethyl]phenyl]-[(7S)-3-(3,5-difluorophenyl)-2,7-dimethyl-5,7-dihydro-4H-pyrazolo[3,4-c]pyridin-6-yl]methanone (Example 104) and [2-chloro-5-fluoro-3-[2-(JH-triazol-4-yl)ethyl]phenyl]-[(7R)-3-(3,5-difluorophenyl)-2,7-dimethyl-5,7-dihydro-4H-pyrazolo[3,4-c]pyridin-6-yl]methanone (Example 105)

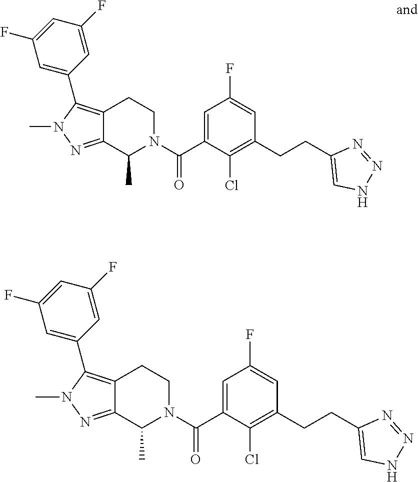 [SMILES: C[C@H]1c2nn(C)c(-c3cc(F)cc(F)c3)c2CCN1C(=O)c1cc(F)cc(CCc2c[nH]nn2)c1Cl ; C[C@@H]1c2nn(C)c(-c3cc(F)cc(F)c3)c2CCN1C(=O)c1cc(F)cc(CCc2c[nH]nn2)c1Cl]

A solution of 2-chloro-5-fluoro-3-[2-(1H-triazol-4-yl)ethyl]benzoic acid;2,2,2-trifluoroacetic acid (C.18; 95.0 mg, 0.25 mmol), 3-(3,5-difluorophenyl)-2,7-dimethyl-4,5,6,7-tetrahydropyrazolo[3,4-c]pyridine;hydrochloride (B.5; 74.22 mg, 0.25 mmol) and HATU (122 mg, 0.32 mmol) in DMF (5 mL) was treated with triethylamine (0.21 mL, 1.49 mmol), at 23° C. The mixture was stirred for 48 h at this temperature, before being purified by RP-HPLC and chiral chromatography, to give Example 104 (20.4 mg, 16.0% yield) as a light grey solid and Example 105 (26.7 mg, 20.94% yield) as a light brown solid. MS (ESI): m/z=515.2 [M+H]⁺

### Example 106

### 4-[[2-chloro-3-[3-(3,5-difluorophenyl)-2,7-dimethyl-5,7-dihydro-4H-pyrazolo[3,4-c]pyridine-6-carbonyl]-5-fluoro-phenyl]methylamino]pyrrolidin-2-one;hydrochloride

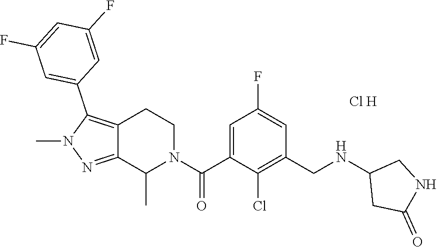 [SMILES: CC1c2nn(C)c(-c3cc(F)cc(F)c3)c2CCN1C(=O)c1cc(F)cc(CNC2CNC(=O)C2)c1Cl ; "Cl H"]

A solution of tert-butyl N-[[2-chloro-3-[3-(3,5-difluorophenyl)-2,7-dimethyl-5,7-dihydro-4H-pyrazolo[3,4-c]pyridine-6-carbonyl]-5-fluoro-phenyl]methyl]-N-(5-oxopyrrolidin-3-yl)carbamate (12.5 mg, 0.02 mmol) in DCM (0.5 mL) was treated with hydrochloric acid (4 M in dioxane) (0.05 mL, 0.2 mmol). The mixture was stirred at 20° C. for 16 h and evaporated, to give the title compound (9.9 mg, 80.14% yield) as a white solid. MS (ESI): m/z=532.2 [M+H]⁺

### Step a): methyl 2-chloro-5-fluoro-3-[[(5-oxopyrrolidin-3-yl)amino]methyl]benzoate

A solution of 4-aminopyrrolidin-2-one;hydrochloride (CAS RN: 167465-93-2; 83.1 mg, 0.6 mmol) in 1:2.5 DCM/IPA (14 mL) was treated with trimethylamine (0.09 mL, 0.7 mmol), methyl 2-chloro-5-fluoro-3-formyl-benzoate (CAS RN: 2384295-46-7; 155 mg, 0.6 mmol), and sodium cyanoborohydride (114.7 mg, 3 mmol), at 23° C. The mixture was stirred for 16 h at this temperature, before being diluted with water and EtOAc. The organic layer was washed with saturated aqueous NaHCO₃ and brine, dried over sodium sulfate, filtered, and evaporated, to give the title compound (185 mg, 86%) as a light brown oil. ¹H NMR (400 MHz, DMSO-d₆) δ=7.58 (dd, J=3.1, 9.4 Hz, 1H), 7.53-7.48 (m, 2H), 3.86 (s, 3H), 3.78 (d, J=3.5 Hz, 2H), 3.48-3.36 (m, 2H), 3.01 (dd, J=3.7, 8.9 Hz, 1H), 2.41-2.31 (m, 1H), 2.03-1.95 ppm (m, 1H)

### Step b): 2-chloro-5-fluoro-3-[[(5-oxopyrrolidin-3-yl)amino]methyl]benzoic acid

A solution of methyl 2-chloro-5-fluoro-3-[[(5-oxopyrrolidin-3-yl)amino]methyl]benzoate (240.0 mg, 0.678 mmol) in MeOH (2.0 mL) was treated with NaOH (32.5 mg, 0.814 mmol), at 23° C. The mixture was stirred for 16 h at this temperature, before being evaporated, to give the title compound (150.0 mg, 62% yield) as a light yellow viscous oil. MS (ESI): m/z=287.1 [M+H]⁺

### Step c): 3-[[tert-butoxycarbonyl-(5-oxopyrrolidin-3-yl)amino]methyl]-2-chloro-5-fluoro-benzoic acid

A solution of 2-chloro-5-fluoro-3-[[(5-oxopyrrolidin-3-yl)amino]methyl]benzoic acid (100.0 mg, 0.35 mmol) in DCM (5 mL) was treated with di-t-butyldicarbonate (76.13 mg, 0.35 mmol). The mixture was cooled down to 0° C. and triethylamine (0.07 mL, 0.52 mmol) was added dropwise. The mixture was warmed to 20° C., and stirred for 16 h before being diluted with water. The organic layer was separated, washed with brine, dried over sodium sulfate and evaporated to give the title compound (96.0 mg, 64.04% yield). MS (ESI): m/z=387.2 [M+H]⁺

### Step d): tert-butyl N-[[2-chloro-3-[3-(3,5-difluorophenyl)-2,7-dimethyl-5,7-dihydro-4H-pyrazolo[3,4-c]pyridine-6-carbonyl]-5-fluoro-phenyl]methyl]-N-(5-oxopyrrolidin-3-yl)carbamate

A solution of 3-[[tert-butoxycarbonyl-(5-oxopyrrolidin-3-yl)amino]methyl]-2-chloro-5-fluoro-benzoic acid (96.0 mg, 0.25 mmol) in DMF (1 mL) was treated with 3-(3,5-difluorophenyl)-2,7-dimethyl-4,5,6,7-tetrahydropyrazolo[3,4-c]pyridin-6-ium;chloride (B.5; 74.4 mg, 0.25 mmol), triethylamine (0.1 mL, 0.74 mmol) and HATU (113.24 mg, 0.3 mmol), at 20° C. The mixture was stirred for 10 h at this temperature, before being purified by RP-HPLC, to give the title compound (11.5 mg, 6.96% yield) as a yellow viscous oil. MS (ESI): m/z=532.2 [M+H-Boc]⁺

### Example 107

### 5-[[2,5-dichloro-3-[(7S)-3-(3,5-difluorophenyl)-2,7-dimethyl-5,7-dihydro-4H-pyrazolo[3,4-c]pyridine-6-carbonyl]phenoxy]methyl]oxazolidin-2-one

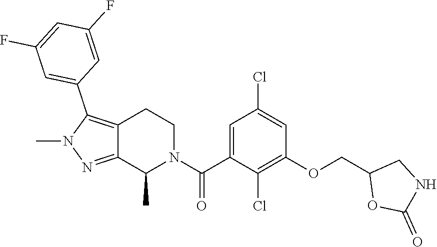 [SMILES: C[C@H]1c2nn(C)c(-c3cc(F)cc(F)c3)c2CCN1C(=O)c1cc(Cl)cc(OCC2CNC(=O)O2)c1Cl]

A solution of 2,5-dichloro-3-[(2-oxooxazolidin-5-yl)methoxy]benzoic acid (205.0 mg, 0.57 mmol) and 3-(3,5-difluorophenyl)-2,7-dimethyl-4,5,6,7-tetrahydropyrazolo[3,4-c]pyridin-6-ium;chloride (B.5; 170.64 mg, 0.57 mmol) in DMF (0.5 mL) was treated with triethylamine (0.24 mL, 1.71 mmol) and HATU (259.74 mg, 0.68 mmol), at 20° C. The mixture was stirred for 10 h at this temperature, before being evaporated. Purification by RP-HPLC and chiral SFC gave the title compound (69 mg, 26%) as a yellow solid. MS (ESI): m/z=551.0 [M+H]⁺

### Step a): ethyl 2,5-dichloro-3-(oxiran-2-ylmethoxy)benzoate

A solution of ethyl 2,5-dichloro-3-hydroxy-benzoate (CAS RN: 1806282-15-4; 850.0 mg, 3.62 mmol) and epichlorohydrin (0.85 mL, 10.85 mmol) in Acetone (30 mL) was treated with potassium carbonate (999.53 mg, 7.23 mmol). The mixture was heated at 60° C. for 16 h, cooled down and filtered. The filtrate was concentrated, the residue was dissolved in EtOAc. The organic layer was washed with aqueous K₂CO₃, water and brine, dried over sodium sulfate and evaporated to give the title compound (1.05 g, 89.76% yield) as a light yellow oil. MS (ESI): m/z=242.0 [M+H]⁺

### Step b): ethyl 2,5-dichloro-3-[(2-oxooxazolidin-5-yl)methoxy]benzoate

A solution of ethyl 2,5-dichloro-3-(oxiran-2-ylmethoxy)benzoate (1050.0 mg, 3.61 mmol), urethane (321 mg, 3.61 mmol) and triethylamine (0.1 mL, 0.72 mmol) in DMF (10 mL) was heated at 130° C. for 15 h. The mixture was then cooled down and evaporated. Purification by RP-HPLC gave the title compound (375.0 mg, 30.59% yield) as a light yellow solid. MS (ESI): m/z=334.0 [M+H]⁺

### Step c): 2,5-dichloro-3-[(2-oxooxazolidin-5-yl)methoxy]benzoic acid

A solution of ethyl 2,5-dichloro-3-[(2-oxooxazolidin-5-yl)methoxy]benzoate (375.0 mg, 1.12 mmol) in THF (3 mL)/water (3 mL) was treated with lithium hydroxide (67.2 mg, 2.81 mmol), at 0° C. The mixture was warmed up to 20° C., stirred for 16 h at that temperature, and evaporated. The residue was dissolved in water (15 mL) and acidified with 1 N aqueous HCl to pH=2. The precipitate was filtered and dried under vacuum to give the title compound (205.0 mg, 50.72% yield) as a white powder. MS (ESI): m/z=307.1 [M+H]⁺

### Example 108 and Example 109 (arbitrary assignment of the stereochemistry)

### (5S)-5-[[2,5-dichloro-3-[(7S)-3-(3,5-difluorophenyl)-2,7-dimethyl-5,7-dihydro-4H-pyrazolo[3,4-c]pyridine-6-carbonyl]phenoxy]methyl]oxazolidin-2-one (Example 108) and (5R)-5-[[2,5-dichloro-3-[(7S)-3-(3,5-difluorophenyl)-2,7-dimethyl-5,7-dihydro-4H-pyrazolo[3,4-c]pyridine-6-carbonyl]phenoxy]methyl]oxazolidin-2-one (Example 109)

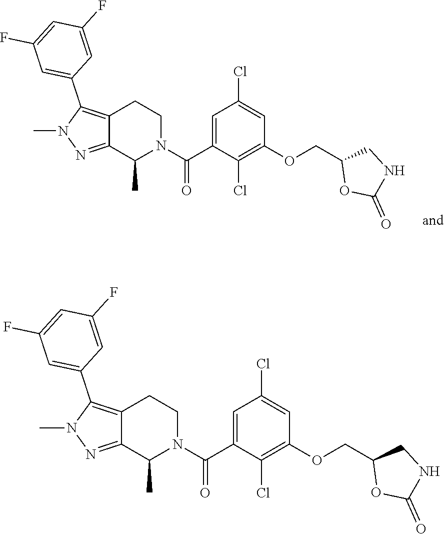 [SMILES: C[C@H]1c2nn(C)c(-c3cc(F)cc(F)c3)c2CCN1C(=O)c1cc(Cl)cc(OC[C@@H]2CNC(=O)O2)c1Cl ; C[C@H]1c2nn(C)c(-c3cc(F)cc(F)c3)c2CCN1C(=O)c1cc(Cl)cc(OC[C@H]2CNC(=O)O2)c1Cl]

Example 107 (5-[[2,5-dichloro-3-[(7S)-3-(3,5-difluorophenyl)-2,7-dimethyl-5,7-dihydro-4H-pyrazolo[3,4-c]pyridine-6-carbonyl]phenoxy]methyl]oxazolidin-2-one) (110 mg, 0.2 mmol) was separated by chiral SFC, to give Example 108 (39.7 mg, 36.1%) and Example 109 (39.2 mg, 35.6%) as white solids. MS (ESI): m/z=551.0 [M+H]⁺

### Example 110

### 5-[[2-chloro-3-[(7S)-3-(3,5-difluorophenyl)-2,7-dimethyl-5,7-dihydro-4H-pyrazolo[3,4-c]pyridine-6-carbonyl]phenoxy]methyl]oxazolidin-2-one

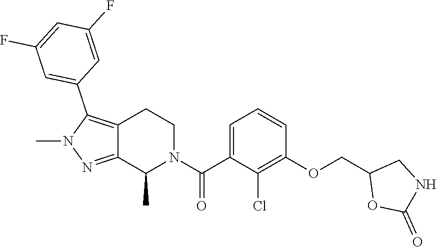 [SMILES: C[C@H]1c2nn(C)c(-c3cc(F)cc(F)c3)c2CCN1C(=O)c1cccc(OCC2CNC(=O)O2)c1Cl]

A solution of 2-chloro-3-[(2-oxooxazolidin-5-yl)methoxy]benzoic acid (200.0 mg, 0.74 mmol) and 3-(3,5-difluorophenyl)-2,7-dimethyl-4,5,6,7-tetrahydropyrazolo[3,4-c]pyridin-6-ium;chloride (B.5; 220.69 mg, 0.74 mmol) in DMF (4 mL) was treated with triethylamine (0.41 mL, 2.94 mmol) and HATU (335.93 mg, 0.88 mmol), at 20° C. The mixture was stirred for 10 h at this temperature, before being evaporated. Purification by RP-HPLC and SFC gave the title compound (122 mg, 39%) as a light brown solid. MS (ESI): m/z=517.2 [M+H]⁺

### Step a): methyl 2-chloro-3-(oxiran-2-ylmethoxy)benzoate

A solution of methyl 2-chloro-3-hydroxy-benzoate (200.0 mg, 1.07 mmol) and epichlorohydrin (198.34 mg, 2.14 mmol) in ACN (5 mL) was treated with potassium carbonate (370.35 mg, 2.68 mmol). The mixture was heated at 80° C. for 15 h, before being cooled down and filtered. The filtrate was evaporated, to give the title compound (170 mg, 56%) as a colorless oil. MS (ESI): m/z=242.0 [M+H]⁺

### Step b): methyl 2-chloro-3-[(2-oxooxazolidin-5-yl)methoxy]benzoate

A solution of methyl 2-chloro-3-(oxiran-2-ylmethoxy)benzoate (170.0 mg, 0.56 mmol), urethane (49.93 mg, 0.56 mmol) and triethylamine (0.02 mL, 0.11 mmol) in DMF (4 mL) was heated at 130° C. for 15 h. The mixture was cooled down and evaporated. Purification by RP-HPLC gave the title compound (64.0 mg, 39.97% yield) as a white solid. MS (ESI): m/z=286.0 [M+H]⁺

### Step c): 2-chloro-3-[(2-oxooxazolidin-5-yl)methoxy]benzoic acid

A solution of methyl 2-chloro-3-[(2-oxooxazolidin-5-yl)methoxy]benzoate (50.0 mg, 0.18 mmol) in THF (1 mL)/water (1 mL) was treated with lithium hydroxide (0.0 mL, 0.53 mmol), at 20° C. The mixture was stirred for 15 h at this temperature, before being evaporated. The residue was dissolved in water (5 mL) and acidified with 1 N HCl (aq.) to pH=2. The precipitate was filtered and dried under vacuum to give the title compound (34.0 mg, 71.51% yield) as a white powder. MS (ESI): m/z=270.0 [M−H]⁻

### Example 111

### [2-chloro-3-(1,6-diazaspiro[3.3]heptan-6-yl)-5-fluoro-phenyl]-[(7S)-3-(3,5-difluorophenyl)-2,7-dimethyl-5,7-dihydro-4H-pyrazolo[3,4-c]pyridin-6-yl]methanone;2,2,2-trifluoroacetic acid

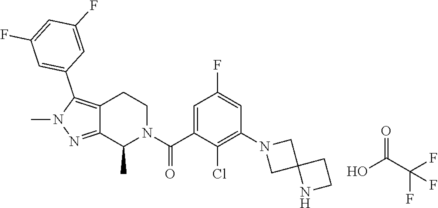

A solution of tert-butyl 6-[2-chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-1,6-diazaspiro[3.3]heptane-1-carboxylate (60.0 mg, 0.1 mmol) in DCM (1 mL) was treated with trifluoroacetic acid (0.04 mL, 0.49 mmol) and stirred at 25° C. for 18 h, before being evaporated. Purification by RP-HPLC gave the title compound (2.8 mg, 4.56% yield) as a light brown solid. MS (ESI): m/z=516.0 [M+H]⁺

### Step a): tert-butyl 6-(2-chloro-5-fluoro-3-methoxycarbonyl-phenyl)-1,6-diazaspiro[3.3]heptane-1-carboxylate

In a sealed tube were added methyl 3-bromo-2-chloro-5-fluoro-benzoate (CAS RN: 1805582-40-4; 1.14 g, 4.26 mmol), 9,9-dimethyl-4,5-bis(diphenylphosphino)xanthene (123.14 mg, 0.210 mmol), tris(dibenzylideneacetone)dipalladium (0) (194.88 mg, 0.210 mmol), tert-butyl 1,6-diazaspiro[3.3]heptane-1-carboxylate;hydrochloride (1998.12 mg, 8.51 mmol), cesium carbonate (5.55 g, 17.03 mmol) and 9,9-dimethyl-4,5-bis(diphenylphosphino)xanthene (123.14 mg, 0.210 mmol) and 1,4-Dioxane (91.08 mL). The mixture was sparged with Argon, heated to 100° C., and stirred for 18 h at this temperature before being cooled down and evaporated. Purification by FC (SiO₂; hexane/MTBE) gave the title (750 mg, 45.79% yield) as a light yellow solid. MS (ESI): m/z=385.0 [M+H]⁺

### Step b): 3-(1-tert-butoxycarbonyl-1,6-diazaspiro[3.3]heptan-6-yl)-2-chloro-5-fluoro-benzoic acid

A solution of tert-butyl 6-(2-chloro-5-fluoro-3-methoxycarbonyl-phenyl)-1,6-diazaspiro[3.3]heptane-1-carboxylate (900.0 mg, 2.34 mmol) in THF (14.4 mL), methanol (14.4 mL) and water (3.6 mL) was treated with hydroxylithium;hydrate (392.53 mg, 9.35 mmol), at 23° C. The mixture was stirred for 18 h, before being diluted with EtOAc and citric acid (aq). The layers were separated, and the organic layer was washed with water. The combined aqueous layers were extracted with EtOAc. The combined organic layers were dried over Na₂SO₄, filtered, and evaporated, to give the title compound (800.0 mg, 85.79% yield) as a light yellow solid. MS (ESI): m/z=371 [M+H]⁺

### Step c): tert-butyl 6-[2-chloro-5-fluoro-3-[rac-(7S)-3-(3,5-difluorophenyl)-2,7-dimethyl-5,7-dihydro-4H-pyrazolo[3,4-c]pyridine-6-carbonyl]phenyl]-1,6-diazaspiro[3.3]heptane-1-carboxylate

A solution of 3-(3,5-difluorophenyl)-2,7-dimethyl-4,5,6,7-tetrahydropyrazolo[3,4-c]pyridine;hydrochloride (B.5; 150.0 mg, 0.5 mmol), in DMF (3 mL) was treated with 3-(1-tert-butoxycarbonyl-1,6-diazaspiro[3.3]heptan-6-yl)-2-chloro-5-fluoro-benzoic acid (204.11 mg, 0.55 mmol), HATU (285.41 mg, 0.75 mmol) and N,N-diisopropylethylamine (0.44 mL, 2.5 mmol), at 23° C. The mixture was stirred at this temperature for 18 h, before being purified by RP-HPLC and chiral SFC, to give the title compound (61.0 mg, 19.79% yield) as a light brown solid. MS (ESI): m/z=514.0 [M+H]⁺

### Example 112

### [(7S)-3-(3,5-difluorophenyl)-2,7-dimethyl-5,7-dihydro-4H-pyrazolo[3,4-c]pyridin-6-yl]-[1-(2-hydroxy-4-pyridyl)-1,2,4-triazol-3-yl]methanone

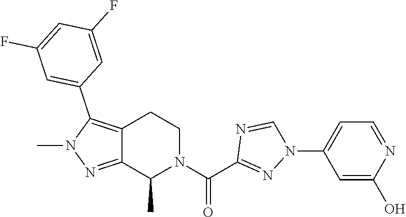 [SMILES: C[C@H]1c2nn(C)c(-c3cc(F)cc(F)c3)c2CCN1C(=O)c1ncn(-c2ccnc(O)c2)n1]

A solution of 1-(2-hydroxy-4-pyridyl)-1,2,4-triazole-3-carboxylic acid (115.0 mg, 0.56 mmol) in DMF (2 mL), 3-(3,5-difluorophenyl)-2,7-dimethyl-4,5,6,7-tetrahydropyrazolo[3,4-c]pyridine;hydrochloride (B.5; 167.21 mg, 0.56 mmol), triethylamine (0.39 mL, 2.79 mmol), and HATU (233.31 mg, 0.61 mmol) in DMF (3 mL) was stirred for 16 h at 20° C. Purification by RP-HPLC and chiral SFC gave the title compound (30.2 mg, 41%) as a yellow solid. MS (ESI): m/z=452.6 [M+H]⁺

### Step a): methyl 1-(2-methoxy-4-pyridyl)-1,2,4-triazole-3-carboxylate

A suspension of methyl 1H-1,2,4-triazole-3-carboxylate (415.52 mg, 3.27 mmol), 2-methoxypyridine-4-boronic acid (500.0 mg, 3.27 mmol), pyridine (0.79 mL, 9.81 mmol), cupric acetate (890.71 mg, 4.9 mmol) and molecular sieves (4 Å, 500 mg) in DCM (20 mL) was stirred at 30° C. for 16 h (open to atmosphere). The resulting mixture was filtered, evaporated and purified by RP-HPLC, to give the title compound (105.0 mg, 13.71% yield) as a light yellow solid. MS (ESI): m/z=235.2 [M+H]⁺

### Step b): 1-(2-hydroxy-4-pyridyl)-1,2,4-triazole-3-carboxylic acid

A solution of methyl 1-(2-methoxy-4-pyridyl)-1,2,4-triazole-3-carboxylate (190.0 mg, 0.81 mmol) in concentrated aqueous hydrochloric acid (4.87 mL, 48.67 mmol) was stirred at 90° C. for 16 h. The resulting precipitate was filtered, washed with water and air-dried to give the title compound (115.0 mg, 65.32% yield) as a white solid. MS (ESI): m/z=286.0 [M+H]⁺

### Example 113 and Example 114

### (4S)-4-[[2-chloro-3-[(7S)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one (Example 113) and (4S)-4-[[2-chloro-3-[(7R)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one (Example 114)

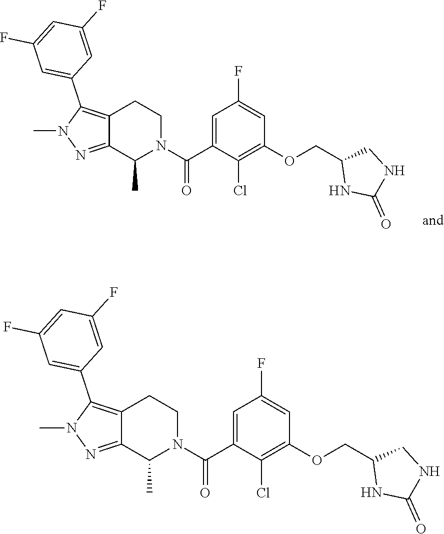

A solution of 2-chloro-5-fluoro-3-[[(4S)-2-oxoimidazolidin-4-yl]methoxy]benzoic acid (45.0 mg, 0.16 mmol) in DMF (1 mL) was treated with HATU (71.13 mg, 0.19 mmol), 3-(3,5-difluorophenyl)-2,7-dimethyl-4,5,6,7-tetrahydropyrazolo[3,4-c]pyridin-6-ium;chloride (B.5; 46.73 mg, 0.16 mmol) and triethylamine (0.09 mL, 0.62 mmol), at 20° C. The mixture was stirred for 15 h at this temperature, before being evaporated. Purification by RP-HPLC (4S)-4-[[2-chloro-3-[3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one (33.9 mg, 40.73% yield) as a pink solid. Further purification by chiral SFC gave Example 113 (6.6 mg, 19.47% yield) and Example 114 (6.9 mg, 20.15% yield) as light brown solids. MS (ESI): m/z=534.2 [M+H]⁺

### Step a): 4-(bromomethyl)imidazolidin-2-one

A solution of triphenylphosphine (3.73 g, 14.21 mmol) in DCM (30 mL) was treated dropwise with bromine (0.73 mL, 14.21 mmol), at −10° C. under Ar. The mixture was stirred for 30 min at this temperature, and 4-(hydroxymethyl)imidazolidin-2-one (CAS RN: 2248182-92-3; 1100.0 mg, 9.47 mmol) was added. The mixture was warmed to 25° C. and stirred for 15 h, before being evaporated. Purification by FC (SiO₂; ACN/MTBE) gave the title compound (330.0 mg, 19.46% yield) as a white solid. MS (ESI): m/z=179.0 [M+H]⁺

### Step b): ethyl 2-chloro-5-fluoro-3-[[(4S)-2-oxoimidazolidin-4-yl]methoxy]benzoate

A solution of ethyl 2-chloro-5-fluoro-3-hydroxy-benzoate (CAS RN: 2090950-46-0; 362.68 mg, 1.66 mmol, 0.9 eq) in DMF (5 mL) was treated with potassium carbonate (509.53 mg, 3.69 mmol). The mixture was stirred at 20° C. for 1 h before 4-(bromomethyl)imidazolidin-2-one (330.0 mg, 1.84 mmol) was added. The resulting mixture was heated at 100° C. for 15 h, cooled down, filtered, and the filtrate was evaporated. Purification by RP-HPLC gave ethyl 2-chloro-5-fluoro-3-[(2-oxoimidazolidin-4-yl)methoxy]benzoate (112.0 mg, 18.28% yield) as a white solid. Further purification by chiral SFC gave the title compound (54 mg, 48%) as a white solid. MS m/z: 317.3/319.3[M+H]⁺

### Step c): 2-chloro-5-fluoro-3-[[(4S)-2-oxoimidazolidin-4-yl]methoxy]benzoic acid

A solution of ethyl 2-chloro-5-fluoro-3-[[(4S)-2-oxoimidazolidin-4-yl]methoxy]benzoate (54.0 mg, 0.17 mmol) in THF (0.5 mL)/water (0.5 mL) was treated with lithium hydroxide (12.2 mg, 0.51 mmol). The mixture was stirred at 20° C. for 15 h, diluted with water, acidified with 1 N HCl (aq.) to pH=3, and evaporated, to give the title compound (45.0 mg, 82.29% yield) as a light yellow solid. MS (ESI): m/z=287.0 [M−H]⁻

### Example 115

### (4R)-4-[[2-chloro-3-[(7S)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one

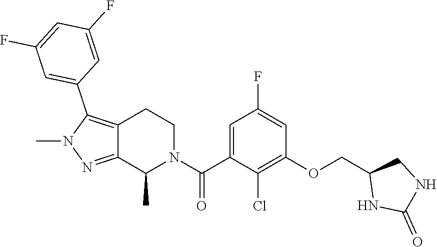 [SMILES: C[C@H]1c2nn(C)c(-c3cc(F)cc(F)c3)c2CCN1C(=O)c1cc(F)cc(OC[C@H]2CNC(=O)N2)c1Cl]

A solution of 2-chloro-5-fluoro-3-[[(4R)-2-oxoimidazolidin-4-yl]methoxy]benzoic acid (80.0 mg, 0.18 mmol) in DMF (0.5 mL) was treated with HATU (82.19 mg, 0.22 mmol), 3-(3,5-difluorophenyl)-2,7-dimethyl-4,5,6,7-tetrahydropyrazolo[3,4-c]pyridin-6-ium;chloride (54.0 mg, 0.18 mmol) and triethylamine (0.1 mL, 0.72 mmol), at 20° C. The mixture was stirred for 10 h at this temperature, before being filtered and evaporated. Purification by RP-HPLC gave (4R)-4-[[2-chloro-3-[3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one (45.0 mg, 45.76% yield) as a white solid. Further purification by chiral SFC gave the title compound (18.6 mg, 36.51% yield). MS (ESI): m/z=534.2 [M+H]⁺

### Step a): 4-(bromomethyl)imidazolidin-2-one

A solution of triphenylphosphine (3.73 g, 14.21 mmol) in DCM (30 mL) was treated dropwise with bromine (0.73 mL, 14.21 mmol), at −10° C. under Ar. The mixture was stirred for 30 min at this temperature, and 4-(hydroxymethyl)imidazolidin-2-one (CAS RN: 2248182-92-3; 1100.0 mg, 9.47 mmol) was added. The mixture was warmed to 25° C. and stirred for 15 h, before being evaporated. Purification by FC (SiO₂; ACN/MTBE) gave the title compound (330.0 mg, 19.46% yield) as a white solid. MS (ESI): m/z=179.0 [M+H]⁺

### Step b): ethyl 2-chloro-5-fluoro-3-[[(4S)-2-oxoimidazolidin-4-yl]methoxy]benzoate

A solution of ethyl 2-chloro-5-fluoro-3-hydroxy-benzoate (CAS RN: 2090950-46-0; 362.68 mg, 1.66 mmol, 0.9 eq) in DMF (5 mL) was treated with potassium carbonate (509.53 mg, 3.69 mmol). The mixture was stirred at 20° C. for 1 h before 4-(bromomethyl)imidazolidin-2-one (330.0 mg, 1.84 mmol) was added. The resulting mixture was heated at 100° C. for 15 h, cooled down, filtered, and the filtrate was evaporated. Purification by RP-HPLC gave ethyl 2-chloro-5-fluoro-3-[(2-oxoimidazolidin-4-yl)methoxy]benzoate (112.0 mg, 18.28% yield) as a white solid. Further purification by chiral SFC gave the title compound (41 mg, 37%) as a white solid. MS m/z: 317.3 [M+H]⁺

### Step c): 2-chloro-5-fluoro-3-[[(4R)-2-oxoimidazolidin-4-yl]methoxy]benzoic acid

To a solution of ethyl 2-chloro-5-fluoro-3-[[(4R)-2-oxoimidazolidin-4-yl]methoxy]benzoate (104.0 mg, 0.33 mmol) in THF (1 mL)/water (1 mL) was treated with lithium hydroxide hydrate (0.01 mL, 0.49 mmol), at 23° C. The mixture was stirred at 35° C. for 10 h, before being treated with a saturated aqueous KHSO₄ solution, to adjust to pH=4. The mixture was extracted with EtOAc. The combined organic layers were dried over sodium sulfate, filtered and evaporated, to give the title compound (90.0 mg, 60.58% yield) as a light yellow foam. MS (ESI): m/z=287.0 [M−H]⁻

### Example 116

### (2-chloro-3-methoxy-phenyl)-[(5S)-9-(3-chlorophenyl)-5-methyl-1,4,7,8-tetrazabicyclo[4.3.0]nona-1(9),6-dien-4-yl]methanone

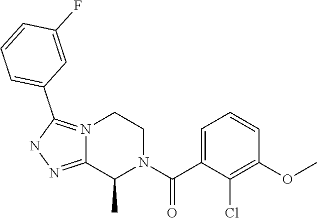

A solution of 3-(3-chlorophenyl)-8-methyl-5,6,7,8-tetrahydro-[1,2,4]triazolo[4,3-a]pyrazine (0.5 g, 2.01 mmol) and 2-chloro-3-methoxybenzoic acid (0.41 g, 2.21 mmol) in N,N-dimethylformamide (15 mL) was treated with HATU (0.99 g, 2.61 mmol) and triethylamine (0.98 mL, 7.04 mmol). The mixture was stirred for 18 h at 23° C., before being poured onto water and extracted with EtOAc. The combined organic layers were washed with water, dried over Na₂SO₄, filtered, and evaporated. Purification by RP-HPLC and chiral SFC gave the title compound (108.0 mg, 12.46% yield) as a white solid. MS (ESI): m/z=417.0 [M+H]⁺

### Step a): 3-chloro-N′-(3-methylpyrazin-2-yl)benzohydrazide

(3-Methylpyrazin-2-yl)hydrazine (CAS RN: 19848-54-5; 9.99 g, 80.48 mmol) was added to a solution of the 3-chlorobenzoic acid (CAS RN: 535-80-8; 10.5 g, 67.06 mmol), 1-hydroxybenzotriazole (13.59 g, 100.59 mmol), 1-(3-dimethylaminopropyl)-3-ethylcarbodiimide hydrochloride (28.93 g, 150.89 mmol), and triethylamine (28.04 mL, 201.19 mmol) in dichloromethane (700 mL). The reaction mixture was stirred at 23° C. for 8 h, before being diluted with water and dichloromethane. The layers were separated and the organic layer was washed with a saturated aqueous NaHCO₃ solution and brine, dried over Na₂SO₄ and evaporated to give the title compound (12.6 g, 61.51% yield) as a brown solid. MS (ESI): m/z=263.0 [M+H]⁺

### Step b): 3-(3-chlorophenyl)-8-methyl-[1,2,4]triazolo[4,3-a]pyrazine

A solution of 3-chloro-N′-(3-methylpyrazin-2-yl)benzohydrazide (12.3 g, 46.82 mmol) in Acetic acid (25 mL) was refluxed for 18 h before being cooled down and evaporated. The residue was dissolved in DCM and an aqueous saturated NaHCO₃ solution. The organic layer was washed with water, dried over sodium sulfate and evaporated. Purification by FC (SiO₂; CHCl₃/ACN) gave the title compound (5.2 g, 43.12% yield) as a light yellow solid. MS (ESI): m/z=317.0 [M+H]⁺

### Step c): 3-(3-chlorophenyl)-8-methyl-5,6,7,8-tetrahydro-[1,2,4]triazolo[4,3-a]pyrazine

A solution of 3-(3-chlorophenyl)-8-methyl-[1,2,4]triazolo[4,3-a]pyrazine (3.0 g, 12.26 mmol) and a catalytic amount of Pt in Methanol (100 mL) was stirred for 18 h at 23° C. under H₂ (50 bar). The reaction mixture was filtered and evaporated. Crystallization from hexanes gave the title compound (1.06 g, 33.02% yield) as a white solid. MS (ESI): m/z=249.0 [M+H]⁺

### Example 117

### (2-chloro-3-methoxy-phenyl)-[(8S)-3-(3-chlorophenyl)-8-methyl-2,5,6,8-tetrahydroimidazo[1,2-a]pyrazin-7-yl]methanone

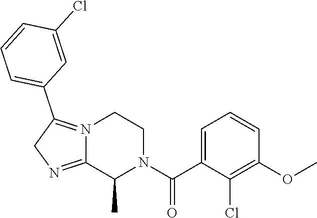

A solution of 3-(3-chlorophenyl)-8-methyl-5,6,7,8-tetrahydroimidazo[1,2-a]pyrazine;hydrochloride (150.0 mg, 0.53 mmol) and 2-chloro-3-methoxybenzoic acid (98.49 mg, 0.53 mmol) in DMF (8 mL) was treated with 2-chloro-3-methoxybenzoic acid (98.49 mg, 0.53 mmol) and triethylamine (0.26 mL, 1.85 mmol). The mixture was stirred for 18 h at 23° C., poured onto water, and extracted with EtOAc. The combined organic layers were washed with water, dried under anhydrous sodium sulfate, and evaporated. Purification by RP-HPLC and chiral SFC gave the title compound (43.8 mg, 19.73% yield) as a white solid. MS (ESI): m/z=418.0 [M+H]⁺

### Step a): tert-butyl 8-methyl-6,8-dihydro-5H-imidazo[1,2-a]pyrazine-7-carboxylate

A solution of 8-methyl-5,6,7,8-tetrahydroimidazo[1,2-a]pyrazine (CAS RN: 91476-83-4; 3.63 g, 26.45 mmol) in CH₂Cl₂ (50 mL) was treated with di-t-butyldicarbonate (6.06 g, 27.78 mmol) at 23° C. The mixture was stirred for 16 h at this temperature, before being evaporated, to give the title compound (4.5 g, 67.69% yield) as a brown oil. MS (ESI): m/z=238.2 [M+H]⁺

### Step b): tert-butyl 3-bromo-8-methyl-6,8-dihydro-5H-imidazo[1,2-a]pyrazine-7-carboxylate

A solution of tert-butyl 8-methyl-6,8-dihydro-5H-imidazo[1,2-a]pyrazine-7-carboxylate (377.69 mg, 1.59 mmol) in CCl₄ (15 mL) was treated with NBS (283.27 mg, 1.59 mmol), at 23° C. under Ar. The mixture was stirred for 1 h at reflux, before being poured onto water (50 mL) and extracted with EtOAc. The combined organic layers were washed with brine (50 mL), dried over anhydrous sodium sulfate and evaporated, to give the title compound (400 mg, 76.3% yield) as a light brown solid. MS (ESI): m/z=316.0 [M+H]⁺

### Step c): tert-butyl 3-(3-chlorophenyl)-8-methyl-6,8-dihydro-5H-imidazo[1,2-a]pyrazine-7-carboxylate

A suspension of tert-butyl 3-bromo-8-methyl-6,8-dihydro-5H-imidazo[1,2-a]pyrazine-7-carboxylate (400.0 mg, 1.27 mmol), 3-chlorophenylboronic acid (296.73 mg, 1.9 mmol) and sodium carbonate (670.41 mg, 6.33 mmol) in DME (16 mL) and water (8 mL) was sparged with Ar, before being treated with 1,1′-bis(diphenylphosphino)ferrocene-palladium(II)dichloride dichloromethane complex (103.23 mg, 0.13 mmol). The mixture was stirred for 16 h at 80° C., before being cooled down, filtered through silica, and washed with DME (50 mL). The filtrate was evaporated and purified by RP-HPLC, to give the title compound (200.0 mg, 43.18% yield). MS (ESI): m/z=348.1 [M+H]⁺

### Step d): 3-(3-chlorophenyl)-8-methyl-5,6,7,8-tetrahydroimidazo[1,2-a]pyrazine;hydrochloride

A solution of tert-butyl 3-(3-chlorophenyl)-8-methyl-6,8-dihydro-5H-imidazo[1,2-a]pyrazine-7-carboxylate (200.0 mg, 0.57 mmol) in 4 N HCl in 1,4-dioxane (5 mL) was stirred for 32 h at 23° C., before being evaporated to give the title compound (150.0 mg, 89.05% yield) as a white powder. MS (ESI): m/z=248.2 [M+H]⁺

### Building Blocks Synthesis

### Building Block A.1 N-[(3-bromo-5-chloro-phenyl)methyl]methanesulfonamide

A solution of (3-bromo-5-chloro-phenyl)methanamine (CAS RN 917388-35-3; 2.0 g, 9.07 mmol) in CH₂Cl₂ (40 mL) was treated dropwise with TEA (1.39 mL, 9.98 mmol) and methanesulfonyl chloride (0.70 mL, 9.07 mmol), at 0° C. under Ar. The mixture was allowed to warm up to 25° C. and stirred at this temperature for 12 h, before being diluted with CH₂Cl₂. The organic layer was washed with saturated aqueous citric acid and brine, dried over Na₂SO₄, filtered, and evaporated, to give the title compound (2.7 g, 94.7%) as a white solid. MS (ESI): m/z=298.0 [M+H]⁺.

### Building Block A.2

### 1-(3-bromo-5-chloro-phenyl)-N-methyl-methanesulfonamide

A solution of (3-bromo-5-chloro-phenyl)methanesulfonyl chloride (CAS RN 1499398-66-1; 300.0 mg, 0.990 mmol) in THE (10 mL) was treated dropwise with monomethylamine (2M in THF; 306.52 mg, 9.87 mmol), at 25° C. under Ar. The mixture was stirred for 18 h at this temperature, before being evaporated and dissolved in EtOAc. The organic layer was washed with aqueous hydrochloric acid solution (1 N) and saturated aqueous sodium chloride solution, dried over Na₂SO₄, filtered and evaporated, to give the title compound (240 mg, 81.45% yield) as a white solid. MS (ESI): m/z=298.0 [M−H]⁻.

### Building Block A.3

### 1-bromo-3-chloro-5-(1-methylsulfonylcyclopropyl)benzene

A solution of methyl(diphenyl)sulfonium;tetrafluoroborate (4.31 g, 14.95 mmol) in dry THE (40 mL) was treated with 1-bromo-3-chloro-5-(1-methylsulfonylvinyl)benzene (3.4 g, 11.5 mmol) and sodium;bis(trimethylsilyl)azanide (9.2 mL, 18.4 mmol), at 0° C. under Ar. The mixture was stirred for 10 min at this temperature, before being allowed to warm up to 25° C. and stirred for another 10 h. The reaction mixture was quenched with aqueous saturated ammonium chloride solution (50 mL), diluted in water (150 mL) and the organics were extracted with ethyl acetate (2×100 mL). The organic layer was washed with water, dried over Na₂SO₄, filtered and evaporated. Purification by FC (SiO₂; hexane/EtOAc) gave the title compound (2 g, 53.35% yield) as a crude light brown solid, which was directly used in the next step.

### Step a): 1-bromo-3-chloro-5-(1-methylsulfonylvinyl)benzene

A solution of 2-(3-bromo-5-chloro-phenyl)-4,4,5,5-tetramethyl-1,3,2-dioxaborolane (CAS RN 488850-91-5; 8.4 g, 26.46 mmol) in 4:1 THF/water (50 mL) was treated with 1-bromo-1-methylsulfonyl-ethylene (5386.63 mg, 29.11 mmol) and potassium carbonate (7315.02 mg, 52.93 mmol), at 25° C. under Ar. The mixture was sparged with Ar for 5 minutes, before being treated with 1,1′-bis(diphenylphosphino)ferrocene-palladium(II)dichloride dichloromethane complex (2159.48 mg, 2.65 mmol). The mixture was stirred for 16 h at 70° C., before being filtered through a pad of silica. The filtrate was evaporated and purified by FC (SiO₂; PE/MTBE), to give the title compound (3.4 g, 41.29% yield) as a crude light brown oil, which was directly used in the next step.

### Building Block C.1

### 2-chloro-3-(2,2-dioxo-226-thia-6-azaspiro[3.3]heptan-6-yl)-5-fluoro-benzoic acid

A solution of methyl 2-chloro-3-(2,2-dioxo-2λ⁶-thia-6-azaspiro[3.3]heptan-6-yl)-5-fluoro-benzoate (900.0 mg, 2.7 mmol) in 4:4:1 THF/MeOH/water (101.25 mL) was treated with LiOH hydrate (339.44 mg, 8.09 mmol) at 23° C., and stirred for 18 h at this temperature.

The reaction mixture was evaporated, before being diluted with water (11.25 mL) and acidified using 2 N HCl until formation of a precipitate. The precipitate was filtered off, washed with water, and dried, to give the title compound (680 mg, 74.93% yield) as a white solid. MS (ESI): m/z 320.2 [M+H]⁺.

### Step a): methyl 2-chloro-3-(2,2-dioxo-2λ6-thia-6-azaspiro[3.3]heptan-6-yl)-5-fluoro-benzoate

A sealed tube was charged with methyl 3-bromo-2-chloro-5-fluoro-benzoate (CAS RN 1805582-40-4; 1.5 g, 5.61 mmol), tris(dibenzylideneacetone)dipalladium (0) (256.76 mg, 0.280 mmol), 2λ6-thia-6-azaspiro[3.3]heptane 2,2-dioxide;hydrochloride (CAS RN 1263182-09-7; 2.06 g, 11.22 mmol), cesium carbonate (7.31 g, 22.43 mmol), 9,9-dimethyl-4,5-bis(diphenylphosphino)xanthene (162.24 mg, 0.280 mmol), and 1,4-dioxane (120 mL), at 23° C. under Ar. The mixture was sparged with Ar for 15 min, before being heated for 18 h at 100° C. The mixture was cooled down and evaporated. Purification by FC (SiO₂; CHCl₃/ACN) gave the title compound (1.1 g, 56.24% yield) as a light yellow solid. MS (ESI): m/z=334.0 [M+H]⁺.

### Building Block C.2

### 2-chloro-5-fluoro-3-(3-methylsulfonylazetidin-1-yl)benzoic acid

A solution of methyl 2-chloro-5-fluoro-3-(3-methylsulfonylazetidin-1-yl)benzoate (1.1 g, 3.42 mmol) in 5:1 THF/water (48 mL) was treated with LiOH monohydrate (358.63 mg, 8.55 mmol), at 23° C. The mixture was heated to 50° C. and stirred for 4 h at this temperature, before being concentrated under reduced pressure and acidified to pH 4 using a saturated aqueous citric acid solution. The resulting suspension was stirred for 30 min at 23° C., before being filtered. The cake was washed with water and dried, to give the title compound (840 mg, 75.85% yield) as a light grey solid. MS (ESI): m/z=308.0 [M+H]⁺.

### Step a): methyl 2-chloro-5-fluoro-3-(3-methylsulfonylazetidin-1-yl)benzoate

A solution of methyl 3-bromo-2-chloro-5-fluoro-benzoate (CAS RN 1805582-40-4; 1250.0 mg) in 1,4-dioxane (60 mL) was treated with 3-methylsulfonylazetidine; hydrochloride (CAS RN 1400764-60-4; 1203.24 mg, 7.01 mmol), Xantphos (216.32 mg, 0.370 mmol), tris(dibenzylideneacetone)dipalladium (213.97 mg, 0.230 mmol) and cesium carbonate (3045.27 mg, 9.35 mmol), at 23° C. under Ar. The mixture was sparged for 10 min with Ar, and the mixture was heated to 90° C. for 24 h, before being cooled down. The mixture was filtered through a silica pad, washed with 1,4-dioxane (100 mL), and evaporated. The residue was diluted with EtOAc (100 mL), and the organic layer was washed with water (20 mL), brine (20 mL), dried over Na₂SO₄, filtered, and evaporated. Purification by FC (SiO₂) gave the title compound (1.1 g, 71.69% yield) as a light yellow solid. MS (ESI): m/z=322.0 [M+H]⁺.

### Building Block C.3

### 2-chloro-5-fluoro-3-[[1-(trifluoromethyl)cyclopropyl]methoxy]benzoic acid

A solution of ethyl 2-chloro-5-fluoro-3-[[1-(trifluoromethyl)cyclopropyl]methoxy]benzoate (0.8 g, 2.35 mmol) in 2:1 EtOH/water was treated with lithium hydroxide (0.04 mL, 4.7 mmol), at 23° C. The mixture was heated to 40° C., and stirred for 12 h at this temperature before being concentrated under reduced pressure. Water and MTBE were added, and the resulting mixture was acidified using saturated citric acid. The aqueous layer was extracted with EtOAc, and the combined organic layers were washed with brine, dried over Na₂SO₄, filtered, and evaporated. The crude product was recrystallized from hexane, to give the title compound (0.400 g, 51.76% yield) as a pink solid. MS (ESI): m/z=311.0 [M−H]⁻.

### Step a): ethyl 2-chloro-5-fluoro-3-[[1-(trifluoromethyl)cyclopropyl]methoxy]benzoate

A solution of ethyl 2-chloro-5-fluoro-3-hydroxy-benzoate (CAS RN 2090950-46-0; 0.63 g, 2.88 mmol) and cesium carbonate (2.82 g, 8.65 mmol) in DMF (10 mL) was treated with [1-(trifluoromethyl)cyclopropyl]methyl 4-methylbenzenesulfonate (CAS RN 865833-72-3; 1.02 g, 3.46 mmol), at 23° C. The reaction mixture was heated to 100° C. and stirred for 24 h at this temperature, before being cooled down and partitioned between EtOAc and water. The organic layer was washed with brine (3×), dried over Na₂SO₄, filtered and evaporated, to give the title compound (0.880 g, 71.7% yield) as a brown liquid. MS (ESI): m/z=341.0 [M+H]⁺.

### Building Block C.4

### 2-chloro-3-(5,5-dimethyl-4H-isoxazol-3-yl)-5-fluoro-benzoic acid

A solution of methyl 2-chloro-3-(5,5-dimethyl-4H-isoxazol-3-yl)-5-fluoro-benzoate (480.0 mg, 1.68 mmol) in 4:4:1 THF/MeOH/water (9 mL) was treated with lithium hydroxide monohydrate (140.99 mg, 3.36 mmol), at 25° C. The mixture was stirred for 18 h at this temperature, before being concentrated under reduced pressure and acidified to pH=4 with HCl (1 N). The resulting suspension was stirred for 30 min, before being filtered. The precipitate was washed with water and dried, to give the title compound (332 mg, 71.28% yield) as a white solid. MS (ESI): m/z=272.0 [M+H]⁺.

### Step a): methyl 2-chloro-5-fluoro-3-formyl-benzoate

A solution of methyl 3-bromo-2-chloro-5-fluoro-benzoate (CAS RN: 1805582-40-4; 10.0 g, 37.39 mmol) in THF (250 mL) was treated dropwise with n-BuLi (17.95 mL, 44.86 mmol), at −78° C. under Ar. The mixture was stirred for 30 min at this temperature, before being treated dropwise with a solution of DMF (8.68 mL, 112.16 mmol) in THE (50 mL). The mixture was allowed to warm up to 25° C. and stirred for 3 h at this temperature, before being treated with saturated aqueous NH₄Cl (50 mL). The layers were separated, and the organic layer was washed with brine (20 mL). The aqueous layers were back-extracted with EtOAc (2×50 mL). The combined organic layers were dried over Na₂SO₄ and evaporated. Purification by FC (SiO₂) gave the title compound (1.2 g, 13.34% yield) as a light yellow solid. MS (ESI): m/z=216.0 [M+H]⁺.

### Step b): methyl 2-chloro-5-fluoro-3-[(E)-hydroxyiminomethyl]benzoate

A solution of methyl 2-chloro-5-fluoro-3-formyl-benzoate (1200.0 mg, 5.54 mmol) (90% purity) in 2:1:1 THF/EtOH/water (28 mL) was treated with hydroxylamine hydrochloride (770.01 mg, 11.08 mmol) and sodium acetate (1363.51 mg, 16.62 mmol), at 23° C. The mixture was stirred for 24 h at this temperature, before being evaporated. The residue was dissolved in DCM (100 mL) and water (20 mL), and the layers were separated. The aqueous layer was extracted with DCM (2×), and the combined organic layers were dried over Na₂SO₄, filtered, and evaporated, to give the title compound (1.1 g, 77.15% yield) as a crude light yellow solid. MS (ESI): m/z=230.2 [M−H]⁻.

### Step c): methyl 2-chloro-3-[(Z)—C-chloro-N-hydroxy-carbonimidoyl]-5-fluoro-benzoate

A solution of methyl methyl 2-chloro-5-fluoro-3-[(E)-hydroxyiminomethyl]benzoate (1.2 g, 5.18 mmol) (85% purity) in DMF (20 mL) was treated portionwise with NCS (0.69 g, 5.18 mmol), at 25° C. under Ar, before being stirred for 3 h at this temperature. The mixture was then diluted with water (100 mL), and extracted with TBME (3×50 mL). The combined organic layers were washed with water (2×10 mL) and brine (10 mL), dried over Na₂SO₄, and evaporated, to give the title compound (1.25 g, 77.08% yield) as a light yellow solid. MS (ESI): m/z=267.2 [M+H]⁺.

### Step d): methyl 2-chloro-3-(5,5-dimethyl-4H-isoxazol-3-yl)-5-fluoro-benzoate

A solution of methyl 2-chloro-3-[(Z)—C-chloro-N-hydroxy-carbonimidoyl]-5-fluoro-benzoate (1.25 g, 4.7 mmol) in DCM (50 mL) was treated with 2-methylpropene (1.05 g, 18.79 mmol) and TEA (1.31 mL, 9.4 mmol), at −10° C. under Ar. The mixture was allowed to warm up to 25° C. and stirred at this temperature for 24 h, before being evaporated. Purification by RP-HPLC gave the title compound (490 mg, 35.77% yield) as a yellow oil. MS (ESI): m/z=286.0 [M+H]⁺.

### Building Block C.5

### 2-chloro-5-fluoro-3-(6-oxo-1H-pyridin-3-yl)benzoic acid

Methyl 2-chloro-5-fluoro-3-(6-methoxy-3-pyridyl)benzoate (2550.0 mg, 8.62 mmol) was treated with 12 N HCl (136.0 mL, 1632 mmol), at 25° C. The mixture was heated to 88° C. and stirred for 18 h at this temperature, before being cooled down and diluted with 400 mL of ice-cold water. The precipitate was filtered and dried, to give the title compound (1027 mg, 40.97% yield) as a light yellow solid. MS (ESI): m/z=268 [M+H]⁺.

### Step a): methyl 2-chloro-5-fluoro-3-(6-methoxy-3-pyridyl)benzoate

A solution of methyl 3-bromo-2-chloro-5-fluoro-benzoate (CAS RN: 1805582-40-4; 4.0 g, 14.95 mmol) and 2-methoxy-5-pyridineboronic acid (CAS RN 163105-89-3; 2.97 g, 19.45 mmol) in 4:1 DME/water (100 mL) was sparged with Ar for 10 min, before being treated with bis(triphenylphosphine)palladium(II) chloride (629.79 mg, 0.900 mmol) and sodium carbonate (3.96 g, 37.39 mmol). The mixture was heated to 100° C., and stirred for 18 h at this temperature before being cooled down, filtered, and evaporated. Purification by FC (SiO₂) gave the title compound (2550 mg, 57.67% yield) as a white solid. MS (ESI): m/z=296 [M+H]⁺.

### Building Block C.6

### chloro-5-fluoro-3-(2-oxo-1H-pyridin-4-yl)benzoic acid

Methyl 2-chloro-5-fluoro-3-(2-methoxy-4-pyridyl)benzoate (1410.0 mg, 4.77 mmol) was treated with 12 N HCl (75.2 mL, 902.4 mmol), at 25° C. The mixture was heated to 88° C. and stirred for 18 h at this temperature, before being cooled down and diluted with 400 mL of ice-cold water. The precipitate was filtered and dried, to give the title compound (524.2 mg, 37.04% yield) as a light yellow solid. MS (ESI): m/z=268 [M+H]⁺.

### Step a): methyl 2-chloro-5-fluoro-3-(2-methoxy-4-pyridyl)benzoate

A solution of 2-methoxypyridine-4-boronic acid (CAS RN 762262-09-9; 2.97 g, 19.44 mmol) in 4:1 DME/water (100 mL) was treated with methyl 3-bromo-2-chloro-5-fluoro-benzoate (CAS RN: 1805582-40-4; 4.0 g, 14.95 mmol), sodium carbonate (3.96 g, 37.39 mmol) and bis(triphenylphosphine)palladium(II) chloride (629.79 mg, 0.900 mmol), at 25° C. under Ar. The mixture was heated to 100° C. and stirred for 18 h at this temperature, before being cooled down, filtered, and evaporated. Purification by RP-HPLC gave the title compound (1.48 g, 32.05% yield) as a light yellow solid. MS (ESI): m/z=296 [M+H]⁺.

Building Block C.7

### 1-(2-oxo-1H-pyridin-3-yl)-1,2,4-triazole-3-carboxylic acid

A solution of methyl 1-(2-methoxy-3-pyridyl)-1,2,4-triazole-3-carboxylate (1090.0 mg, 4.65 mmol) in 12 N HCl (50 mL) was stirred for 18 h at 70° C., before being cooled down. The precipitate was filtered, washed with water, and dried, to give the title compound (603.2 mg, 59.73% yield) as a light grey solid. MS (ESI): m/z=207.0 [M+H]⁺.

### Step a): methyl 1-(2-methoxy-3-pyridyl)-1,2,4-triazole-3-carboxylate

A solution of methyl 1H-1,2,4-triazole-3-carboxylate (CAS RN: 4928-88-5; 2.49 g, 19.61 mmol) and 2-methoxypyridine-3-boronic acid (CAS RN: 163105-90-6; 3.0 g, 19.61 mmol) in DCM (180 mL) was treated with pyridine (4.76 mL, 58.83 mmol), 4 Å MS (3 g), and Cu(OAc)₂ (3.56 g, 19.61 mmol), at 23° C. The mixture was heated to 30° C. and stirred for 48 h at this temperature, before being filtered over celite. The filtrate was washed with a saturated aqueous ammonium hydroxide solution, and the the organic layer was dried over Na₂SO₄, filtered, and evaporated, to give the title compound (1.4 g, 27.74% yield) as a crude white solid. MS (ESI): m/z=235.0 [M+H]⁺.

### Building Block C.8

### 1-(6-oxo-1H-pyridin-3-yl)-1,2,4-triazole-3-carboxylic acid

A solution of methyl 1-(6-methoxy-3-pyridyl)-1,2,4-triazole-3-carboxylate (1.1 g, 4.7 mmol) in 12 N HCl (50 mL) was heated to 70° C. and stirred for 18 h at this temperature, before being cooled down. The precipitate was filtered, washed with water, and dried, to give the title compound (572.9 mg, 56.21% yield) as a light grey solid. MS (ESI): m/z=207.0 [M+H]⁺.

### Step a): methyl 1-(6-methoxy-3-pyridyl)-1,2,4-triazole-3-carboxylate

A solution of methyl 1H-1,2,4-triazole-3-carboxylate (CAS RN: 4928-88-5; 2.5 g, 19.7 mmol) and 2-methoxy-5-pyridineboronic acid (CAS RN: 163105-89-3; 3.01 g, 19.67 mmol) in DCM (150 mL) was treated with pyridine (4.77 mL, 59.01 mmol), 4 Å MS (2.5 g), and Cu(OAc)₂ (3.57 g, 19.67 mmol), at 25° C. The mixture was heated to 30° C. and stirred for 48 h at this temperature, before being filtered over celite. The filtrate was washed with a saturated aqueous ammonium hydroxide solution, and the organic layer was dried over Na₂SO₄, filtered, and evaporated, to give the title compound (2.2 g, 42.5% yield) as a crude white solid. MS (ESI): m/z=235.2 [M+H]⁺.

### Building Block C.9

### 1-(5-fluoro-2-hydroxy-3-pyridyl)-1,2,4-triazole-3-carboxylic acid

A solution of sodium;1-(5-fluoro-2-methoxy-3-pyridyl)-1,2,4-triazole-3-carboxylate (435.0 mg, 1.34 mmol) in 12 N HCl (8.03 mL, 80.26 mmol) was heated to 90° C. and stirred for 16 h at this temperature, before being cooled down. The precipitate was filtered, washed with water, and dried, to give the title compound (220 mg, 66.04% yield) as a white solid. MS (ESI): m/z=225.0 [M+H]⁺.

### Step a): methyl 1-(5-fluoro-2-methoxy-3-pyridyl)-1,2,4-triazole-3-carboxylate

A solution of methyl 1H-1,2,4-triazole-3-carboxylate (CAS RN: 4928-88-5; 1.6 g, 12.58 mmol) in DCM (100 mL) was treated with (5-fluoro-2-methoxy-3-pyridyl)boronic acid (CAS RN: 957120-32-0; 2.15 g, 12.58 mmol), pyridine (3.05 mL, 37.73 mmol), cupric acetate (3.43 g, 18.87 mmol) and 4 AMS (3 g), at 23° C. The mixture was heated to 30° C. and stirred for 16 h at this temperature, before being filtered and evaporated. Purification by RP-HPLC gave the title compound (282 mg, 8.53% yield) as a white solid. MS (ESI): m/z=253.2 [M+H]⁺.

### Step b): sodium;1-(5-fluoro-2-methoxy-3-pyridyl)-1,2,4-triazole-3-carboxylate

A solution of methyl 1-(5-fluoro-2-methoxy-3-pyridyl)-1,2,4-triazole-3-carboxylate (393.0 mg, 1.56 mmol) in MeOH (2 mL) was treated with a solution of NaOH (93.5 mg, 2.34 mmol) in water (1 mL), at 20° C. The mixture was stirred for another 16 h at this temperature, before being evaporated. Purification by RP-HPLC gave the title compound (435 mg, 85.84% yield) as a white solid. MS (ESI): m/z=239.2 [M+H]⁺.

### Building Block C.10

### 1-(5-chloro-2-hydroxy-3-pyridyl)-1,2,4-triazole-3-carboxylic acid

A solution of sodium;1-(5-chloro-2-methoxy-3-pyridyl)-1,2,4-triazole-3-carboxylate (210.0 mg, 0.610 mmol) in 12 N HCl (3.04 mL, 30.37 mmol) was heated to 90° C. and stirred for 16 h at this temperature, before being cooled down. The precipitate was filtered, washed with water, and dried, to give the title compound (102.6 mg, 70.21% yield) as a white solid. MS (ESI): m/z=241.0 [M+H]⁺.

### Step a): methyl 1-(5-chloro-2-methoxy-3-pyridyl)-1,2,4-triazole-3-carboxylate

A solution of methyl 1H-1,2,4-triazole-3-carboxylate (CAS RN: 4928-88-5; 0.68 g, 5.34 mmol) in DCM (100 mL) was treated 5-chloro-2-methoxypyridine-3-boronic acid (CAS RN: 943153-22-8; 1.0 g, 5.34 mmol), pyridine (1.29 mL, 16.01 mmol), cupric acetate (1453.89 mg, 8 mmol) and 4 AMS (3 g), at 20° C. The mixture was heated to 30° C. and stirred for 16 h at this temperature, before being evaporated. The residue was diluted with aqueous HCl (5%; 50 mL), and the resulting precipitate was filtered and dried, to give the title compound (210 mg, 13.48% yield) as a white solid. MS (ESI): m/z=269.2 [M+H]⁺.

### Step b): sodium;1-(5-chloro-2-methoxy-3-pyridyl)-1,2,4-triazole-3-carboxylate

A solution of methyl 1-(5-chloro-2-methoxy-3-pyridyl)-1,2,4-triazole-3-carboxylate (210.0 mg, 0.720 mmol) in MeOH (2 mL) was treated with a solution of NaOH (43.15 mg, 1.08 mmol) in water (1 mL), at 20° C. The mixture was stirred for another 16 h at this temperature, before being evaporated, to give the title compound (210 mg, 84.46% yield) as a crude white solid. MS (ESI): m/z=255 [M+H]⁺.

### Building Block C.11

### 2-chloro-3-(5-cyano-1H-pyrrol-3-yl)-5-fluoro-benzoic acid

A solution of 2-chloro-3-(5-cyano-1H-pyrrol-3-yl)-5-fluoro-benzoic acid methyl ester (2730 mg, 9.8 mmol) in 1:1 1,4-dioxane/water (30 mL) was treated with LiOH monohydrate (739.95 mg, 17.63 mmol), at 23° C. The mixture was stirred for 6.5 h at this temperature, before being concentrated under reduced pressure. The remaining aqueous solution was treated dropwise with 1M HCl (17.63 mL, 17.63 mmol) and water (20 mL).

The obtained thick slurry was filtered, and the cake was washed with water and dried, to give the title compound (2.489 g, 96.0% yield) as a white solid. MS (ESI): m/z=263.1 [M−H]⁻.

### Step a): methyl 2-chloro-5-fluoro-3-(4,4,5,5-tetramethyl-1,3,2-dioxaborolan-2-yl)benzoate

A solution of methyl methyl 3-bromo-2-chloro-5-fluoro-benzoate (CAS RN: 1805582-40-4; 58.0 g, 216.84 mmol) in 1,4-dioxane (966.67 mL) was treated with bis(pinacolato)diboron (60.57 g, 238.52 mmol), potassium acetate (63.84 g, 650.52 mmol), and 1,1′-bis(diphenylphosphino)ferrocene-palladium(II)dichloride dichloromethane complex (17.69 g, 21.68 mmol), at 23° C. under Ar. The mixture was sparged with Ar, before being heated to 100° C. and stirred for 16 h at this temperature. The mixture was then cooled down, filtered over SiO₂, and evaporated. Purification by FC (SiO₂; hexane/MTBE) gave the title compound (18385 mg, 25.34% yield) as a light yellow oil. MS (ESI): m/z=314.0 [M+H]⁺.

### Step b): methyl 2-chloro-3-(5-cyano-1H-pyrrol-3-yl)-5-fluoro-benzoate

A solution of 4-bromo-1H-pyrrole-2-carbonitrile (CAS RN: 1221435-18-2; 1.55 g, 9.06 mmol) in 2-MeTHF (20 mL) was treated with methyl 2-chloro-5-fluoro-3-(4,4,5,5-tetramethyl-1,3,2-dioxaborolan-2-yl)benzoate (3.0 g, 9.54 mmol) and a solution of sodium carbonate (1.52 g, 14.31 mmol) in water (6 mL), at 23° C. under Ar. The mixture was sparged with Ar, before being treated with ditert-butyl(cyclopenta-1,4-dien-1-yl)phosphane;dichloropalladium;iron (249.42 mg, 0.380 mmol). The mixture was heated to 60° C. and stirred for 3 h at this temperature, before being cooled down. The organic layer was separated, and the aqueous layer was extracted with EtOAc (5 mL). The combined organic layers were washed with brine, dried over Na₂SO₄, and evaporated. Purification by FC (SiO₂; hexane/EtOAc) gave the title compound (1600 mg, 58.93% yield) as a white powder. MS (ESI): m/z=277.0/279.0 [M−H]⁻.

### Building Block C.12

### chloro-5-fluoro-3-[(5-oxopyrrolidin-3-yl)methoxy]benzoic acid

A solution of ethyl 2-chloro-5-fluoro-3-[(5-oxopyrrolidin-3-yl)methoxy]benzoate (774.0 mg, 2.45 mmol) in MeOH (5 mL) was treated with a solution of NaOH (147.09 mg, 3.68 mmol) in water (5 mL), at 23° C. The mixture was stirred for 16 h, before being evaporated. The residue was dissolved in HCl (pH=3). The resulting precipitate was filtered off and dried, to give the title compound (503 mg, 68.47% yield) as a white solid. MS (ESI) m/z=288.0 [M+H]f.

### Step a): ethyl 2-chloro-5-fluoro-3-[(5-oxopyrrolidin-3-yl)methoxy]benzoate

A solution of 4-(bromomethyl)pyrrolidin-2-one (814.37 mg, 4.57 mmol) in DMF (10 mL) was treated with ethyl 2-chloro-5-fluoro-3-hydroxy-benzoate (1.0 g, 4.57 mmol) and cesium carbonate (2.98 g, 9.15 mmol), at 23° C. The mixture was heated to 90° C. and stirred for 16 h at this temperature, before being filtered and evaporated. Purification by RP-HPLC gave the title compound (774 mg, 53.59% yield) as a light brown solid. MS (ESI): m/z=316.0 [M+H]⁺.

### Building Block C.13

### 3-chloro-2-[(2-oxooxazolidin-5-yl)methoxy]pyridine-4-carboxylic acid

A solution of 3-chloro-2-fluoro-pyridine-4-carboxylic acid (CAS RN: 741683-19-2; 824.48 mg, 4.7 mmol) in DMF (30 mL) was treated with 5-(hydroxymethyl)oxazolidin-2-one (CAS RN: 7517-99-9; 500.0 mg, 4.27 mmol) and cesium carbonate (2.78 g, 8.50 mmol), at 23° C. The mixture was heated to 80° C. and stirred for 10 h at this temperature, before being cooled down. The mixture was acidified quantitatively with NaHSO₄, and extracted with EtOAc/water. The organic layer was separated, washed with water and brine, dried over Na₂SO₄, filtered, and evaporated. Purification by RP-HPLC gave the title compound (234 mg, 18.09% yield). as a white solid. MS (ESI): m/z=273.0 [M+H]⁺.

Building block C.14

### 2-chloro-4-[2-(5-oxopyrrolidin-3-yl)ethyl]benzoic acid

A solution of methyl 2-chloro-4-[2-(5-oxopyrrolidin-3-yl)ethyl]benzoate (1050.0 mg, 3.73 mmol) in 4:4:1 THF/MeOH/water (36 mL) was treated with lithium hydroxide monohydrate (203.3 mg, 4.85 mmol), at 25° C. The mixture was stirred for 18 h at this temperature, before being concentrated under reduced pressure and acidified to pH 4 with 1M HCl. The resulting suspension was stirred for 30 minutes and filtered. The precipitate was washed with water and dried, to give the title compound (738.8 mg, 70.34% yield) as a white solid. MS (ESI): m/z=266.0 [M−H]⁻.

### Step a): methyl 2-chloro-4-[2-(5-oxopyrrolidin-3-yl)ethynyl]benzoate

A solution of methyl 2-chloro-4-iodo-benzoate (CAS RN: 156573-32-9; 1600.0 mg, 5.4 mmol) in ACN (70 mL) was treated with TEA (0.75 mL, 5.4 mmol) and 4-ethynylpyrrolidin-2-one (CAS RN: 2098062-96-3; 588.92 mg, 5.4 mmol), at 23° C. under Ar, The mixture was sparged with Ar for 3 min, before being treated with Pd(PPh₃)₄ (438.19 mg, 0.380 mmol) and CuI (102.78 mg, 0.540 mmol). The mixture was heated to 50° C. and stirred for 16 h at this temperature, before being filtered through a silica pad and evaporated. Purification by FC (SiO₂; CHCl₃/ACN) gave the title compound (1050 mg, 66.56% yield) as light grey solid. MS (ESI): m/z=278.0/280.0 [M+H]⁺.

### Step b): methyl 2-chloro-4-[2-(5-oxopyrrolidin-3-yl)ethyl]benzoate

A solution of methyl 2-chloro-4-[2-(5-oxopyrrolidin-3-yl)ethynyl]benzoate (1050.0 mg, 3.78 mmol) in EtOAc (70 mL) was treated with Rh/C (10%) (300.0 mg) and hydrogen gas. The mixture was stirred for 18 h at atmospheric pressure, before being put back under Ar, filtered, and evaporated, to give the title compound (1000 mg, 87.3% yield) as a light yellow oil. MS (ESI): m/z=282.0/284.0 [M+H]⁺.

### Building Block C.15

### 2-chloro-5-fluoro-3-[(2-oxooxazolidin-5-yl)methoxy]benzoic acid

A solution of ethyl 2-chloro-5-fluoro-3-[(2-oxooxazolidin-5-yl)methoxy]benzoate (430.0 mg, 1.35 mmol) in 1:1 MeOH/water (1 mL) was treated with NaOH (81.21 mg, 2.03 mmol), at 20° C. The mixture was stirred for 10 h at this temperature, before being evaporated. The residue was dissolved in water (3 mL) and acidified with aq. H₃PO₄ to pH=4. The mixture was extracted with EtOAc (3×3 mL). The combined organic layers were dried over Na₂SO₄, filtered, and evaporated, to give the title compound (248 mg, 44.79% yield) as a yellow solid. MS (ESI): m/z=290.0/292.0 [M+H]⁺.

### Step a): ethyl 2-chloro-5-fluoro-3-[(2-oxooxazolidin-5-yl)methoxy]benzoate

A solution of ethyl 2-chloro-5-fluoro-3-hydroxy-benzoate (CAS RN: 2090950-46-0; 815.0 mg, 3.73 mmol) in DMA (10 mL) was treated with 5-(bromomethyl)-1,3-oxazolidin-2-one (CAS RN: 51337-32-7; 1006.59 mg, 5.59 mmol) and cesium carbonate (2429.38 mg, 7.46 mmol), at 23° C. The mixture was heated to 90° C. and stirred for 16 h, before being filtered. The filtrate was evaporated. Purification by RP-HPLC gave the title compound (597 mg, 50.4% yield) as a dark brown solid. MS (ESI): m/z=318.0/320.0 [M+H]⁺.

### Building Block C.16

### 2-chloro-5-fluoro-3-(3-sulfamoylazetidin-1-yl)benzoic acid

A solution of the methyl 2-chloro-5-fluoro-3-(3-sulfamoylazetidin-1-yl)benzoate (220.0 mg, 0.680 mmol) in 2.5:2.5:1 THF/MeOH/water (6 mL) was treated with LiOH monohydrate (42.9 mg, 1.02 mmol) at 25° C. The mixture was heated to 50° C. and stirred for 16 h at this temperature, before being evaporated. The residue was dissolved in water (20 mL), and pH was adjusted to pH=5 by adding HCl (1 M). The aqueous layer was extracted with DCM (2×30 mL), and the combined organic layers were dried with over Na₂SO₄, filtered, and evaporated, to give the title compound (33.8 mg, 15.26% yield) as a light brown powder. MS (ESI): m/z=309.0 [M+H]⁺.

### Step a): tert-butyl 3-[bis[(4-methoxyphenyl)methyl]sulfamoyl]azetidine-1-carboxylate

A solution of bis(4-methoxybenzyl)amine (2.21 g, 8.6 mmol) in DCM (8 mL) was treated with TEA (1.64 mL, 11.73 mmol) and 3-(chlorosulfonyl)azetadine-1-carboxylate (2.0 g, 7.82 mmol), at 0° C. The mixture was allowed to warm up to 25° C. and stirred for 12 h at this temperature, before being treated with 1 N HCl solution. The mixture was extracted with DCM, dried over Na₂SO₄ and evaporated, to give the title compound (3.9 g, 99.4% yield) as a white solid. MS (ESI): 377.2 [M-Boc+H]⁺.

### Step b): N,N-bis[(4-methoxyphenyl)methyl]azetidine-3-sulfonamide;4-methylbenzenesulfonic acid

A solution of tert-butyl 3-[bis[(4-methoxyphenyl)methyl]sulfamoyl]azetidine-1-carboxylate (5.2 g, 10.91 mmol) in ACN (10 mL) was treated with PTSA (2.28 g, 12 mmol), at 23° C. The mixture was stirred for 10 h at this temperature. The formed precipitate was collected by filtration, to give the title compound (4.6 g, 73% yield) as a white solid. MS (ESI): m/z=377 [M+H]⁺.

### Step c) methyl 3-[3-[bis[(4-methoxyphenyl)methyl]sulfamoyl]azetidin-1-yl]-2-chloro-5-fluoro-benzoate

A sealed tube was charged with N,N-bis[(4-methoxyphenyl)methyl]azetidine-3-sulfonamide;4-methylbenzenesulfonic acid (2.0 g, 3.65 mmol), methyl 3-bromo-2-chloro-5-fluoro-benzoate (0.98 g, 3.65 mmol), tris(dibenzylideneacetone)dipalladium (0) (166.9 mg, 0.180 mmol), cesium carbonate (3.92 g, 12.0 mmol) and 9,9-dimethyl-4,5-bis(diphenylphosphino)xanthene (168.74 mg, 0.290 mmol), under Ar. 1,4-Dioxane (30 mL) was added, and the mixture was degassed with Ar for 15 min, before being heated to 100° C. The mixture was stirred for 18 h at this temperature, before being evaporated. Purification by FC (SiO₂; CHCl₃/ACN) gave the title compound (1 g, 48.72% yield) as a yellow solid. MS (ESI): m/z=563.2 [M+H]⁺.

### Step d): methyl 2-chloro-5-fluoro-3-(3-sulfamoylazetidin-1-yl)benzoate

A solution of methyl 3-[3-[bis[(4-methoxyphenyl)methyl]sulfamoyl]azetidin-1-yl]-2-chloro-5-fluoro-benzoate (600.0 mg, 1.07 mmol) in DCM (25 mL) was treated with trifluoromethanesulfonic acid (79.97 mg, 0.530 mmol), at 23° C. The mixture was stirred for 16 h at this temperature, before being diluted with DCM (15 mL) and saturated NaHCO₃ (15 mL). The organic layer was separated, washed with brine (5 mL), dried over MgSO₄, filtered, and evaporated, to give the title compound (200 mg, 55.24% yield) as a red solid. MS (ESI): m/z=323.0 [M+H]⁺.

### Building Block C.17

### 2-chloro-5-fluoro-3-(1H-1,2,4-triazol-3-yl)benzoic acid;hydrochloride

A solution of methyl 2-chloro-5-fluoro-3-(1-trityl-1,2,4-triazol-3-yl)benzoate (500.0 mg, 0.870 mmol) (87% purity) in HCl (12 N, 25 mL) was stirred at 70° C. for 12 h. The resulting precipitate was filtered off, and the filtrate was evaporated to dryness. Purification by RP-HPLC gave the title compound (106.1 mg, 39.75% yield) as a light yellow solid. MS (ESI): m/z=242.0 [M+H]⁺.

### Step a): methyl 2-chloro-5-fluoro-3-(4,4,5,5-tetramethyl-1,3,2-dioxaborolan-2-yl)benzoate

A MW vials was charged with methyl 3-bromo-2-chloro-5-fluorobenzoate (CAS RN: 1805582-40-4; 14.0 g, 52.34 mmol), 4,4,4′,4′,5,5,5′,5′-octamethyl-2,2′-bi(1,3,2-dioxaborolane) (15.95 g, 62.81 mmol), KOAc (15.4 g, 157.02 mmol), and 1,4-dioxane (200 mL), at 23° C. under Ar. The mixture was sparged with Ar for 10 min, before being treated with 1,1′-bis(diphenylphosphino)ferrocene-palladium(II)dichloride dichloromethane complex (4.27 g, 5.23 mmol). The mixture was heated to 110° C. and stirred for 16 h at this temperature, before being cooled down and filtered. The filtrate was evaporated. Purification by FC (SiO₂; hexane/MTBE) gave the title compound (11.0 g, 60.8%) as a yellow oil. MS (ESI): m/z=314.1 [M]⁺.

### Step b): methyl 2-chloro-5-fluoro-3-(I-trityl-1,2,4-triazol-3-yl)benzoate

A solution of 3-bromo-1-trityl-1,2,4-triazole (CAS RN: 151899-63-7; 2.1 g, 5.38 mmol) in 10:1 1,4-dioxane/water (66 mL) was treated with methyl 2-chloro-5-fluoro-3-(4,4,5,5-tetramethyl-1,3,2-dioxaborolan-2-yl)benzoate (1.69 g, 5.38 mmol) and cesium carbonate (3.51 g, 10.76 mmol), at 23° C. under Ar. The mixture was sparged with Ar for 10 min, before being treated with 1,1′-bis(diphenylphosphino)ferrocene-palladium(II)dichloride dichloromethane complex (658.6 mg, 0.810 mmol). The mixture was heated to 80° C. and stirred for 24 h at this temperature, before being cooled down, filtered through silica, and evaporated. Purification by FC (SiO₂; CHCl₃/ACN) gave the title compound (1.9 g, 64.53% yield) as a crude light yellow solid, which was directly used in the next step.

### Example 118

A compound of formula (II) can be used in a manner known per se as the active ingredient for the production of tablets of the following composition:

| | Per tablet |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example 119

A compound of formula (II) can be used in a manner known per se as the active ingredient for the production of capsules of the following composition:

| | Per capsule |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

1. A compound of formula (II) 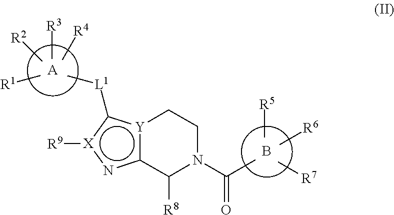
or a pharmaceutically acceptable salt thereof, wherein:
L¹ is selected from a covalent bond, NHCH₂, and CH₂NH;
R⁸ is selected from C₁-C₆-alkyl, halo-C₁-C₆-alkyl, hydroxy-C₁-C₆-alkyl, C₃-C₁₀-cycloalkyl, and halo-C₃-C₁₀-cycloalkyl;
R⁹ is selected from hydrogen, C₁-C₆-alkyl, halo-C₁-C₆-alkyl, hydroxy-C₁-C₆-alkyl, C₃-C₁₀-cycloalkyl, and halo-C₃-C₁₀-cycloalkyl;
(a) X is NR⁹ and Y is C; and
(i) A is selected from furanyl, thienyl, phenyl, and pyridyl;
B is selected from phenyl, pyridyl, pyrimidinyl, pyrazinyl, triazolyl, and imidazolyl;
L² is selected from a covalent bond CH₂O, OCH₂, CH₂NH, NHCH₂, CH₂, CH₂CH₂, CF₂CH₂, and CH₂CF₂;
C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, and oxazolidine;
R¹ is selected from hydrogen, halogen, C₁-C₆-alkyl, halo-C₁-C₆-alkyl, C₁-C₆-alkoxy, and halo-C₁-C₆-alkoxy;
R², R³, and R⁴ are each independently either absent or selected from hydrogen, halogen, cyano, and C₁-C₆-alkyl;
R⁵ is a group 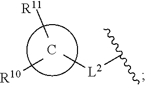
R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, oxo, and C₁-C₆-alkyl; or
(ii) A is selected from furanyl, thienyl, phenyl, and pyridyl;
B is selected from phenyl, pyridyl, pyrimidinyl, pyrazinyl, triazolyl, and imidazolyl;
L² is selected from a covalent bond CH₂O, OCH₂, CH₂NH, NHCH₂, CH₂, CH₂CH₂, CF₂CH₂, and CH₂CF₂;
C is selected from phenyl and pyridyl;
R¹ is selected from hydrogen, halogen, C₁-C₆-alkyl, halo-C₁-C₆-alkyl, C₁-C₆-alkoxy, and halo-C₁-C₆-alkoxy;
R², R³, and R⁴ are each independently either absent or selected from hydrogen, halogen, cyano, and C₁-C₆-alkyl;
R⁵ is a group 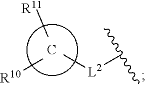
R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from cyano and hydroxy; and
R¹¹ is selected from hydrogen, hydroxy, and C₁-C₆-alkyl; or
(iii) A is selected from oxazolyl, pyrrolyl, pyrazinyl, cyclopropyl, 1,3,4-oxadiazolyl, pyrimidinyl;
B is selected from phenyl, pyridyl, pyrimidinyl, pyrazinyl, triazolyl, and imidazolyl;
L² is selected from a covalent bond, CH₂O, OCH₂, CH₂NH, NHCH₂, CH₂, CH₂CH₂, CF₂CH₂, and CH₂CF₂;
C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine, phenyl, and pyridyl;
R¹ is selected from hydrogen, halogen, C₁-C₆-alkyl, halo-C₁-C₆-alkyl, C₁-C₆-alkoxy, and halo-C₁-C₆-alkoxy;
R², R³, and R⁴ are each independently either absent or selected from hydrogen, halogen, cyano, and C₁-C₆-alkyl;
R⁵ is a group 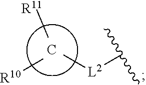
R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, and C₁-C₆-alkyl; or
(iv) A is selected from oxazolyl, furanyl, thienyl, pyrrolyl, 1,3,4-oxadiazolyl, cyclopropyl, phenyl, pyrazinyl, pyridyl, and pyrimidinyl;
B is selected from phenyl, pyridyl, pyrimidinyl, pyrazinyl, triazolyl, and imidazolyl;
L² is selected from a covalent bond, CH₂O, OCH₂, CH₂NH, NHCH₂, CH₂, CH₂CH₂, CF₂CH₂, and CH₂CF₂;
C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine, phenyl, and pyridyl;
R¹ is selected from hydroxy, carbamoyl-C₁-C₆-alkyl, hydroxy-C₁-C₆-alkyl, C₁-C₆-alkoxy-C₁-C₆-alkoxy, C₁-C₆-alkoxy-C₁-C₆-alkyl, C₃-C₁₀-cycloalkyl, NH₂SO₂—, C₁-C₆-alkyl-SO₂—, C₁-C₆-alkyl-SO₂—C₁-C₆-alkyl-, (C₁-C₆-alkyl)₂-PO—C₁-C₆-alkyl-, C₁-C₆-alkyl-PO₂—C₁-C₆-alkyl-, C₁-C₆-alkyl-sulfonimidoyl-C₁-C₆-alkyl-, halo-C₁-C₆-alkyl-sulfonimidoyl-C₁-C₆-alkyl-, C₁-C₆-alkyl-NH—SO₂—C₁-C₆-alkyl-, and C₁-C₆-alkyl-SO₂—NH—C₁-C₆-alkyl-; wherein said C₃-C₁₀-cycloalkyl is optionally substituted with one substituent selected from carbamoyl, C₁-C₆-alkyl-SO₂—, and C₁-C₆-alkyl-SO₂—NH—;
R², R³, and R⁴ are each independently either absent or selected from hydrogen, halogen, cyano, and C₁-C₆-alkyl;
R⁵ is a group 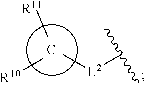
R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, and C₁-C₆-alkyl; or
(b) X is CR⁹ or N and Y is N;
L² is selected from a covalent bond, CH₂O, OCH₂, CH₂NH, NHCH₂, CH₂, CH₂CH₂, CF₂CH₂, and CH₂CF₂;
A is selected from furanyl, thienyl, oxazolyl, 1,3,4-oxadiazolyl, cyclopropyl, phenyl, pyrazinyl, pyridyl, and pyrimidinyl;
B is selected from triazolyl, imidazolyl, phenyl, and pyridyl;
C is selected from azetidine, cyclopropyl, piperidine, piperazine, pyridyl, pyrazine, pyrimidine, 1,2-dihydropyridine, 2-thia-6-azaspiro[3.3]heptane, 1,6-diazaspiro[3.3]heptane, 2,6-diazaspiro[3.3]heptane, 4,5-dihydroisoxazole, imidazolidine, oxazolidine, phenyl, pyrrolyl, pyrrolidinyl, pyrazolyl, and triazolyl;
R¹ is selected from halogen, hydroxy, carbamoyl-C₁-C₆-alkyl, C₁-C₆-alkyl, halo-C₁-C₆-alkyl, hydroxy-C₁-C₆-alkyl, C₁-C₆-alkoxy, halo-C₁-C₆-alkoxy, C₁-C₆-alkoxy-C₁-C₆-alkoxy, C₃-C₁₀-cycloalkyl, NH₂SO₂—, C₁-C₆-alkyl-SO₂—, C₁-C₆-alkyl-SO₂—C₁-C₆-alkyl-, (C₁-C₆-alkyl)₂-PO—C₁-C₆-alkyl-, C₁-C₆-alkyl-PO₂—C₁-C₆-alkyl-, C₁-C₆-alkyl-sulfonimidoyl-C₁-C₆-alkyl-, halo-C₁-C₆-alkyl-sulfonimidoyl-C₁-C₆-alkyl-, C₁-C₆-alkyl-NH—SO₂—C₁-C₆-alkyl-, and C₁-C₆-alkyl-SO₂—NH—C₁-C₆-alkyl-; wherein said C₃-C₁₀-cycloalkyl is optionally substituted with one substituent selected from carbamoyl, C₁-C₆-alkyl-SO₂—, and C₁-C₆-alkyl-SO₂—NH—;
R², R³, and R⁴ are each independently absent or selected from hydrogen, halogen, and cyano;
R⁵ is selected from C₁-C₆-alkoxy, C₁-C₆-alkoxy-C₁-C₆-alkyl, and a group 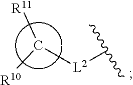
R⁶ is selected from hydrogen, halogen, and C₁-C₆-alkyl;
R⁷ is selected from hydrogen and halogen;
R⁸ is selected from C₁-C₆-alkyl, halo-C₁-C₆-alkyl, hydroxy-C₁-C₆-alkyl, C₃-C₁₀-cycloalkyl, and halo-C₃-C₁₀-cycloalkyl;
R⁹ is selected from hydrogen, C₁-C₆-alkyl, halo-C₁-C₆-alkyl, hydroxy-C₁-C₆-alkyl, C₃-C₁₀-cycloalkyl, and halo-C₃-C₁₀-cycloalkyl;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, oxo, and C₁-C₆-alkyl.

2. The compound of formula (II) according to claim 1, or a pharmaceutically acceptable salt thereof, wherein X is NR⁹, Y is C, represented by formula (I)

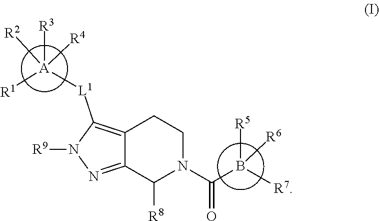

3. The compound of formula (II) according to claim 1, or a pharmaceutically acceptable salt thereof, wherein X is CR⁹ and Y is N, represented by formula (IIa)

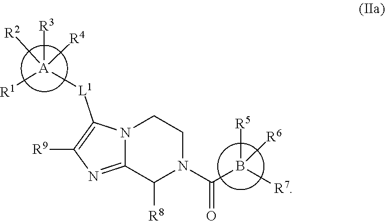

4. The compound of formula (II) according to claim 1, or a pharmaceutically acceptable salt thereof, wherein X and Y are both N, represented by formula (IIb)

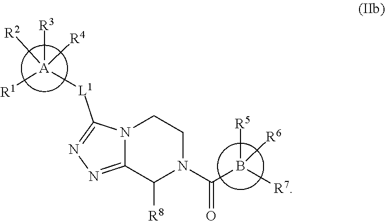

5. The compound of formula (II) according to any one of claims 1 to 4, or a pharmaceutically acceptable salt thereof, wherein
A is selected from furanyl, thienyl, phenyl, and pyridyl;
B is selected from phenyl, pyridyl, pyrimidinyl, pyrazinyl, triazolyl, and imidazolyl;
L² is selected from a covalent bond CH₂O, OCH₂, CH₂NH, NHCH₂, CH₂, CH₂CH₂, CF₂CH₂, and CH₂CF₂;
C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, and oxazolidine;
R¹ is selected from hydrogen, halogen, C₁-C₆-alkyl, halo-C₁-C₆-alkyl, C₁-C₆-alkoxy, and halo-C₁-C₆-alkoxy;
R², R³, and R⁴ are each independently either absent or selected from hydrogen, halogen, cyano, and C₁-C₆-alkyl;
R⁵ is a group 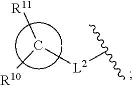
R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, oxo, and C₁-C₆-alkyl.

6. The compound of formula (II) according to any one of claims 1 to 4, or a pharmaceutically acceptable salt thereof, wherein
A is selected from furanyl, thienyl, phenyl, and pyridyl;
B is selected from phenyl, pyridyl, pyrimidinyl, pyrazinyl, triazolyl, and imidazolyl;
L² is selected from a covalent bond CH₂O, OCH₂, CH₂NH, NHCH₂, CH₂, CH₂CH₂, CF₂CH₂, and CH₂CF₂;
C is selected from phenyl and pyridyl;
R¹ is selected from hydrogen, halogen, C₁-C₆-alkyl, halo-C₁-C₆-alkyl, C₁-C₆-alkoxy, and halo-C₁-C₆-alkoxy;
R², R³, and R⁴ are each independently either absent or selected from hydrogen, halogen, cyano, and C₁-C₆-alkyl;
R⁵ is a group 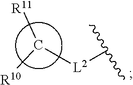
R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from cyano and hydroxy; and
R¹¹ is selected from hydrogen, hydroxy, and C₁-C₆-alkyl.

7. The compound of formula (II) according to any one of claims 1 to 4, or a pharmaceutically acceptable salt thereof, wherein
A is selected from oxazolyl, pyrrolyl, pyrazinyl, cyclopropyl, 1,3,4-oxadiazolyl, pyrimidinyl;
B is selected from phenyl, pyridyl, pyrimidinyl, pyrazinyl, triazolyl, and imidazolyl;
L² is selected from a covalent bond, CH₂O, OCH₂, CH₂NH, NHCH₂, CH₂, CH₂CH₂, CF₂CH₂, and CH₂CF₂;
C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine, phenyl, and pyridyl;
R¹ is selected from hydrogen, halogen, C₁-C₆-alkyl, halo-C₁-C₆-alkyl, C₁-C₆-alkoxy, and halo-C₁-C₆-alkoxy;
R², R³, and R⁴ are each independently either absent or selected from hydrogen, halogen, cyano, and C₁-C₆-alkyl;
R⁵ is a group 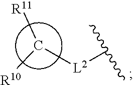
R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, and C₁-C₆-alkyl.

8. The compound of formula (II) according to any one of claims 1 to 4, or a pharmaceutically acceptable salt thereof, wherein
A is selected from oxazolyl, furanyl, thienyl, pyrrolyl, 1,3,4-oxadiazolyl, cyclopropyl, phenyl, pyrazinyl, pyridyl, and pyrimidinyl;
B is selected from phenyl, pyridyl, pyrimidinyl, pyrazinyl, triazolyl, and imidazolyl;
L² is selected from a covalent bond, CH₂O, OCH₂, CH₂NH, NHCH₂, CH₂, CH₂CH₂, CF₂CH₂, and CH₂CF₂;
C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine, phenyl, and pyridyl;
R¹ is selected from hydroxy, carbamoyl-C₁-C₆-alkyl, hydroxy-C₁-C₆-alkyl, C₁-C₆-alkoxy-C₁-C₆-alkoxy, C₁-C₆-alkoxy-C₁-C₆-alkyl, C₃-C₁₀-cycloalkyl, NH₂SO₂—, C₁-C₆-alkyl-SO₂—, C₁-C₆-alkyl-SO₂—C₁-C₆-alkyl-, (C₁-C₆-alkyl)₂-PO—C₁-C₆-alkyl-, C₁-C₆-alkyl-PO₂—C₁-C₆-alkyl-, C₁-C₆-alkyl-sulfonimidoyl-C₁-C₆-alkyl-, halo-C₁-C₆-alkyl-sulfonimidoyl-C₁-C₆-alkyl-, C₁-C₆-alkyl-NH—SO₂—C₁-C₆-alkyl-, and C₁-C₆-alkyl-SO₂—NH—C₁-C₆-alkyl-; wherein said C₃-C₁₀-cycloalkyl is optionally substituted with one substituent selected from carbamoyl, C₁-C₆-alkyl-SO₂—, and C₁-C₆-alkyl-SO₂—NH—;
R², R³, and R⁴ are each independently either absent or selected from hydrogen, halogen, cyano, and C₁-C₆-alkyl;
R⁵ is a group 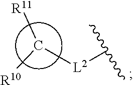
R⁶ is selected from hydrogen, halogen, C₁-C₆-alkyl, halo-C₁-C₆-alkyl, C₁-C₆-alkoxy, and halo-C₁-C₆-alkoxy;
R⁷ is selected from hydrogen and halogen;
R¹⁰ is selected from hydrogen, cyano, hydroxy, halogen, oxo, C₁-C₆-alkyl, C₁-C₆-alkyl-SO₂—, NH₂SO₂—, and halo-C₁-C₆-alkyl; and
R¹¹ is selected from hydrogen, hydroxy, and C₁-C₆-alkyl.

9. The compound of formula (II) according to claim 1, or a pharmaceutically acceptable salt thereof, wherein
(i) L² is selected from a covalent bond, OCH₂, CH₂O, NHCH₂, CH₂CH₂,
A is phenyl;
B is selected from phenyl, pyridyl, triazolyl, imidazolyl;
C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine;
R¹ is halogen;
R² is halogen;
R³ is hydrogen or halogen;
R⁴ is hydrogen or halogen;
R⁵ is a group 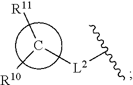
R⁶ is halogen or C₁-C₆-alkyl;
R⁷ is hydrogen or halogen;
R¹⁰ is selected from hydrogen, C₁-C₆-alkyl, halo-C₁-C₆-alkyl, C₁-C₆-alkyl-SO₂—, oxo, hydroxy, and cyano; and
R¹¹ is selected from hydrogen, C₁-C₆-alkyl, oxo; or
(ii) L² is a covalent bond;
A is phenyl;
B is triazolyl or phenyl;
C is pyridyl or phenyl;
R¹ is halogen;
R² is halogen;
R³ is hydrogen;
R⁴ is hydrogen;
R⁵ is a group 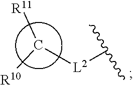
R⁶ is hydrogen or halogen;
R⁷ is hydrogen;
R¹⁰ is hydroxy or cyano; and
R¹¹ is hydrogen or halogen; or
(iii) L² is CH₂ or CH₂CH₂;
A is phenyl;
B is phenyl;
C is triazolyl or pyrazolyl;
R¹ is halogen;
R² is halogen;
R³ is hydrogen;
R⁴ is hydrogen;
R⁵ is a group 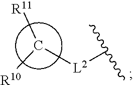
R⁶ is hydrogen or halogen;
R⁷ is hydrogen or halogen;
R¹⁰ is hydrogen; and
R¹¹ is hydrogen; or
(iv) A is selected from pyrazinyl, cyclopropyl, 1,3,4-oxadiazolyl, and pyrimidinyl;
B is phenyl;
R¹ is halo-C₁-C₆-alkyl;
R² is hydrogen;
R³ is hydrogen;
R⁴ is hydrogen;
R⁵ is C₁-C₆-alkoxy;
R⁶ is halogen; and
R⁷ is hydrogen; or
(v) L² is a covalent bond;
A is pyridyl or phenyl;
B is phenyl, triazolyl;
C is triazolyl, pyrazolyl, pyridyl;
R¹ is hydroxy, hydroxy-C₁-C₆-alkyl, C₁-C₆-alkoxy-C₁-C₆-alkoxy, C₁-C₆-alkyl-SO₂—, C₁-C₆-alkyl-SO₂—C₁-C₆-alkyl-, C₁-C₆-alkyl-SO₂—NH—C₁-C₆-alkyl-, C₁-C₆-alkyl-NH—SO₂—C₁-C₆-alkyl-, carbamoyl-C₁-C₆-alkyl, NH₂SO₂—, C₃-C₁₀-cycloalkyl; wherein said C₃-C₁₀-cycloalkyl is optionally substituted with one substituent selected from carbamoyl and C₁-C₆-alkyl-SO₂—NH—;
R² is hydrogen, halogen;
R³ is hydrogen;
R⁴ is hydrogen;
R⁵ is C₁-C₆-alkoxy or a group 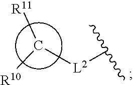
R⁶ is halogen;
R⁷ is hydrogen or halogen;
R¹⁰ is hydrogen, hydroxy; and
R¹¹ is hydrogen, halogen; or
(vi) L² is a covalent bond;
A is pyrazinyl;
B is phenyl or triazolyl;
C is selected from azetidinyl, 1,2-dihydropyridine, pyrrolyl, and 2-thia-6-azaspiro[3.3]heptane;
R¹ is halo-C₁-C₆-alkyl;
R² is hydrogen;
R³ is hydrogen;
R⁴ is hydrogen;
R⁵ is a group 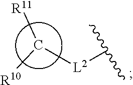
R⁶ is halogen;
R⁷ is halogen;
R¹⁰ is selected from NH₂SO₂—, cyano, and oxo; and
R¹¹ is hydrogen or oxo; or
(vii) L² is selected from a covalent bond, CH₂O, and CH₂CH₂;
A is phenyl;
B is phenyl or triazolyl;
C is selected from 1,2-dihydropyridine, pyrrolidinyl, pyrrolyl, 2-thia-6-azaspiro[3.3]heptane, and triazolyl;
R¹ is selected from NH₂SO₂—, C₁-C₆-alkyl-PO₂—C₁-C₆-alkyl-, C₃-C₁₀-cycloalkyl; wherein said C₃-C₁₀-cycloalkyl is optionally substituted with one substituent selected from carbamoyl, C₁-C₆-alkyl-SO₂—, and C₁-C₆-alkyl-SO₂—NH—;
R² is halogen;
R³ is hydrogen;
R⁴ is hydrogen;
R⁵ is a group 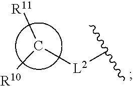
R⁶ is halogen;
R⁷ is halogen;
R¹⁰ is oxo or cyano; and
R¹¹ is hydrogen or oxo.

10. The compound of formula (II) according to claim 9, or a pharmaceutically acceptable salt thereof, wherein:
L² is selected from a covalent bond, OCH₂, CH₂O, NHCH₂, CH₂CH₂,
A is phenyl;
B is selected from phenyl, pyridyl, triazolyl, imidazolyl;
C is selected from azetidine, cyclopropyl, 2-thia-6-azaspiro[3.3]heptane, 1,6-diazaspiro[3.3]heptane, 2,6-diazaspiro[3.3]heptane, pyrrolidinyl, pyrrolyl, 1,2-dihydropyridine, 4,5-dihydroisoxazole, imidazolidine, oxazolidine;
R¹ is halogen;
R² is halogen;
R³ is hydrogen or halogen;
R⁴ is hydrogen or halogen;
R⁵ is a group 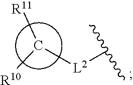
R⁶ is halogen or C₁-C₆-alkyl;
R⁷ is hydrogen or halogen;
R¹⁰ is selected from hydrogen, C₁-C₆-alkyl, halo-C₁-C₆-alkyl, C₁-C₆-alkyl-SO₂—, oxo, hydroxy, and cyano; and
R¹¹ is selected from hydrogen, C₁-C₆-alkyl, oxo.

11. The compound of formula (II) according to claim 9, or a pharmaceutically acceptable salt thereof, wherein:
L² is a covalent bond;
A is phenyl;
B is triazolyl or phenyl;
C is pyridyl or phenyl;
R¹ is halogen;
R² is halogen;
R³ is hydrogen;
R⁴ is hydrogen;
R⁵ is a group 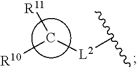
R⁶ is hydrogen or halogen;
R⁷ is hydrogen;
R¹⁰ is hydroxy or cyano; and
R¹¹ is hydrogen or halogen.

12. The compound of formula (II) according to claim 9, or a pharmaceutically acceptable salt thereof, wherein:
L² is CH₂ or CH₂CH₂;
A is phenyl;
B is phenyl;
C is triazolyl or pyrazolyl;
R¹ is halogen;
R² is halogen;
R³ is hydrogen;
R⁴ is hydrogen;
R⁵ is a group 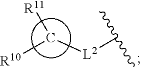
R⁶ is hydrogen or halogen;
R⁷ is hydrogen or halogen;
R¹⁰ is hydrogen; and
R¹¹ is hydrogen.

13. The compound of formula (II) according to claim 9, or a pharmaceutically acceptable salt thereof, wherein:
A is selected from pyrazinyl, cyclopropyl, 1,3,4-oxadiazolyl, and pyrimidinyl;
B is phenyl;
R¹ is halo-C₁-C₆-alkyl;
R² is hydrogen;
R³ is hydrogen;
R⁴ is hydrogen;
R⁵ is C₁-C₆-alkoxy;
R⁶ is halogen; and
R⁷ is hydrogen.

14. The compound of formula (II) according to claim 9, or a pharmaceutically acceptable salt thereof, wherein:
L² is a covalent bond;
A is pyridyl or phenyl;
B is phenyl, triazolyl;
C is triazolyl, pyrazolyl, pyridyl;
R¹ is hydroxy, hydroxy-C₁-C₆-alkyl, C₁-C₆-alkoxy-C₁-C₆-alkoxy, C₁-C₆-alkyl-SO₂—, C₁-C₆-alkyl-SO₂—C₁-C₆-alkyl-, C₁-C₆-alkyl-SO₂—NH—C₁-C₆-alkyl-, C₁-C₆-alkyl-NH—SO₂—C₁-C₆-alkyl-, carbamoyl-C₁-C₆-alkyl, NH₂SO₂—, C₃-C₁₀-cycloalkyl; wherein said C₃-C₁₀-cycloalkyl is optionally substituted with one substituent selected from carbamoyl and C₁-C₆-alkyl-SO₂—NH—;
R² is hydrogen, halogen;
R³ is hydrogen;
R⁴ is hydrogen;
R⁵ is C₁-C₆-alkoxy or a group 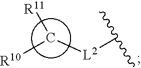
R⁶ is halogen;
R⁷ is hydrogen or halogen;
R¹⁰ is hydrogen, hydroxy; and
R¹¹ is hydrogen, halogen.

15. The compound of formula (II) according to claim 9, or a pharmaceutically acceptable salt thereof, wherein:
L² is a covalent bond;
A is pyrazinyl;
B is phenyl or triazolyl;
C is selected from azetidinyl, 1,2-dihydropyridine, pyrrolyl, and 2-thia-6-azaspiro[3.3]heptane;
R¹ is halo-C₁-C₆-alkyl;
R² is hydrogen;
R³ is hydrogen;
R⁴ is hydrogen;
R⁵ is a group 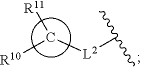
R⁶ is halogen;
R⁷ is halogen;
R¹⁰ is selected from NH₂SO₂—, cyano, and oxo; and
R¹¹ is hydrogen or oxo.

16. The compound of formula (II) according to claim 9, or a pharmaceutically acceptable salt thereof, wherein:
L² is selected from a covalent bond, CH₂O, and CH₂CH₂;
A is phenyl;
B is phenyl or triazolyl;
C is selected from 1,2-dihydropyridine, pyrrolidinyl, pyrrolyl, 2-thia-6-azaspiro[3.3]heptane, and triazolyl;
R¹ is selected from NH₂SO₂—, C₁-C₆-alkyl-PO₂—C₁-C₆-alkyl-, C₃-C₁₀-cycloalkyl;
wherein said C₃-C₁₀-cycloalkyl is optionally substituted with one substituent selected from carbamoyl, C₁-C₆-alkyl-SO₂—, and C₁-C₆-alkyl-SO₂—NH—;
R² is halogen;
R³ is hydrogen;
R⁴ is hydrogen;
R⁵ is a group 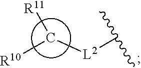
R⁶ is halogen;
R⁷ is halogen;
R¹⁰ is oxo or cyano; and
R¹¹ is hydrogen or oxo.

17. The compound of formula (II) according to claim 9, or a pharmaceutically acceptable salt thereof, wherein
(i) L² is selected from a covalent bond, CH₂O, and NHCH₂;
A is phenyl;
B is phenyl;
C is selected from pyrrolidinyl, 1,2-dihydropyridine, imidazolidine, oxazolidine;
R¹ is halogen;
R² is halogen;
R³ is hydrogen;
R⁴ is hydrogen;
R⁵ is a group 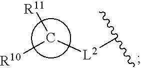
R⁶ is halogen;
R⁷ is halogen;
R¹⁰ is oxo; and
R¹¹ is hydrogen; or
(ii) L² is a covalent bond;
A is phenyl;
B is triazolyl;
C is pyridyl;
R¹ is halogen;
R² is halogen;
R³ is hydrogen;
R⁴ is hydrogen;
R⁵ is a group 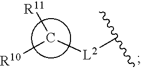
R⁶ is hydrogen;
R⁷ is absent;
R¹⁰ is hydroxy; and
R¹¹ is hydrogen or halogen; or
(iii) L² is CH₂CH₂;
A is phenyl;
B is phenyl;
C is triazolyl or pyrazolyl;
R¹ is halogen;
R² is halogen;
R³ is hydrogen;
R⁴ is hydrogen;
R⁵ is a group 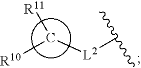
R⁶ is halogen;
R⁷ is halogen;
R¹⁰ is hydrogen; and
R¹¹ is hydrogen; or
(iv) L² is a covalent bond;
A is phenyl;
B is phenyl;
C is pyrrolyl;
R¹ is C₃-C₁₀-cycloalkyl; wherein said C₃-C₁₀-cycloalkyl is substituted with one C₁-C₆-alkyl-SO₂—NH— substituent;
R² is halogen;
R³ is hydrogen;
R⁴ is hydrogen;
R⁵ is a group 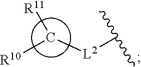
R⁶ is halogen;
R⁷ is halogen;
R¹⁰ is cyano; and
R¹¹ is hydrogen.

18. The compound of formula (II) according to claim 17, or a pharmaceutically acceptable salt thereof, wherein
(i) L² is selected from a covalent bond, CH₂O, and NHCH₂;
A is phenyl;
B is phenyl;
C is selected from pyrrolidinyl, 1,2-dihydropyridine, imidazolidine, oxazolidine;
R¹ is fluoro;
R² is fluoro;
R³ is hydrogen;
R⁴ is hydrogen;
R⁵ is a group 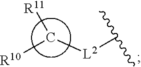
R⁶ is fluoro;
R⁷ is chloro;
R¹⁰ is oxo; and
R¹¹ is hydrogen; or
(ii) L² is a covalent bond;
A is phenyl;
B is triazolyl;
C is pyridyl;
R¹ is fluoro;
R² is fluoro;
R³ is hydrogen;
R⁴ is hydrogen;
R⁵ is a group 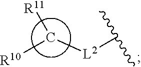
R⁶ is hydrogen;
R⁷ is absent;
R¹⁰ is hydroxy; and
R¹¹ is hydrogen or chloro; or
(iii) L² is CH₂CH₂;
A is phenyl;
B is phenyl;
C is triazolyl or pyrazolyl;
R¹ is fluoro;
R² is fluoro;
R³ is hydrogen;
R⁴ is hydrogen;
R⁵ is a group 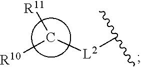
R⁶ is fluoro;
R⁷ is chloro;
R¹⁰ is hydrogen; and
R¹¹ is hydrogen; or
(iv) L² is a covalent bond;
A is phenyl;
B is phenyl;
C is pyrrolyl;
R¹ is cyclopropyl; wherein said cyclopropyl is substituted with one methyl-SO₂—NH— substituent;
R² is chloro;
R³ is hydrogen;
R⁴ is hydrogen;
R⁵ is a group 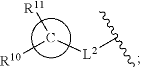
R⁶ is fluoro;
R⁷ is chloro;
R¹⁰ is cyano; and
R¹¹ is hydrogen.

19. The compound of formula (I) according to any one of claims 1 to 18, or a pharmaceutically acceptable salt thereof, wherein L¹ is selected from a covalent bond and and NHCH₂.

20. The compound of formula (I) according to claim 19, or a pharmaceutically acceptable salt thereof, wherein L¹ is a covalent bond.

21. The compound of formula (I) according to any one of claims 1 to 20, or a pharmaceutically acceptable salt thereof, wherein R⁸ is C₁-C₆-alkyl.

22. The compound of formula (I) according to claim 21, or a pharmaceutically acceptable salt thereof, wherein R⁸ is methyl.

23. The compound of formula (I) according to any one of claims 1 to 22, or a pharmaceutically acceptable salt thereof, wherein R⁹ is C₁-C₆-alkyl.

24. The compound of formula (I) according to claim 23, or a pharmaceutically acceptable salt thereof, wherein R⁹ is methyl.

25. The compound of formula (II) according to claim 1, or a pharmaceutically acceptable salt thereof, selected from:
4-[[2-Chloro-3-[3-(3,5-difluorophenyl)-2,7-dimethyl-5,7-dihydro-4H-pyrazolo[3,4-c]pyridine-6-carbonyl]-5-fluoro-phenoxy]methyl]pyrrolidin-2-one;
5-[2-Chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-1H-pyridin-2-one;
5-[[2-Chloro-3-[3-(3,5-difluorophenyl)-2,7-dimethyl-5,7-dihydro-4H-pyrazolo[3,4-c]pyridine-6-carbonyl]-5-fluoro-phenoxy]methyl]oxazolidin-2-one;
4-[3-[(7S)-3-(3,5-Difluorophenyl)-2,7-dimethyl-5,7-dihydro-4H-pyrazolo[3,4-c]pyridine-6-carbonyl]-5-fluoro-phenyl]-1H-pyrrole-2-carbonitrile;
4-[2-Chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-1H-pyridin-2-one;
N-[1-[3-Chloro-5-[6-[2-chloro-5-fluoro-3-(6-oxo-1H-pyridin-3-yl)benzoyl]-2,7-dimethyl-5,7-dihydro-4H-pyrazolo[3,4-c]pyridin-3-yl]phenyl]cyclopropyl]methanesulfonamide;
4-[2-Chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-1H-pyrrole-2-carbonitrile;
3-Chloro-5-[6-[2-chloro-5-fluoro-3-(6-oxo-1H-pyridin-3-yl)benzoyl]-2,7-dimethyl-5,7-dihydro-4H-pyrazolo[3,4-c]pyridin-3-yl]benzenesulfonamide;
4-[2-Chloro-3-[(7S)-2,7-dimethyl-3-[6-(trifluoromethyl)pyrazin-2-yl]-5,7-dihydro-4H-pyrazolo[3,4-c]pyridine-6-carbonyl]-5-fluoro-phenyl]-1H-pyrrole-2-carbonitrile;
N-[[3-Chloro-5-[(7S)-6-(2-chloro-3-methoxy-benzoyl)-2,7-dimethyl-5,7-dihydro-4H-pyrazolo[3,4-c]pyridin-3-yl]phenyl]methyl]methanesulfonamide;
N-[1-[3-Chloro-5-[(7S)-6-(2-chloro-3-methoxy-benzoyl)-2,7-dimethyl-5,7-dihydro-4H-pyrazolo[3,4-c]pyridin-3-yl]phenyl]cyclopropyl]methanesulfonamide;
5-[[3-Chloro-4-[(7S)-3-(3,5-difluorophenyl)-2,7-dimethyl-5,7-dihydro-4H-pyrazolo[3,4-c]pyridine-6-carbonyl]-2-pyridyl]oxymethyl]oxazolidin-2-one;
[2-Chloro-3-(2,2-dioxo-2λ⁶-thia-6-azaspiro[3.3]heptan-6-yl)-5-fluoro-phenyl]-[(7S)-3-(3,5-difluorophenyl)-2,7-dimethyl-5,7-dihydro-4H-pyrazolo[3,4-c]pyridin-6-yl]methanone;
[2-Chloro-5-fluoro-3-(3-methylsulfonylazetidin-1-yl)phenyl]-[(7S)-3-(3,5-difluorophenyl)-2,7-dimethyl-5,7-dihydro-4H-pyrazolo[3,4-c]pyridin-6-yl]methanone;
3-Chloro-5-[(7S)-6-(2-chloro-3-methoxy-benzoyl)-2,7-dimethyl-5,7-dihydro-4H-pyrazolo[3,4-c]pyridin-3-yl]benzenesulfonamide;
2-[3-Chloro-5-[(7S)-6-(2-chloro-3-methoxy-benzoyl)-2,7-dimethyl-5,7-dihydro-4H-pyrazolo[3,4-c]pyridin-3-yl]phenyl]acetamide;
[2-Chloro-3-(2,2-dioxo-2λ⁶-thia-6-azaspiro[3.3]heptan-6-yl)-5-fluoro-phenyl]-[3-(3,5-difluorophenyl)-2,7-dimethyl-5,7-dihydro-4H-pyrazolo[3,4-c]pyridin-6-yl]methanone;
5-[[3-Chloro-4-[3-(3,5-difluorophenyl)-2,7-dimethyl-5,7-dihydro-4H-pyrazolo[3,4-c]pyridine-6-carbonyl]-2-pyridyl]oxymethyl]oxazolidin-2-one;
(2-Chloro-3-methoxy-phenyl)-[(7S)-3-[3-chloro-5-(1-methylsulfonylcyclopropyl)phenyl]-2,7-dimethyl-5,7-dihydro-4H-pyrazolo[3,4-c]pyridin-6-yl]methanone;
1-[3-Chloro-5-[(7S)-6-(2-chloro-3-methoxy-benzoyl)-2,7-dimethyl-5,7-dihydro-4H-pyrazolo[3,4-c]pyridin-3-yl]phenyl]-N-methyl-methanesulfonamide;
[2-Chloro-5-fluoro-3-(3-methylsulfonylazetidin-1-yl)phenyl]-[3-(3,5-difluorophenyl)-2,7-dimethyl-5,7-dihydro-4H-pyrazolo[3,4-c]pyridin-6-yl]methanone;
3-Chloro-5-[6-[2-chloro-5-fluoro-3-(1H-1,2,4-triazol-3-yl)benzoyl]-2,7-dimethyl-5,7-dihydro-4H-pyrazolo[3,4-c]pyridin-3-yl]benzenesulfonamide;
(2-Chloro-3-methoxy-phenyl)-[(7S)-3-[3-chloro-5-(methylsulfonylmethyl)phenyl]-2,7-dimethyl-5,7-dihydro-4H-pyrazolo[3,4-c]pyridin-6-yl]methanone;
3-[3-[3-(3,5-Difluorophenyl)-2,7-dimethyl-5,7-dihydro-4H-pyrazolo[3,4-c]pyridine-6-carbonyl]-4-fluoro-phenyl]benzonitrile;
[2-Chloro-5-fluoro-3-[[1-(trifluoromethyl)cyclopropyl]methoxy]phenyl]-[3-(3,5-difluorophenyl)-2,7-dimethyl-5,7-dihydro-4H-pyrazolo[3,4-c]pyridin-6-yl]methanone;
4-[2-[3-Chloro-4-[(7S)-3-(3,5-difluorophenyl)-2,7-dimethyl-5,7-dihydro-4H-pyrazolo[3,4-c]pyridine-6-carbonyl]phenyl]ethyl]pyrrolidin-2-one;
1-[3-Chloro-5-[(7S)-6-(2-chloro-3-methoxy-benzoyl)-2,7-dimethyl-5,7-dihydro-4H-pyrazolo[3,4-c]pyridin-3-yl]phenyl]cyclopropanecarboxamide;
[1-(5-Chloro-2-hydroxy-3-pyridyl)-1,2,4-triazol-3-yl]-[(7S)-3-(3,5-difluorophenyl)-2,7-dimethyl-5,7-dihydro-4H-pyrazolo[3,4-c]pyridin-6-yl]methanone;
N-[1-[3-Chloro-5-[6-[2-chloro-5-fluoro-3-(2-oxo-1H-pyridin-4-yl)benzoyl]-2,7-dimethyl-5,7-dihydro-4H-pyrazolo[3,4-c]pyridin-3-yl]phenyl]cyclopropyl]methanesulfonamide;
5-[2-Chloro-3-[3-[3-chloro-5-(1-methylsulfonylcyclopropyl)phenyl]-2,7-dimethyl-5,7-dihydro-4H-pyrazolo[3,4-c]pyridine-6-carbonyl]-5-fluoro-phenyl]-1H-pyridin-2-one;
[(7S)-3-(3,5-Difluorophenyl)-2,7-dimethyl-5,7-dihydro-4H-pyrazolo[3,4-c]pyridin-6-yl]-[1-(5-fluoro-2-hydroxy-3-pyridyl)-1,2,4-triazol-3-yl]methanone;
2-[3-Chloro-5-[(7R)-6-(2-chloro-3-methoxy-benzoyl)-2,7-dimethyl-5,7-dihydro-4H-pyrazolo[3,4-c]pyridin-3-yl]phenyl]acetamide;
[(7S)-3-(3,5-Difluorophenyl)-2,7-dimethyl-5,7-dihydro-4H-pyrazolo[3,4-c]pyridin-6-yl]-[1-(6-hydroxy-3-pyridyl)-1,2,4-triazol-3-yl]methanone;
5-[2-Chloro-3-[(7R)-3-(3,5-difluorophenyl)-2,7-dimethyl-5,7-dihydro-4H-pyrazolo[3,4-c]pyridine-6-carbonyl]-5-fluoro-phenyl]-1H-pyridin-2-one;
(2-Chloro-3-methoxy-phenyl)-[(7S)-2,7-dimethyl-3-[6-(trifluoromethyl)pyrazin-2-yl]-5,7-dihydro-4H-pyrazolo[3,4-c]pyridin-6-yl]methanone;
2-[3-[(7S)-6-(2-Chloro-3-methoxy-benzoyl)-2,7-dimethyl-5,7-dihydro-4H-pyrazolo[3,4-c]pyridin-3-yl]-5-fluoro-phenyl]acetamide;
[(7S)-3-(3,5-Difluorophenyl)-2,7-dimethyl-5,7-dihydro-4H-pyrazolo[3,4-c]pyridin-6-yl]-[1-(2-hydroxy-3-pyridyl)-1,2,4-triazol-3-yl]methanone;
4-[2-[3-Chloro-4-[3-(3,5-difluorophenyl)-2,7-dimethyl-5,7-dihydro-4H-pyrazolo[3,4-c]pyridine-6-carbonyl]phenyl]ethyl]pyrrolidin-2-one;
1-[2-Chloro-3-[2,7-dimethyl-3-[6-(trifluoromethyl)pyrazin-2-yl]-5,7-dihydro-4H-pyrazolo[3,4-c]pyridine-6-carbonyl]-5-fluoro-phenyl]azetidine-3-sulfonamide;
(2-Chloro-3-methoxy-phenyl)-[(7S)-2,7-dimethyl-3-[2-(trifluoromethyl)pyrimidin-5-yl]-5,7-dihydro-4H-pyrazolo[3,4-c]pyridin-6-yl]methanone;
3-Chloro-5-[(7S)-2,7-dimethyl-6-[1-(2-oxo-1H-pyridin-3-yl)-1,2,4-triazole-3-carbonyl]-5,7-dihydro-4H-pyrazolo[3,4-c]pyridin-3-yl]benzenesulfonamide;
[2-Chloro-3-(5,5-dimethyl-4H-isoxazol-3-yl)-5-fluoro-phenyl]-[3-(3,5-difluorophenyl)-2,7-dimethyl-5,7-dihydro-4H-pyrazolo[3,4-c]pyridin-6-yl]methanone;
(2-Chloro-3-methoxy-phenyl)-[(7S)-2,7-dimethyl-3-[5-(trifluoromethyl)-1,3,4-oxadiazol-2-yl]-5,7-dihydro-4H-pyrazolo[3,4-c]pyridin-6-yl]methanone;
(2-Chloro-3-methoxy-phenyl)-[(7S)-2,7-dimethyl-3-[[[1-(trifluoromethyl)cyclopropyl]amino]methyl]-5,7-dihydro-4H-pyrazolo[3,4-c]pyridin-6-yl]methanone;
3-Chloro-5-[(7R)-2,7-dimethyl-6-[1-(2-oxo-1H-pyridin-3-yl)-1,2,4-triazole-3-carbonyl]-5,7-dihydro-4H-pyrazolo[3,4-c]pyridin-3-yl]benzenesulfonamide;
(2-Chloro-3-methoxy-phenyl)-[(7S)-3-(6-hydroxy-2-pyridyl)-2,7-dimethyl-5,7-dihydro-4H-pyrazolo[3,4-c]pyridin-6-yl]methanone;
[3-(3,5-Difluorophenyl)-2,7-dimethyl-5,7-dihydro-4H-pyrazolo[3,4-c]pyridin-6-yl]-(2-pyrrolidin-3-yl-1,2,4-triazol-3-yl)methanone;hydrochloride;
1-[3-[3-(3,5-Difluorophenyl)-2,7-dimethyl-5,7-dihydro-4H-pyrazolo[3,4-c]pyridine-6-carbonyl]-2-methyl-phenyl]imidazolidin-2-one;
3-Chloro-5-[2,7-dimethyl-6-[1-(2-oxo-1H-pyridin-3-yl)-1,2,4-triazole-3-carbonyl]-5,7-dihydro-4H-pyrazolo[3,4-c]pyridin-3-yl]benzenesulfonamide;
[3-(3,5-Difluorophenyl)-2,7-dimethyl-5,7-dihydro-4H-pyrazolo[3,4-c]pyridin-6-yl]-[2-(triazol-1-ylmethyl)phenyl]methanone;
5-[[3-Chloro-4-[(7R)-3-(3,5-difluorophenyl)-2,7-dimethyl-5,7-dihydro-4H-pyrazolo[3,4-c]pyridine-6-carbonyl]-2-pyridyl]oxymethyl]oxazolidin-2-one;
[2-Chloro-3-(2,2-dioxo-2λ⁶-thia-6-azaspiro[3.3]heptan-6-yl)-5-fluoro-phenyl]-[(7R)-3-(3,5-difluorophenyl)-2,7-dimethyl-5,7-dihydro-4H-pyrazolo[3,4-c]pyridin-6-yl]methanone;
N-[[3-Chloro-5-[(7R)-6-(2-chloro-3-methoxy-benzoyl)-2,7-dimethyl-5,7-dihydro-4H-pyrazolo[3,4-c]pyridin-3-yl]phenyl]methyl]methanesulfonamide;
4-[2-[3-Chloro-4-[(7R)-3-(3,5-difluorophenyl)-2,7-dimethyl-5,7-dihydro-4H-pyrazolo[3,4-c]pyridine-6-carbonyl]phenyl]ethyl]pyrrolidin-2-one;
[2-Chloro-3-(2,2-dioxo-2λ⁶-thia-6-azaspiro[3.3]heptan-6-yl)-5-fluoro-phenyl]-[(7S)-2,7-dimethyl-3-[6-(trifluoromethyl)pyrazin-2-yl]-5,7-dihydro-4H-pyrazolo[3,4-c]pyridin-6-yl]methanone;
2-[5-[(7S)-6-(2-Chloro-3-methoxy-benzoyl)-2,7-dimethyl-5,7-dihydro-4H-pyrazolo[3,4-c]pyridin-3-yl]-3-pyridyl]acetamide;
[2-Chloro-3-(2,2-dioxo-2λ⁶-thia-6-azaspiro[3.3]heptan-6-yl)-5-fluoro-phenyl]-[(7R)-2,7-dimethyl-3-[6-(trifluoromethyl)pyrazin-2-yl]-5,7-dihydro-4H-pyrazolo[3,4-c]pyridin-6-yl]methanone;
5-[2-Chloro-3-[(7S)-2,7-dimethyl-3-[6-(trifluoromethyl)pyrazin-2-yl]-5,7-dihydro-4H-pyrazolo[3,4-c]pyridine-6-carbonyl]-5-fluoro-phenyl]-1H-pyridin-2-one;
3-[3-[(7S)-2,7-Dimethyl-3-[6-(trifluoromethyl)pyrazin-2-yl]-5,7-dihydro-4H-pyrazolo[3,4-c]pyridine-6-carbonyl]-1,2,4-triazol-1-yl]-1H-pyridin-2-one;
(2-Chloro-3-methoxy-phenyl)-[(7S)-2,7-dimethyl-3-(5-methylsulfonyl-3-pyridyl)-5,7-dihydro-4H-pyrazolo[3,4-c]pyridin-6-yl]methanone;
[(7S)-3-[3-chloro-5-(2-methoxyethoxy)phenyl]-2,7-dimethyl-5,7-dihydro-4H-pyrazolo[3,4-c]pyridin-6-yl]-(2-chloro-3-methoxy-phenyl)methanone;
1-[3-chloro-5-[6-[2-chloro-5-fluoro-3-[(5-oxopyrrolidin-3-yl)methoxy]benzoyl]-2,7-dimethyl-5,7-dihydro-4H-pyrazolo[3,4-c]pyridin-3-yl]phenyl]cyclopropanecarboxamide;
1-[3-chloro-5-[(7S)-6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropanecarboxamide;
1-[3-chloro-5-[(7R)-6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropanecarboxamide;
5-[2-chloro-3-[(7S)-3-[3-chloro-5-(1-methylsulfonylcyclopropyl)phenyl]-2,7-dimethyl-5,7-dihydro-4H-pyrazolo[3,4-c]pyridine-6-carbonyl]-5-fluoro-phenyl]-1H-pyridin-2-one;
5-[2-chloro-3-[(7R)-3-[3-chloro-5-(1-methylsulfonylcyclopropyl)phenyl]-2,7-dimethyl-5,7-dihydro-4H-pyrazolo[3,4-c]pyridine-6-carbonyl]-5-fluoro-phenyl]-1H-pyridin-2-one;
1-[3-chloro-5-[(7S)-6-[2-chloro-3-(2,2-dioxo-2λ⁶-thia-6-azaspiro[3.3]heptan-6-yl)-5-fluoro-benzoyl]-2,7-dimethyl-5,7-dihydro-4H-pyrazolo[3,4-c]pyridin-3-yl]phenyl]cyclopropanecarboxamide;
1-[3-chloro-5-[(7R)-6-[2-chloro-3-(2,2-dioxo-2λ⁶-thia-6-azaspiro[3.3]heptan-6-yl)-5-fluoro-benzoyl]-2,7-dimethyl-5,7-dihydro-4H-pyrazolo[3,4-c]pyridin-3-yl]phenyl]cyclopropanecarboxamide;
3-[5-chloro-4-[(7S)-3-(3,5-difluorophenyl)-2,7-dimethyl-5,7-dihydro-4H-pyrazolo[3,4-c]pyridine-6-carbonyl]imidazol-1-yl]-1H-pyridin-2-one;
3-[3-[(7S)-3-[3-chloro-5-(1-methylsulfonylcyclopropyl)phenyl]-2,7-dimethyl-5,7-dihydro-4H-pyrazolo[3,4-c]pyridine-6-carbonyl]-1,2,4-triazol-1-yl]-1H-pyridin-2-one;
[(7R)-3-[3-chloro-5-(2-methoxyethoxy)phenyl]-2,7-dimethyl-5,7-dihydro-4H-pyrazolo[3,4-c]pyridin-6-yl]-(2-chloro-3-methoxy-phenyl)methanone;
N-[[2-chloro-6-[6-(2-chloro-3-methoxy-benzoyl)-2,7-dimethyl-5,7-dihydro-4H-pyrazolo[3,4-c]pyridin-3-yl]-4-pyridyl]methyl]methanesulfonamide;
(2-chloro-3-methoxy-phenyl)-[(7S)-2,7-dimethyl-3-[2-(trifluoromethyl)pyrimidin-4-yl]-5,7-dihydro-4H-pyrazolo[3,4-c]pyridin-6-yl]methanone;
4-[2-chloro-5-fluoro-3-[(7S)-3-[3-fluoro-5-[[methyl(dioxo)-λ⁶-phosphanyl]methyl]phenyl]-2,7-dimethyl-5,7-dihydro-4H-pyrazolo[3,4-c]pyridine-6-carbonyl]phenyl]-1H-pyrrole-2-carbonitrile;
4-[2-chloro-5-fluoro-3-[(7R)-3-[3-fluoro-5-[[methyl(dioxo)-λ⁶-phosphanyl]methyl]phenyl]-2,7-dimethyl-5,7-dihydro-4H-pyrazolo[3,4-c]pyridine-6-carbonyl]phenyl]-1H-pyrrole-2-carbonitrile;
N-[1-[3-chloro-5-[6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropyl]methanesulfonamide;
N-[1-[3-chloro-5-[(7S)-6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropyl]methanesulfonamide;
N-[1-[3-chloro-5-[(7R)-6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropyl]methanesulfonamide;
N-[1-[3-chloro-5-[6-[2-chloro-5-fluoro-3-[2-(1H-triazol-4-yl)ethyl]benzoyl]-2,7-dimethyl-5,7-dihydro-4H-pyrazolo[3,4-c]pyridin-3-yl]phenyl]cyclopropyl]methanesulfonamide;
N-[1-[3-chloro-5-[(7S)-6-[2-chloro-5-fluoro-3-[2-(1H-triazol-4-yl)ethyl]benzoyl]-2,7-dimethyl-5,7-dihydro-4H-pyrazolo[3,4-c]pyridin-3-yl]phenyl]cyclopropyl]methanesulfonamide;
N-[1-[3-chloro-5-[(7R)-6-[2-chloro-5-fluoro-3-[2-(1H-triazol-4-yl)ethyl]benzoyl]-2,7-dimethyl-5,7-dihydro-4H-pyrazolo[3,4-c]pyridin-3-yl]phenyl]cyclopropyl]methanesulfonamide;
4-[2-chloro-3-[(7S)-2,7-dimethyl-3-(2,3,4,5-tetrafluorophenyl)-5,7-dihydro-4H-pyrazolo[3,4-c]pyridine-6-carbonyl]-5-fluoro-phenyl]-1H-pyrrole-2-carbonitrile;
N-[1-[3-chloro-5-[(7S)-6-[1-(5-chloro-2-hydroxy-3-pyridyl)-1,2,4-triazole-3-carbonyl]-2,7-dimethyl-5,7-dihydro-4H-pyrazolo[3,4-c]pyridin-3-yl]phenyl]cyclopropyl]methanesulfonamide;
4-[2-chloro-3-[(7S)-2,7-dimethyl-3-(3,4,5-trifluorophenyl)-5,7-dihydro-4H-pyrazolo[3,4-c]pyridine-6-carbonyl]-5-fluoro-phenyl]-1H-pyrrole-2-carbonitrile;
4-[2-chloro-3-[(7R)-2,7-dimethyl-3-(3,4,5-trifluorophenyl)-5,7-dihydro-4H-pyrazolo[3,4-c]pyridine-6-carbonyl]-5-fluoro-phenyl]-1H-pyrrole-2-carbonitrile;
N-[1-[3-chloro-5-[(7S)-6-[2,5-difluoro-3-(1H-pyrazol-4-yl)benzoyl]-2,7-dimethyl-5,7-dihydro-4H-pyrazolo[3,4-c]pyridin-3-yl]phenyl]cyclopropyl]methanesulfonamide;
[(7S)-3-[3-chloro-5-(2-hydroxy-2-methyl-propyl)phenyl]-2,7-dimethyl-5,7-dihydro-4H-pyrazolo[3,4-c]pyridin-6-yl]-(2-chloro-3-methoxy-phenyl)methanone;
3-[3-[(7R)-2,7-dimethyl-3-[6-(trifluoromethyl)pyrazin-2-yl]-5,7-dihydro-4H-pyrazolo[3,4-c]pyridine-6-carbonyl]-1,2,4-triazol-1-yl]-1H-pyridin-2-one;
3-chloro-5-[2-chloro-3-[3-(3,5-difluorophenyl)-2,7-dimethyl-5,7-dihydro-4H-pyrazolo[3,4-c]pyridine-6-carbonyl]-5-fluoro-phenyl]-3H-pyridin-2-one;
3-chloro-5-[2-chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-3H-pyridin-2-one;
3-chloro-5-[2-chloro-3-[(7R)-3-(3,5-difluorophenyl)-2,7-dimethyl-5,7-dihydro-4H-pyrazolo[3,4-c]pyridine-6-carbonyl]-5-fluoro-phenyl]-3H-pyridin-2-one;
[1-(5-chloro-2-hydroxy-3-pyridyl)-1,2,4-triazol-3-yl]-[(7S)-3-(3,5-difluorophenyl)-2,7-dimethyl-5,7-dihydro-4H-pyrazolo[3,4-c]pyridin-6-yl]methanone;
[1-(5-chloro-2-hydroxy-3-pyridyl)-1,2,4-triazol-3-yl]-[(7R)-3-(3,5-difluorophenyl)-2,7-dimethyl-5,7-dihydro-4H-pyrazolo[3,4-c]pyridin-6-yl]methanone;
[2-chloro-5-fluoro-3-[2-(1H-pyrazol-4-yl)ethyl]phenyl]-[(7S)-3-(3,5-difluorophenyl)-2,7-dimethyl-5,7-dihydro-4H-pyrazolo[3,4-c]pyridin-6-yl]methanone;
[2-chloro-5-fluoro-3-[2-(1H-pyrazol-4-yl)ethyl]phenyl]-[(7R)-3-(3,5-difluorophenyl)-2,7-dimethyl-5,7-dihydro-4H-pyrazolo[3,4-c]pyridin-6-yl]methanone;
[2-chloro-5-fluoro-3-[2-(1H-triazol-4-yl)ethyl]phenyl]-[(7S)-3-(3,5-difluorophenyl)-2,7-dimethyl-5,7-dihydro-4H-pyrazolo[3,4-c]pyridin-6-yl]methanone;
[2-chloro-5-fluoro-3-[2-(1H-triazol-4-yl)ethyl]phenyl]-[(7R)-3-(3,5-difluorophenyl)-2,7-dimethyl-5,7-dihydro-4H-pyrazolo[3,4-c]pyridin-6-yl]methanone;
4-[[2-chloro-3-[3-(3,5-difluorophenyl)-2,7-dimethyl-5,7-dihydro-4H-pyrazolo[3,4-c]pyridine-6-carbonyl]-5-fluoro-phenyl]methylamino]pyrrolidin-2-one;hydrochloride;
5-[[2,5-dichloro-3-[(7S)-3-(3,5-difluorophenyl)-2,7-dimethyl-5,7-dihydro-4H-pyrazolo[3,4-c]pyridine-6-carbonyl]phenoxy]methyl]oxazolidin-2-one;
(5S)-5-[[2,5-dichloro-3-[(7S)-3-(3,5-difluorophenyl)-2,7-dimethyl-5,7-dihydro-4H-pyrazolo[3,4-c]pyridine-6-carbonyl]phenoxy]methyl]oxazolidin-2-one;
(5R)-5-[[2,5-dichloro-3-[(7S)-3-(3,5-difluorophenyl)-2,7-dimethyl-5,7-dihydro-4H-pyrazolo[3,4-c]pyridine-6-carbonyl]phenoxy]methyl]oxazolidin-2-one;
4-[[2-chloro-3-[(7S)-3-(3,5-difluorophenyl)-2,7-dimethyl-5,7-dihydro-4H-pyrazolo[3,4-c]pyridine-6-carbonyl]phenoxy]methyl]pyrrolidin-2-one;
[2-chloro-3-(1,6-diazaspiro[3.3]heptan-6-yl)-5-fluoro-phenyl]-[(7S)-3-(3,5-difluorophenyl)-2,7-dimethyl-5,7-dihydro-4H-pyrazolo[3,4-c]pyridin-6-yl]methanone;2,2,2-trifluoroacetic acid;
[(7S)-3-(3,5-difluorophenyl)-2,7-dimethyl-5,7-dihydro-4H-pyrazolo[3,4-c]pyridin-6-yl]-[1-(2-hydroxy-4-pyridyl)-1,2,4-triazol-3-yl]methanone;
(4S)-4-[[2-chloro-3-[(7S)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one;
(4R)-4-[[2-chloro-3-[(7S)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one;
(4S)-4-[[2-chloro-3-[(7R)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one;
(2-chloro-3-methoxy-phenyl)-[(8S)-3-(3-chlorophenyl)-8-methyl-6,8-dihydro-5H-[1,2,4]triazolo[4,3-a]pyrazin-7-yl]methanone; and
(2-chloro-3-methoxy-phenyl)-[(8S)-3-(3-chlorophenyl)-8-methyl-6,8-dihydro-5H-imidazo[1,2-a]pyrazin-7-yl]methanone.

26. The compound of formula (II) according to claim 19, or a pharmaceutically acceptable salt thereof, selected from:
4-[[2-Chloro-3-[3-(3,5-difluorophenyl)-2,7-dimethyl-5,7-dihydro-4H-pyrazolo[3,4-c]pyridine-6-carbonyl]-5-fluoro-phenoxy]methyl]pyrrolidin-2-one;
5-[2-Chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-1H-pyridin-2-one;
5-[[2-Chloro-3-[3-(3,5-difluorophenyl)-2,7-dimethyl-5,7-dihydro-4H-pyrazolo[3,4-c]pyridine-6-carbonyl]-5-fluoro-phenoxy]methyl]oxazolidin-2-one;
4-[2-Chloro-3-[(7S)-3-(3,5-difluorophenyl)-2,7-dimethyl-5,7-dihydro-4H-pyrazolo[3,4-c]pyridine-6-carbonyl]-5-fluoro-phenyl]-1H-pyridin-2-one;
[1-(5-Chloro-2-hydroxy-3-pyridyl)-1,2,4-triazol-3-yl]-[(7S)-3-(3,5-difluorophenyl)-2,7-dimethyl-5,7-dihydro-4H-pyrazolo[3,4-c]pyridin-6-yl]methanone;
[(7S)-3-(3,5-Difluorophenyl)-2,7-dimethyl-5,7-dihydro-4H-pyrazolo[3,4-c]pyridin-6-yl]-[1-(2-hydroxy-3-pyridyl)-1,2,4-triazol-3-yl]methanone;
N-[1-[3-chloro-5-[(7S)-6-[2-chloro-3-(5-cyano-1H-pyrrol-3-yl)-5-fluoro-benzoyl]-2,7-dimethyl-5,7-dihydro-4H-pyrazolo[3,4-c]pyridin-3-yl]phenyl]cyclopropyl]methanesulfonamide;
[2-chloro-5-fluoro-3-[2-(1H-pyrazol-4-yl)ethyl]phenyl]-[(7S)-3-(3,5-difluorophenyl)-2,7-dimethyl-5,7-dihydro-4H-pyrazolo[3,4-c]pyridin-6-yl]methanone;
[2-chloro-5-fluoro-3-[2-(1H-triazol-4-yl)ethyl]phenyl]-[(7R)-3-(3,5-difluorophenyl)-2,7-dimethyl-5,7-dihydro-4H-pyrazolo[3,4-c]pyridin-6-yl]methanone;
4-[[2-chloro-3-[3-(3,5-difluorophenyl)-2,7-dimethyl-5,7-dihydro-4H-pyrazolo[3,4-c]pyridine-6-carbonyl]-5-fluoro-phenyl]methylamino]pyrrolidin-2-one;hydrochloride;
(4S)-4-[[2-chloro-3-[(7S)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one; and
(4S)-4-[[2-chloro-3-[(7R)-3-(3,5-difluorophenyl)-2,7-dimethyl-5,7-dihydro-4H-pyrazolo[3,4-c]pyridine-6-carbonyl]-5-fluoro-phenoxy]methyl]imidazolidin-2-one.

27. A process of manufacturing a compound of formula (II) according to claim 1, or a pharmaceutically acceptable salt thereof, comprising:
(a) reacting an amine 1 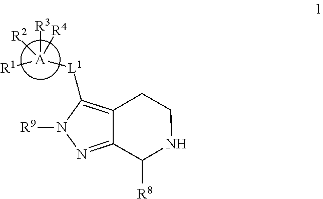
wherein R¹ to R⁴, R¹, R⁹, A, and L¹ are as described in claim 1;
with a carboxylic acid 2 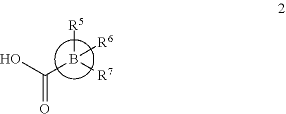
wherein R⁵ to R⁷ and B are as described in claim 1;
in a solvent and in the presence of a base and a coupling agent to form said compound of formula (II); and optionally
(b) contacting said compound of formula (II) with an acid to form a pharmaceutically acceptable salt thereof.

28. A compound of formula (II) according to any one of claims 1 to 26, when manufactured according to the process of claim 27.

29. A compound of formula (II) according to any one of claims 1 to 26, or a pharmaceutically acceptable salt thereof, for use as therapeutically active substance.

30. A pharmaceutical composition comprising a compound of formula (II) according to any one of claims 1 to 26, or a pharmaceutically acceptable salt thereof, and a therapeutically inert carrier.

31. A method for the treatment or prophylaxis of neuroinflammation, neurodegenerative diseases, pain, cancer, mental disorders, inflammatory bowel disease, irritable bowel syndrome and/or diarrhea in a mammal, comprising administering a therapeutically effective amount of a compound of formula (II) according to any one of claims 1 to 26, or of a pharmaceutically acceptable salt thereof, or of a pharmaceutical composition according to claim 30 to said mammal.

32. A compound of formula (II) according to any one of claims 1 to 26, or a pharmaceutically acceptable salt thereof, or a pharmaceutical composition according to claim 30 for use in a method according to claim 31.

33. Use of a compound of formula (II) according to any one of claims 1 to 26, or of a pharmaceutically acceptable salt thereof, or of a pharmaceutical composition according to claim 30 in a method according to claim 31.

34. Use of a compound of formula (II) according to any one of claims 1 to 26, or of a pharmaceutically acceptable salt thereof, for the manufacture of a medicament for the treatment or prophylaxis of neuroinflammation, neurodegenerative diseases, pain, cancer, mental disorders, inflammatory bowel disease, irritable bowel syndrome and/or diarrhea in a mammal.

35. The invention as described hereinbefore.
